(19) United States
(12) Patent Application Publication
(10) Pub. No.: US 2023/0323463 A1
(43) Pub. Date: Oct. 12, 2023

(54) COMPOSITIONS AND METHODS FOR DETECTING BCL2L14 AND ETV6 GENE FUSIONS FOR DETERMINING INCREASED DRUG RESISTANCE

(71) Applicant: UNIVERSITY OF PITTSBURGH-OF THE COMMONWEALTH SYSTEM OF HIGHER EDUCATION, Pittsburgh, PA (US)

(72) Inventor: Xiaosong WANG, Sewickley, PA (US)

(21) Appl. No.: 17/907,774

(22) PCT Filed: Feb. 26, 2021
(86) PCT No.: PCT/US2021/019846

§ 371 Date: Aug. 29, 2022

Related U.S. Application Data
(60) Provisional application No. 62/982,985, filed on Feb. 28, 2020.

(51) Int. Cl. C12Q 1/6886; C07K 16/18

(52) U.S. Cl. CPC C12Q 1/6886; C07K 16/18; C12Q 2600/106; C12Q 2600/156

(57) ABSTRACT

Disclosed herein are compositions and methods for detecting BCL2L14/ETV6 gene fusions relating to cancer. Also disclosed herein are compositions and methods for diagnosing and treating cancers that include detecting a BCL2L14/ETV6 gene fusion.

### CROSS-REFERENCE TO RELATED APPLICATIONS

This application claims the benefit of U.S. Provisional Application No. 62/982,985, filed Feb. 28, 2020, which is expressly incorporated herein by reference.

### STATEMENT REGARDING FEDERALLY SPONSORED RESEARCH OR DEVELOPMENT

This invention was made with government support under grant numbers CA181368 and CA183976 awarded by the National Institutes of Health. The government has certain rights in the invention.

### FIELD

The present disclosure relates to cancer treatment and diagnosis.

### BACKGROUND

Triple-negative breast cancer (TNBC) accounts for 10-20% of breast cancer, with chemotherapy as its mainstay of treatment due to lack of well-defined targets. Recurrent gene fusions comprise a class of viable genetic targets in solid tumors, however, their role in breast cancer remains underappreciated due to the complexity of genomic rearrangements in this cancer. Identification of cancer-specific genetic events that can guide the treatments represents an unmet clinical need. Therefore, what is needed are compositions and methods for determining the gene rearrangement specific for breast cancer patients. The compositions and methods disclosed herein address these and other needs.

### SUMMARY

Provided herein are methods of diagnosing a subject with increased taxane resistance (such as increased resistance to paclitaxel and/or docetaxel), comprising: obtaining a biological sample from the subject; and detecting a BCL2L14/ETV6 gene fusion in the sample, wherein the detection indicates the subject has increased taxane resistance (such as increased resistance to paclitaxel and/or docetaxel) and the subject is diagnosed with increased taxane resistance (such as increased resistance to paclitaxel and/or docetaxel). In some embodiments, the BCL2L14/ETV6 gene fusion is selected from the group consisting of a E2-E3 fusion, a E2-E6 fusion, a E4-E2 fusion, a E4-E3 fusion, and an E5-E5 fusion. In some aspects, the E2-E3 fusion comprises SEQ ID NO: 23, the E2-E6 fusion comprises SEQ ID NO: 20, the E4-E2 fusion comprises SEQ ID NO:22, the E4-E3 fusion comprises SEQ ID NO:24, and the E5-E5 fusion comprises SEQ ID NO:21.

The method of detection can comprise contacting the biological sample with a reaction mixture comprising a probe specific for one of SEQ ID NO: 23, SEQ ID NO:20, SEQ ID NO: 24 and SEQ ID NO:21. The method of detection can alternatively or further comprise contacting the biological sample with a reaction mixture comprising two primers, wherein the first primer is complementary to a BCL2L14 polynucleotide sequence and the second primer is complementary to a ETV6 polynucleotide sequence, wherein the BCL2L14/ETV6 gene fusion is detectable by the presence of an amplicon generated by the first primer and the second primer. The method of detection can also comprise contacting the biological sample with a reaction mixture comprising two primers, wherein the first primer is complementary to a BCL2L14 polynucleotide sequence and the second primer is complementary to a ETV6 polynucleotide sequence, wherein hybridization of the two primers on a BCL2L14/ETV6 gene fusion sequence provides a detectable signal, and the BCL2L14/ETV6 gene fusion is detectable by the presence of the signal. In some embodiments, a first of the one or more primers is selected from the group consisting of SEQ ID NO: 1, SEQ ID NO: 3, SEQ ID NO: 7, SEQ ID NO: 9, SEQ ID NO: 11, SEQ ID NO: 17, and SEQ ID NO: 19 and a second of the one or more primers is selected from the group consisting of SEQ ID NO: 2, SEQ ID NO: 4, SEQ ID NO: 8, SEQ ID NO: 10, SEQ ID NO: 12, and SEQ ID NO: 18. In some embodiments, the primers are SEQ ID NO:3 and SEQ ID NO:4. In some embodiments, the primers are SEQ ID NO: 11 and SEQ ID NO:12. In some embodiments, the primers are SEQ ID NO:17 and SEQ ID NO:18. In some embodiments, the primers are SEQ ID NO: 19 and SEQ ID NO: 18.

The methods described herein can be used to detect a BCL2L14/ETV6 gene fusion in a subject that has a cancer, such as a breast cancer and including a triple negative breast cancer. The methods can further comprise administering to the subject one or more of capecitabine, doxorubicin, cyclophosphamide, fluorouracil, epirubicin, cisplatin, carboplatin, olaparib, and talazoparib. The methods can still further comprise administering to the subject a PD-L1 inhibitor or other immune checkpoint inhibitor.

Also included herein are methods of treating a cancer in a subject comprising: detecting a BCL2L14/ETV6 gene fusion in a sample obtained from the subject; and administering to the subject a therapeutically effective amount of one or more of an immune checkpoint inhibitor (e.g., a PD-L1 inhibitor), capecitabine, doxorubicin, cyclophosphamide, fluorouracil, epirubicin, cisplatin, carboplatin, olaparib, and talazoparib. The BCL2L14/ETV6 gene fusion can be selected from the group consisting of a E2-E3 fusion, a E2-E6 fusion, a E4-E2 fusion, a E4-E3 fusion, and an E5-E5 fusion or other fusion variations. The E2-E3 fusion can comprise SEQ ID NO: 23, the E2-E6 fusion can comprise SEQ ID NO: 20, the E4-E2 fusion can comprise SEQ ID NO:22, the E4-E3 fusion can comprise SEQ ID NO:24, and the E5-E5 fusion can comprise SEQ ID NO:21.

Further included are methods for detecting a BCL2L14/ETV6 gene fusion comprising: obtaining a biological sample from a subject; and detecting the fusion in the sample. In some embodiments, the detection can comprise contacting the biological sample with a reaction mixture comprising a probe specific for one of SEQ ID NO: 23, SEQ ID NO:20, SEQ ID NO: 24 and SEQ ID NO:21. A detectable moiety can be covalently bonded to the probe. Kits comprising one or more probes are included, wherein each probe specifically hybridizes to a fusion point nucleotide sequence selected from SEQ ID NO: 23, SEQ ID NO:20, SEQ ID NO: 24 and SEQ ID NO:21.

### DESCRIPTION OF DRAWINGS

FIGS. 1(A-F). Landscape of recurrent adjacent gene rearrangements in breast cancer revealed by whole genome sequencing data. (A) Frequency chart of experimentally validated inter-chromosome, intra-chromosome distant, and intra-chromosome adjacent translocations in 9 breast cancer cell lines and 15 breast tumors revealed by WGS data (Stephens PJ, et al. (2009)). Among 9,408 confirmed somatic translocations, about half are intra-chromosomal translocations within 500 kb distance. (B) CIRCOS plot showing the landscape of 99 recurrent gene rearrangements detected in 215 breast tumors based on WGS data from ICGC. The histogram inside the circus plot represents the recurrence of the gene rearrangements in the chromosome position, indicating the number of patients that harbor the gene fusions. (C) Genomic hotspots of colinear and non-colinear AGR or intra-chromosomal gene rearrangements. Adjacent and intra-chromosomal rearrangements in the genomes are displayed in rainfall plot with each dot represent a respective positive sample. X-axis shows 24 chromosomes in the human genome, and y-axis shows the distance between the rearrangement points (base pairs at log10 scale). The horizontal line indicates the cutoff for adjacent gene rearrangements (500 kb in distance). (D) Scatter plot showing the incidences of 99 recurrent gene rearrangements and their concept signature scores, which were detected from 215 ICGC breast tumors profiled by WGS. The x-axis indicates the incidence of gene rearrangements in the cohort. The y-axis indicates the max ConSig scores of 5′ or 3′ partner genes. (E) Tile plot showing the top recurrent AGRs and the known breast cancer oncogenes including ER, PR, HER2, and PI3KCA mutations in TCGA 92 breast tumors. The AGRs detected in at least two TCGA tumors and >1% of all ICGC tumors are shown in the figure. Group-wise mutual exclusivity test of using a discrete independence statistics called “Discover”, that takes account of the distribution of all somatic gene rearrangements, shows that there are significant number of tumors that harbor only one of these AGRs (p<0.001). (F) Bar graph showing the association between BCL2L14-ETV6 fusion and different clinicopathological features of 608 breast tumors in the TCGA (92 tumors) and COSMIC (516 tumors) cohort. Y-axis shows the incidence of BCL2L14-ETV6 fusion in different clinicopathological groups. *** P<0.001. Significance was determined using Fisher’s exact test (two tailed).

FIGS. 2(A-C). Characterization of the BCL2L14-ETV6 fusions in 134 triple-negative breast tumors from two different patient cohorts. (A) RT-PCR analyses of BCL2L14-ETV6 fusion and wild-type ETV6 in triple negative tumors from the Pitt cohort (n=89). A 5-donor normal breast pool (NB) was used as a negative control. Representative gel images are shown. Fusion-positive cases are marked with red asterisks. The chromatograms in the lower panel show the junction sequences of BCL2L14-ETV6 fusion variants detected in Pitt-TN49, Pitt- TN134, Pitt-TN138 and Pitt-TN144 tumors. (B) RT-PCR analyses of BCL2L14-ETV6 fusion, wild-type BCL2L14, wild-type ETV6, and GAPDH in 45 triple negative breast tumors from BCM cohort. A 5-donor normal breast pool (NB) was used as a negative control. Fusion-positive cases are labeled with red asterisks. Chromatograms in the lower panel show the junction sequences of BCL2L14-ETV6 fusion variants detected in BCM-TN13 and BCM-TN35 tumors. (C) Genomic PCR analysis of the BCL2L14-ETV6-positive TNBC tumor samples from BCM cohort (BCM-TN13 and BCM-TN35) identified the precise genomic fusion points. Left panel shows the schematic of the genomic breakpoints identified in BCM-TN13 and BCM-TN35 tumors. Right panel shows the gel images and chromatograms of BCL2L14-ETV6 genomic PCR products. Genomic DNA from MCF10A cells was used as a negative control.

FIGS. 3(A-E). Characterization of the protein products encoded by BCL2L14-ETV6 fusion variants. (A) Schematic of BCL2L14-ETV6 fusion variants and encoded proteins identified in the positive cases of the BCM and Pitt cohorts (BCM-TN13, BCN-TN35, Pitt-TN49, Pitt-TN134, Pitt-TN138, Pitt-TN144 and BCM-2147). Open-reading frames (ORFs) of BCL2L14 and ETV6 are depicted in dark shades. Amino acid numbers of BCL2L14 and ETV6 are derived from reference sequence NP_620048 and NP_001978, respectively. Functional protein domains are annotated on top of each gene. (B-C) Western blots detecting BCL2L14-ETV6 fusions (E2E3, E4E3 and E4E2), wild-type ETV6 (ectopic or endogenous) and endogenous BCL2L14 in the engineered BT20 triple-negative breast cancer cells (B) and engineered MCF10A benign mammary epithelial cells (C). Oblique arrow denotes the band for E4E2 or E2E3 fusion protein. The fusion variants E4E2 and E2E3 were detected by both polyclonal antibodies of BCL2L14 and ETV6 (Sigma), while the E4E3 variant which does not have ETV6-encoded sequence was detected only by the BCL2L14 polyclonal antibody (Sigma). The E4E3 fusion variant encodes a much smaller protein (27 kD) than the E4E2 (74 kD) and E2E3 (62 kD) proteins, which is hard to detect on the same blot, and is thus detected separately. * Here the wild-type BCL2L14 protein was detected by the BCL2L14 monoclonal antibody (Abcam) which identifies a unique band. (D) Western blot using anti-ETV6 polyclonal antibody (Sigma) detected the endogenous protein (pointed by the arrow) encoded by BCL2L14-ETV6 E4E2 variant in the BCM-2147 triple-negative PDX sample. (E) Subcellular localization of wild-type ETV6, BCL2L14 and BCL2L14-ETV6 fusion proteins, in engineered BT20 and MCF10A cells. Oblique arrow points out the fusion protein (E4E2, E2E3). The nuclear protein ORC2 and cytoplasmic protein GAPDH are used as positive controls for fractionation. C, cytoplasm; N, nucleus. * Here the wild-type BCL2L14 protein was detected by the BCL2L14 monoclonal antibody (Abcam) which identifies a unique band.

FIGS. 4(A-D). Ectopic expression of BCL2L14-ETV6 endows increased cell migration, invasion, and paclitaxel resistance. (A-B) Ectopic expression of BCL2L14-ETV6 fusion variants in BT20 TNBC cells (A) and MCF10A benign mammary epithelial cells (B) significantly enhanced cell migration as revealed by Boyden chamber assay (left), and increased cell invasion as revealed by transwell Matrigel assay (right), relative to the vector control. Results were summarized from experimental triplicates. (C) BCL2L14-ETV6 fusions endows clonal resistance in BT20 cells following prolonged paclitaxel treatment for one month as shown by clonogenic assay. Here a low dosage of 5 nM paclitaxel is used for treatment to observe long-term treatment effect. (D) BCL2L14-ETV6 fusions endows clonal resistance in MCF10A cells following prolonged paclitaxel treatment for one month as shown by clonogenic assay. Here 15 nM paclitaxel is used for treatment since MCF10A is less sensitive to paclitaxel. The quantitative results in the upper panels of C-D are based on two replicates of each condition. The vehicle-treated cells were harvested in 14 days for BT20 model, and 7 days for MCF10A model, while the PTX-treated cells were harvested in one month due to their different growth rates. The comparing cell models (i.e. vector, wtETV6, fusion variants) were harvested at the same time point. Vehicle: 0.1% DMSO; PTX: Paclitaxel. *P<0.05, **P<0.01. ***P<0.001, significance was determined using Student’s t-test (two-tailed) and error bars reflect mean ± standard deviation.

FIGS. 5(A-F). BCL2L14-ETV6 fusions induce coherent gene expression changes distinctive from wtETV6, and prime partial epithelial-mesenchymal transition. (A) Unsupervised principal component analysis (PCA) separated the BT20 cells expressing BCL2L14-ETV6 variants and the BT20 cells expressing the vector or wtETV6 into distinct clusters. We used the first three principal components to present the samples in the 3-dimentional PCA plot. (B) Hierarchical clustering showing the global gene expression differences between the engineered BT20 cells expressing vector, wtETV6, or BCL2L14-ETV6 fusion variants. (C) Gene expression heatmap of the 73 core enrichment genes of EMT signature in BCL2L14-ETV6 fusion variant expressing BT20 cells compared to vector- and wtETV6-expressing BT20 cells. The genes are sorted by their ranks from GSEA analysis. (D-F) Western blots detecting the EMT markers including E-Cadherin, N-Cadherin, Vimentin, and EMT transcription factors including SNAI1 and SNAI2 in the engineered stable cell lines of (D) MCF10A cells, (E) BT20 cells and (F) TGFβ-1 and EGF-treated BT20 cells. Engineered BT20 cells were treated with 10 ng/ml of TGFβ-1 and 20 ng/ml of EGF for 72 h before being harvested. GAPDH was used as the loading control. * indicates non-specific band.

FIG. 6. Clinicopathological associations of the total number of intergenic rearrangements. Boxplot showing the total number of intergenic rearrangements in the different clinicopathological subtypes of breast tumors. A total of 92 TCGA breast tumors included in the ICGC dataset have available clinical and histopathological data obtained from Heng et al. (PMID: 27861902). *P<0.05, **P<0.01, ***P<0.001 (unpaired Wilcoxon Rank Sum Test).

FIG. 7. Correlation of the top recurrent AGRs with genomic instability index and DNA Damage Repair (DDR) scores. The top AGRs detected in at least two TCGA tumors and >1% of all ICGC tumors are shown in the figure. The weighted genome integrity index (wGII) and DDR deficiency scores are from Marquard et al. (PMID: 26015868). BRCA1 mutation are based on Yost et al. (PMID: 31360904). NtAI, telomeric allelic imbalance; LST, large scale transition; LOH, loss of heterozygosity; Nmut, total number of mutations per sample; FLOH, frequency of LOH.

FIG. 8. The landscape of recurrent fusion partner genes in breast cancer. The incidence (%) of fusion partner genes in TCGA clinicopathological tumor entities are shown in the figure. Only the cases that harbor nonprivate fusions are counted. The partner genes with total frequency count > 4 (1.86 %) were displayed in the figure.

FIG. 9. Clinicopathological associations with fusion frequency in the four most frequent AGRs. The frequency of the top four AGRs were calculated in each clinical data type of the 92 TCGA breast tumors. The clinical and histopathological data were obtained from Heng et al. (PMID: 27861902).

FIG. 10. ETV6 expression in BCL2L14-ETV6 negative or positive TNBC tumors in TCGA and COSMIC cohorts. *P<0.05 (unpaired Wilcoxon Rank Sum Test).

FIGS. 11(A-B). Detecting TTC6-MIPOLI by RT-PCR in breast cancer cell lines and tumors. (A) RT-PCR analyses of TTC6-MIPOL1 fusion in a panel of 141 ER+ breast tumors from the University of Pittsburgh cohort, with GAPDH as the loading control. Chromatogram in the lower panel shows the junction sequence of TTC6-MIPOL1I fusion variant detected in the ER103 tumor sample. Asterisk denotes ER103. (B) RT-PCR analyses of TTC6-MIPOL1 fusion in a panel of 44 breast cancer cell lines. Chromatograms in the lower panel show the junction sequences of two TTC6-MIPOL1I fusion variants detected in MDA-MB-361. The sequence in FIG. 11A is TGGAAGTGAGTTTACACAAA (SEQ ID NO: 27). The sequences in FIG. 11B are CTAAGAGCAGTTTACACAAA (SEQ ID NO: 28), and CTAAGAGCAGGTTGGAAAGG (SEQ ID NO: 29)

FIGS. 12(A-B) AKAP8-BRD4 expression in patient-derived xenografts and breast cancer cell lines. (A) RT-PCR analyses of AKAP8-BRD4 fusion in a panel of patient-derived xenografts with GAPDH as the control. Chromatogram in the lower panel shows the junction sequence of AKAP8-BRD4 fusion variant detected in the BCM-2147 PDX sample. Asterisk denote BCM-2147. (B) RT-PCR analyses of AKAP8-BRD4 fusion in a panel of breast cancer cell lines. The sequence in FIG. 12A is AGACACCCAGAGTGCCTGGT (SEQ ID NO: 30).

FIGS. 13(A-B). (A) RT-PCR analyses of BCL2L14-ETV6 fusion, wild-type (WT) BCL2L14 and ETV6, and GAPDH in 34 triple-negative PDX breast tumors. The BCL2L14-ETV6- positive PDX is marked in asterisks (BCM-2147). Chromatogram on the right shows the junction sequence of the fusion transcript detected in BCM-2147. For wtETV6, blue asterisks denote cases with ETV6 exon duplications, BCM-3611, BCM-3807 and BCM-5998, from left to right, respectively. (B) RT-PCR Screening of BCL2L14-ETV6 fusion in a panel of 44 breast cancer cell lines. No cell line was identified with the fusion existence. Asterisk denotes the cell line with ETV6 exon duplication. The sequences in FIG. 13A is GTTGGAAAGAAAGCAGGAACGAATTT (SEQ ID NO: 22) (E4-E2 fusion point).

FIG. 14. RT-PCR analyses of BCL2L14-ETV6 fusion, wide-type BCL2L14, ETV6, and GAPDH in 200 ER-positive breast tumors from the BCM patient cohort.

FIG. 15. Histopathology of BCL2L14-ETV6 fusion-positive cases from Pitt cohort. Hematoxylin and eosin (H&E) images showing extensive necrosis in two fusion positive case, Pitt-TN49, Pitt-TN134, and focal necrosis in Pitt-TN138 and Pitt-TN144. Regions in the red boxes indicate necrosis areas. All tumors show high nuclear grade.

FIGS. 16(A-B). Copy number data at the ETV6/BCL2L14 (A) and TTC6-MIPOL1 (B) loci in the fusion positive TCGA cases, and in the TCGA cases that harbor duplications delineating the fusion partner genes. Log2 transformed copy number data for breast tumors and paired normal blood samples are from TCGA. The fusion positive cases detected by WGS data are positioned above the dash line.

FIGS. 17(A-B).The effect of ectopic expression of BCL2L14-ETV6 fusion variants in BT20 on cell viability and cell cycle progression. (A-B) Ectopic expression of BCL2L14-ETV6 fusion variants in BT20 did not result in significant changes in cell viability (A) or cell cycles (B).

FIGS. 18(A-B). The effect of paclitaxel treatment on the viability and apoptosis of the engineered BT20 cells. (A) BT20 cells overexpressing BCL2L14-ETV6 but not vector or wtETV6 showed increased resistance to paclitaxel in short-term (72 h) treatment. (B) Apoptotic biomarkers were detected by immunoblotting in the engineered BT20 cells following vehicle (DMSO) or paclitaxel treatment for 48 hours. Veh, vehicle. PTX, paclitaxel.

FIGS. 19(A-B). The characteristic of pathway signatures in BCL2L14-ETV6 expressing BT20 cells. (A) Top enriched pathways characteristic of BCL2L14-ETV6 expressing BT20 cells revealed by GSEA. The FDR q-values (-log10) comparing the engineered BT20 cells expressing BCL2L14-ETV6 fusion variants or wtETV6 with the vector control are shown. The 10 pathways shown in the chart have significant FDR q-value < 0.2 (>0.69 in -log 10 number) in the comparison between BCL2L14-ETV6 fusion variant vs. vector expressing BT20 cells, but not in the comparison between wtETV6 vs. vector expressing BT20 cells. (B) The enrichment plot of the Epithelial Mesenchymal Transition (EMT) pathway characteristic of the BT20 cells expressing BCL2L14-ETV6 fusion variants, compared with the vector control. The EMT gene signature is from Hallmark gene sets.

FIG. 20. Heatmap of the expression pattern of the top master regulators. 13 transcription factors were predicted by MRA as master regulators of which expression levels were altered by BCL2L14-ETV6 gene fusion in BT20 cells. SNAI2 was identified as one of the top master regulators that regulate EMT gene signatures in BCL2L14-ETV6 fusion variant expressing breast cancer cells. The heatmap shows the different gene expression levels between vector, wtETV6 and BCL2L14-ETV6 fusion variant expressing BT20 cells. The bar graph shows the distribution of positively (red) or negatively (blue) correlated target genes in the individual master regulators (MR) (Spearman’s correlation between the expression levels of the MR and its targets). The black bars within the heatmap indicate EMT genes. The mode explains whether BCL2L14-ETV6 fusion variants positively (+) or negatively (-) affected the expression of the individual MRs.

FIGS. 21(A-C). Expression of breast cancer stem cell markers CD44 and ALDH1A3 in BCL2L14-ETV6 fusion-expressing BT20 cells. (A) Box plots showing the expression level of CD44 and ALDH1A3 transcripts by RNA-seq analysis in vector-, wtETV6-, or BCL2L14-ETV6 fusion variant-expressing BT20 cells. CD44 and ALDH1A3 were over-expressed in BCL2L14-ETV6 fusion-expressing BT20 cells compared to the vector or wtETV6 controls. (B) Representative density plot for detection of CD44 surface marker and ALDH activity by flow cytometry to reveal breast cancer stem cell populations in the engineered BT20 cells. CD44-high and ALDH-high cells are gated as trapeziums and indicated in percentages. (C) Percentages of cells expressing CD44 (CD44+) and cells with high ALDH activity (ALDH^{high}) cells in wtETV6-and fusion-expressing BT20 cells, relative to vector control.

FIG. 22. RNAseq data revealed that among genes MMP3, PF4, EGR1, TRAF1 (57), BBC3, CDKN1A, IGFBP5, MAD2L1,TWIST1, CLIC5, ANGPTL2, BIRC7, and WBP1L that CDKN1A and IGFBP5 are repressed by BCL2L14-ETV6 but activated by wtETV6.

### DETAILED DESCRIPTION

Recurrent gene fusions comprise a class of viable genetic targets in solid tumors, however, their role in breast cancer remains underappreciated due to the complexity of genomic rearrangements in this cancer. Disclosed herein are a set of gene rearrangements preferentially found in the more aggressive forms for breast cancers that lack well-defined genetic targets. Notably, these fusion positive tumors exhibit more aggressive histopathological features such as gross necrosis and high tumor grade. This shows BCL2L14-ETV6 as a recurrent gene fusion in TNBC (e.g., a more aggressive form of TNBC).

Accordingly, disclosed herein is a method for detecting BCL2L14/ETV6 gene fusion. The fusion can be detected by contacting the sample with one or more primers specific for the fusion, performing an amplification reaction, and detecting an amplification product or amplicon. In some examples, the detection of the fusion indicates an increased resistance to paclitaxel in the subject.

Also disclosed herein is a method of diagnosing or treating a subject with increased taxane resistance, such as increased resistance to paclitaxel and/or docetaxel. The subject with increased taxane resistance is detected of having a BCL2L14/ETV6 gene fusion. In some embodiments, the subject is administered with a therapeutically effective amount of one or more of an immune checkpoint (i.e., PD-L1) inhibitor, capecitabine, doxorubicin, cyclophosphamide, fluorouracil, epirubicin, cisplatin, carboplatin, olaparib, and talazoparib.

Terms used throughout this application are to be construed with ordinary and typical meaning to those of ordinary skill in the art. However, Applicants desire that the following terms be given the particular definition as provided below.

### TERMINOLOGY

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Amplifying,” “amplification,” and grammatical equivalents thereof refers to any method by which at least a part of a target nucleic acid sequence is reproduced in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Qreplicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA)(TwistDx, Cambridg, UK), and self-sustained sequence replication (3SR), including multiplex versions or combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other places, Sambrook et al. Molecular Cloning, 3rd Edition; Ausbel et al.; PCR Primer: A Laboratory Manual, Diffenbach, Ed., Cold Spring Harbor Press (1995); The Electronic Protocol Book, Chang Bioscience (2002), Msuih et al., J. Clin. Micro. 34:501-07 (1996); The Nucleic Acid Protocols Handbook, R. Rapley, ed., Humana Press, Totowa, N.J. (2002).

“Administration” of “administering” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “control” is an alternative subject or sample used in an experiment for comparison purposes. A control can be “positive” or “negative.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Accordingly, it should be understood that “encode” or “encoding”.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence (i.e., exon), non-coding sequence (e.g., intron), or control sequence, fragments thereof, and combinations thereof. The term “gene” or “gene sequence” includes, for example, control sequences upstream of the coding sequence (for example, the ribosome binding site).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

As used herein, the term “immune checkpoint inhibitor” or “checkpoint inhibitor” refers to a molecule that completely or partially reduces, inhibits, interferes with or modulates one or more checkpoint proteins. Checkpoint proteins include, but are not limited to, PD-1, PD-L1 and CTLA-4.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” or “antagonist” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Control samples (untreated with inhibitors) are assigned a relative activity value of 100%. Inhibition of a described target protein is achieved when the activity value relative to the control is about 80%, optionally 50% or 25, 10%, 5%, or 1% or less.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

Unless otherwise specified, a “nucleotide sequence encoding an amino acid sequence” includes all nucleotide sequences that are degenerate versions of each other and that encode the same amino acid sequence. The nucleotide sequence that encodes a protein or an RNA may also include introns to the extent that the nucleotide sequence encoding the protein may in some version contain an intron(s).

The term “PD-L1 inhibitor” refers to refers to a composition that binds to PD-1 and reduces or inhibits the interaction between the bound PD-L1 and PD-1. In some embodiments, the PD-L1 inhibitor is a monoclonal antibody that is specific for PD-L1 and that reduces or inhibits the interaction between the bound PD-L1 and PD-1. Non-limiting examples of PD-L1 inhibitors are atezolizumab, avelumab and durvalumab. In some embodiments, the atezolizumab is TECENTRIQ or a bioequivalent. In some embodiments, the atezolizumab has the Unique Ingredient Identifier (UNII) of the U.S. Food and Drug Administration of 52CMI0WC3Y. In some embodiments, the atezolizumab is that described in U.S. Pat. No. 8217149, which is incorporated by reference in its entirety. In some embodiments, the avelumab is BAVENCIO or a bioequivalent. In some embodiments, the avelumab has the Unique Ingredient Identifier (UNII) of the U.S. Food and Drug Administration of KXG2PJ551I. In some embodiments, the avelumab is that described in U.S. Pat. App. Pub. No. 2014321917, which is incorporated by reference in its entirety. In some embodiments, the durvalumab is IMFINZI or a bioequivalent. In some embodiments, the durvalumab has the Unique Ingredient Identifier (UNII) of the U.S. Food and Drug Administration of 28X28X9OKV. In some embodiments, the durvalumab is that described in U.S. Pat. No. 8779108, which is incorporated by reference in its entirety.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington’s Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “polynucleotide” refers to a single or double stranded polymer composed of nucleotide monomers. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The term “primer” or “amplification primer” refers to an oligonucleotide that is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature. The most widely used target amplification procedure is PCR, first described for the amplification of DNA by Muliis et al. in U.S. Pat. No. 4,683,195 and Mullis in U.S. Pat. No. 4,683,202 and is well known to those of ordinary skill in the art.

A “primer” or “primer sequence” hybridizes to a target nucleic acid sequence (for example, a DNA template to be amplified) to prime a nucleic acid synthesis reaction. The primer may be a DNA oligonucleotide, a RNA oligonucleotide, or a chimeric sequence. The primer may contain natural, synthetic, or modified nucleotides. Both the upper and lower limits of the length of the primer are empirically determined. The lower limit on primer length is the minimum length that is required to form a stable duplex upon hybridization with the target nucleic acid under nucleic acid amplification reaction conditions. Very short primers (usually less than 3-4 nucleotides long) do not form thermodynamically stable duplexes with target nucleic acids under such hybridization conditions. The upper limit is often determined by the possibility of having a duplex formation in a region other than the pre-determined nucleic acid sequence in the target nucleic acid. Generally, suitable primer lengths are in the range of about 10 to about 40 nucleotides long. In certain embodiments, for example, a primer can be 10-40, 15-30, or 10-20 nucleotides long. A primer is capable of acting as a point of initiation of synthesis on a polynucleotide sequence when placed under appropriate conditions. The primer will be completely or substantially complementary to a region of the target polynucleotide sequence to be copied. Therefore, under conditions conducive to hybridization, the primer will anneal to the complementary region of the target sequence. Upon addition of suitable reactants, including, but not limited to, a polymerase, nucleotide triphosphates, etc., the primer is extended by the polymerizing agent to form a copy of the target sequence. The primer may be single-stranded or alternatively may be partially double-stranded.

The term “primer pair” as used herein means a pair of oligonucleotide primers that are complementary to the sequences flanking a target sequence. The primer pair consists of a forward primer and a reverse primer. The forward primer has a nucleic acid sequence that is complementary to a sequence upstream, i.e., 5′ of the target sequence. The reverse primer has a nucleic acid sequence that is complementary to a sequence downstream, i.e., 3′ of the target sequence.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, “decrease”, or “decreased” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

“Reporter probe” refers to a molecule used in an amplification reaction, typically for quantitative or real-time PCR analysis, as well as end-point analysis. Such reporter probes can be used to monitor the amplification of the target nucleic acid sequence. In some embodiments, reporter probes present in an amplification reaction are suitable for monitoring the amount of amplicon(s) produced as a function of time. Such reporter probes include, but are not limited to, the 5′-exonuclease assay (e.g., U.S. Pat. No. 5,538,848) various stem-loop molecular beacons (see for example, U.S. Pat. Nos. 6,103,476 and 5,925,517), stemless or linear beacons (see, e.g., WO 99/21881), PNA MOLECULAR BEACONS (see, e.g., U.S. Pat. Nos. 6,355,421 and 6,593,091), linear PNA beacons, non-FRET probes (see, for example, U.S. Pat. No. 6,150,097), SUNRISE/AMPLIFLUOR probes (U.S. Pat. No. 6,548,250), stem-loop and duplex Scorpion probes (U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB ECLIPSE probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901. Reporter probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The terms “treat,” “treating,” “treatment,” and grammatical variations thereof as used herein, include partially or completely alleviating, mitigating or reducing the intensity of one or more attendant symptoms of a disorder or condition and/or alleviating or mitigating one or more causes of a disorder or condition. Treatments according to the invention may be applied preventively, prophylactically, pallatively or remedially.

Prophylactic administrations are given to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition refers to an amount that is effective to achieve a desired therapeutic result. In some embodiments, a desired therapeutic result is a reduction of tumor size. In some embodiments, a desired therapeutic result is a reduction of cancer metastasis. In some embodiments, a desired therapeutic result is a reduction of a breast cancer, or a symptom of a breast cancer. In some embodiments, a desired therapeutic result is a reduction of a triple negative breast cancer, or a symptom thereof. In some embodiments, a desired therapeutic result is the prevention of cancer relapse. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as control of tumor growth. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### METHODS OF DETECTING, DIAGNOSING AND TREATING

Disclosed herein are methods of detecting a BCL2L14-ETV6 gene fusion, said methods comprising obtaining a sample from a subject, and detecting whether the fusion is present in the sample. In some embodiments, a BCL2L14- ETV6 gene fusion is detected in a sample derived from a subject having breast cancer and the detection indicates that the breast cancer has decreased sensitivity to taxane (such as paclitaxel and docetaxel). Accordingly, the present invention includes methods of diagnosing a breast cancer in a subject having decreased sensitivity to taxane (such as paclitaxel and docetaxel).

Also disclosed herein is a method of treating a breast cancer in a subject, said method comprising detecting a BCL2L14-ETV6 gene fusion in a breast tissue sample obtained from the subject, and administering to the subject a therapeutically effective amount of one or more of capecitabine, doxorubicin, cyclophosphamide, fluorouracil, epirubicin, cisplatin, carboplatin, olaparib, and talazoparib..

As used herein, “gene fusion” refers to a chimeric genomic DNA resulting from the fusion of at least a portion of a first gene to a portion of a second gene. The point of transition between the sequence from the first gene in the fusion to the sequence from the second gene in the fusion is referred to as the “fusion point.” Transcription of the gene fusion results in a chimeric mRNA.

“BCL2L14” or “BCL2 Like 14” refers herein to a polypeptide that is involved in apoptosis, and in humans, is encoded by the BCL2L14 gene. In some embodiments, the BCL2L14 polypeptide is that identified in one or more publicly available databases as follows: HGNC: 16657, Entrez Gene: 79370, Ensembl: ENSG00000121380, OMIM: 606126, UniProtKB: Q9BZR8. In some embodiments, the BCL2L14 polypeptide comprises the sequence of SEQ ID NO: 31, or a polypeptide sequence having at or greater than about 80%, about 85%, about 90%, about 95%, or about 98% homology with SEQ ID NO: 31, or a polypeptide comprising a portion of SEQ ID NO: 31. The BCL2L14 polypeptide of SEQ ID NO: 31 may represent an immature or pre-processed form of mature BCL2L14, and accordingly, included herein are mature or processed portions of the BCL2L14 polypeptide in SEQ ID NO: 31.

The term “BCL2L14 polynucleotide” refers to a polynucleotide that encodes a BCL2L14 polypeptide, or any fragment thereof. In some embodiments, the BCL2L14 polynucleotide is an BCL2L14 exon 1 polynucleotide having a sequence of nucleotides 12070939-12071137 of SEQ ID NO: 32, or a polynucleotide having at or greater than about 80%, about 85%, about 90%, about 95%, or about 98% homology with nucleotides 12070939-12071137 of SEQ ID NO: 32, or a polynucleotide comprising a portion of nucleotides 12070939-12071137 of SEQ ID NO: 32. In some embodiments, the BCL2L14 polynucleotide is a BCL2L14 exon 1 polynucleotide having a sequence of nucleotides SEQ ID NO: 35, or a polynucleotide having at or greater than about 80%, about 85%, about 90%, about 95%, or about 98% homology with SEQ ID NO: 35, or a polynucleotide comprising a portion of SEQ ID NO: 35. In some embodiments, the BCL2L14 polynucleotide is an BCL2L14 exon 2 polynucleotide having a sequence of nucleotides 12079299-12079738 of SEQ ID NO: 32, or a polynucleotide having at or greater than about 80%, about 85%, about 90%, about 95%, or about 98% homology with nucleotides 12079299-12079738 of SEQ ID NO: 32, or a polynucleotide comprising a portion of nucleotides 12079299-12079738 of SEQ ID NO: 32. In some embodiments, the BCL2L14 polynucleotide is an BCL2L14 exon 2 polynucleotide having a sequence of nucleotides SEQ ID NO: 36, or a polynucleotide having at or greater than about 80%, about 85%, about 90%, about 95%, or about 98% homology with SEQ ID NO: 36, or a polynucleotide comprising a portion of SEQ ID NO: 36. In some embodiments, the BCL2L14 polynucleotide is an BCL2L14 exon 3 polynucleotide having a sequence of nucleotides 12087213-12087386 of SEQ ID NO: 32, or a polynucleotide having at or greater than about 80%, about 85%, about 90%, about 95%, or about 98% homology with nucleotides 12087213-12087386 of SEQ ID NO: 32, or a polynucleotide comprising a portion of nucleotides 12087213-12087386 of SEQ ID NO: 32. In some embodiments, the BCL2L14 polynucleotide is a BCL2L14 exon 3 polynucleotide having a sequence of nucleotides SEQ ID NO: 37, or a polynucleotide having at or greater than about 80%, about 85%, about 90%, about 95%, or about 98% homology with SEQ ID NO: 37, or a polynucleotide comprising a portion of SEQ ID NO: 37. In some embodiments, the BCL2L14 polynucleotide is an BCL2L14 exon 4 polynucleotide having a sequence of nucleotides 12090779-12090849 of SEQ ID NO: 32, or a polynucleotide having at or greater than about 80%, about 85%, about 90%, about 95%, or about 98% homology with nucleotides 12090779-12090849 of SEQ ID NO: 32, or a polynucleotide comprising a portion of nucleotides 12090779-12090849 of SEQ ID NO: 32. In some embodiments, the BCL2L14 polynucleotide is a BCL2L14 exon 4 polynucleotide having a sequence of nucleotides SEQ ID NO: 38, or a polynucleotide having at or greater than about 80%, about 85%, about 90%, about 95%, or about 98% homology with SEQ ID NO: 38, or a polynucleotide comprising a portion of SEQ ID NO: 38. In some embodiments, the BCL2L14 polynucleotide is an BCL2L14 exon 5 polynucleotide having a sequence of nucleotides 12094664-12094930 of SEQ ID NO: 32, or a polynucleotide having at or greater than about 80%, about 85%, about 90%, about 95%, or about 98% homology with nucleotides 12094664-12094930 of SEQ ID NO: 32, or a polynucleotide comprising a portion of nucleotides 12094664-12094930 of SEQ ID NO: 32. In some embodiments, the BCL2L14 polynucleotide is a BCL2L14 exon 5 polynucleotide having a sequence of nucleotides SEQ ID NO: 39, or a polynucleotide having at or greater than about 80%, about 85%, about 90%, about 95%, or about 98% homology with SEQ ID NO: 39, or a polynucleotide comprising a portion of SEQ ID NO: 39. In some embodiments, the BCL2L14 polynucleotide is an BCL2L14 exon 6 polynucleotide having a sequence of nucleotides 12098950-12099695 of SEQ ID NO: 32, or a polynucleotide having at or greater than about 80%, about 85%, about 90%, about 95%, or about 98% homology with nucleotides 12098950-12099695 of SEQ ID NO: 32, or a polynucleotide comprising a portion of nucleotides 12098950-12099695 of SEQ ID NO: 32. In some embodiments, the BCL2L14 polynucleotide is a BCL2L14 exon 6 polynucleotide having a sequence of nucleotides SEQ ID NO: 40, or a polynucleotide having at or greater than about 80%, about 85%, about 90%, about 95%, or about 98% homology with SEQ ID NO: 40, or a polynucleotide comprising a portion of SEQ ID NO: 40.

“ETV6” or “ETS Variant Transcription Factor 6” refers herein to a polypeptide that is a transcriptional repressor, and in humans, is encoded by the ETV6 gene. In some embodiments, the ETV6 polypeptide is that identified in one or more publicly available databases as follows: HGNC: 3495, Entrez Gene: 2120, Ensembl: ENSG00000139083, OMIM: 600618, UniProtKB: P41212. In some embodiments, the ETV6 polypeptide comprises the sequence of SEQ ID NO: 33 or a polypeptide sequence having at or greater than about 80%, about 85%, about 90%, about 95%, or about 98% homology with SEQ ID NO: 33, or a polypeptide comprising a portion of SEQ ID NO: 33. The ETV6 polypeptide of SEQ ID NO: 33 may represent an immature or pre-processed form of mature ETV6, and accordingly, included herein are mature or processed portions of the ETV6 polypeptide in SEQ ID NO: 33.

The term “ETV6 polynucleotide” refers to a polynucleotide that encodes a ETV6 polypeptide, or any fragment thereof. In some embodiments, the ETV6 polynucleotide is an ETV6 exon 1 polynucleotide having a sequence of nucleotides 11649674-11650160 of SEQ ID NO: 34, or a polynucleotide having at or greater than about 80%, about 85%, about 90%, about 95%, or about 98% homology with nucleotides 11649674-11650160 of SEQ ID NO: 34, or a polynucleotide comprising a portion of nucleotides 11649674-11650160 of SEQ ID NO: 34. In some embodiments, the ETV6 polynucleotide is an ETV6 exon 1 polynucleotide having a sequence of SEQ ID NO: 41, or a polynucleotide having at or greater than about 80%, about 85%, about 90%, about 95%, or about 98% homology with SEQ ID NO: 41, or a polynucleotide comprising a portion of SEQ ID NO: 41. In some embodiments, the ETV6 polynucleotide is an ETV6 exon 2 polynucleotide having a sequence of nucleotides 11752450-11752579 of SEQ ID NO: 34, or a polynucleotide having at or greater than about 80%, about 85%, about 90%, about 95%, or about 98% homology with nucleotides 11752450-11752579 of SEQ ID NO: 34, or a polynucleotide comprising a portion of nucleotides 11752450-11752579 of SEQ ID NO: 34. In some embodiments, the ETV6 polynucleotide is an ETV6 exon 2 polynucleotide having a sequence of SEQ ID NO: 42, or a polynucleotide having at or greater than about 80%, about 85%, about 90%, about 95%, or about 98% homology with SEQ ID NO: 42, or a polynucleotide comprising a portion of SEQ ID NO: 42. In some embodiments, the ETV6 polynucleotide is an ETV6 exon 3 polynucleotide having a sequence of nucleotides 11839140-11839304 of SEQ ID NO: 34, or a polynucleotide having at or greater than about 80%, about 85%, about 90%, about 95%, or about 98% homology with nucleotides 11839140-11839304 of SEQ ID NO: 34, or a polynucleotide comprising a portion of nucleotides 11839140-11839304 of SEQ ID NO: 34. In some embodiments, the ETV6 polynucleotide is an ETV6 exon 3 polynucleotide having a sequence of SEQ ID NO: 43, or a polynucleotide having at or greater than about 80%, about 85%, about 90%, about 95%, or about 98% homology with SEQ ID NO: 43, or a polynucleotide comprising a portion of SEQ ID NO: 43. In some embodiments, the ETV6 polynucleotide is an ETV6 exon 4 polynucleotide having a sequence of nucleotides 11853427-11853561 of SEQ ID NO: 34, or a polynucleotide having at or greater than about 80%, about 85%, about 90%, about 95%, or about 98% homology with nucleotides 11853427-11853561 of SEQ ID NO: 34, or a polynucleotide comprising a portion of nucleotides 11853427-11853561 of SEQ ID NO: 34. In some embodiments, the ETV6 polynucleotide is an ETV6 exon 4 polynucleotide having a sequence of SEQ ID NO: 44, or a polynucleotide having at or greater than about 80%, about 85%, about 90%, about 95%, or about 98% homology with SEQ ID NO: 44, or a polynucleotide comprising a portion of SEQ ID NO: 44. In some embodiments, the ETV6 polynucleotide is an ETV6 exon 5 polynucleotide having a sequence of nucleotides 11869424-11869969 of SEQ ID NO: 34, or a polynucleotide having at or greater than about 80%, about 85%, about 90%, about 95%, or about 98% homology with nucleotides 11869424-11869969 of SEQ ID NO: 34, or a polynucleotide comprising a portion of nucleotides 11869424-11869969 of SEQ ID NO: 34. In some embodiments, the ETV6 polynucleotide is an ETV6 exon 5 polynucleotide having a sequence of SEQ ID NO: 45, or a polynucleotide having at or greater than about 80%, about 85%, about 90%, about 95%, or about 98% homology with SEQ ID NO: 45, or a polynucleotide comprising a portion of SEQ ID NO: 45. In some embodiments, the ETV6 polynucleotide is an ETV6 exon 6 polynucleotide having a sequence of nucleotides 11884445-11884587 of SEQ ID NO: 34, or a polynucleotide having at or greater than about 80%, about 85%, about 90%, about 95%, or about 98% homology with nucleotides 11884445-11884587 of SEQ ID NO: 34, or a polynucleotide comprising a portion of nucleotides 11884445-11884587 of SEQ ID NO: 34. In some embodiments, the ETV6 polynucleotide is an ETV6 exon 6 polynucleotide having a sequence of SEQ ID NO: 46, or a polynucleotide having at or greater than about 80%, about 85%, about 90%, about 95%, or about 98% homology with SEQ ID NO: 46, or a polynucleotide comprising a portion of SEQ ID NO: 46. In some embodiments, the ETV6 polynucleotide is an ETV6 exon 7 polynucleotide having a sequence of nucleotides 11885926-11886026 of SEQ ID NO: 34, or a polynucleotide having at or greater than about 80%, about 85%, about 90%, about 95%, or about 98% homology with nucleotides 11885926-11886026 of SEQ ID NO: 34, or a polynucleotide comprising a portion of nucleotides 11885926-11886026 of SEQ ID NO: 34. In some embodiments, the ETV6 polynucleotide is an ETV6 exon 7 polynucleotide having a sequence of SEQ ID NO: 47, or a polynucleotide having at or greater than about 80%, about 85%, about 90%, about 95%, or about 98% homology with SEQ ID NO: 47, or a polynucleotide comprising a portion of SEQ ID NO: 47. In some embodiments, the ETV6 polynucleotide is an ETV6 exon 8 polynucleotide having a sequence of nucleotides 11890941-11895377 of SEQ ID NO: 34, or a polynucleotide having at or greater than about 80%, about 85%, about 90%, about 95%, or about 98% homology with nucleotides 11890941-11895377 of SEQ ID NO: 34, or a polynucleotide comprising a portion of nucleotides 11890941-11895377 of SEQ ID NO: 34. In some embodiments, the ETV6 polynucleotide is an ETV6 exon 8 polynucleotide having a sequence of SEQ ID NO: 48, or a polynucleotide having at or greater than about 80%, about 85%, about 90%, about 95%, or about 98% homology with SEQ ID NO: 48, or a polynucleotide comprising a portion of SEQ ID NO: 48.

It should be understood that the term “fusion” as used herein refers to a polynucleotide or polypeptide made by joining parts of two previously independent polynucleotides or polypeptides of BCL2L14 and ETV6. In some embodiments, a fusion is formed by joining parts of two previously independent genes through translocation, interstitial deletion, or chromosomal inversion. Accordingly, “a fusion of a BCL2L14 polynucleotide sequence and a ETV6 polynucleotide sequence” refers herein to a fusion of a BCL2L14 DNA sequence and a ETV6 DNA sequence or a fusion mRNA transcribed from the fusion DNA. “BCL2L14- ETV6 polynucleotide fusion” is used interchangeably herein with “fusion of a BCL2L14 polynucleotide sequence and a ETV6 polynucleotide sequence.” “BCL2L14- ETV6 fusion” refers to a “BCL2L14- ETV6 polynucleotide fusion” and/or a “BCL2L14- ETV6 polypeptide fusion.”

In some embodiments, the phrase “a fusion of a BCL2L14 polynucleotide sequence and a ETV6 polynucleotide sequence” herein refers to a fusion of any BCL2L14 exon and any ETV6 exon. In some embodiments, the fusion described herein is: a fusion of exons 1-2 of a BCL2L14 polynucleotide with exons 3-8 of a ETV6 polynucleotide (referred to herein as an “E2-E3 fusion”); a fusion of exons 1-2 of a BCL2L14 polynucleotide with exons 6-8 of a ETV6 polynucleotide (referred to herein as an “E2-E6 fusion”); a fusion of exons 1-4 of a BCL2L14 polynucleotide with exons 2-8 of a ETV6 polynucleotide (referred to herein as an “E4-E2 fusion”); a fusion of exons 1-4 of a BCL2L14 polynucleotide with exons 3-8 of a ETV6 polynucleotide (referred to herein as an “E4-E3 fusion”); or a fusion of exons 1-5 of a BCL2L14 polynucleotide with exons 5-8 of a ETV6 polynucleotide (referred to herein as an “E5-E5 fusion”).

The fusions described herein can be detected by contacting the sample with one or more primers specific for the fusion, performing an amplification reaction, and detecting an amplification product or amplicon. It should be understood and herein contemplated that the term “amplification reaction” of polynucleotide as used herein means the use of an amplification reaction (e.g., PCR) to increase the concentration of a particular nucleic acid sequence within a mixture of nucleic acid sequences. The term “PCR” as used herein refers to the polymerase chain reaction, a laboratory technique used to make multiple copies of a segment of a polynucleotide, as is well- known in the art. The term “PCR” includes all forms of PCR, such as real-time PCR, quantitative reverse transcription PCR (qRT-PCR), multiplex PCR, nested PCR, hot start PCR, or GC-Rich PCR. In some embodiments, the amplification reaction is real-time PCR. Exemplary procedures for real-time PCR can be found in “Quantitation of DNA/RNA Using Real-Time PCR Detection” published by Perkin Elmer Applied Biosystems (1999) and to PCR Protocols (Academic Press New York, 1989), incorporated by reference herein in their entireties. The amplification reaction can also be a loop-mediated isothermal amplification (LAMP), a reaction at a constant temperature using primers recognizing the distinct regions of target DNA for a highly specific amplification reaction. In some embodiments, the BCL2L14- ETV6 polynucleotide fusion disclosed herein is detected by methods such as the Nanostring nCounter assay which directly measures target molecules without PCR amplification using ghost probes against one fusion partner gene, and reporter probes against the other fusion partner gene. In some embodiments, a fusion protein encoded by the fusion polynucleotide disclosed herein is detected by one or more protein detection assays including, for example, Western blotting, immunoblotting, ELISA, immunohistochemistry, or an electrophoresis method (e.g., SDS-PAGE).

The fusion can also be detected by any RNA or DNA based methods known in the art, such as Nanostring assay or whole transcriptome, whole genome or targeted transcriptome or genome sequencing.

In some embodiments, the one or more primers or Nanostring probes comprise a sequence selected from the group consisting of SEQ ID NO: 1-4, 7-12 and 17-19, or a polynucleotide sequence having at or greater than about 80%, about 85%, about 90%, about 95%, or about 98% homology with a sequence selected from the group consisting of SEQ ID NO: 1-4, 7-12 and 17-19, or a polynucleotide comprising a portion of a sequence selected from the group consisting of SEQ ID NO: 1-4, 7-12 and 17-19. In some embodiments, a first primer or Nanostring probe comprises a sequence selected from the group consisting of SEQ ID NOs: 1, 3, 7, 9, 11, 17 and 19, or a polynucleotide sequence having at or greater than about 80%, about 85%, about 90%, about 95%, about 98%, or about 99% homology with the sequence selected from SEQ ID NOs: 1, 3, 7, 9, 11, 17 and 19, or a polynucleotide comprising a portion of with the sequence selected from SEQ ID NOs: 1, 3, 7, 9, 11, 17 and 19, and second primer or Nanostring probe comprises a sequence selected from the group consisting of SEQ ID NOs: 2, 4, 8, 10, 12, and 18, or a polynucleotide sequence having at or greater than about 80%, about 85%, about 90%, about 95%, about 98%, or about 99% homology with the sequence selected from SEQ ID NOs: 2, 4, 8, 10, 12, and 18, or a polynucleotide comprising a portion of with the sequence selected from SEQ ID NOs: 2, 4, 8, 10, 12, and 18. In some embodiments, the one or more primers or Nanostring probes comprise a sequence selected from the group consisting of SEQ ID NO: 1-19, or a polynucleotide sequence having at or greater than about 80%, about 85%, about 90%, about 95%, or about 98% homology with a sequence selected from the group consisting of SEQ ID NO: 1-19, or a polynucleotide comprising a portion of a sequence selected from the group consisting of SEQ ID NO: 1-19.

As used herein, the term “detecting” refers to detection of a level of a fusion (e.g., the fusion of a BCL2L14 polynucleotide sequence and a ETV6 polynucleotide) that is at least about 5% (e.g., at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1000%, at least about 2000%, at least about 3000%, or at least about 5000%) or at least about 5 times (e.g., at least about 6 times, at least about 7 times, at least about 8 times, at least about 9 times, at least about 10 times, at least about 20 times, at least about 30 times, at least about 40 times, at least about 50 times, or at least about 100 times) higher as compared to a sample from a subject in general or a study population (e.g., healthy control).

In certain embodiments the primers are used in DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, regular PCR, real-time PCR, DNA sequencing, DNA extension, DNA polymerization, RNA transcription, and reverse transcription. Techniques and conditions that amplify the primer in a sequence specific manner are preferred. In certain embodiments, the primers are used for the DNA or RNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. In some embodiments, the primers are used for gene array analysis. Typically, the disclosed primers hybridize with a region of the disclosed nucleic acids (e.g., BCL2L14 or ETV6) or they hybridize with the complement of the nucleic acids or complement of a region of the nucleic acids.

In some embodiments, subject has a cancer. The cancer can be any of breast cancer, prostate cancer, ovarian cancer, cervical cancer, skin cancer, pancreatic cancer, colorectal cancer, renal cancer, liver cancer, brain cancer, lymphoma, leukemia, and lung cancer. In certain aspects, the cancer is a breast cancer. In certain aspects the cancer is a triple negative breast cancer.

The “sample” referred to herein is a fluid or tissue sample. In some embodiments, the sample is a breast tissue sample. In some embodiments, the breast tissue is cancerous. Included herein are methods that comprise detection of an increased amount of the BCL2L14- ETV6 fusion in a breast tissue sample as compared to a control, wherein the control can be a normal breast tissue or any normal tissue other than testis tissue, and wherein the control can be obtained from the same subject or a different subject. In some embodiments, the control is a level or amount of the BCL2L14- ETV6 fusion in a general or study population. In some embodiments, the cancerous breast tissue exhibits an increased amount of the fusion of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a control, or at least about a 2-fold, or at least about a 3-fold, or at least about a 4-fold, or at least about a 5-fold, or at least about a 10-fold, at least about a 20-fold, at least about a 50-fold, at least about a 100-fold, at least about a 500-fold, or at least about a 1000-fold as compared to a control.

It should be understood and herein contemplated that detection of the BCL2L14- ETV6 fusion or an increase in the amount of the BCL2L14- ETV6 fusion as compared to a control indicates a decreased sensitivity of the tissue sample, cancer cell or tumor to taxane (such as paclitaxel and docetaxel). The BCL2L14- ETV6 can be detected using any method described herein. In some embodiments, the decreased sensitivity of a cancer cell or tumor refers to a more significant increase in tumor growth, a larger increase in tumor volume or size, a slower clearance of tumor, a decrease in cancer cell death, an increase in cell migration, metastasis, and/or proliferation as compared to a control cancer cell or tumor, wherein the control tumor or cancer cell does not have the BCL2L14- ETV6fusion disclosed herein. In some embodiments, the tumor or cancer cell comprising the BCL2L14- ETV6fusion exhibits a decreased sensitivity to taxane (such as paclitaxel and docetaxel) of at least about at least about 10%, at least about 20%, or at least about 30%, or at least about 40%, or at least about 50%, or at least about 60%, or at least about 70%, or at least about 80%, or at least about 90% or at least about 100%, or a decreased sensitivity to taxane (such as paclitaxel and docetaxel) of at least about a 2-fold, or at least about a 3-fold, or at least about a 4-fold, or at least about a 5-fold, or at least about a 10-fold, at least about a 20-fold, at least about a 50-fold, at least about a 100-fold, or at least about a 500-fold as compared to a control. Taxane is a class of compounds know in the art. See, e.g., U.S. Pat. NOs: 6,677,456 and 9,284,327, incorporated by reference herein in their entireties.

As used herein, “paclitaxel” refers to a composition having the below chemical structure.

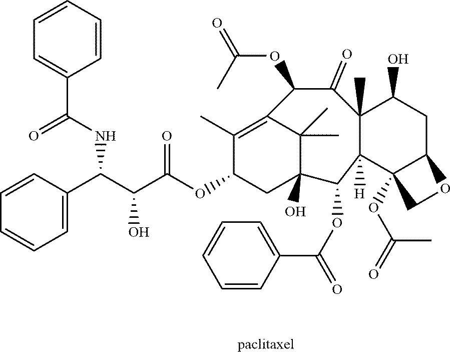

As used herein, “docetaxel” refers to a composition having the below chemical structure.

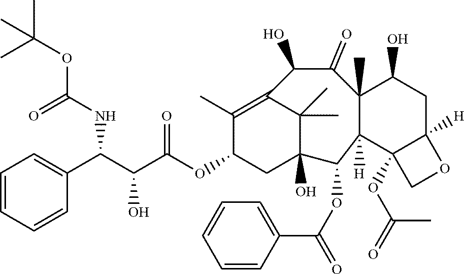 [SMILES: CC(=O)O[C@@]12CO[C@@H]1C[C@H](O)[C@@]1(C)C(=O)[C@H](O)C3=C(C)[C@@H](OC(=O)[C@H](O)[C@@H](NC(=O)OC(C)(C)C)c4ccccc4)C[C@@](O)([C@@H](OC(=O)c4ccccc4)[C@H]21)C3(C)C]

In some embodiments, detection of the BCL2L14- ETV6 fusion or an increase in the amount of the BCL2L14- ETV6 fusion as compared to a control indicates a decreased sensitivity of the tissue sample, cancer cell or tumor to paclitaxel bioequivalent.

Since detection of a BCL2L14- ETV6 fusion indicates an increased resistance to taxane (such as paclitaxel and docetaxel), or a decrease in the effectiveness of taxane (such as paclitaxel and docetaxel) in the subject, certain embodiment further include treating the subject with an alternative to taxane (such as paclitaxel and docetaxel). The subject can be administered one or more of capecitabine, doxorubicin, cyclophosphamide, fluorouracil, epirubicin, cisplatin, carboplatin, olaparib, and talazoparib for the treatment of a cancer in a subject having a BCL2L14-ETV6 fusion.

In one example, method further comprises administering to the subject a therapeutically effective amount of capecitabine. The term “capecitabine” refers to a composition having the below chemical structure.

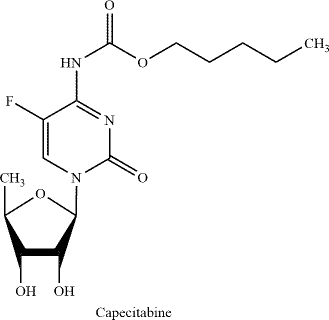

In one example, the method further comprises administering to the subject a therapeutically effective amount of cisplatin. The term “cisplatin” refers to a composition having the below chemical structure.

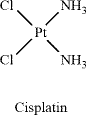

In one example, the method further comprises administering to the subject a therapeutically effective amount of carboplatin. The term “carboplatin” refers to a composition having the below chemical structure.

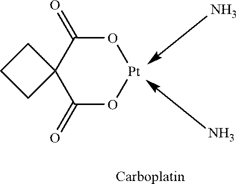

In one example, the method further comprises administering to the subject a therapeutically effective amount of olaparib. The term “olaparib” refers to a composition having the below chemical structure.

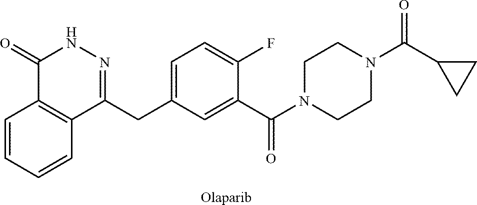

In one example, the method further comprises administering to the subject a therapeutically effective amount of talazoparib. The term “talazoparib” refers to a composition having the below chemical structure.

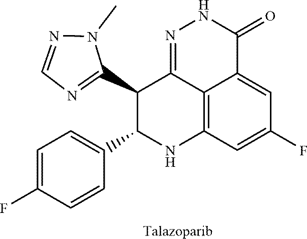

In one example, the method further comprises administering to the subject a therapeutically effective amount of doxorubicin. The term “doxorubicin” refers to a composition having the below chemical structure.

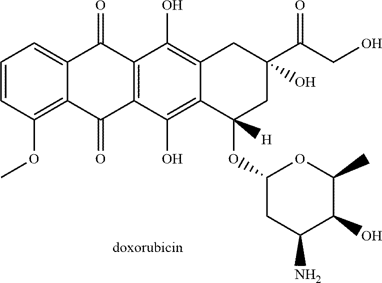

In one example, the method further comprises administering to the subject a therapeutically effective amount of cyclophosphamide. The term “cyclophosphamide” refers to a composition having the below chemical structure.

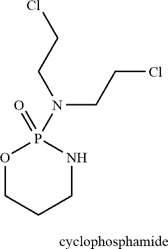

In one example, the method further comprises administering to the subject a therapeutically effective amount of fluorouracil. The term “fluorouracil” refers to a composition having the below chemical structure.

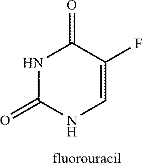

In one example, the method further comprises administering to the subject a therapeutically effective amount of epirubicin. The term “epirubicin” refers to a composition having the below chemical structure.

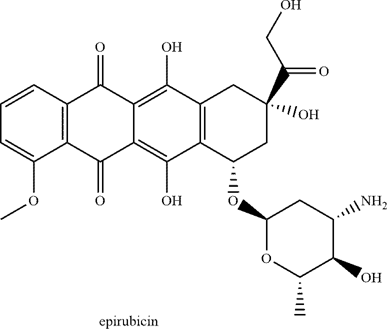

In some embodiments, the method further comprises administering to the subject a therapeutically effective amount of an immune checkpoint inhibitor. In some examples, the immune checkpoint inhibitor is a PD-1 inhibitor. In some examples, the immune checkpoint inhibitor is a PD-L1 inhibitor. In some examples, the immune checkpoint inhibitor is a PD-L2 inhibitor. In some examples, the immune checkpoint inhibitor is a CTLA-4 inhibitor.

As used herein, the term “PD-1 inhibitor” refers to a composition that binds to PD-1 and reduces or inhibits the interaction between the bound PD-1 and PD-L1. In some embodiments, the PD-1 inhibitor is a monoclonal antibody that is specific for PD-1 and that reduces or inhibits the interaction between the bound PD-1 and PD-L1. Non-limiting examples of PD-1 inhibitors are pembrolizumab, nivolumab, and cemiplimab. In some embodiments, the pembrolizumab is KEYTRUDA or a bioequivalent. In some embodiments, the pembrolizumab is that described in U.S. Pat. No. 8952136, U.S. Pat. No. 8354509, or U.S. Pat. No. 8900587, all of which are incorporated by reference in their entireties. In some embodiments, the pembrolizumab has the Unique Ingredient Identifier (UNII) of the U.S. Food and Drug Administration of DPT0O3T46P. In some embodiments, the nivolumab is OPDIVO or a bioequivalent. In some embodiments, the nivolumab has the Unique Ingredient Identifier (UNII) of the U.S. Food and Drug Administration of 31YO63LBSN. In some embodiments, the nivolumab is that described in U.S. Pat. No. 7595048, U.S. Pat. No. 8738474, U.S. Pat. No. 9073994, U.S. Pat. No. 9067999, U.S. Pat. No. 8008449, or U.S. Pat. No. 8779105, all of which are incorporated by reference in their entireties. In some embodiments, the cemiplimab is LIBTAYO or a bioequivalent. In some embodiments, the cemiplimab has the Unique Ingredient Identifier (UNII) of the U.S. Food and Drug Administration of 6QVL057INT. In some embodiments, the cemiplimab is that described in U.S. Pat. No. 10844137, which is incorporated by reference in its entirety.

The term “PD-L1 inhibitor” refers to refers to a composition that binds to PD-1 and reduces or inhibits the interaction between the bound PD-L1 and PD-1. In some embodiments, the PD-L1 inhibitor is a monoclonal antibody that is specific for PD-L1 and that reduces or inhibits the interaction between the bound PD-L1 and PD-1. Non-limiting examples of PD-L1 inhibitors are atezolizumab, avelumab and durvalumab. In some embodiments, the atezolizumab is TECENTRIQ or a bioequivalent. In some embodiments, the atezolizumab has the Unique Ingredient Identifier (UNII) of the U.S. Food and Drug Administration of 52CMI0WC3Y. In some embodiments, the atezolizumab is that described in U.S. Pat. No. 8217149, which is incorporated by reference in its entirety. In some embodiments, the avelumab is BAVENCIO or a bioequivalent. In some embodiments, the avelumab has the Unique Ingredient Identifier (UNII) of the U.S. Food and Drug Administration of KXG2PJ551I. In some embodiments, the avelumab is that described in U.S. Pat. App. Pub. No. 2014321917, which is incorporated by reference in its entirety. In some embodiments, the durvalumab is IMFINZI or a bioequivalent. In some embodiments, the durvalumab has the Unique Ingredient Identifier (UNII) of the U.S. Food and Drug Administration of 28X28X9OKV. In some embodiments, the durvalumab is that described in U.S. Pat. No. 8779108, which is incorporated by reference in its entirety.

The term “CTLA-4 inhibitor” refers to a composition that binds to CTLA-4 and reduces or inhibits the interaction between the bound CTLA-4 and B7. In some embodiments, the CTLA-4 inhibitor is a monoclonal antibody that is specific for CTLA-4 and that reduces or inhibits the interaction between the bound CTLA-4 and B7. A non-limiting example of a CTLA-4 inhibitor is ipilimumab. In some embodiments, the ipilimumab is YERVOY or a bioequivalent. In some embodiments, the ipilimumab has the Unique Ingredient Identifier (UNII) of the U.S. Food and Drug Administration of 6T8C155666. In some embodiments, the ipilimumab is that described in U.S. Pat. No. 7605238, U.S. Pat. No. 6984720, U.S. Pat. No. 5811097, U.S. Pat. No. 5855887, or U.S. Pat. No. 6051227, all of which are incorporated by reference in their entireties.

As the timing of a cancer can often not be predicted, it should be understood the disclosed methods of treating, preventing, reducing, and/or inhibiting the disease or disorder described herein can be used prior to or following the onset of the disease or disorder, to treat, prevent, inhibit, and/or reduce the disease or disorder or symptoms thereof. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24,23, 22, 21, 20, 19, 18, 17, 16, 15, 14, 13, 12, 11, 10, 9, 8, 7, 6, 5, 4, 3 days, 60, 48, 36, 30, 24, 18,15, 12, 10, 9, 8, 7, 6, 5, 4, 3, 2 hours, 60, 45, 30, 15, 10, 9, 8, 7, 6, 5, 4, 3, 2, or 1 minute prior to onset of the disease or disorder; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6,7, 8, 9, 10, 11, 12, 13, 14, 15, 16, 17, 18, 19, 20, 21, 22, 23, 24, 25, 26, 27, 28, 29, 30 days, 2, 3,4, 5, 6, 7, 8, 9, 10, 11, 12 months, 2, 3, 4, 5, 6, 7, 8, 9, 10, 15, 20, 25, 30, 35, 40, 45, 50, 55, 60 or more years after onset of the disease or disorder.

Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

### KITS

Included herein are kits comprising a probe or a set of probes, for example, a detectable probe or a set of amplification primers that specifically recognize a nucleic acid comprising a fusion point or break point. The kit can further include, in the same vessel, or in a separate vessel, a component from an amplification reaction mixture, such as a polymerase, typically not from human origin, dNTPs, and/or UDG. In some embodiments, the amplification primers are selected from the group consisting of SEQ ID NO: 1, SEQ ID NO: 3, SEQ ID NO: 7, SEQ ID NO: 9, SEQ ID NO: 11, SEQ ID NO: 17, SEQ ID NO: 19, SEQ ID NO: 2, SEQ ID NO: 4, SEQ ID NO: 8, SEQ ID NO: 10, SEQ ID NO: 12, and SEQ ID NO: 18. In some embodiments, the detectable probe is selected from polynucleotide sequence that specifically hybridizes to a fusion point nucleotide sequence selected from SEQ ID NO: 23, SEQ ID NO:20, SEQ ID NO: 24 and SEQ ID NO:21. In some embodiments, the kit comprises a detectable moiety that is covalently bonded to the probe. Furthermore, the kit can include a control nucleic acid. For example the control nucleic acid can include a sequence that includes a fusion point sequence selected from the group of SEQ ID NO: 23, SEQ ID NO:20, SEQ ID NO: 24 and SEQ ID NO:21.

All patents, patent applications, and publications referenced herein are incorporated by reference in their entirety for all purposes.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Landscape Analysis of Adjacent Gene Rearrangements Reveals BCL2L14-ETV6 Gene Fusions in More Aggressive Triple-Negative Breast Cancer

Recurrent gene fusions that result from chromosome translocations comprise a critical class of genetic cancer-causing aberrations, which have fueled modern cancer therapeutics. In the past decade, the discovery of novel gene fusions in epithelial tumors have generated great therapeutic impact in recent years. This is represented by the discovery of an EML4-ALK fusion in ~4% of lung cancer and the FGFR-TACC fusion in ~3% of glioblastomas that have culminated in effective targeted therapies in these tumors (Koivunen JP, et al. (2008), Singh D, et al. (2012)). Most recently, larotrectinib targeting the NTRK gene fusions accounting for up to ~1 % of solid tumors have received FDA approval for pan-cancer use, which is considered as the first targeted therapy with tissue-agnostic indication (Cocco E (2018)). Although low in percentages, these neoplastic gene fusions can move toward genetic subtyping of solid tumors that can be curable by fusion-targeted therapies.

Analysis of a TCGA RNAseq dataset identified a recurrent gene fusion between the 5′ region of ESR1 and the coding region of the adjacent CCDC170 gene, which was subsequently verified by several other studies (Matissek KJ, et al. (2018), Hartmaier RJ, et al. (2018), Giltnane JM, et al. (2017), Fimereli D, et al. (2018), Lei JT, et al. (2018)). This fusion represents a cryptic class of genomic rearrangements between adjacent genes (genes within 500 kb in distance), which is termed as adjacent gene rearrangements (AGRs). ESR1- CCDC170 is detected in 6-8% of luminal B breast tumors and promotes increased aggressiveness (Veeraraghavan J, et al. (2014)), which shows that AGRs can meaningfully contribute to breast cancer development, pathogenesis, and resistance to cancer therapies. Nonetheless, AGRs have been frequently overlooked by fusion detection tools based on RNAseq data due to the overwhelming number of adjacent chimeras resulting from intergenic splicing events. In addition, such cryptic genomic changes cannot be detected by conventional cytogenetic assays such as spectral karyotyping (SKY) or fluorescence in situ hybridization (FISH) due to the proximity of the rearranged DNAs and the limited resolutions of these assays. For these reasons, AGRs remain an under-explored area of breast cancer genetics.

Here, a landscape study of adjacent gene rearrangements was performed in breast cancer cataloged by whole-genome sequencing (WGS) data, and a novel recurrent fusion, BCL2L14-ETV6, that is preferentially present in triple-negative breast cancer (TNBC) was identified. The fusion partners, an ETS family transcription factor gene ETV6, and an apoptosis facilitator Bcl-2-like protein 14 gene (BCL2L14) are neighboring genes of approximately 154 kb apart on the same strand of chromosome 12, with BCL2L14 positioned at the 3′ of ETV6. BCL2L14 encodes a protein member of the Bcl-2 family and was previously described as a novel pro-apoptotic factor (Guo B, Godzik A, & Reed JC (2001)). ETV6 encodes a ubiquitously expressed transcriptional repressor that is generally considered as a tumor suppressor unless it forms oncogenic fusions (Rasighaemi P & Ward AC (2017)) (i.e. ETV6-NTRK3 fusion in secretory breast carcinoma (Tognon C, et al. (2002)). In this study, the pathological role of BCL2L14-ETV6 was further investigated in triple-negative breast cancer.

### Example 2. AGRs Comprise The Most Frequent Form of Intergenic Rearrangements in Breast Cancer

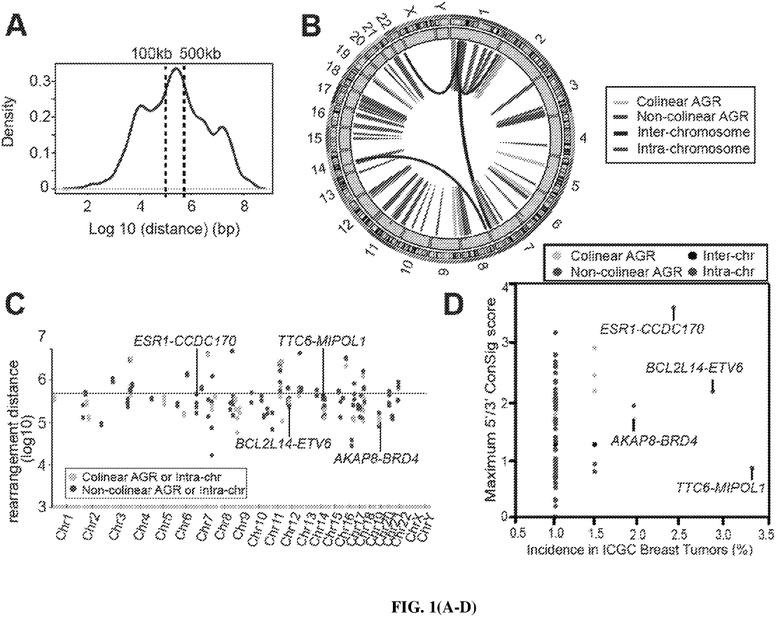

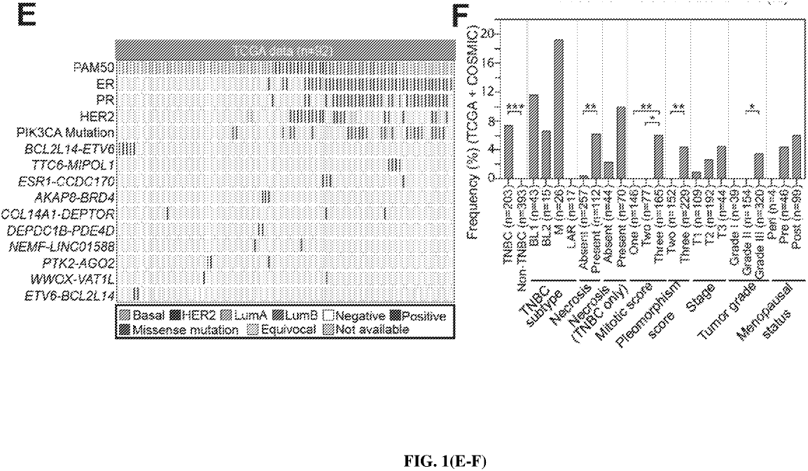

To provide a systematic picture of AGR events in breast cancer, we first analyzed the full spectrum of experimentally confirmed somatic translocations in 9 breast cancer cell lines and 15 breast tumors cataloged from Whole Genome Sequencing (WGS) data in a previous study (Stephens PJ, et al. (2009)). Among 9,408 authentic somatic rearrangements, about half are intra-chromosomal rearrangements between adjacent genes located within 500 kb in distance to each other on the chromosome (FIG. 1A). As shown in FIG. 1A, a majority of the intra-chromosomal translocations are within 500 kb apart, with a median distance of ~100 kb. This shows that AGRs may be a more frequent genetic event than realized. Although most of these rearrangements are likely the consequence of genomic instability, it is plausible that a subset of them could be recurrent genetic events that are pathological in breast cancer.

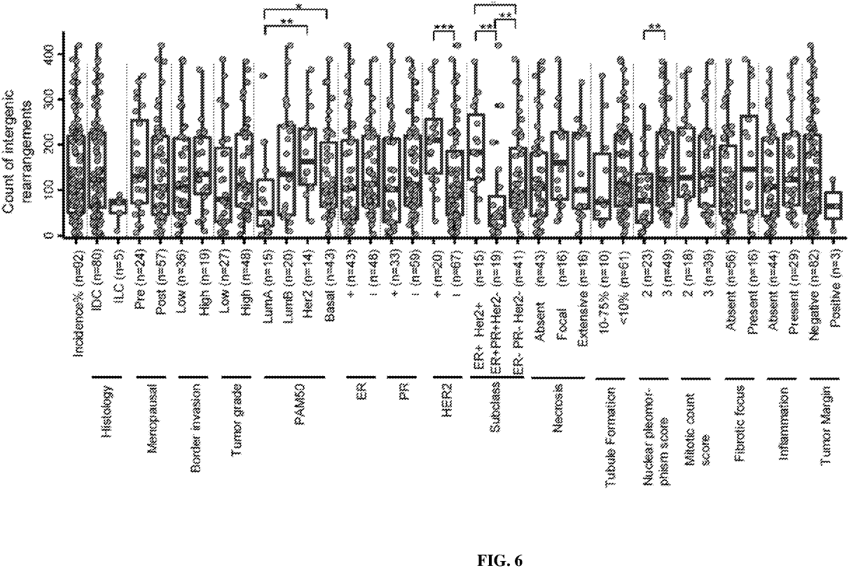

To discover AGRs in breast cancer systematically, the somatic structural mutations cataloged were further analyzed by the International Cancer Genome Consortium (ICGC) based on WGS data for 215 breast tumors. The somatic structural mutations were first mapped with the human exome to reveal genes and exons affected by the rearrangements. The fusion partners were determined based on the strands and genomic regions retained in the rearrangements. To explore if the intergenic rearrangements are enriched in specific breast cancer subtypes, the 92 ICGC breast tumors contributed by The Cancer Genome Atlas (TCGA) that have detailed histopathological data from a recent report were isolated (Heng YJ, et al. (2017)) (FIG. 6). Overall Her2 and Basal subtypes show significantly higher total number of rearrangements compared to Luminal A tumors. Luminal B tumors also exhibit a trend of increased total rearrangements than luminal A tumors. In addition, the breast tumors with high nuclear pleomorphism show significantly higher number of rearrangements. Next, the recurrent gene fusions were identified and classified into AGRs (local rearrangements involving genes of less than 500 kb apart), distant intra-chromosomal rearrangements (involving partner genes of more than 500 kb apart), or interchromosomal rearrangements (FIG. 1B). In total, 99 recurrent gene fusions that occur in at least two breast tumors were identified, including 57 adjacent gene fusions (57.6%), 35 intra-chromosome fusions (35.4%), and 7 interchromosome fusions (7.1%) (Table 2). The AGR events spread throughout the genome, with some genomic regions harboring higher incidence of recurrent gene rearrangements (FIG. 1C). Among the 57 recurrent AGRs, 20 are between colinear genes with 5′ located upstream of 3′ partner (35.1%), 35 are between non-colinear genes with 5′ partner located downstream of the 3′ partner (61.4%), which are the results of intergenic deletions or tandem duplications, respectively.

### Example 3. Systematic Discovery of Recurrent Agrs in Breast Cancer

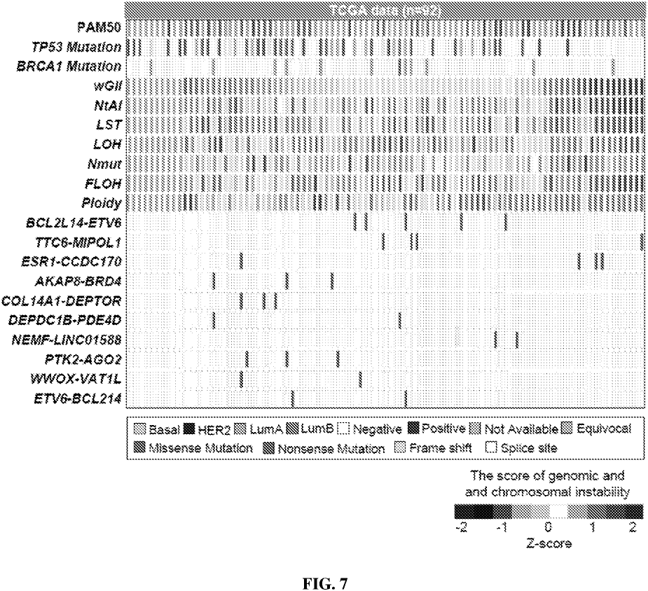

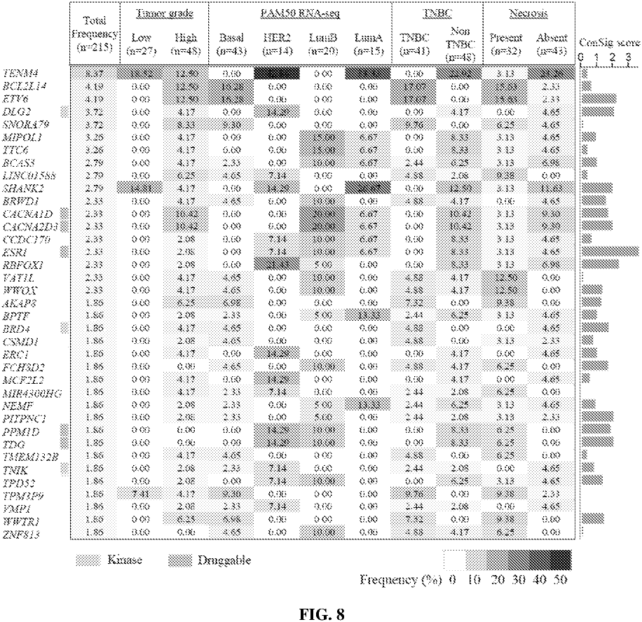

The recurrent gene rearrangements were ranked based on their incidence in the ICGC breast tumor patient cohort, and their concept signature scores (FIG. 1D and Table 2). The concept signature (ConSig) scores are developed in the previous study to compute the functional relevance of fusion genes underlying cancer based on their associations with molecular concepts associated with known cancer causal genes (Wang XS, et al. (2009)). The top four most frequent gene fusions identified by our analysis include BCL2L14-ETV6, TTC6-MIPOL1, ESR1-CCDC170, and AKAP8-BRD4, all of which are AGRs (FIG. 1D). To test if the top recurrent AGRs can be a function of genomic instability, the 92 TCGA breast tumors that have available DNA damage repair (DDR) deficiency scores were isolated (Marquard AM, et al. (2015)), and these tumors were sorted by their genomic instability index (GII) (FIG. 7). Overall, these fusions showed modest enrichment in the tumors with high GII scores, indicating that DDR deficiency can facilitate formation of a subset of the rearrangements generating these fusions. Further clinicopathological association analysis of these lead recurrent AGRs revealed their preferential presence in the more aggressive forms of breast cancers including basal-like and luminal B breast cancers (FIG. 1E and Table 3). Among the top AGRs, BCL2L14-ETV6 and AKAP8-BRD4 are exclusively found in basallike breast cancers, while TTC6-MIPOL1 and ESR1-CCDC170 (Veeraraghavan J, et al. (2014)) are preferentially present in luminal B tumors. While basal-like and luminal B tumors tend to have higher number of rearrangements, the specific enrichment of these fusions in either of these subtypes but not in all genomically unstable entities implies their potential function in these tumors. To test if the lead recurrent AGRs display alteration patterns in which most tumors only have one of these fusions, mutual exclusivity tests were performed using a discrete independence statistics called “Discover” that accounts for the heterogeneous rearrangement rates across tumors (Canisius S, Martens JW, & Wessels LF (2016)). Group-wise mutual exclusivity test for the top recurrent AGRs shows that there are significant number of tumors that harbor only one of these rearrangements (p<0.001, FIG. 1E). This indicates that these recurrent AGRs tend not to co-occur in the same tumor, as opposed to typical DDR-driven rearrangements coexisting in DDR-deficient tumors. Next, the incidences of rearrangements were surveyed based on fusion partner genes and stratified these incidences based on TCGA clinicopathological features (FIG. 8). The result revealed that most of the lead fusion genes are preferentially present in high grade tumors, except TENN4, SHANK2, and TPM3P9. Among these lead fusion genes, several kinase fusion genes were detected, such as DLG2, BRD4, TNIK. Taken together, the preferential presence of these recurrent AGRs in specific aggressive forms of breast tumors and their tendency not to coexist in genomically unstable tumors show their pathological roles in breast cancer.

### Example 4. Characterization of the Lead Recurrent Agrs in Breast Cancer Samples

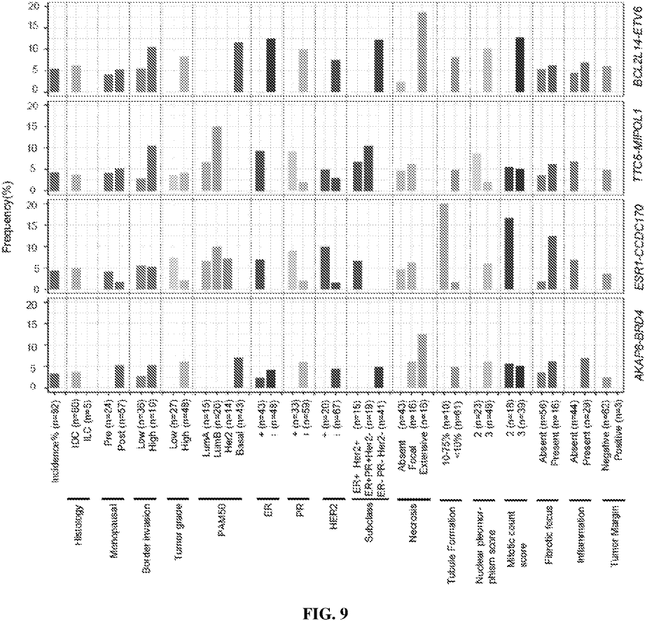

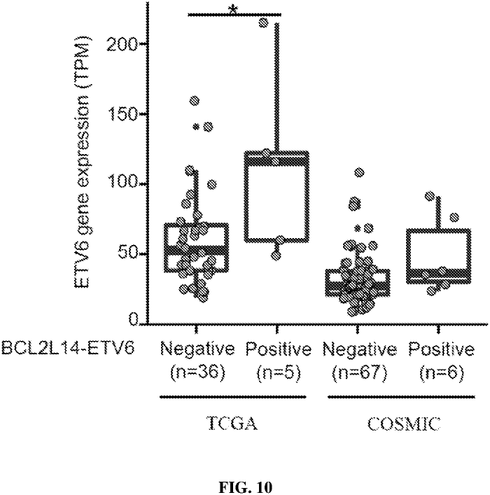

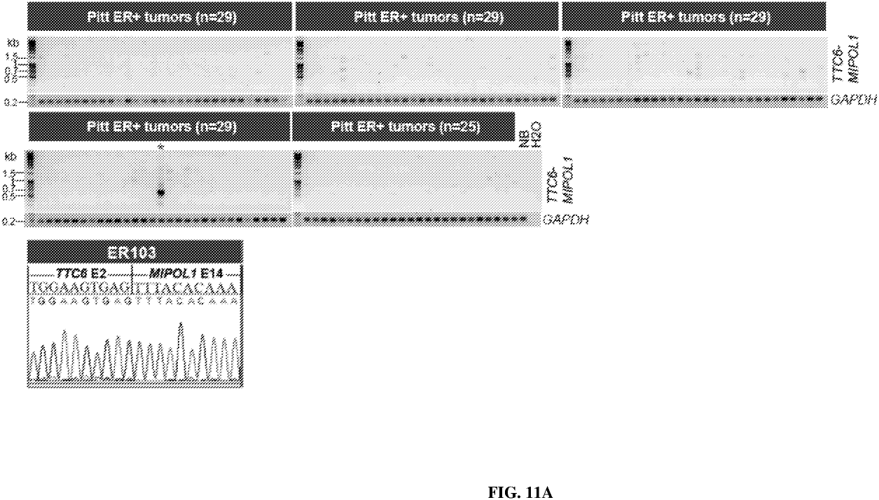

To explore if the most frequent gene rearrangements are significantly associated with specific histopathological features, the detailed histopathological data of TCGA breast tumors available from a recent report were analyzed (Heng YJ, et al. (2017)). The analysis revealed that BCL2L14-ETV6 and AKAP8-BRD4 tend to occur in breast tumors with gross necrosis (particularly, extensive necrosis), higher tubule formation score, and higher nuclear pleomorphism (FIG. 9). Tumor necrosis is defined as the morphological changes following cell death (Van Cruchten S & Van Den Broeck W (2002)). The presence of necrosis in breast cancer indicates more aggressive tumors that are associated with early recurrence, poor prognosis (Leek RD, Landers RJ, Harris AL, & Lewis CE (1999)), and approximately 35% of TNBC tumors present necrosis features (Urru SAM, et al. (2018)). To further verify the above histopathological associations in a larger cohort of TNBC tumors, we analyzed the somatic rearrangements detected by WGS data in 516 breast tumors, which are provided by the Catalogue of Somatic Mutations in Cancer (COSMIC) (Nik-Zainal S, et al. (2016), Forbes SA, et al. (2016)). From a total of 162 TNBCs in this cohort, ten BCL2L14-ETV6 positive cases were detected, but there is no AKAP8-BRD4 positive case (Table 1). In both TCGA and COSMIC cohorts of TNBC tumors, the BCL2L14-ETV6 positive tumors tend to have a higher level of ETV6 expression than fusion negative cases, but not all ETV6 overexpressing tumors harbor BCL2L14-ETV6 fusion (FIG. 10). The BCL2L14-ETV6 fusions are exclusively detected in TNBC, and correlate with more aggressive features, including presence of necrosis, high mitotic and nuclear pleomorphism scores, advanced tumor stage, and high pathology grade, consistent with the above findings (FIG. 1F). In addition, among TNBC subtypes, BCL2L14-ETV6 fusions most frequently present in the mesenchymal (M) subtype characterized by enriched cell motility and epithelial-to-mesenchymal transition (EMT) pathways, accounting for approximately 19.2% of these tumors in the TCGA+COSMIC cohort (FIG. 1F). In addition, BCL2L14-ETV6 is also detected in 11.6% of the basal-like 1 (BL1) tumors characterized by enriched cell cycle and cell division pathways (Lehmann BD, et al. (2011)).

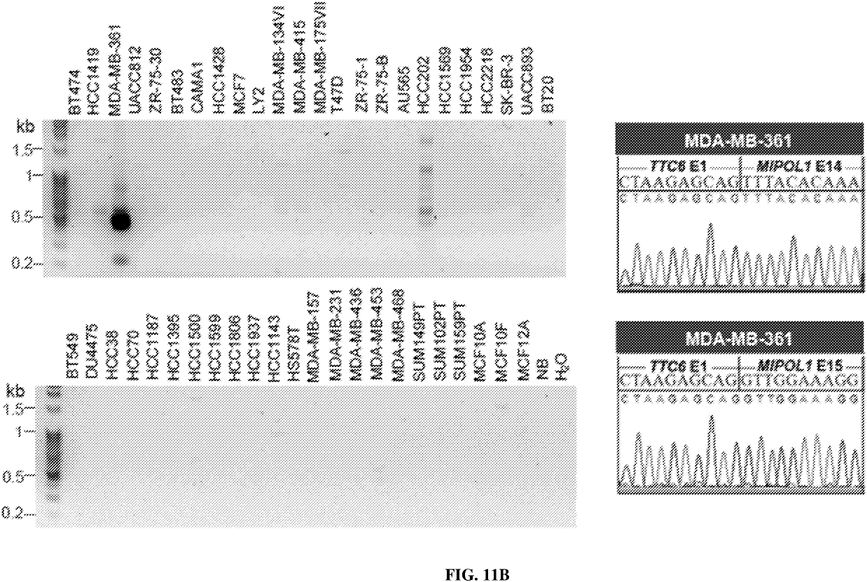

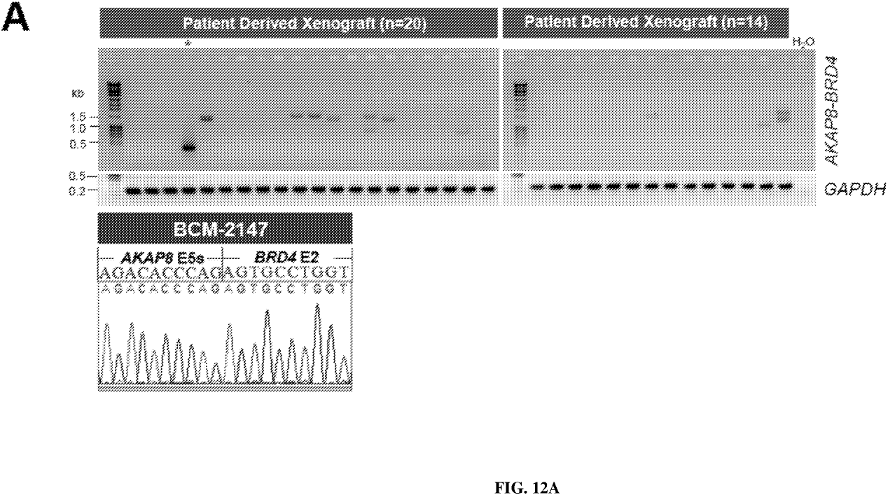

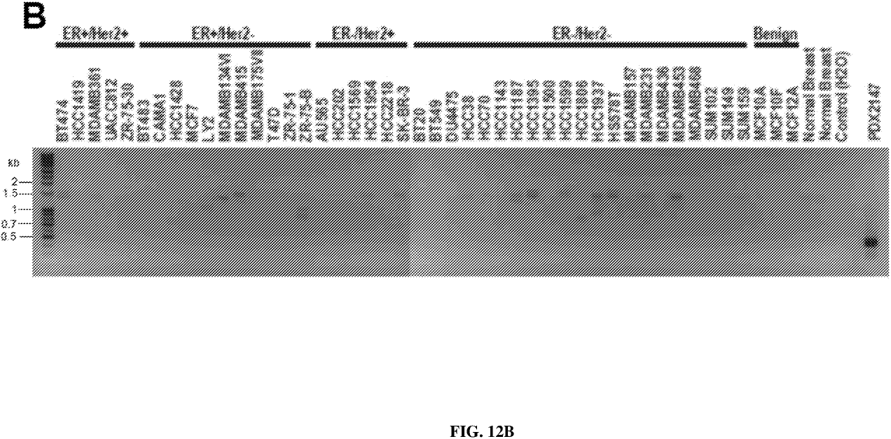

The lead recurrent AGR fusions were validated, including BCL2L14-ETV6, TTC6-MIPOL1, and AKAP8-BRD4, in a panel of breast cancer cell lines and human breast cancer tissues by reverse transcription PCR (RT-PCR). The validation of the most frequent AGR, BCL2L14-ETV6, can be detailed in the below section. Since TTC6-MIPOL1 is preferentially expressed in luminal breast tumors, this fusion was first screened in 141 ER+ breast tumors from the University of Pittsburgh (Pitt) cohort using primers located on the first exon of TTC6 and the last exon of MIPOL1, which identified one positive case in this cohort (FIG. 11A). In addition, we also detected this fusion in one luminal cell line (MDA-MB-361) (FIG. 11B). In addition, the presence of the AKAP8-BRD4 fusion was also verified in one patient-derived xenograft (PDX) tumor through screening a panel of 34 TNBC PDX tumors (Zhang X, et al. (2013), Neelakantan D, et al. (2017)) (FIG. 12A).

### Example 5. BCL2L14-ETV6 is Exclusively Detected in Triple-Negative Breast Cancer

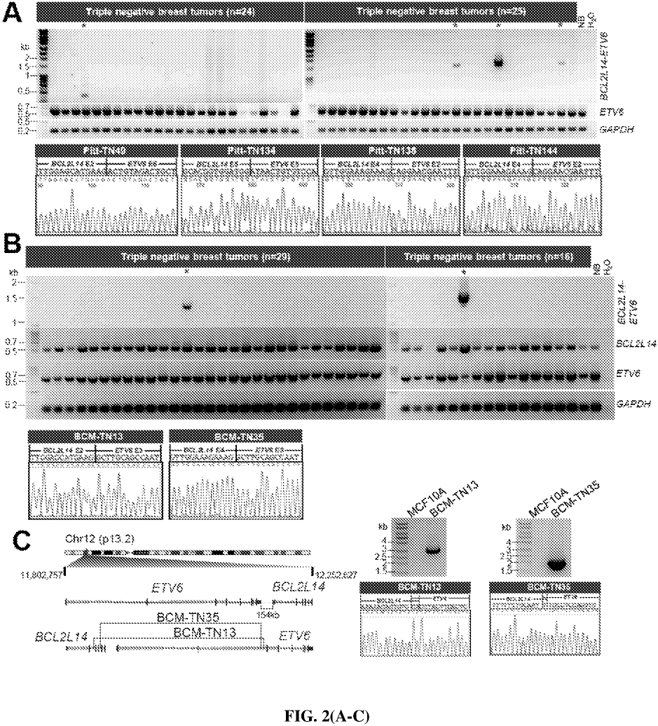

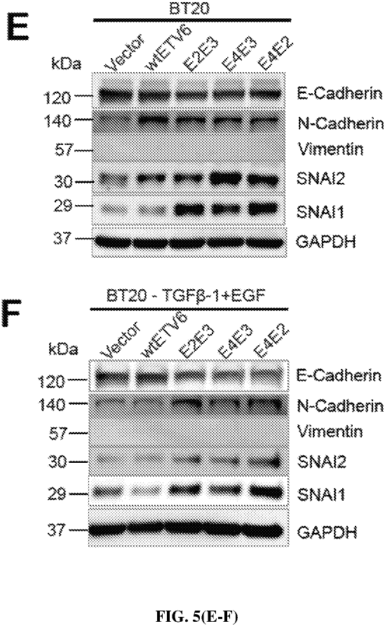

Next, the BCL2L14-ETV6 rearrangements were assessed, which were identified in 12.2% and 6.2% of TNBC cases in the TCGA and COSMIC cohorts respectively (FIG. 1F and Table 1). BCL2L14-ETV6 fusion transcripts were first detected by RT-PCR in 134 TNBC tumors from two available patient cohorts. To detect most fusion variations, a pair of primers located on exon 2 of BCL2L14 and the last exon of ETV6 were designed respectively. This primer set detected BCL2L14-ETV6 fusion in four of the 89 TNBC tumors from the University of Pittsburgh (Pitt) cohort (FIG. 2A), and two of the 45 TNBC tumors from Baylor College of Medicine (BCM) cohort (FIG. 2B). The fusion point sequences in FIG. 2A are TTGGAGCATGAAGACTGTAGACTGCT (SEQ ID NO: 20) (E2-E6 fusion point), GCACGGTGGATGGATAACTGTGTCCA (SEQ ID NO: 21) (E5-E5 fusion point), GTTGGAAAGAAAGCAGGAACGAATTT (SEQ ID NO: 22) (E4-E2 fusion point). The sequences in FIG. 2B are TTGGAGCATGAAGGCTTGCAGCCAAT (SEQ ID NO: 23) (E2-E3 fusion point), and GTTGGAAAGAAAGGCTTGCAGCCAAT (SEQ ID NO: 24) (E4-E3 fusion point). The sequences in FIG. 5C are AAAAAGAGTGTGCACCTACTTCACTC (SEQ ID NO: 25) and TTTTTCTGCAATTTGCCTCCAGGTG (SEQ ID NO: 26).

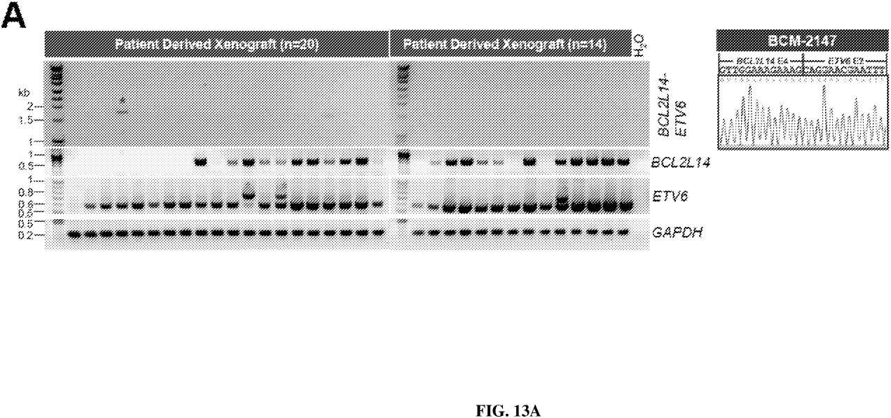

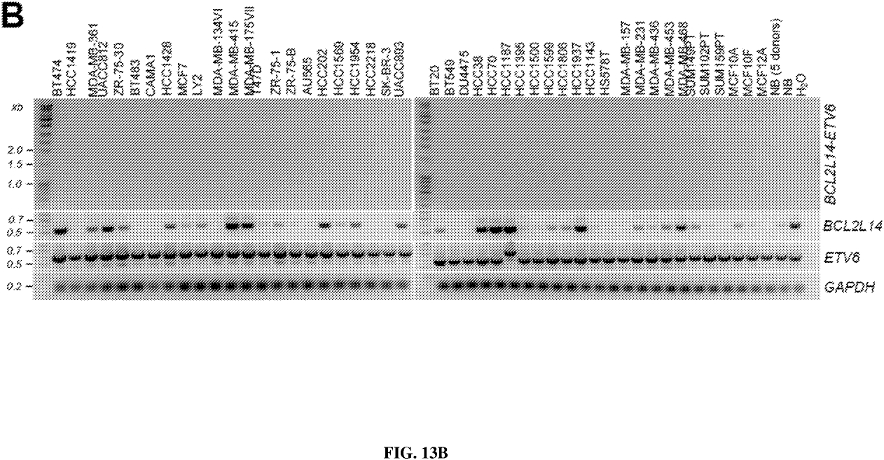

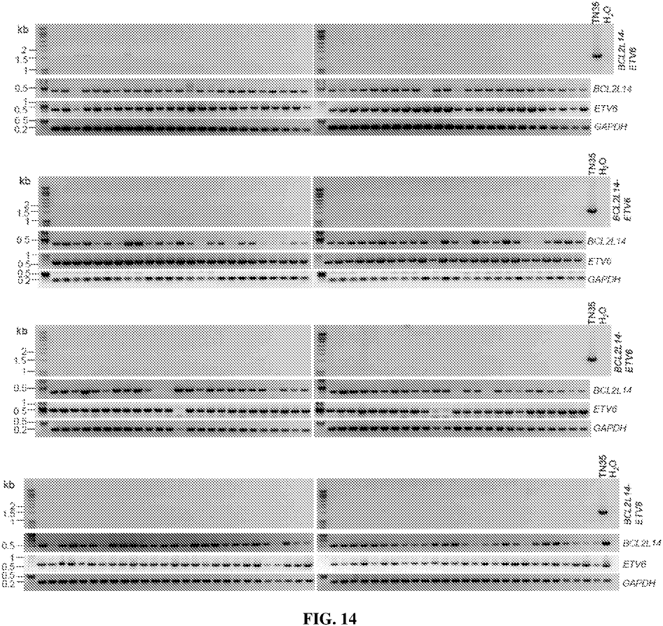

The clinicopathology features for all the 134 TNBC patients from Pitt and BCM cohorts are provided in Table 4. The fusion positive cases were subsequently verified by capillary sequencing. Next, the expression of BCL2L14-ETV6 was tested in a panel of 44 breast cancer cell lines and 34 TNBC PDX tumors. One PDX tumor that expresses BCL2L14-ETV6 was detected but not in the cell lines tested (FIGS. 13A and 13B). The most common fusion variant detected is the fusion between exon 4 of BCL2L14 and exon 2 of ETV6 (referred to as E4E2) that present in two patient cases and one PDX tumor. BCL2L14- ETV6 was also tested by RT-PCR in 200 ER+ breast tumor tissues from the BCM cohort but no fusion-positive ER+ tumors were detected, which supports its TNBC specificity (FIG. 14).

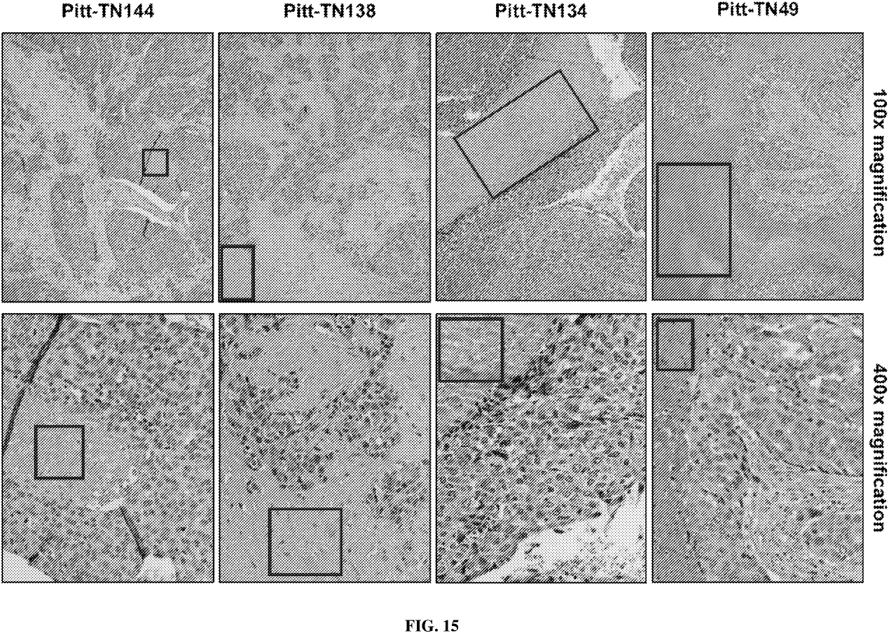

To assess if the BCL2L14-ETV6 positive tumors present the histopathological features discussed above, histopathological evaluations were performed for the four index tumors from the Pitt cohort for which the tissue sections are available. All four tumors are reported as grade 3 tumors with high nuclear pleomorphism score and high mitotic count score (Table 5). In addition, two out of four fusion-positive tumors present extensive necrosis and the remaining two fusion-positive tumors present focal necrosis (FIG. 15), consistent with the above findings.

### Example 6. Characterization Of BCL2L14-ETV6 Genomic Rearrangements and Protein Products

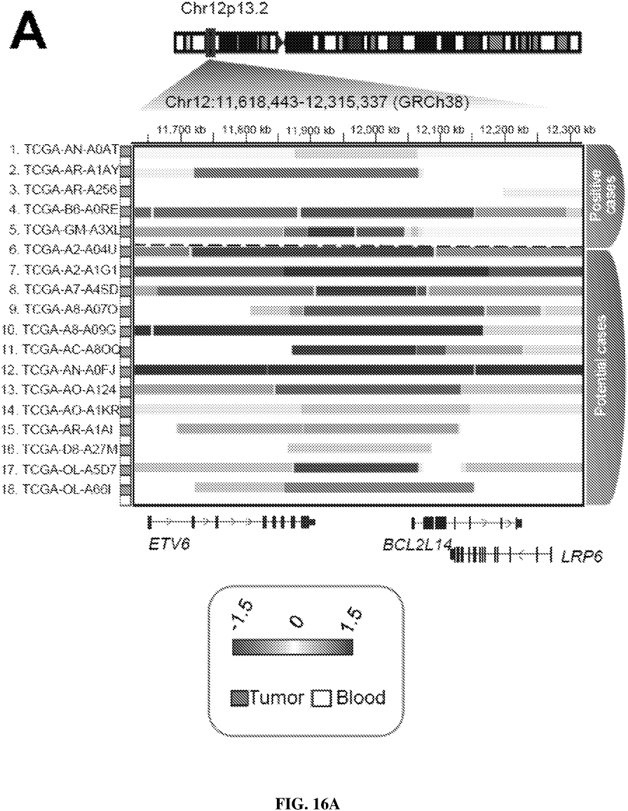

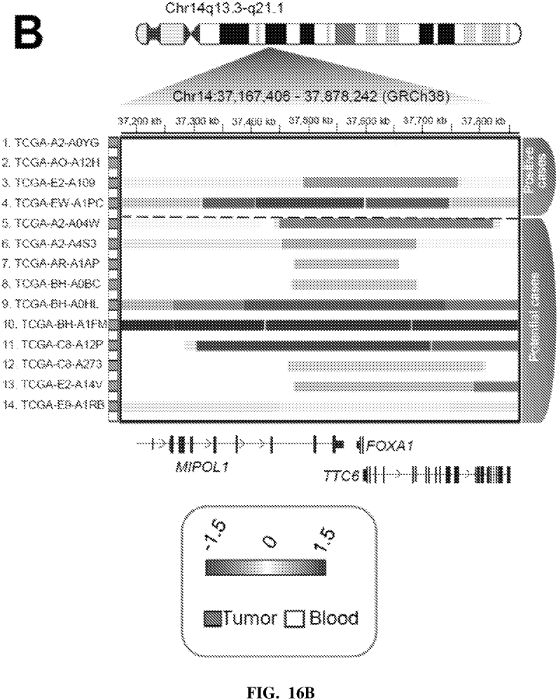

To verify the genomic origin of BCL2L14-ETV6 in the positive cases, genomic PCR was performed using tiling primers designed specifically for BCL2L14 or ETV6 intron regions predicted to harbor the rearrangement based on the fusion variants detected in the index cases from BCM cohort. This assay successfully amplified the genomic fusion points in both of the BCL2L14-ETV6 positive tumors in the BCM cohort (FIG. 2C). The breakpoint junctions in the genomic DNA were further verified by capillary sequencing. Next, the association of BCL2L14-ETV6 with copy number aberrations in the TCGA cohort was explored. Copy number data revealed frequent somatic tandem duplications in the ETV6/BCL2L14 loci, which are present in four out of the five positive TCGA tumors detected by WGS data (FIG. 16A). In addition, copy number data also revealed tandem duplications delineating the ETV6/BCL2L14 loci in the TCGA tumors that were not profiled by WGS, indicating these as positive cases. These data indicate that BCL2L14-ETV6 fusions can be the result of either tandem duplications, or reciprocal rearrangements that generate both BCL2L14-ETV6 and ETV6-BCL2L14 fusions (FIG. 1E), as with the ESR1-CCDC170 fusion we identified (Veeraraghavan J, et al. (2014)).

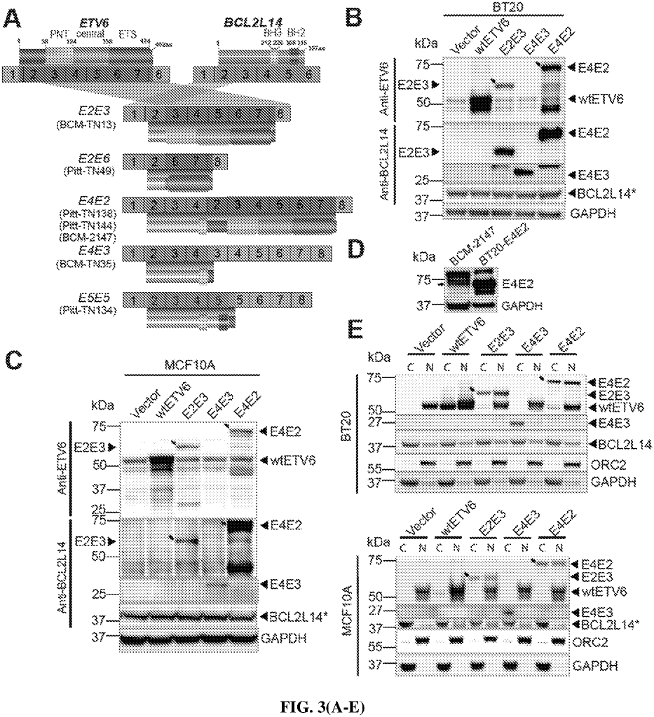

Next, the structure of BCL2L14-ETV6 proteins was investigated. Among five variants detected, three 10 variants (E2E3, E2E6 and E4E2) encode chimeric proteins containing the amino-terminus (N-terminus) of BCL2L14 and the carboxyl-terminus (C-terminus) of ETV6 (FIG. 3A). The ETV6 protein contains an N-terminal pointed (PNT) domain responsible for protein partner binding, and a C-terminal DNA-binding (ETS) domain critical for DNA binding-dependent transcriptional repressor function. Both the most common variant E4E2 and the E2E3 variants retain the PNT domain and ETS domain, whereas the E2E6 protein only retains the ETS domain. E4E3 and E5E5, on the other hand, do not translate the protein sequence of ETV6 due to a frameshift after the fusion junction, resulting in expression of C-terminus truncated BCL2L14 proteins.

Next, the open reading frames (ORFs) of the fusion variants E2E3, E4E3 and E4E2 were ectopically expressed in the fusion-negative MCF10A breast epithelial cell line and the BT20 basal-like breast cancer cell line, both of which are triple-negative in (ER, PR and HER2) receptor expression (Chavez KJ, Garimella SV, & Lipkowitz S (2010)). Cells transduced with the vector containing the lacZ gene or the vector containing the wtETV6 ORF were used as controls. Western blot using polyclonal antibodies against the C-terminus of ETV6 or the N-terminus of BCL2L14 detected strong expression of the E2E3 (62 kD) and E4E2 (74 kD) proteins in the transduced BT20 and MCF10A cells (FIGS. 3B and 3C). The 27 kDa E4E3 fusion protein was detected by the BCL2L14 antibody, but not by the ETV6 antibody, indicating that this variant encodes a truncated BCL2L14 protein, which does not contain the ETV6 protein sequence. Next, the endogenous BCL2L14-ETV6 fusion protein was detected in the PDX tumor expressing the E4E2 variant (BCM-2147). Western blot using the ETV6 antibody detected the same-sized band of E4E2 protein as in the engineered BT20 cells (FIG. 3D).

Since gene fusions tend to translocate to abnormal cellular compartments (9), the cellular localization of the fusion proteins was investigated compared to wild-type (wt) ETV6 protein in the transduced BT20 and MCF10A cells. Due to the lack of specific antibody against BCL2L14-ETV6 that can be used for immunofluorescence, we performed fractionation of the fusion overexpressing cells and detected the fusion protein localizations by western blots. Interestingly, the E2E3 and E4E2 fusion proteins tend to be enriched in the cytoplasm fraction, while wtETV6 mainly presents in the nucleus, in line with its role as a transcription factor. The E4E3 fusion that expresses the truncated BCL2L14 protein was found to be enriched in the cytoplasm as well (FIG. 3E). Differential localization of the fusion proteins from wtETV6 indicates that BCL2L14-ETV6 fusion proteins can function in a distinct cellular mechanism compared to wtETV6. The BCL2L14 portion of the fusion variants can promote cytoplasm localization of the fusion proteins.

### Example 7. BCL2L14-ETV6 Endows Enhanced Invasiveness and Paclitaxel Resistance

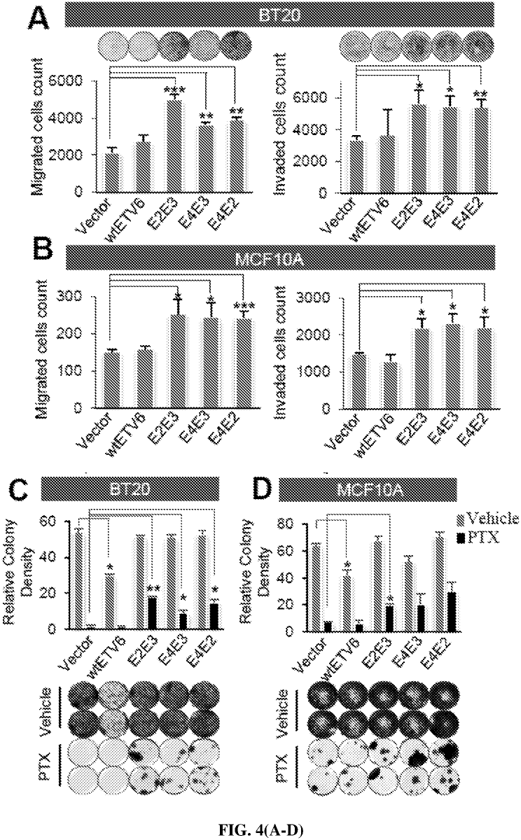

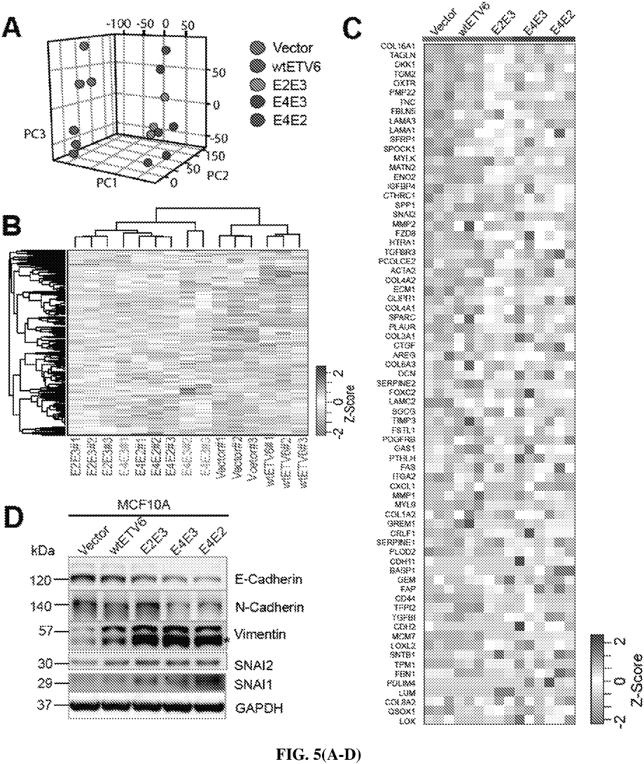

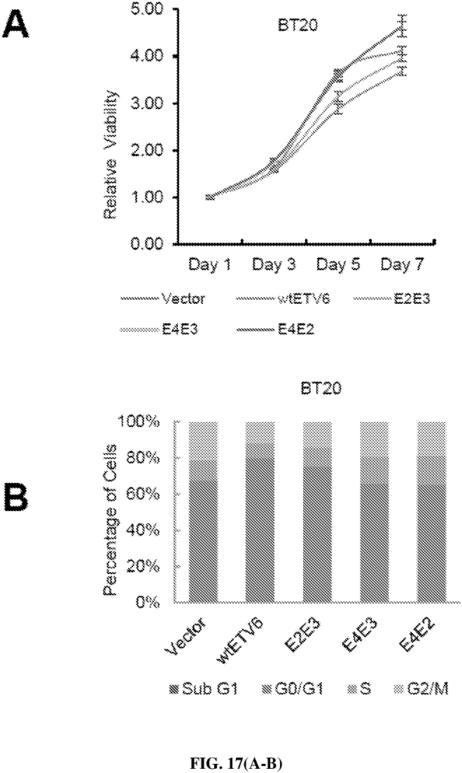

The function of the BCL2L14-ETV6 fusion was examined in the engineered BT20 and MCF10A cell lines. Among TNBC cell lines, BT20 is a non-metastatic, chemo-sensitive line (Ottewell PD (2015), Lucantoni F (2018)) overexpressing Ecadherin (Hajra KM (2002)). This line was thus selected for studying the more aggressive and chemo-resistant phenotypes driven by this fusion. MCF10A is an immortal but untransformed Human Mammary Epithelial Cell (HMEC) line. Both MCF10A and BT20 cell lines express endogenous ETV6 and BCL2L14 proteins (FIGS. 3B and 3C). Transwell migration and invasion assays revealed that ectopic expression of the E2E3, E4E3 or E4E2 fusion variants but not wtETV6 significantly enhanced cell motility and invasion in BT20 cells, when compared to vector control (FIG. 4A). Similarly, enhanced cell motility and invasion (FIG. 4B) were also observed in the engineered MCF10A cells expressing these fusion variants. On the other hand, ectopic expression of the fusion variants in BT20 cells did not result in significant changes in cell viability or cell cycle progression, whereas the wtETV6-expressing BT20 cells showed decreased viability and increased G0/G1 phase (FIGS. 17A and 17B).

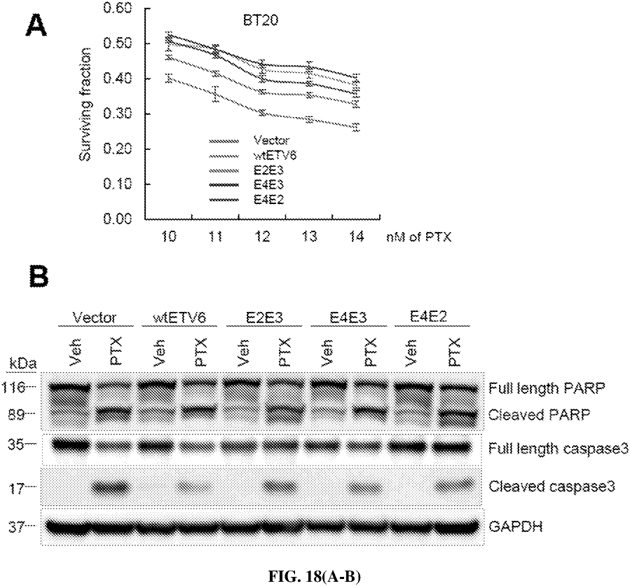

Taxane-based chemotherapy remains the cornerstone for the treatment of TNBC patients, however, the effectiveness is severely limited by intrinsic and acquired resistance. Since BCL2L14-ETV6 mostly frequently present in the mesenchymal TNBC tumors that are relatively resistant to chemotherapy (Park JH, Ahn JH, & Kim SB (2018)), the role of BCL2L14-ETV6 in chemoresistance was explored. First, the engineered BT20 cells were treated with various doses of paclitaxel, a widely used taxane drug for TNBC patients. BCL2L14-ETV6 fusion-expressing BT20 cells displayed modest reduced sensitivity to paclitaxel following short-term (72 h) treatment, compared to the vector or wtETV6-expressing cells (FIG. 18A). The effect of low-dose prolonged paclitaxel treatment was then tested to observe acquired resistance. Following paclitaxel treatment for one month, BT20 cells expressing wtETV6 or vector control were almost eradicated, whereas all fusion expressing BT20 cells showed evident clonal resistance (FIG. 4C). Similarly, the engineered MCF10A cells expressing BCL2L14-ETV6 fusions also showed increased clonal resistance to paclitaxel, compared to vector- or wtETV6-expressing MCF10A cells (FIG. 4D). These results indicate the role of BCL2L14-ETV6 fusions in endowing paclitaxel resistance in TNBC. Since BCL2L14 is an apoptosis facilitator, if the fusion can act through impairing the apoptotic pathway was tested. The changes in apoptosis biomarkers were thus examined following paclitaxel treatment. The BT20 cells overexpressing BCL2L14-ETV6 fusions did not show evident reduced apoptosis compared to wtETV6- expressing cells (FIG. 18B). This indicates that the paclitaxel resistance driven by this fusion is not attributed to the apoptotic pathway.

### Example 8. BCL2L14-ETV6 Fusions Induce Distinctive Expression Changes from wtETV6

To systematically profile the expression changes induced by BCL2L14-ETV6, transcriptome sequencing of BT20 cells stably expressing the vector, wtETV6, or BCL2L14-ETV6 variants, was performed. Principal Component Analysis (PCA) revealed that the vector- and wtETV6-expressing cells form distinctive and independent clusters, whereas the BT20 cells expressing the different fusion variants are clustered together far from both the vector- and wtETV6-expressing cells (FIG. 5A). Further, hierarchical clustering analysis revealed that the engineered BT20 cells were clustered into two main clusters, with the vector control or wtETV6-expressing cells as one major cluster and fusion-expressing cells as the other major cluster (FIG. 5B). These data indicate that BCL2L14-ETV6 fusions induced distinct gene expression changes from wtETV6 and vector control in BT20 cells. It is interesting to note that while the E4E3 fusion variant encodes the C-terminus truncated BCL2L14 protein, this variant induced a similar pattern of expression changes as the E2E3 and E4E2 variants that encode chimeric BCL2L14-ETV6 protein, indicating that these distinct fusion variants can play a coherent functional role.

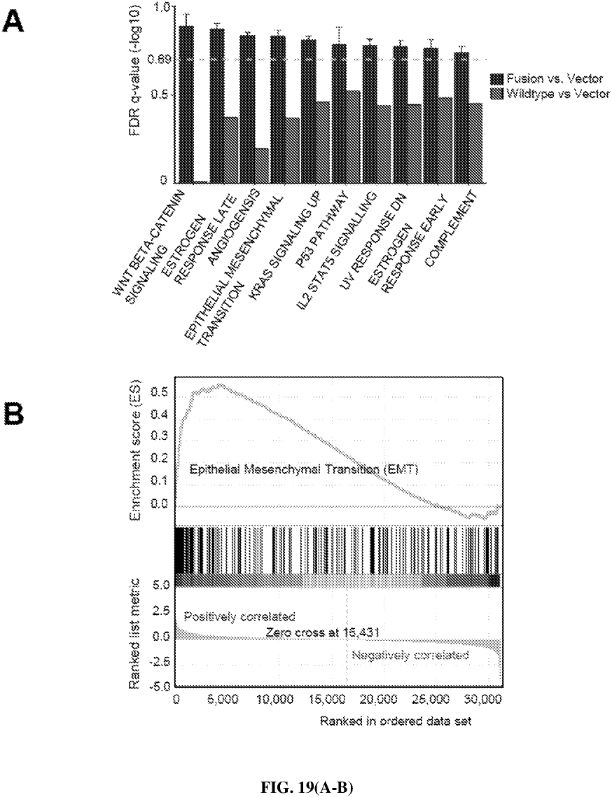

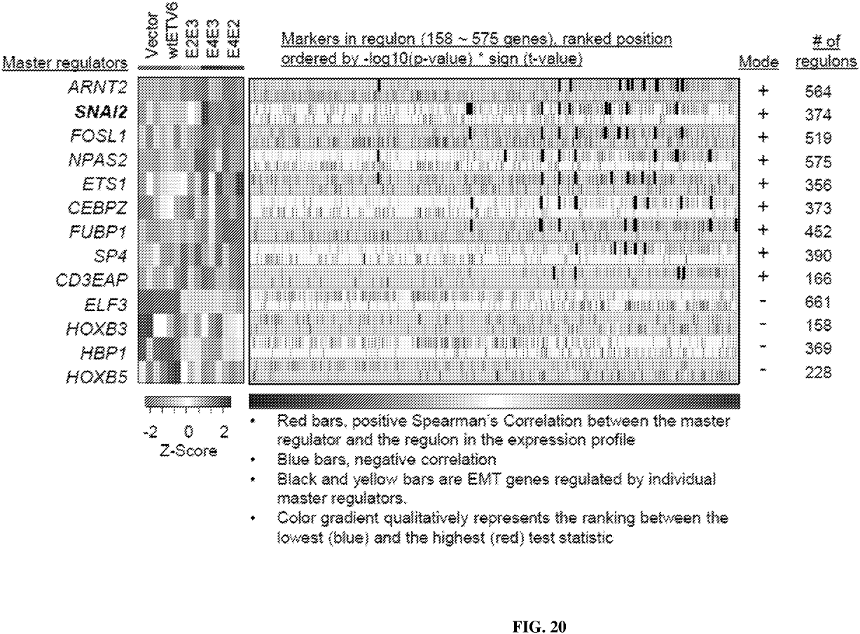

To identify the pathways characteristic of BCL2L14-ETV6 expressing BT20 cells, Gene Set Enrichment Analysis (GSEA) was performed comparing the three fusion variants with the vector control in pairwise. The epithelial mesenchymal transition (EMT) pathway known to promote paclitaxel resistance and invasiveness is among the top upregulated pathways in BT20 cells expressing BCL2L14-ETV6 (FIGS. 19A and 19B). Among the core enrichment genes, 73 EMT pathway genes were up-regulated in the fusion-expressing BT20 cells (FIG. 5C). These results indicate that BCL2L14- ETV6 fusions can induce upregulation of EMT gene signature. To investigate the transcriptional regulatory mechanisms that regulate the EMT gene signature driven by BCL2L14-ETV6, breast cancer cell line BT20-specific transcriptional regulatory network was constructed using ARACNe algorithm (31), and master regulator analysis (MRA) was performed. Among the 13 predicted master regulator candidates, SNAI2 is an established EMT inducing transcription factor (FIG. 20). The snail family genes SNAI1 (also denoted as SNAIL) and SNAI2 (also denoted as SLUG) are known to activate EMT and repress epithelial genes in tumors, including in breast cancer (Hajra KM (2002), Mani SA, et al. (2008)).

### Example 9. BCL2L14-ETV6 Fusions Prime Epithelial Mesenchymal Transition

Next, the expression of EMT biomarkers was explored in the engineered MCF10A and BT20 cells by Western blots, including E-Cadherin, N-Cadherin, and vimentin. Loss of E-cadherin represents the first step of EMT transition (Tsubakihara Y & Moustakas A (2018)). Both MCF10A and BT20 expressing vector control strongly express E-cadherin, indicateing their epithelial states (FIGS. 5D-5F). In fusion expressing MCF10A cells, the expression level of E-cadherin is repressed, whereas the expression level of vimentin, an end-stage marker in EMT (Brabletz T, 2018), but not N-Cadherin, was increased (FIG. 5D). In addition, consistent with MRA result, increased protein levels of SNAI2 and its family member SNAI1 were observed in fusion-expressing MCF10A cells. In the engineered BT20 cells, E-Cadherin is repressed in all fusion-expressing models, but not in the wtETV6 model. Upregulation of N-Cadherin and SNAI1/SNAI2 were also observed in fusion-expressing BT20 cells, however, there is no induction of vimentin following fusion overexpression (FIG. 5E). The fusion-specific induction of SNAI½ transcriptional factors and EMT markers became more obvious when the BT20 cells were treated with TGFβ-1 and EGF known to induce EMT (Buonato JM, Lan IS, & Lazzara MJ (2015)) (FIG. 5F). Loss of the epithelial marker E-cadherin and gain of one of the mesenchymal markers, N-cadherin or vimentin in MCF10A or BT20 cells indicate that the cells are having partial instead of full activation of EMT.

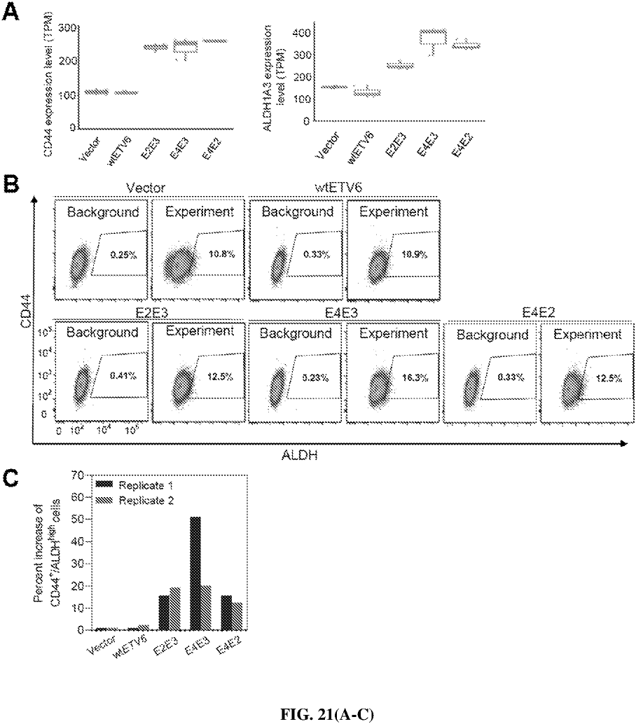

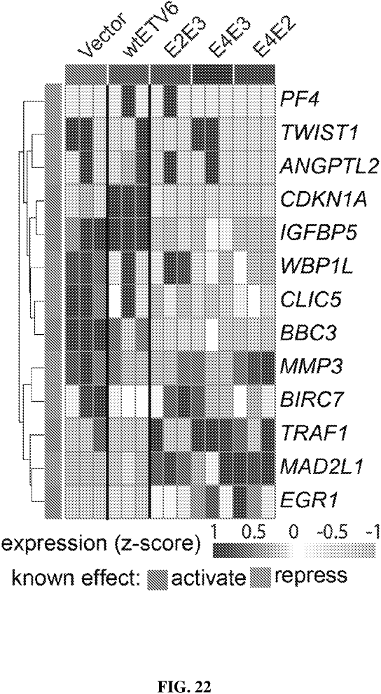

Since EMT is often associated with sternness properties (Brabletz T, et al. (2001)) known to promote clonal chemoresistance (Al-Ejeh F, et al. (2011)), the expression of the known stemness biomarkers, CD44 and ALDH1A3, for breast cancer was examined (de Beca FF, et al. (2013)) in the BT20 models. The RNA-seq data revealed increased expression of CD44 and ALDH1A3 in fusion expressing BT20 cells compared to vector or wtETV6-expressing BT20 cells (FIG. 21A). Consistently, flowcytometry analysis revealed higher number of CD44+/ALDH1^{high} cell populations in fusion expressing BT20 cells, compared to vector or wtETV6 controls (FIGS. 22B and 22C). Together, these results support the role of BCL2L14-ETV6 in inducing partial EMT in TNBC cells.

TNBC comprises 10-20% of all breast cancers. Due to lack of well-defined molecular targets, treatment of TNBC tumors relies on taxane and platinum-based chemotherapies. Despite the distinctive receptor status, recent genomic sequencing studies have revealed a paucity of TNBC-specific mutations, apart from a distinctive mutational enrichment pattern from other breast cancers such as more frequent TP53 mutations and less frequent PIK3CA mutations (Shi Y (2018)). While recent transcriptomic and genomic sequencing studies have revealed oncogenic gene fusions in TNBC patients, some of these can be non-recurrent and can be considered individual fusions, such as MAGI3-AKT3 and FGFR3-TACC3 (Shaver TM, et al. (2016), Mosquera JM, et al. (2015), Banerji S, et al. (2012)), whereas others tend to fuse with promiscuous partners such as Notch and MAST fusions, which can be considered as gene family fusions (Robinson DR, et al. (2011)). Until date, canonical gene fusions of the same fusion partners that recur in a significant subset of TNBC patients have not been reported. Identification of TNBC-specific genetic events that can guide the treatment decisions in this aggressive subtype of breast cancer represents an unmet clinical need.

Despite the complexity and heterogeneity of structural rearrangements in breast cancer (Fimereli D, et al. (2018), Stephens PJ, et al. (2009)), the systematic analyses of somatic structural rearrangements based on WGS data cataloged 99 recurrent gene fusions in breast cancer. Among the different types of rearrangements, it was found that AGR represents a special type of cryptic rearrangement that can occur more frequently than realized in breast cancer. Such cryptic genomic changes are hardly detectable by conventional cytogenetic assays or by transcriptome sequencing. For these reasons, AGRs can only be confidently detected from WGS datasets. Further studies revealed that the top recurrent AGRs are more frequently enriched in specific more aggressive forms of breast cancer that lack well defined drivers, such as basal or luminal B breast cancer. These AGRs tend not to aggregate in the genomically unstable tumors indicating them as pathological events instead of merely the consequence of genomic instability. Among the top four confirmed recurrent gene rearrangements BCL2L14-ETV6, AKAP8-BRD4, TTC6- MIPOL1 and ESR1-CCDC170, BCL2L14-ETV6 is frequently and specifically detected in TNBC which we chose to perform further functional studies. For the TTC6-MIPOL1 rearrangement, while the tandem duplication delineating this fusion encompasses the immediately proximal FOXA1 gene, it is unlikely that one copy number gain can significantly enhance FOXA1 expression. In addition, two out of four TTC6-MIPOL1 positive TCGA tumors do not exhibit copy number changes in the FOXA1 locus (FIG. 15B). Future studies can be required to further evaluate the function of this fusion in luminal breast cancer.

Next, in-depth functional studies were performed on the BCL2L14-ETV6 fusion. This fusion was first experimentally validated in two independent TNBC patient cohorts, which identified six BCL2L14-ETV6 positive cases out of a total of 134 TNBC cases. Taking together WGS data and RT-PCR validation results, this fusion was detected in 4.4-12.2% of TNBC tumors (with an average of 6.2%) from four independent patient cohorts (Table 1). Further investigation of histopathological associations in the TCGA and COSMIC cohorts revealed that this fusion is preferentially present in the TNBC tumors with gross necrosis and more aggressive histopathological features such as marked nuclear pleomorphism, numerous mitoses and high tumor grade (FIG. 1F). Such association is further verified by evaluating pathological slides for the fusion positive cases from the Pitt cohort. All these cases are grade III TNBCs with extensive or focal necrosis. It is interesting to note that, RT-PCR of wild-type ETV6 also revealed ETV6 exon duplications in TNBC cell lines or PDX tumors. These include exon 2 duplication of ETV6 in two PDX tumors and in HCC1187, and exon 4 duplication of ETV6 in one PDX tumor (FIGS. 13A and 13B). This indicates that ETV6 genetic aberrations can involve both intergenic and intragenic rearrangements.

While it remains to be addressed whether DNA repair deficiency can promote the formation of this fusion, our biological studies indicate that BCL2L14-ETV6 fusions appear to enhance cell mobility and invasiveness, and promote paclitaxel resistance when ectopically expressed in basal-like HMEC cell line and non-metastatic, chemo-sensitive TNBC cell line models. In addition, transcriptome sequencing revealed that despite encoding distinct protein products, the three fusion variants induced coherent transcriptional program that is distinctive from wild-type ETV6. Of note, while TCGA copy number data indicate genomic amplifications of the ETV6 genomic loci in a subset of breast tumors harboring BCL2L14-ETV6 tandem duplications (FIG. 16A), ectopic overexpression of wild-type ETV6 did not elicit increased cell migration, invasion, or paclitaxel resistance in TNBC cells (FIG. 4). The observed genomic amplifications can be secondary events following formation of this fusion to enhance its function.

Furthermore, the data indicate that the breast cancer cells overexpressing BCL2L14-ETV6 show a characteristic enrichment of EMT signature. EMT is known to confer stemness features and thus induce invasiveness and chemoresistance in TNBC (Mani SA, et al. (2008), Fedele M, Cerchia L, & Chiappetta G (2017)). The data indicate that BCL2L14-ETV6 fusion proteins can prime for partial EMT instead of full activation of EMT. Tumor cells in partial EMT state are in a state of plasticity that favor metastasis and chemoresistance (Karaosmanoglu O (2018)), and are frequently observed in TNBC (Sarrio D, et al. (2008)). Consistently, BCL2L14-ETV6 fusions are mostly frequently detected in the mesenchymal (M) subtype of TNBC tumors that is closely associated with EMT (Lehmann BD, et al. (2011), Park JH, Ahn JH, & Kim SB (2018)). In this study, the function of BCL2L14-ETV6 was compared with wtETV6 as the major fusion variant E4-E2 and E2-E3 retain most of the ETV6 domains whereas the c terminal truncated BCL2L14 portion lacks intact BCL2-like domain. Further the paclitaxel resistance driven by this fusion does not seem to be attributable to the changes in apoptosis signaling (FIG. 18B).

While it can be interesting to study the endogenously expressed fusion protein in the BCM-2147 PDX model, technical difficulties exist for genetic inhibition studies in many PDX tumors, including BCM-2147. First, the knockdown studies can require rescue experiments to verify the specificity of the siRNAs, which need to be performed on stable cell lines. There are no less than six laboratories attempt to generate cell lines from our BCM PDX models, including laboratories that have generated stable cell lines from primary tissue previously. Thus far, it has not been possible to generate cell lines from any PDX model tested. Although methods have been established for lentiviral transduction for shRNA-mediated knockdown in PDX, the transduction rate is about 30-50% - unlike established cell lines where the infection rate typically exceeds 95%. Given this low transduction rate, shRNA mediated knockdown and genome editing with CRISPR is very inefficient. Further, whereas a majority PDX models can re-transplant after dissociation to single cells, which is required for lentiviral transduction, BCM- 2147 does not re-transplant under all the dissociation conditions tested.

In summary, the data herein revealed adjacent gene rearrangements as class of cryptic genetic events that is more frequent than realized in breast cancer.

### Example 10. Modulation of ETV6 Target Genes By BCL2L14-ETV6

Next, it was determined whether BCL2L14-ETV6 differentially modulates ETV6 target genes compared to wild-type ETV6. To date, most if not all of the studies of ETV6 target genes focus on leukemia. Literature investigation revealed 13 established ETV6 target genes: MMP3, PF4, EGR1, TRAF1, BBC3, CDKN1A, IGFBP5, MAD2L1,TWIST1, CLIC5, ANGPTL2, BIRC7, and WBP1L. RNAseq data revealed that among these genes, CDKN1A and IGFBP5, are repressed by BCL2L14-ETV6, but activated by wtETV6 (FIG. 22). As CDKN1A (p21) is known to induce cell cycle arrest, this modulatory effect is consistent with the repression of cell growth by wtETV6 but not by BCL2L14-ETV6. On the other hand, WBP1L, CLIC5, and BBC3 are more potently repressed by BCL2L14-ETV6 compared to wtETV6, whereas BIRC7, TRAF1, MAD2L1, and EGR1 are activated by BCL2L14-ETV6 but not by wtETV6. The activation or repression of ETV6 target genes by BCL2L14-ETV6 or wtETV6 do not follow the previous reported regulatory effects. These suggest that: a) BCL2L14-ETV6 may lead to re-programing of ETV6 target genes, b) the regulation of ETV6 target genes in the context of breast cancer cells could be distinct from leukemia. It is notable that the E4E3 variant encoding truncated BCL2L14 showed relative consistent regulatory pattern of ETV6 target genes as other variants.

### Example 11. Materials and Methods

To systematically characterize recurrent AGRs in breast cancer, the somatic structural mutation (StSM) data cataloged by the ICGC were analyzed based on WGS data for 215 breast tumors. To detect BCL2L14- ETV6 fusion transcripts, a pair of primers located on exon 2 of BCL2L14 and the last exon of ETV6 were designed respectively, and RT-PCR was performed on 134 triple negative breast tumors, including 45 tumors procured from the Tumor Bank at Baylor College of Medicine, and 89 tumors procured from the Health Sciences Tissue Bank of University of Pittsburgh. The primer sequences and PCR conditions are provided in Table 6. The full-length cDNAs of BCL2L14-ETV6 fusion variants (E2E3, E4E3 and E4E2) were amplified from fusion positive tumors, and engineered into a lentiviral pLenti7.3 vector (Invitrogen). BCL2L14-ETV6 protein products were detected by western blots and the antibodies are provided in Table 7. Transwell migration and Matrigel invasion assays were performed to assess cell invasiveness, and clonogenic assays were performed to assess cell viability following paclitaxel treatment. Transcriptome sequencing of the engineered BT20 cells was performed on the NovaSeq 6000 system. The RNAseq data are made available through Gene Expression Omnibus (GSE120919).

Analyses of whole genome sequencing data. To systematically catalog recurrent AGRs in breast cancer, we analyzed the somatic structural mutation (StSM) data cataloged from WGS data for 215 breast tumor patient cohort released by the ICGC. The StSM variant calling files (.vcf) are downloaded from ICGC portal (dcc.icgc.org/repositories, files labeled “dRanger_snowman” or “svfix2”). Using customized Perl scripts, the somatic structural mutations annotated as “PASS” in the “FILTER” column were first mapped with the human exome to reveal the genes and exons affected by the rearrangements (genome build GRCh37), then the fusion partners were determined based on the strands and genomic regions retained in the rearrangements. For mapping the exons, a merged exon database was created based on the exon annotations from GENCODE (www.gencodegenes.org/) and UCSC genome browser (genome.ucsc.edu/) (V27lift37). The exon numbers for each are assigned based on their starting and ending positions with the exon closest to 5′ of the gene assigned as exon 1. The promoter region for each gene is defined as 3 kb upstream of its transcription starting site. As authentic recurrent gene fusions usually present distinct genomic breakpoints in different patients, we assessed the median absolute deviations of the genomic breakpoint locations for each recurrent gene fusion. The gene fusions with breakpoint deviations of less than 10 bp on each fusion partner gene are excluded from the following analyses, which are the result of misalignments. The gene fusions between known homolog genes are also excluded from the following analyses. The resulting recurrent gene fusions were then classified as AGRs, distant intra-chromosomal rearrangements, or inter-chromosomal rearrangements. AGRs are defined as intrachromosomal rearrangements involving genes of less than 500 Kb apart.

Next, the resulting gene rearrangements were ranked by their incidence in the ICGC breast cancer patient cohort, and their concept signature (ConSig) scores (www.cagenome.org/consig/, release 2) which indicate their functional relations underlying cancer computed based on the molecular concepts characteristic of known cancer genes, including ontologies, pathways, interactions, and domains (Wang X-S, et al. (2009)). Here the max ConSig score of the two fusion partner genes is used to represent each gene fusion. Next, the 92 TCGA cases were selected from the 215 ICGC breast cancer cases and the clinicopathological associations of these recurrent gene fusions were explored. For these cases PAM50 subtype and receptor status were obtained from Xena Browser data hub (xenabrowser.net/), histopathological classifications from Heng et al. (Heng YJ, et al. (2017)), weighted genomic instability index (GII) and DDR deficiency scores from Marquard, et al. (Marquard AM, et al. (2015)), TP53, PIK3CA mutation data from cBioPortal (www.cbioportal.org/), and BRCA1 mutation from Yost et al. 2019. The tumor grade is deduced for TCGA tumors using the Nottingham metric (Galea MH (1992)). Using the same pipeline described above, the somatic structural rearrangements detected by WGS data for 516 breast tumors were also analyzed, which are provided by the Catalogue of Somatic Mutations in Cancer (COSMIC) (Nik-Zainal S, et al. (2016), Forbes SA, et al. (2016)). TCGA TNBC subtyping data were obtained from Lehmann et al. 2016 and Bareche et al. 2018 studies. For COSMIC TNBC subtyping, the online tool, TNBCtype (Chen X, et al. (2012)), was applied on the gene expression data of COSMIC tumors following the TNBC4 subtyping system (BL1, BL2, M, and LAR) (Lehmann BD, et al. (2016)).

Tissue procurement and RNA extraction. 45 triple-negative and 200 ER+ breast tumor tissues were obtained from the Tumor Bank of Lester and Sue Smith Breast Center at Baylor College of Medicine. 34 triple-negative patient-derived xenografts were kindly provided by Dr. Michael Lewis (Neelakantan D, et al. (2017)). 89 triple-negative and 141 ER+ breast tumors were gained from the Health Sciences Tissue Bank of University of Pittsburgh. Total RNA for normal breast tissues (5-Donor Pool) was purchased from BioChain. Cell lines’ RNA were prepared from the breast cancer cell lines previously obtained from the NCI-ATTC ICBP 45 cell line kit. Total RNA was extracted from the tissues or cell lines using TRIzol reagent (Invitrogen) according to the manufacturer’s instruction.

RT-PCR and genomic PCR. Complementary DNA was synthesized using SuperScript IV Reverse Transcriptase (Invitrogen). For amplification of GAPDH, RT-PCR was performed with GoTaq G2 DNA Polymerase (Promega), for amplification of BCL2L14, ETV6, AKAP8-BRD4 and TTC6-MIPOL1, RT-PCR was performed using Platinum Taq DNA Polymerase High Fidelity (Invitrogen), for amplification of BCL2L14-ETV6 fusions, RT-PCR or genomic PCR was performed with Expand Long Range dNTPack (Roche). PCR products from genomic PCR were purified for capillary sequencing (Macrogen). The primer sequences and PCR conditions are provided in Table 6.

Cell culture. MCF10A human breast epithelial cells and BT20 breast cancer cells were obtained from and authenticated by American Type Culture Collection (ATCC). 293 FT cells used for lentivirus packaging were purchased from Invitrogen. MCF10A and 293 FT cells were cultured as previously described (Veeraraghavan J, et al. (2014)). BT20 cells were cultured in EMEM (ATCC) with 10% fetal bovine serum (FBS, HyClone).

Stable BCL2L14-ETV6 expression vector and stable cell lines. The full-length cDNAs of BCL2L14-ETV6 fusion variants (E2E3, E4E3 and E4E2) containing the full-length ORFs were amplified from fusion-positive tumors (BCM-TN13, BCM-TN35 and BCM-2147), using Expand Long Range dNTPack (Roche) and cloning primer sequences provided in Table S10. Wild-type ETV6 full-length cDNA was amplified from ETV6 (NM_001987) human cDNA clone (sc118922, OriGene) using Phusion Hot Start Flex DNA Polymerase (NEB) and cloning primers (Table 6). The BCL2L14-ETV6 fusion or wtETV6 cDNA was subcloned into a lentiviral pLenti7.3 vector (Invitrogen). A control lacZ gene-containing pLenti7.3 vector was provided by the manufacturer (Invitrogen). After validation by capillary sequencing (Eurofins), these constructs were infected by lentivirus into MCF10A or BT20 cells, and stable cell lines containing the constructs were selected using Flow cytometry sorting against GFP selection marker.

Western blot. For immunoblot analysis, total proteins were extracted by homogenizing the cells in NP40 Lysis Buffer supplemented with complete protease inhibitor cocktail tablet (Roche), 1 mM DTT, and 1 mM PMSF. 20~50 micrograms of protein extracts were denatured in sample buffer, separated by SDS-PAGE, and transferred onto a PVDF membrane (GE). The membranes were blocked and then incubated for 1 h at room temperature or overnight at 4° C. with primary antibodies, followed by incubation with respective horseradish peroxidaseconjugated secondary antibody. The signals were then visualized by the enhanced chemiluminescence system (Clarity Western ECL Substrate and ChemiDoc imaging system, Bio-Rad). The list of antibodies used for western blots is available in Table 7.

Cellular fractionation assay. Engineered stable MCF10A and BT20 cells transduced with lacZ gene, wtETV6 or BCL2L14-ETV6 fusion-containing vectors were freshly harvested for cellular fractionation assay. Cytoplasmic and nuclear proteins of the cells were separated and extracted using NE-PER Nuclear and Cytoplasmic Extraction Reagents (Thermo Fisher Scientific) as per the manufacturer’s instructions. The extracted proteins were then used for immunoblot analysis.

Transwell cell migration and Matrigel invasion assays. After serum starvation for 24 h in the starvation medium of DMEM/F12 containing 100 ng/ml cholera toxin, 500 ng/ml hydrocortisone and 2% of horse serum, stable MCF10A cells were then seeded at 3.5X10₄ cells for migration or 4X10₅ cells for invasion assay in the reduced growth medium of DMEM/F12 containing 100 ng/ml cholera toxin, 500 ng/ml hydrocortisone and 0.1% BSA in the Boyden chamber insert without or with Matrigel coating (Corning 354480), respectively. Serumenriched medium (DMEM/F12 containing150 ng/ml cholera toxin, 750 ng/ml hydrocortisone, 30 ng/ml EGF, 0.015 mg/ml human insulin and 10% horse serum) was added to the bottom well of the 24-well plate as attractant. Stable BT20 cells were directly seeded at 2.5X10⁴ cells for migration or 5 X10⁴ cells for invasion assay in the reduced growth medium of EMEM containing 0.1% BSA in the upper Boyden chamber without or with Matrigel coating (Corning 354480), respectively. Serum-enriched medium (EMEM containing 20% FBS) was added to the bottom well of the 24-well plate. After 18 h of incubation, migrated/invaded MCF10A or BT20 cells were stained with 0.1% crystal violet in 50% methanol for counting using CCD camera associated microscopy (Olympus) and ImageJ software.

Cell proliferation and clonogenic assays. Engineered stable BT20 cells were seeded at a density of 3,000 cells/well in a 96-well plate. Cell proliferation was measured by MTS assay at different time points using CellTiter 96 AQueous One Solution Cell Proliferation Assay (Promega). For paclitaxel dose curve, stable BT20 cells were seeded at a density of 5000 cells/well in a 96-well plate and treated with vehicle or different doses of paclitaxel. Cell proliferation was measured by MTS assay after 72 hours of treatment. For clonogenic assay, stable BT20 or MCF10A cells were seeded at a density of 10,000 cells/well in a 24-well plate. After attachment to the plate, cells were treated with 0.1% DMSO (vehicle) or paclitaxel at 5 nM for BT20 cells for 6 days or 15 nM for MCF10A cells for 5 days before replacement of the chemical with fresh growth medium. The remaining colonies were growing in the plate for one month and then stained with 0.5% crystal violet in 50% ethanol and counted using ChemiDoc photography (Bio-Rad) and ImageJ.

Flow cytometry. For cell cycle analysis, cells were stained with propidium iodide (Sigma) and analyzed using Accuri C6cell analyzer (BD Biosciences). Cell cycle phases were then calculated using FlowJo software. Assessment for the presence of breast cancer stem cells in MCF10A or BT20 cells stably expressing the vector, wtETV6 or BCL2L14-ETV6 fusion was performed via FACS analysis using the AldeRed ALDH detection assay (Millipore Sigma) for detection of ALDH activity and subsequent staining for CD44 cell surface marker using anti-CD44, clone IM7 (eFluor 450, ThermoFisher Scientific) according to the manufacturers’ protocols. Following the staining process, cells were then analyzed with LSRFortessa cell analyzer (BD Biosciences) and FlowJo software.

RNA sequencing and data analysis. The standard procedure of Qiagen RNeasy kit was used to extract total RNA from the BT20 cells stably expressing BCL2L14-ETV6 variants, wtETV6 cDNA or pLenti7.3 vector containing the lacZ gene as control in triplicate experiments. The NovaSeq 6000 library for DNA sequencing was prepared using TruSeq Stranded mRNA Library Prep Kit (Illumina) following the protocol provided by the manufacturer. The final libraries were normalized by quantification with LightCycler 480 II (Roche Applied Science, Indianapolis, IN, USA) and quantification with Bioanalyzer (Agilent, Palo Alto, CA, USA). Final loading concentration was adjusted to 10 pM following the NovaSeq 6000 loading protocol and NovaSeq 6000 S2 Reagent Kit (Illumina) was used for paired-end reads (2×150 bp) sequencing reactions. Sequencing data was given as raw data with a Phred Q30 score of 80 or better. For analysis we used Rsubread (Bioconductor release 3.8) (Liao Y, Smyth GK, & Shi W (2013)) to align sequence reads to reference genome and used edgeR (McCarthy DJ (2012)) and limma (Ritchie ME, et al. (2015)) R packages (Bioconductor release 3.8) to normalize gene expression level to log2 transcripts per million (TPM) (Wagner GP, Kin K, & Lynch VJ (2012)). Sequence reads were aligned to GRCh38 human genome reference sequence and the aligned sequences were mapped to Entrez Genes. After normalization, genes of which expression level is zero across all samples were removed to get 31,084 genes for further pathway analysis.

Principle component, clustering, and pathway analyses. To explore the expression clusters of the engineered BT20 cells, unsupervised hierarchical clustering analysis and Principal Component Analysis (PCA) were performed. Euclidean distance metric was used in hierarchical clustering, and the first three components in PCA. In addition, gene set enrichment analysis (GSEA) (Subramanian A, et al. (2005)) was performed to identify the signaling pathways characteristic of the BT20 cells expressing BCL2L14-ETV6 variants. GSEA analyses comparing BCL2L14-ETV6 variants vs. pLenti73 vector in pairwise, or wtETV6 vs pLenti73 vector were performed using the Hallmark and canonical pathways (C2CP) downloaded from Molecular Signature DataBase (MSigDB) (Liberzon A, et al. (2011)). The mean of normalized enrichment score (NES) and false discovery ate (FDR) was calculated from the pairwise GSEA and set the mean FDR q-value to 0.2 (20%) as the threshold to identify significantly enriched pathways.

Master regulator analysis (MRA). Breast cancer cell line BT20-specific interactome was constructed by aggregating microarray or RNA-seq samples publicly available. A total of 13 data sets were obtained from GEO (including GSE120919), which are comprised of 50 microarray samples, 39 RNA-seq samples, and 12 beadchip samples. For the data normalization, we used SCAN.UPC (Piccolo SR (2013)) R package (release 3.8) on Affymetrix microarray platform datasets, and used Rsubread (Liao Y, Smyth GK, & Shi W (2013)), edgeR (McCarthy DJ (2012)), and Limma (Ritchie ME, et al. (2015)) R packages (release 3.8) on Illumina HiSeq platform datasets as described above. The expression profile datasets were combined with common genes across all samples and corrected batch effects (Johnson WE, Li C, & Rabinovic A (2007)). The combined BT20 expression profile data is available through GEO (GSE123917). Human TFs were collected from Animal Transcription Factor Database 2.0 (Hu H, et al. (2019)), and ARACNe algorithm (Margolin AA, et al. (2006)) was used to construct breast cancer cell line BT20-specific interactome. MRA-Fisher’s exact test (FET) (Lefebvre C, et al. (2010)) inferred the candidate master regulators that regulate EMT gene signature.

Statistical analysis. The associations between BCL2L14-ETV6 fusion and different clinicopathological features of the 516 breast tumors available in COSMIC were analyzed via Fisher’s exact test and P-values were calculated with two-tails. Group wise mutual exclusivity test for the lead recurrent AGRs shown in FIG. 1E was performed with the “Discover” package (Canisius S, Martens JW, & Wessels LF (2016)), using the exclusivity statistics and all somatic gene rearrangements as background. The results of all in vitro experiments were analyzed by Student’s t-tests, and all data are shown as mean ± standard deviation.

Availability of data and materials. The RNA-seq data on BT20 models and combined BT20 expression profile data are available through Gene Expression Omnibus (GSE120919 and GSE123917, respectively).

### REFERENCES

1. Koivunen JP, et al. (2008) EML4-ALK fusion gene and efficacy of an ALK kinase inhibitor in lung cancer. Clin Cancer Res 14(13):4275-4283.
2. Singh D, et al. (2012) Transforming fusions of FGFR and TACC genes in human glioblastoma. Science 337(6099):1231-1235.
3. Cocco E, Scaltriti M, & Drilon A (2018) NTRK fusion-positive cancers and TRK inhibitor therapy. Nat Rev Clin Oncol 15(12):731-747.
4. Matissek KJ, et al. (2018) Expressed Gene Fusions as Frequent Drivers of Poor Outcomes in Hormone Receptor-Positive Breast Cancer. Cancer Discov 8(3):336-353.
5. Hartmaier RJ, et al. (2018) Recurrent hyperactive ESR1 fusion proteins in endocrine therapy-resistant breast cancer. Ann Oncol 29(4):872-880.
6. Giltnane JM, et al. (2017) Genomic profiling of ER(+) breast cancers after short-term estrogen suppression reveals alterations associated with endocrine resistance. Sci Transl Med 9(402).
7. Fimereli D, et al. (2018) Genomic hotspots but few recurrent fusion genes in breast cancer. Genes Chromosomes Cancer 57(7):331-338.
8. Lei JT, et al. (2018) Functional Annotation of ESR1 Gene Fusions in Estrogen Receptor-Positive Breast Cancer. Cell Rep 24(6):1434-1444 e1437.
9. Veeraraghavan J, et al. (2014) Recurrent ESR1-CCDC170 rearrangements in an aggressive subset of oestrogen receptor-positive breast cancers. Nat Commun 5:4577.
10. Guo B, Godzik A, & Reed JC (2001) Bcl-G, a novel pro-apoptotic member of the Bcl-2 family. J Biol Chem 276(4):2780-2785.
11. Rasighaemi P & Ward AC (2017) ETV6 and ETV7: Siblings in hematopoiesis and its disruption in disease. Crit Rev Oncol Hematol 116:106-115.
12. Tognon C, et al. (2002) Expression of the ETV6-NTRK3 gene fusion as a primary event in human secretory breast carcinoma. Cancer Cell 2(5):367-376.
13. Stephens PJ, et al. (2009) Complex landscapes of somatic rearrangement in human breast cancer genomes. Nature 462(7276):1005-1010.
14. Heng YJ, et al. (2017) The molecular basis of breast cancer pathological phenotypes. J Pathol 241(3):375-391.
15. Wang XS, et al. (2009) An integrative approach to reveal driver gene fusions from paired-end sequencing data in cancer. Nat Biotechnol 27(11):1005-1011.
16. Marquard AM, et al. (2015) Pan-cancer analysis of genomic scar signatures associated with homologous recombination deficiency suggests novel indications for existing cancer drugs. Biomark Res 3:9.
17. Canisius S, Martens JW, & Wessels LF (2016) A novel independence test for somatic alterations in cancer shows that biology drives mutual exclusivity but chance explains most co-occurrence. Genome Biol 17(1):261.
18. Van Cruchten S & Van Den Broeck W (2002) Morphological and biochemical aspects of apoptosis, oncosis and necrosis. Anatomia, histologia, embryologia 31(4):214-223.
19. Leek RD, Landers RJ, Harris AL, & Lewis CE (1999) Necrosis correlates with high vascular density and focal macrophage infiltration in invasive carcinoma of the breast. Br J Cancer 79(5-6):991-995.
20. Urru SAM, et al. (2018) Clinical and pathological factors influencing survival in a large cohort of triplenegative breast cancer patients. BMC Cancer 18(1):56.
21. Nik-Zainal S, et al. (2016) Landscape of somatic mutations in 560 breast cancer whole-genome sequences. Nature 534(7605):47-54.
22. Forbes SA, et al. (2016) COSMIC: High-Resolution Cancer Genetics Using the Catalogue of Somatic Mutations in Cancer. Curr Protoc Hum Genet 91:10 11 11-10 11 37.
23. Lehmann BD, et al. (2011) Identification of human triple-negative breast cancer subtypes and preclinical models for selection of targeted therapies. J Clin Invest 121(7):2750-2767.
24. Zhang X, et al. (2013) A renewable tissue resource of phenotypically stable, biologically and ethnically diverse, patient-derived human breast cancer xenograft models. Cancer research 73(15):4885-4897.
25. Neelakantan D, et al. (2017) EMT cells increase breast cancer metastasis via paracrine GLI activation in neighbouring tumour cells. Nat Commun 8:15773.
26. Chavez KJ, Garimella SV, & Lipkowitz S (2010) Triple negative breast cancer cell lines: one tool in the search for better treatment of triple negative breast cancer. Breast Dis 32(1-2):35-48.
27. Ottewell PD, O’Donnell L, & Holen I (2015) Molecular alterations that drive breast cancer metastasis to bone. Bonekey Rep 4:643.
28. Lucantoni F, Lindner AU, O’Donovan N, Dussmann H, & Prehn JHM (2018) Systems modeling accurately predicts responses to genotoxic agents and their synergism with BCL-2 inhibitors in triple negative breast cancer cells. Cell Death Dis 9(2):42.
29. Hajra KM, Chen DY, & Fearon ER (2002) The SLUG zinc-finger protein represses E-cadherin in breast cancer. Cancer Res 62(6):1613-1618.
30. Park JH, Ahn JH, & Kim SB (2018) How shall we treat early triple-negative breast cancer (TNBC): from the current standard to upcoming immuno-molecular strategies. ESMO Open 3(Suppl 1):e000357.
31. Margolin AA, et al. (2006) ARACNE: an algorithm for the reconstruction of gene regulatory networks in a mammalian cellular context. BMC Bioinformatics 7 Suppl 1:S7.
32. Mani SA, et al. (2008) The epithelial-mesenchymal transition generates cells with properties of stem cells. Cell 133(4):704-715.
33. Tsubakihara Y & Moustakas A (2018) Epithelial-Mesenchymal Transition and Metastasis under the Control of Transforming Growth Factor beta. Int J Mol Sci 19(11).
34. Brabletz T, Kalluri R, Nieto MA, & Weinberg RA (2018) EMT in cancer. Nat Rev Cancer 18(2): 128-134.
35. Buonato JM, Lan IS, & Lazzara MJ (2015) EGF augments TGFbeta-induced epithelial-mesenchymal transition by promoting SHP2 binding to GAB1. J Cell Sci 128(21):3898-3909.
36. Brabletz T, et al. (2001) Variable beta-catenin expression in colorectal cancers indicates tumor progression driven by the tumor environment. Proc Natl Acad Sci U S A 98(18):10356-10361.
37. Al-Ejeh F, et al. (2011) Breast cancer stem cells: treatment resistance and therapeutic opportunities. Carcinogenesis 32(5):650-658.
38. de Beca FF, et al. (2013) Cancer stem cells markers CD44, CD24 and ALDH1 in breast cancer special histological types. J Clin Pathol 66(3):187-191.
39. Shi Y, Jin J, Ji W, & Guan X (2018) Therapeutic landscape in mutational triple negative breast cancer. Mol Cancer 17(1):99.
40. Shaver TM, et al. (2016) Diverse, biologically relevant, and targetable gene rearrangements in triplenegative breast cancer and other malignancies. Cancer research 76(16):4850-4860.
41. Mosquera JM, et al. (2015) MAGI3-AKT3 fusion in breast cancer amended. Nature 520(7547):E11-12.
42. Banerji S, et al. (2012) Sequence analysis of mutations and translocations across breast cancer subtypes. Nature 486(7403):405-409.
43. Robinson DR, et al. (2011) Functionally recurrent rearrangements of the MAST kinase and Notch gene families in breast cancer. Nat Med 17(12):1646-1651.
44. Wang XS, et al. (2011) Characterization of KRAS rearrangements in metastatic prostate cancer. Cancer Discov 1(1):35-43.
45. Fedele M, Cerchia L, & Chiappetta G (2017) The Epithelial-to-Mesenchymal Transition in Breast Cancer: Focus on Basal-Like Carcinomas. Cancers (Basel) 9(10).
46. Karaosmanoglu O, Banerjee S, & Sivas H (2018) Identification of biomarkers associated with partial epithelial to mesenchymal transition in the secretome of slug overexpressing hepatocellular carcinoma cells. Cell Oncol (Dordr) 41(4):439-453.
47. Sarrio D, et al. (2008) Epithelial-mesenchymal transition in breast cancer relates to the basal-like phenotype. Cancer Res 68(4):989-997.
48. Schmid P, et al. (2018) Atezolizumab and Nab-Paclitaxel in Advanced Triple-Negative Breast Cancer. N Engl J Med 379(22):2108-2121.

### ADDITIONAL REFERENCES

1. Wang X-S, et al. (2009) An integrative approach to reveal driver gene fusions from paired-end sequencing data in cancer. Nature biotechnology 27(11):1005.
2. Heng YJ, et al. (2017) The molecular basis of breast cancer pathological phenotypes. J Pathol 241(3):375-391.
3. Marquard AM, et al. (2015) Pan-cancer analysis of genomic scar signatures associated with homologous recombination deficiency suggests novel indications for existing cancer drugs. Biomark Res 3:9.
4. Yost S, Ruark E, Alexandrov LB, & Rahman N (2019) Insights into BRCA Cancer Predisposition from Integrated Germline and Somatic Analyses in 7632 Cancers. JNCI Cancer Spectr 3(2):pkz028.
5. Galea MH, Blamey RW, Elston CE, & Ellis IO (1992) The Nottingham Prognostic Index in primary breast cancer. Breast cancer research and treatment 22(3):207-219.
6. Nik-Zainal S, et al. (2016) Landscape of somatic mutations in 560 breast cancer whole-genome sequences. Nature 534(7605):47-54.
7. Forbes SA, et al. (2016) COSMIC: High-Resolution Cancer Genetics Using the Catalogue of Somatic Mutations in Cancer. Curr Protoc Hum Genet 91:10 11 11-10 11 37.
8. Lehmann BD, et al. (2016) Refinement of triple-negative breast cancer molecular subtypes: implications for neoadjuvant chemotherapy selection. PLoS One 11(6):e0157368.
9. Bareche Y, et al. (2018) Unravelling triple-negative breast cancer molecular heterogeneity using an integrative multiomic analysis. Annals of Oncology 29(4):895-902.
10. Chen X, et al. (2012) TNBCtype: a subtyping tool for triple-negative breast cancer. Cancer informatics 11:CIN. S9983.
11. Neelakantan D, et al. (2017) EMT cells increase breast cancer metastasis via paracrine GLI activation in neighbouring tumour cells. Nat Commun 8:15773.
12. Veeraraghavan J, et al. (2014) Recurrent ESR1-CCDC170 rearrangements in an aggressive subset of oestrogen receptor-positive breast cancers. Nat Commun 5:4577.
13. Liao Y, Smyth GK, & Shi W (2013) The Subread aligner: fast, accurate and scalable read mapping by seed-and-vote. Nucleic Acids Res 41(10):e108.
14. McCarthy DJ, Chen Y, & Smyth GK (2012) Differential expression analysis of multifactor RNA-Seq experiments with respect to biological variation. Nucleic Acids Res 40(10):4288-4297.
15. Ritchie ME, et al. (2015) limma powers differential expression analyses for RNA-sequencing and microarray studies. Nucleic Acids Res 43(7):e47.
16. Wagner GP, Kin K, & Lynch VJ (2012) Measurement of mRNA abundance using RNA-seq data: RPKM measure is inconsistent among samples. Theory Biosci 131(4):281-285.
17. Subramanian A, et al. (2005) Gene set enrichment analysis: a knowledge-based approach for interpreting genome-wide expression profiles. Proc Natl Acad Sci U S A 102(43): 15545-15550.
18. Liberzon A, et al. (2011) Molecular signatures database (MSigDB) 3.0. Bioinformatics 27(12):1739-1740.
19. Piccolo SR, Withers MR, Francis OE, Bild AH, & Johnson WE (2013) Multiplatform single-sample estimates of transcriptional activation. Proc Natl Acad Sci U S A110(44): 17778-17783.
20. Johnson WE, Li C, & Rabinovic A (2007) Adjusting batch effects in microarray expression data using empirical Bayes methods. Biostatistics 8(1):118-127.
21. Hu H, et al. (2019) AnimalTFDB 3.0: a comprehensive resource for annotation and prediction of animal transcription factors. Nucleic Acids Res 47(D1):D33-D38.
22. Margolin AA, et al. (2006) ARACNE: an algorithm for the reconstruction of gene regulatory networks in a mammalian cellular context. BMC Bioinformatics 7 Suppl 1:S7. 22
23. Lefebvre C, et al. (2010) A human B-cell interactome identifies MYB and FOXM1 as master regulators of proliferation in germinal centers. Mol Syst Biol 6:377.
24. Canisius S, Martens JW, & Wessels LF (2016) A novel independence test for somatic alterations in cancer shows that biology drives mutual exclusivity, but chance explains most co-occurrence. Genome Biol 17(1):261.

TABLE 1

Incidence of BCL2L14-ETV6 gene fusion detected in four different patient cohorts of 942 breast tumors

| | | | Fusion positive frequency by TNBC (%) | | | Frequency by Tumor Grade in TNBC (%) | | Frequency by TNBC subtypes (%) | | | |
|---|---|---|---|---|---|---|---|---|---|---|---|
| Cohort | Method | Total | | | | | | | | | |
| | | | non-TNBC | TNBC | necrotic TNBC | Low | High | BL1 | BL2 | M | LAR |
| TCGA | WGS | 92 | 0/48(0) | 5/41(12.2) | 3/23(13.0) | 0/5(0) | 4/29(13.8) | 3/16(18.8) | 0/5(0) | 2/11(18.2) | 0/7(0) |
| COSMIC | WGS | 516 | 0/345(0) | 10/162(6.2) | 4/48(8.3) | 0/14(0) | 7/133(5.3) | 2/27(7.4) | ⅒(10) | 3/15(20.0) | 0/10(0) |
| PITT | RT-PCR | 89 | – | 4/89(4.5) | 4/4* | 0/10(0) | 4/79(5.1) | – | | | |
| BCM | RT-PCR | 245 | 0/200(0) | 2/45(4.4) | – | 0/12(0) | 2/26(7.7) | – | | | |
| Total | | 942 | 0/593(0) | 21/337 (6.2) | 7/71(9.9) | 0/41(0) | 17/267(6.4) | 5/43(11.6) | 1/15(6.7) | 5/26(19.2) | 0/17(0) |

*only four BCL2L14-ETV6 positive cases from the Pitt cohort are analyzed for pathological features which are not counted in the overall frequencies in necrotic TNBC.

TABLE 2

The list of somatic structural rearrangements detected in 215 breast tumors of the ICGC breast cancer patient cohort

| No. | Gene Fusion | Fusion Type | Chromosome Type | Recurrence (n=215) | 5′-3′ placement¹ | 5′-3′ Distance (Kb) | ICGC_DonorID-and_Location_of_Somatic_Structural_Rearrangement² | 5′_ConSig | 3′_ConSig |
|---|---|---|---|---|---|---|---|---|---|
| 1 | TTC6-MIPOL1 | AGR | adjacent | 0.032 | << | 43 | DO217826(Primarytumor):intron1-intron38(14:38065729[14:38004172[) DO218605(Primarytumor):intron6-intron28(14:38095961[14:37788175[) DO218611(Primarytumor):intron1-intron35(14:38073154[14:37932702[) DO3158(Primarytumor):intron13-intron28(14:38198896[14:37825724[) DO4036(Primarytumor):intron7-intron9(14:38150022[14:37693157[) DO4766(Primarytumor):intron23-intron35(14:38260989[14:37920697[) DO6231(Primarytumor):intron20-intron28(14:38224160[14:37796881[) | 0.299 | 0.839 |
| 2 | BCL2L14-ETV6 | AGR | neighbor | 0.028 | << | 154 | DO2509(Primarytumor):intron7-intron3(12:12212245[12:11864954[) DO2783(Primarytumor):intron7-intron12(12:12209588[12:12021796[) DO3482(Primarytumor):intron7-intron13(12:12217725[12:12025877[) DO4155(Primarytumor):promoter-exon17(12:12201582[12:12044506[) DO44111(Primarytumor):intron7-intron12(12:12212154]12:12007006]) D052556(Primarytumor):intron8-intron4(12:12222514[12:11871872[) | 0.540 | 2.152 |
| 3 | ESR1-CCDC170 | AGR | neighbor | 0.023 | << | 35 | DO2078(Primarytumor):intron3-intron1(6:152073376[6:151852057[) DO218684(Primarytumor):intron3-intron7(6:152024963[6:151874146[) DO2706(Primarytumor):intron3-intron6(6:152091431[6:151867735[) DO3037(Primarytumor):intron25-intron6(6:152362257[6:151869392[) DO3412(Primarytumor):intron1-intron8(6:151992865[6:151905264[) | 3.583 | 0.578 |
| 4 | AKAP8-BRD4 | AGR | adjacent | 0.018 | >> | 21 | DO1010(Primarytumor):intron16-intron5(]19:15442115]19:15476571) DO1328(Primarytumor):intron21-intron5(]19:15407775]19:15467249) DO2551(Primarytumor):intron16-intron5(]19:15434015]19:15477600) DO44273(Primarytumor):exon23-intron5(]19:15409685]19:15464471) | 1.189 | 1.680 |
| 5 | TENM4-SHANK2 | Intra-chr | intrachr | 0.018 | >> | 7405 | DO2593(Primarytumor):intron7-intron44(]11:70350717]11:78899878) DO3037(Primarytumor):intron16-intron22(11:70717629]11:78760493]) DO3182(Primarytumor):intron34-intron24(]11:70658609]11:7894682) DO3500(Primarytumor):ntron37-intron22([11:70692323[11:78457052) | 0.303 | 1.904 |
| 6 | COL14A1-DEPTOR | AGR | adjacent | 0.014 | << | 9 | DO2706(Primarytumor):intron2-intron9(8:121117275[8:121046021[) DO3614(Primarytumor):intron5-intron1(8:121129065[8:120929323[) DO5745(Primarytumor):exon62-intron3(8:121357633[8:120954697[) | 0.984 | 1.248 |
| 7 | DEPDC1B-PDE4D | AGR | adjacent | 0.014 | >> | 75 | DO1287(Primarytumor):intron6-intron5(]5:59780414]5:59975944) DO1328(Primarytumor):intron6-intron8(]5:59560170]5:59966417) DO52557(Primarytumor):intron14-intron7([5:59637460[5:59923390) | 0.650 | 2.418 |
| 8 | NEMF-LINC01588 | AGR | adjacent | 0.014 | << | 74 | DO1249(Primarytumor):intron12-intron42(14:50440900[14:50307746[) DO2455(Primarytumor):intron50-intron42(14:50431894[14:50257845[) DO52554(Primarytumo):exon1-intron51(14:50395096[14:50319552[) DO52554(Primarytumor):intron6-intron51(14:50395096[14:50319552[) | 0.795 | 0.299 |
| 9 | NPAS3-AKAP6 | AGR | adjacent | 0.014 | << | 97 | DO1001(Primarytumor):intron4-intron37(14:33488181[14:33236778[) DO1287(Primarytumor):intron4-intron42(14:33476337[14:33295402[) DO218651(Primarytumor):intron27-intron41(14:34224215]14:33284108]) | 1.332 | 1.289 |
| 10 | PTK2-AGO2 | AGR | adjacent | 0.014 | >> | 22 | DO2719(Primarytumor):intron48-intron3(]8:141627540]8:141879595) DO3874(Primarytumor):intron41-intron17(]8:141565034]8:141895751) DO44273(Primarytumor):intron21-intron3(]8:141631125]8:141968093) | 2.873 | 1.268 |
| 11 | WWOX-VAT1L | AGR | adjacent | 0.014 | << | 119 | DO1010(Primarytumor):intron20-intron8(16:78243574[16:77938840[) DO2694(Primarytumor):intron34-intron8(16:78496939]16:78000221]) DO2706(Primarytumor):intron37-intron8([16:79223602[16:77930947) | 1.257 | 0.000 |
| 12 | ETV6-BCL2L14 | AGR | neighbor | 0.014 | >> | 158 | DO218684(Primarytumor):intron13-intron32(]12:12029907]12:12303424) DO2995(Primarytumor):intron13-intron30(]12:12025955]12:12264916) DO44111(Primarytumor):intron12-intron7(12:12007003]12:12212157]) | 2.152 | 0.540 |
| 13 | ADIPOR2-ERC1 | AGR | adjacent | 0.009 | << | 193 | DO4359(Primarytumor):intron4-exon64(12:1813904[12:1602975[) DO52561(Primarytumor):intron4-intron56([12:1834351[12:1557756) | 1.006 | 0.920 |
| 14 | ANK3-RHOBTB1 | AGR | adjacent | 0.009 | << | 138 | DO4359(Primarytumor):intron2-intron2(10:62739851[10:62467390[) DO5347(Primarytumor):intron5-intron4([10:62698358[10:62280786) | 2.041 | 1.657 |
| 15 | BAGE2-KMT2C | Inter-chr | interchr | 0.009 | – | – | DO1328(Primarytumor):intron3-intron2(]7:152074596]21:11078187) DO3110(Primarytumor):intron3-intron2(]7:152089903]21:11093661) | 0.410 | 0.000 |
| 16 | BCAS3-DGKE | Intra-chr | intrachr | 0.009 | << | 3814 | DO3188(Primarytumor):intron65-intron16(17:59160086[17:54937986[) DO4473(Primarytumor):intron29-intron14(17:58892284]17:54929860]) | 0.884 | 0.420 |
| 17 | BCAS3-PPM1D | AGR | neighbor | 0.009 | << | 11 | DO218176(Primarytumor):intron30-intron6([17:58914614[17:58724943) DO3188(Primarytumor):intron68-intron6(17:59209102[17:58711734[) | 0.884 | 1.763 |
| 18 | BPTF-PITPNC1 | AGR | adjacent | 0.009 | << | 128 | DO1257(Primarytumor):intron35-intron20(17:65932145[17:65687912[) DO5745(Primarytumor):intron52-intron17(17:65949440[17:65640752[) | 0.907 | 1.952 |
| 19 | BRWD1- DSCAM | Intra-chr | adjacent | 0.009 | << | 691 | DO1018(Primarytumor):promoter-intron1(21:42159541]21:40695140]) DO4018(Primarytumor):intron31-intron13([21:41606647[21:40632488) | 1.476 | 1.848 |
| 20 | BRWD1-ERG | Intra-chr | adjacent | 0.009 | >> | 522 | DO3804(Primarytumor):intron47-intron6(]21:39946153]21:40695518) DO3804(Primarytumor):promoter-intron6(]21:39946153]21:40695518) DO4018(Primarytumor):intron34-intron12(]21:39839490]21:40625418) | 1.476 | 2.775 |
| 21 | CACNA2D3-CACNA1D | AGR | adjacent | 0.009 | << | 309 | DO2629(Metastatictumour):intron11-intron1(3:54357938[3:53389771[) DO5200(Primarytumor):intron26-intron1(3:54960947[3:53535327[) DO5200(Primarytumor):intron37-intron1(3:54960947[3:53535327[) DO5200(Primarytumor):intron37-intron8(3:54960947[3:53535327[) DO5200(Primarytumor):intron46-intron1(3:54960947[3:53535327[) | 1.909 | 1.632 |
| 22 | CCDC82-MAML2 | AGR | neighbor | 0.009 | >> | 10 | DO218828(Primarytumor):intron20-intron1(]11:96054401]11:96095279) DO4233(Primarytumor):intron22-intron1(]11:96048552]11:96091572) | 0.524 | 1.758 |
| 23 | CFDP1-BCAR1 | AGR | adjacent | 0.009 | >> | 26 | DO4155(Primarytumor):intron26-intron39(]16:75268213]16:75328768) DO52538(Primarytumor):intron22-exon2(]16:75300663]16:75344682) | 1.089 | 2.154 |
| 24 | CHMP4B-CBFA2T2 | AGR | adjacent | 0.009 | << | 161 | DO4138(Primarytumor):intron1-intron14(20:32430101[20:32207682[) DO4155(Primarytumor):intron1-intron25(20:32405232[20:32231059[) | 0.532 | 2.054 |
| 25 | CLTC-VMP1 | AGR | adjacent | 0.009 | >> | 10 | DO2706(Primarytumor):intron58-intron14(17:57771059]17:57810513]) DO3188(Primarytumor):intron5-intron19(]17:57709464]17:57815375) | 2.001 | 0.732 |
| 26 | CPEB1-FSD2 | AGR | adjacent | 0.009 | << | 107 | DO2995(Primarytumor):intron7-exon18(15:83427588[15:83315587[) DO3182(Primarytumor):intron31-exon14(15:83434729[15:83225300[) | 0.585 | 0.100 |
| 27 | CSMD1- SNORA79 | Inter-chr | interchr | 0.009 | – | – | DO217907(Primarytumor):intron6-intron1([14:46491214[8:3876296) DO3662(Primarytumor):intron2-intron1([14:65850096[8:4565437) | 0.685 | 0.000 |
| 28 | CSMD3-FAM135B | Intra-chr | intrachr | 0.009 | << | 24693 | DO5012(Primarytumor):intron44-intron40(8:139145230[8:113422263[) DO5200(Primarytumor):intron6-intron16([8:139304761[8:114364750) | 1.910 | 0.232 |
| 29 | DCAF6-MPZL1 | AGR | adjacent | 0.009 | << | 144 | DO4036(Primarytumor):intron8-intron8(1:167908295[1:167729603[) DO4359(Primarytumor):intron21-intron8(1:167979437[1:167728156[) | 0.634 | 1.426 |
| 30 | DLG2-TENM4 | Intra-chr | intrachr | 0.009 | >> | 4014 | DO218168(Primarytumor):intron18-intron3(]11:79037504]11:84520432) DO218168(Primarytumor):intron19-intron5(]11:79037504]11:84520432) DO218168(Primarytumor):intron54-intron3(]11:79037504]11:84520432) DO2712(Primarytumor):intron13-intron18(11:78686696]11:84744114]) | 2.018 | 0.303 |
| 31 | EFCAB10-ATXN7L1 | AGR | adjacent | 0.009 | << | 4 | DO2341(Primarytumor):intron3-intron31(7:105248818[7:105226522[) DO5012(Primarytumor):promoter-exon33(7:105245825]7:105244014]) | 0.000 | 0.285 |
| 32 | EIF4G3-HP1BP3 | AGR | neighbor | 0.009 | >> | 19 | DO2694(Primarytumor):intron12-intron31(]1:21075977]1:21453610) DO52550(Primarytumor):intron17-intron13(]1:21104277]1:21390893) | 0.832 | 0.610 |
| 33 | ERC1-B4GALNT3 | AGR | adjacent | 0.009 | << | 427 | DO2995(Primarytumor):intron45-intron2(12:1459965[12:595783[) DO52561(Primarytumor):intron45-intron2([12:1463569[12:577479) | 0.920 | 0.428 |
| 34 | EXOC4-LRGUK | AGR | adjacent | 0.009 | >> | 61 | DO1006(Primarytumor):intron32-intron3(]7:133445929]7:133822441) DO2694(Primarytumor):intron32-intron12(]7:133411391]7:133865644) | 0.556 | 0.205 |
| 35 | FAM222B-PHF12 | AGR | adjacent | 0.009 | << | 50 | DO1016(Primarytumor):promoter-intron28(17:27243723]17:27183082]) DO5745(Primarytumor):intron13-intron12(17:27261482[17:27109806[) | 0.702 | 2.185 |
| 36 | FBXL20-IKZF3 | AGR | adjacent | 0.009 | << | 355 | DO1013(Primarytumor):intron4-intron5(17:37970402]17:37504933]) DO1013(Primarytumor):intron6-intron8(17:37970402]17:37504933]) DO3188(Primarytumor):intron4-intron3(17:37993548[17:37551608[) | 1.310 | 1.139 |
| 37 | FCHSD2-MIR4300HG | Intra-chr | intrachr | 0.009 | << | 8738 | DO2078(Primarytumor):intron16-intron3([11:82204917[11:72653527) DO52561(Primarytumor):intron23-intron10(11:81782136]11:72573716]) | 1.636 | 0.000 |
| 38 | FGF12-ATP13A4 | Intra-chr | adjacent | 0.009 | << | 634 | DO1287(Primarytumor):intron13-intron39(3:193153986[3:191897805[) DO218168(Primarytumor):intron9-intron8(3:193224639[3:192212217[) | 2.310 | 0.141 |
| 39 | GSE1-KIAA0513 | AGR | adjacent | 0.009 | << | 75 | DO2706(Primarytumor):intron12-intron2(16:85687339[16:85062465[) DO3188(Primarytumor):intron5-intron2([16:85403577[16:85068329) | 0.000 | 0.629 |
| 40 | GTF2IRD1-CLIP2 | AGR | neighbor | 0.009 | << | 48 | DO2252(Primarytumor):intron31-intron1(7:74008603[7:73706329[) DO4155(Primarytumor):intron3-exon32(7:73882660[7:73818591[) | 0.936 | 0.504 |
| 41 | HIBADH-JAZF1 | AGR | adjacent | 0.009 | << | 168 | DO2995(Primarytumor):intron6-intron12(7:27930012[7:27678298[) DO52552(Primarytumor):intron9-intron5(7:28044558[7:27659586[) | 1.083 | 1.984 |
| 42 | IMMP2L-PPP1R3A | Intra-chr | intrachr | 0.009 | << | 2314 | DO218212(Primarytumor):intron14-intron1(7:113595014[7:111049493[) DO2995(Primarytumor):intron7-intron1(7:113713586[7:111174886[) | 0.822 | 0.817 |
| 43 | INTS4-TENM4 | Intra-chr | adjacent | 0.009 | << | 663 | DO218168(Primarytumor):intron10-intron51(11:78377275[11:77695471[) DO2712(Primarytumor):intron44-intron3(11:79077645]11:77595746]) | 0.221 | 0.303 |
| 44 | IQCK-RBFOX1 | Intra-chr | intrachr | 0.009 | << | 120 06 | DO1015(Primarytumor):promoter-intron21(16:19725322[16:7073601[) DO1663(Primarytumor):intron16-intron5(16:19767855[16:5752450[) DO1663(Primarytumor):intron9-intron2(16:19767855[16:5752450[) | 0.680 | 2.330 |
| 45 | KMT2E-LHFPL3 | AGR | adjacent | 0.009 | << | 107 | DO218212(Primarytumor):intron26-intron3(7:104712039[7:104462063[) DO2719(Primarytumor):intron8-intron3(7:104671988[7:104454374[) | 2.117 | 0.798 |
| 46 | KSR2-TAOK3 | AGR | adjacent | 0.009 | << | 181 | DO3958(Primarytumor):intron20-intron9([12:118789278[12:117923774) DO4155(Primarytumor):exon27-intron36(12:118642526[12:117903289[) | 0.788 | 0.846 |
| 47 | LINC00535-FLJ46284 | AGR | adjacent | 0.009 | >> | 334 | DO1016(Primarytumor):intron10-intron2(]8:93832083]8:94379425) DO4473(Primarytumor):intron13-intron4(]8:93777643]8:94354114) | 0.000 | 0.084 |
| 48 | LPIN1-GREB1 | AGR | adjacent | 0.009 | << | 35 | DO2719(Primarytumor):intron1-intron45(2:11831796[2:11773920[) DO52538(Primarytumor):intron40-intron39(2:11942051[2:11759103[) | 0.963 | 1.231 |
| 49 | LRBASH3D19 | AGR | adjacent | 0.009 | << | 87 | DO2455(Primarytumor):intron24-intron2(4:152151154[4:151793256[) DO52550(Primarytumor):intron8-intron2(4:152175098[4:151860027[) | 0.862 | 0.744 |
| 50 | LTBP1-TTC27 | AGR | adjacent | 0.009 | << | 126 | DO4036(Primarytumor):intron21-intron23([2:33483610[2:33006743) DO52559(Primarytumor):intron42-intron15(2:33558125[2:32956413[) | 2.035 | 0.531 |
| 51 | MAP2K4-DNAH9 | AGR | adjacent | 0.009 | << | 51 | DO218560(Primarytumor):intron8-intron84(17:11955867]17:11826001]) DO4766(Primarytumor):intron26-intron91(17:12034860[17:11850961[) | 1.504 | 0.679 |
| 52 | MCF2L2-TNIK | Intra-chr | intrachr | 0.009 | >> | 11718 | DO2341(Primarytumor):intron31-intron5([3:171042167[3:182957839) DO5626(Primarytumor):intron48-intron3(3:171108775]3:182907828]) | 0.447 | 0.729 |
| 53 | MIR4300HG-FCHSD2 | Intra-chr | intrachr | 0.009 | >> | 8738 | DO2078(Primarytumor):intron3-intron16([11:72653527[11:82204917) DO52561(Primarytumor):intron10-intron23(11:72573716]11:81782136]) | 0.000 | 1.636 |
| 54 | MTAP-CDKN2B-AS1 | AGR | adjacent | 0.009 | >> | 57 | DO2694(Primarytumor):exon36-intron17(]9:21933516]9:22069291) DO44273(Primarytumor):exon36-intron5(]9:21934714]9:22025454) | 1.140 | 0.092 |
| 55 | MYO16-TNFSF13B | AGR | adjacent | 0.009 | << | 288 | DO2783(Primarytumor):intron26-intron1(13:109631637[13:108912208[) DO4155(Primarytumor):intron5-intron8(13:109371761[13:108944803[) | 0.590 | 1.004 |
| 56 | NFIX-DAND5 | AGR | neighbor | 0.009 | << | 21 | DO1020(Primarytumor):intron2-intron4(19:13109877[19:13083419[) DO1249(Primarytumor):intron17-intron4(19:13170237[19:13081354[) | 2.567 | 1.032 |
| 57 | NLGN1-NAALADL2 | AGR | adjacent | 0.009 | >> | 152 | DO1384(Primarytumor):intron12-intron16(]3:173415424]3:174506219) DO218404(Primarytumor):intron14-intron10(]3:173804626]3:174250850) | 0.724 | 1.147 |
| 58 | NSD1-ZNF346 | AGR | adjacent | 0.009 | << | 52 | DO218168(Primarytumor):intron31-intron13(5:176692413[5:176473601[) DO4359(Primarytumor):intron14-exon1(5:176614917[5:176449860[) | 2.028 | 0.473 |
| 59 | PDCD4-RBM20 | AGR | adjacent | 0.009 | << | 32 | DO4155(Primarytumor):intron5-intron5(10:112632475[10:112550147[) DO52538(Primarytumor):intron21-intron1([10:112648435[10:112518044) | 1.883 | 0.145 |
| 60 | PGAP3-ASIC2 | Intra-chr | intrachr | 0.009 | >> | 5496 | DO1663(Primarytumor):exon21-intron10(]17:31375458]17:37829425) DO3140(Primarytumor):intron1-intron3([17:32337950[17:37850377) | 0.424 | 0.000 |
| 61 | PITPNC1-BPTF | AGR | adjacent | 0.009 | >> | 128 | DO2706(Primarytumor):intron15-intron26(]17:65593811]17:65913797) DO4473(Primarytumor):intron17-intron59(]17:65660429]17:65971189) | 1.952 | 0.907 |
| 62 | PLEKHF2-NDUFAF6 | AGR | adjacent | 0.009 | << | 17 | DO2078(Primarytumor):intron2-intron3(8:96159845[8:95935270[) DO3182(Primarytumor):intron2-intron22(8:96158359[8:96038422[) | 0.777 | 0.287 |
| 63 | POLE2-LINC01588 | AGR | adjacent | 0.009 | << | 239 | DO1017(Primarytumor):intron11-intron19(14:50482300[14:50135541[) DO1392(Primarytumor):intron10-intron42(14:50436439[14:50137299[) | 1.151 | 0.299 |
| 64 | PPM1D-BCAS3 | AGR | neighbor | 0.009 | >> | 11 | DO1249(Primarytumor):intron3-intron45(]17:58695444]17:59054234) DO218176(Primarytumor):intron6-intron30([17:58724943[17:58914614) | 1.763 | 0.884 |
| 65 | PPM1E-VMP1 | Intra-chr | adjacent | 0.009 | >> | 722 | DO218428(Primarytumor):intron1-intron24(]17:56976434]17:57849335) DO2712(Primarytumor):intron1-intron20(]17:57013855]17:57837692) | 2.002 | 0.000 |
| 66 | PPP2R5D-PTK7 | AGR | adjacent | 0.009 | >> | 64 | DO1291(Primarytumor):intron6-intron7(]6:42966628]6:43056559) DO218669(Primarytumor):intron25-intron7(6:42978187]6:43052277]) | 1.306 | 1.862 |
| 67 | PUS7-SRPK2 | AGR | adjacent | 0.009 | >> | 40 | DO2551(Primarytumor):intron8-intron6(]7:105016285]7:105139286) DO52544(Primarytumor):intron27-intron11(]7:104908647]7:105084644) | 0.749 | 2.017 |
| 68 | RBFOX1-SNX29 | Intra-chr | intrachr | 0.009 | >> | 4266 | DO1663(Primarytumor):intron5-intron27([16:5752452[16:12439276) DO4766(Primarytumor):intron6-intron29(]16:5932502]16:12453023) | 2.330 | 0.540 |
| 69 | RCAN1-RUNX1 | AGR | adjacent | 0.009 | << | 173 | DO218168(Primarytumor):intron4-intron31(21:36230579[21:35963449[) DO4155(Primarytumor):intron6-intron36(21:36198443[21:35910274[) | 1.353 | 3.157 |
| 70 | RECQL4-CPSF1 | AGR | adjacent | 0.009 | >> | 102 | DO1392(Primarytumor):promoter-exon47(8:145620134]8:145743669]) DO2509(Primarytumor):exon24-intron32(]8:145623493]8:145739401) | 1.174 | 0.990 |
| 71 | RIMS1-ADGRB3 | Intra-chr | intrachr | 0.009 | << | 2497 | DO1392(Primarytumor):intron2-intron18(6:72660412[6:69836896[) DO218167(Primarytumor):intron3-intron4(6:72735749]6:69435708]) | 1.435 | 1.676 |
| 72 | RRP7BP-RRP7A | AGR | adjacent | 0.009 | >> | 35 | DO218062(Primarytumor):exon14-exon13([22:42908741[22:42970409) DO44103(Primarytumor):exon11-intron11(]22:42910608]22:42971993) | 0.000 | 0.653 |
| 73 | SGO1AS1-SATB1-AS1 | Intra-chr | intrachr | 0.009 | << | 1254 | DO52554(Primarytumor):intron12-intron127(3:20370113[3:18836872[) DO5312(Primarytumor):intron27-intron120(3:20899192[3:18779999[) | 0.000 | 0.000 |
| 74 | SHANK2-TENM4 | Intra-chr | intrachr | 0.009 | << | 7405 | DO3037(Primarytumor):intron22-intron16(11:78760490]11:70717632]) DO3500(Primarytumor):intron22-intron37(11:78462638[11:70718575[) | 1.904 | 0.303 |
| 75 | SNORA79-CSMD1 | Inter-chr | interchr | 0.009 | – | – | DO217907(Primarytumor):intron1-intron6([8:3876299[14:46491211) DO3662(Primarytumor):intron1-intron2([8:4565437[14:65850096) | 0.000 | 0.685 |
| 76 | SNORA79-TPD52 | Inter-chr | interchr | 0.009 | – | – | DO218457(Primarytumor):intron1-intron43(8:80961869[14:55900957[) DO44111(Primarytumor):intron1-intron5(8:81114312]14:28388262]) | 0.000 | 1.285 |
| 77 | STRBP-DENND1A | AGR | adjacent | 0.009 | << | 111 | DO4138(Primarytumor):intron3-intron29(9:126216788[9:125987528[) DO5745(Primarytumor):intron5-intron6(9:126545648[9:125970671[) | 1.152 | 0.866 |
| 78 | STXBP4-MSI2 | Intra-chr | intrachr | 0.009 | >> | 2082 | DO1015(Primarytumor):promoter-intron42([17:53044297[17:55749922) DO3188(Primarytumor):intron26-intron31(]17:53152080]17:55680068) | 0.561 | 1.988 |
| 79 | TAF4-LAMA5 | AGR | adjacent | 0.009 | << | 242 | DO1392(Primarytumor):intron2-exon18(20:60913295[20:60611742[) DO1663(Primarytumor):intron28-exon6(20:60927094]20:60572582]) | 1.059 | 1.281 |
| 80 | TANC2-EFCAB3 | Intra-chr | adjacent | 0.009 | << | 593 | DO3458(Primarytumor):intron17-intron20(17:61402226]17:60471884]) DO52551(Primarytumor):intron9-intron12(17:61246837[17:60455871[) | 0.513 | 0.181 |
| 81 | TANGO6-CDH1 | AGR | adjacent | 0.009 | << | 8 | DO1392(Primarytumor):intron10-intron27(16:68908101[16:68854753[) DO3110(Primarytumor):intron27-intron9(16:68990318[16:68808339[) | 0.089 | 2.461 |
| 82 | TBC1D31-ZHX2 | AGR | adjacent | 0.009 | << | 67 | DO2455(Primarytumor):intron1-intron1(8:124065471[8:123812786[) DO4138(Primarytumor):intron29-intron1(8:124122822[8:123871053[) | 0.892 | 2.597 |
| 83 | TCF12-TEX9 | AGR | adjacent | 0.009 | << | 473 | DO52544(Primarytumor):intron10-intron1(15:57230074]15:56644799]) DO5696(Primarytumor):intron10-intron1(15:57232311[15:56550337[) | 2.651 | 0.060 |
| 84 | TDG-TMEM132B | Intra-chr | intrachr | 0.009 | >> | 21198 | DO1291(Primarytumor):exon10-intron1([12:104373609[12:125801147) DO4449(Primarytumor):exon1-intron1([12:104359630[12:125801191) | 1.973 | 0.280 |
| 85 | TENM4-DLG2 | Intra-chr | intrachr | 0.009 | << | 4014 | DO218168(Primarytumor):intron10-intron61(11:85279212[11:79072465[) D0218168(Primarytumor):intron3-intron54(11:85279212[11:79072465[) DO218168(Primarytumor):intron3-intron7(11:85279212[11:79072465[) DO2712(Primarytumor):intron18-intron13(11:84744111]11:78686699]) | 0.303 | 2.018 |
| 86 | TENM4-XRRA1 | Intra-chr | intrachr | 0.009 | >> | 3709 | DO2078(Primarytumor):intron29-intron55(]11:74535963]11:78540587) DO2539(Primarytumor):intron22-intron36(11:74615810]11:78606432]) | 0.303 | 0.500 |
| 87 | THADA- ZFP36L2 | AGR | adjacent | 0.009 | >> | 4 | DO1392(Primarytumor):intron53-exon2(]2:43449890]2:43644336) DO3662(Primarytumor):intron49-exon2(]2:43450148]2:43702693) | 0.764 | 1.676 |
| 88 | TM4SF18-WWTR1 | AGR | adjacent | 0.009 | << | 183 | DO2783(Primarytumor):intron11-intron22(3:149288600[3:149042498[) DO2995(Primarytumor):intron9-intron20(3:149367963[3:149043146[) | 0.311 | 1.371 |
| 89 | TMEM132B-TDG | Intra-chr | intrachr | 0.009 | << | 21198 | DO1291(Primarytumor):intron1-exon10([12:125801147[12:104373609) DO4449(Primarytumor):intronl-exon1([12:125801191[12:104359630) | 0.280 | 1.973 |
| 90 | TNIK-MCF2L2 | Intra-chr | intrachr | 0.009 | << | 11718 | DO2341(Primarytumor):intron5-intron31(3:182964904]3:171047362]) DO5626(Primarytumor):intron3-intron48(3:182907825]3:171108778]) | 0.729 | 0.447 |
| 91 | TPD52-SNORA79 | Inter-chr | interchr | 0.009 | – | – | DO218457(Primarytumor):intron43-intron1([14:55901051[8:80961869) DO44111(Primarytumor):intron5-intron 1(]14:28388262]8:81114114) | 1.285 | 0.000 |
| 92 | TPM3P6-ZNF761 | AGR | adjacent | 0.009 | << | 21 | DO1076(Primarytumor):promoter-intron1(19:53981075]19:53943469]) DO5312(Primarytumor):promoter-intron1(19:53982535]19:53946005]) | 0.000 | 0.303 |
| 93 | TPM3P9-ZNF813 | AGR | adjacent | 0.009 | >> | 25 | DO1076(Primarytumor):intron1-intron2(19:53943469]19:53981075]) DO3110(Primarytumor):intron1-intron2(]19:53936713]19:53973069) | 0.000 | 0.000 |
| 94 | UHRF1BP1L-ANKS1B | AGR | adjacent | 0.009 | >> | 44 | DO2694(Primarytumor):intron15-intron14(]12:100076346]12:100485102) DO4155(Primarytumor):exon3-intron21(]12:99745552]12:100536515) | 0.412 | 0.615 |
| 95 | VAT1L-WWOX | AGR | adjacent | 0.009 | >> | 119 | DO2694(Primarytumor):intron8-intron34(16:78000218]16:78496942]) DO2706(Primarytumor):intron8-intron37([16:77930949[16:79223600) | 0.000 | 1.257 |
| 96 | WWTR1-ANKUB1 | AGR | adjacent | 0.009 | << | 24 | DO2341(Primarytumor):intron20-intron5(3:149511973[3:149356030[) DO4359(Primarytumor):intron5-intron 1(3:149630974[3:149421301[) | 1.317 | 0.145 |
| 97 | XPO1-USP34 | AGR | adjacent | 0.009 | >> | 7 | DO2551(Primarytumor):exon49-intron2(]2:61670928]2:61720703) DO44273(Primarytumor):intron25-intron2(]2:61688196]2:61750545) | 2.286 | 1.674 |
| 98 | ZNF143 -IPO7 | AGR | adjacent | 0.009 | << | 12 | DO1392(Primarytumor):intron45-intron34(11:9546356]11:9458003]) DO1392(Primarytumor):intron45-intron35(11:9546356]11:9458003]) DO4695(Primarytumor):exon3-intron38(11:9482600[11:9463027[) | 1.339 | 1.663 |
| 99 | ZNF813 TPM3P9 | AGR | adjacent | 0.009 | << | 25 | DO1076(Primarytumor):intron2-intron1(19:53981075]19:53943469]) DO1537(Primarytumor):intron2-intron1(19:53973597[19:53936784[) | 0.000 | 0.000 |

Note. The values of Recurrence, 5′_ConSig, and 3′_ConSig were rounded off to three decimal places. The table is sorted the largest to the smallest Recurrence.
¹5′-3′_Placement indicates colinear (») or non-colinear (<<) of fusion genes.
² The numbers of intron or exon were based on the positions of all Ensemble exons within each gene.

TABLE 3

Clinical and mutation data of 92 TCGA Tumors of which IDs are matched to the donor IDs of ICGC cohort

| No. | ICGC_ DonorID | TCGA_ID | TumorGrade | PAM50RNAseq | ER_ihc | PR_ihc | HER2_ihc | TNBC_YES/NO | TNBCsubtypes |
|---|---|---|---|---|---|---|---|---|---|
| 1 | DO2783 | TCGA-AN-A0AT | G3 | Basal | – | – | – | YES | BL1 |
| 2 | DO2509 | TCGA-AR-A1AY | G3 | Basal | – | – | – | YES | M |
| 3 | DO4155 | TCGA-AR-A256 | NA | Basal | – | – | – | YES | BL1 |
| 4 | DO3482 | TCGA-B6-A0RE | G3 | Basal | – | – | – | YES | M |
| 5 | DO44111 | TCGA-GM-A3XL | G3 | Basal | – | – | – | YES | BL1 |
| 6 | DO2995 | TCGA-A2-A0D0 | G3 | Basal | – | – | – | YES | BL1 |
| 7 | DO2341 | TCGA-EW-A3U0 | G1 | Basal | – | – | – | YES | UNC |
| 8 | DO2897 | TCGA-BH-A0WA | G3 | Basal | – | – | – | YES | M |
| 9 | DO1559 | TCGA-AO-AOJ4 | NA | Basal | – | – | – | YES | BL1 |
| 10 | DO44103 | TCGA-A2-A3XX | G3 | Basal | – | – | – | YES | BL1 |
| 11 | DO4359 | TCGA-A7-A26G | G2 | Basal | – | – | – | YES | BL2 |
| 12 | DO5312 | TCGA-AQ-A04J | G3 | Basal | – | – | – | YES | BL1 |
| 13 | DO1301 | TCGA-BH-A0B3 | G3 | Basal | – | – | – | YES | LAR |
| 14 | DO5745 | TCGA-GI-A2C9 | NA | Basal | – | – | – | YES | M |
| 15 | DO1384 | TCGA-BH-A0E0 | G3 | Basal | – | – | – | YES | LAR |
| 16 | DO2842 | TCGA-EW-A1P8 | G3 | Basal | – | – | – | YES | BL2 |
| 17 | DO2222 | TCGA-AN-A04D | G2 | Basal | – | – | – | YES | BL1 |
| 18 | DO4695 | TCGA-EW-A1PB | G3 | Basal | – | – | – | YES | BL2 |
| 19 | DO2323 | TCGA-AN-AOGO | G3 | Basal | – | – | – | YES | M |
| 20 | DO1274 | TCGA-D8-A27F | G3 | Basal | – | – | – | YES | M |
| 21 | DO4233 | TCGA-AO-AOJ6 | NA | Basal | – | – | – | YES | BL1 |
| 22 | DO3840 | TCGA-GM-A2DF | G3 | Basal | – | – | – | YES | BL1 |
| 23 | DO5661 | TCGA-BH-A1FC | G3 | Basal | – | – | – | YES | LAR |
| 24 | DO2694 | TCGA-E2-A1LL | G3 | Basal | – | – | – | YES | BL2 |
| 25 | DO3874 | TCGA-E2-A14X | G3 | Basal | – | – | – | YES | BL1 |
| 26 | DO2719 | TCGA-E2-A1LK | G3 | Basal | – | – | – | YES | BL1 |
| 27 | DO4O18 | TCGA-E2-A1LG | NA | Basal | – | – | – | YES | BL2 |
| 28 | DO5375 | TCGA-B6-A0RT | G3 | Basal | – | – | – | YES | BL1 |
| 29 | DO4635 | TCGA-BH-AOAV | G3 | Basal | – | – | – | YES | M |
| 30 | DO1954 | TCGA-B6-A0RU | G3 | Basal | – | – | – | YES | M |
| 31 | DO1537 | TCGA-B6-A0WX | G1 | Basal | – | – | – | YES | M |
| 32 | DO4963 | TCGA-A2-A04P | G3 | Basal | – | – | – | YES | LAR |
| 33 | DO5696 | TCGA-A2-A04T | G3 | Basal | – | – | – | YES | BL1 |
| 34 | DO4080 | TCGA-A7-AOCE | G3 | Basal | – | – | – | YES | LAR |
| 35 | DO3662 | TCGA-BH-AOBW | G3 | Basal | – | – | – | YES | UNC |
| 36 | DO4138 | TCGA-B6-A0l1 | NA | Basal | – | – | NA | NA | NA |
| 37 | DO4557 | TCGA-B6-A0l6 | G2 | Basal | – | – | NA | NA | NA |
| 38 | DO1249 | TCGA-D8-A27H | G3 | Basal | – | – | – | YES | M |
| 39 | DO1287 | TCGA-AO-A124 | NA | Basal | – | – | – | YES | BL1 |
| 40 | DO1328 | TCGA-AC-A2BK | G3 | Basal | – | – | – | YES | M |
| 41 | DO2551 | TCGA-EW-A1PH | G3 | Basal | – | – | – | YES | BL1 |
| 42 | DO44273 | TCGA-A2-A3Y0 | G3 | Basal | + | – | – | NO | NA |
| 43 | DO1392 | TCGA-A7-A13D | G3 | Basal | – | + | – | NO | NA |
| 44 | DO2647 | TCGA-A8-A09X | G2 | HER2 | – | – | – | YES | LAR |
| 45 | DO2252 | TCGA-AO-AOJ2 | NA | HER2 | – | – | – | YES | LAR |
| 46 | DO3110 | TCGA-A8-A08L | G3 | HER2 | + | – | – | NO | NA |
| 47 | DO2593 | TCGA-A8-A094 | G3 | HER2 | + | – | – | NO | NA |
| 48 | DO2055 | TCGA-C8-A12L | G3 | HER2 | – | – | + | NO | NA |
| 49 | DO3958 | TCGA-A2-AOD1 | NA | HER2 | – | – | + | NO | NA |
| 50 | DO3182 | TCGA-C8-A12Q | G3 | HER2 | – | – | + | NO | NA |
| 51 | DO2455 | TCGA-E2-A14P | G3 | HER2 | – | – | + | NO | NA |
| 52 | DO1663 | TCGA-AR-AOTX | G2 | HER2 | + | + | + | NO | NA |
| 53 | DO5249 | TCGA-A2-A04X | G2 | HER2 | + | + | + | NO | NA |
| 54 | DO5012 | TCGA-A8-A08B | G3 | HER2 | + | – | + | NO | NA |
| 55 | DO4449 | TCGA-E2-A152 | G2 | HER2 | + | – | + | NO | NA |
| 56 | DO2712 | TCGA-A8-A07l | G3 | HER2 | + | – | + | NO | NA |
| 57 | DO2078 | TCGA-BH-A18R | G3 | HER2 | Equivocal | – | + | NO | NA |
| 58 | DO27O6 | TCGA-A2-A25B | G2 | LumB | + | + | – | NO | NA |
| 59 | DO3412 | TCGA-AN-AOXR | NA | LumB | + | – | – | NO | NA |
| 60 | DO3188 | TCGA-A8-A09l | G2 | LumB | + | + | + | NO | NA |
| 61 | DO3140 | TCGA-A8-A08S | G1 | LumB | + | + | + | NO | NA |
| 62 | DO5626 | TCGA-AO-AOJM | NA | LumB | + | + | + | NO | NA |
| 63 | DO5200 | TCGA-BH-A18U | G3 | LumB | + | + | + | NO | NA |
| 64 | DO4185 | TCGA-A8-A075 | G3 | LumB | + | + | – | NO | NA |
| 65 | DO1281 | TCGA-AR-A2LK | NA | LumB | + | + | Equivocal | NA | NA |
| 66 | DO3804 | TCGA-C8-A130 | G3 | LumB | + | + | Equivocal | NO | NA |
| 67 | DO3352 | TCGA-E2-A15K | G3 | LumB | + | + | – | NO | NA |
| 68 | DO5808 | TCGA-AO-A03N | NA | LumB | + | + | – | NO | NA |
| 69 | DO4796 | TCGA-A8-A092 | G3 | LumB | + | + | – | NO | NA |
| 70 | DO2114 | TCGA-BH-AOHO | G2 | LumB | + | + | – | NO | NA |
| 71 | DO1291 | TCGA-AR-A24Z | NA | LumB | + | + | – | NO | NA |
| 72 | DO5347 | TCGA-A2-A0D4 | G2 | LumB | + | + | – | NO | NA |
| 73 | DO2O96 | TCGA-B6-A0X5 | G2 | LumB | + | + | – | NO | NA |
| 74 | DO3614 | TCGA-A2-A0EY | G2 | LumB | + | – | + | NO | NA |
| 75 | DO4036 | TCGA-A2-A0YG | G2 | LumB | + | + | + | NO | NA |
| 76 | DO6231 | TCGA-EW-A1PC | G3 | LumB | + | + | – | NO | NA |
| 77 | DO4766 | TCGA-E2-A109 | G3 | LumB | + | – | – | NO | NA |
| 78 | DO3158 | TCGA-AO-A12H | NA | LumA | + | + | – | NO | NA |
| 79 | DO3037 | TCGA-A1-A0SM | G2 | LumA | + | – | + | NO | NA |
| 80 | DO5046 | TCGA-E2-A156 | G2 | LumA | + | + | – | NO | NA |
| 81 | DO2503 | TCGA-BH-AODT | G1 | LumA | + | + | – | NO | NA |
| 82 | DO3152 | TCGA-BH-AOEA | G1 | LumA | + | + | – | NO | NA |
| 83 | DO6144 | TCGA-EW-A1J5 | NA | LumA | + | + | – | NO | NA |
| 84 | DO1290 | TCGA-E9-A1NH | G1 | LumA | + | + | – | NO | NA |
| 85 | DO4719 | TCGA-A2-A259 | G2 | LumA | + | + | – | NO | NA |
| 86 | DO2084 | TCGA-BH-A0H6 | G1 | LumA | + | + | – | NO | NA |
| 87 | DO2629 | TCGA-E2-A15E | G3 | LumA | + | + | + | NO | NA |
| 88 | DO2539 | TCGA-A8-A07B | G2 | LumA | + | + | + | NO | NA |
| 89 | DO3500 | TCGA-A2-A3KC | G1 | LumA | + | + | Equivocal | NO | NA |
| 90 | DO3458 | TCGA-AO-A03L | NA | LumA | + | + | – | NO | NA |
| 91 | DO4473 | TCGA-E2-A15H | G3 | LumA | + | + | + | NO | NA |
| 92 | DO1257 | TCGA-BH-AODG | G2 | LumA | + | – | – | NO | NA |

TABLE 3 (Cont.)

Clinical and mutation data of 92 TCGA Tumors of which IDs are matched to the donor IDs of ICGC cohort

| No. | ICGC_DonorID | Necrosis_in_invasive_portion | PlK3CA_Mutation | BCL2L14-ETV6 | TTC6-MIPOL1 | ESR1-CCDC170 | AKAP8-BRD4 | COL14A1-DEPTOR | DEPDC1B-PDE4D | NEMF-LlNC01588 | PTK2-AGO2 | WWOX-VAT1K | ETV6-BCL2L14 |
|---|---|---|---|---|---|---|---|---|---|---|---|---|---|
| 1 | DO2783 | Ex | – | + | – | – | – | – | – | – | – | – | – |
| 2 | DO2509 | Ex | – | + | – | – | – | – | – | – | – | – | – |
| 3 | DO4155 | NA | – | + | – | – | – | – | – | – | – | – | – |
| 4 | DO3482 | Ab | – | + | – | – | – | – | – | – | – | – | – |
| 5 | DO44111 | Ex | – | + | – | – | – | – | – | – | – | – | + |
| 6 | DO2995 | F | – | – | – | – | – | – | – | – | – | – | + |
| 7 | DO2341 | F | – | – | – | – | – | – | – | – | – | – | – |
| 8 | DO2897 | Ex | – | – | – | – | – | – | – | – | – | – | – |
| 9 | DO1559 | NA | – | – | – | – | – | – | – | – | – | – | – |
| 10 | DO44103 | Ex | – | – | – | – | – | – | – | – | – | – | – |
| 11 | DO4359 | Ab | – | – | – | – | – | – | – | – | – | – | – |
| 12 | DO5312 | F | – | – | – | – | – | – | – | – | – | – | – |
| 13 | DO1301 | Ab | – | – | – | – | – | – | – | – | – | – | – |
| 14 | DO5745 | NA | – | – | – | – | – | + | – | – | – | – | – |
| 15 | DO1384 | Ab | – | – | – | – | – | – | – | – | – | – | – |
| 16 | DO2842 | F | – | – | – | – | – | – | – | – | – | – | – |
| 17 | DO2222 | Ex | – | – | – | – | – | – | – | – | – | – | – |
| 18 | DO4695 | F | – | – | – | – | – | – | – | – | – | – | – |
| 19 | DO2323 | Ex | – | – | – | – | – | – | – | – | – | – | – |
| 20 | DO1274 | Ex | – | – | – | – | – | – | – | – | – | – | – |
| 21 | DO4233 | NA | – | – | – | – | – | – | – | – | – | – | – |
| 22 | DO3840 | Ab | – | – | – | – | – | – | – | – | – | – | – |
| 23 | DO5661 | Ab | – | – | – | – | – | – | – | – | – | – | – |
| 24 | DO2694 | Ex | – | – | – | – | – | – | – | – | – | + | – |
| 25 | DO3874 | F | – | – | – | – | – | – | – | – | + | – | – |
| 26 | DO2719 | Ex | – | – | – | – | – | – | – | – | + | – | – |
| 27 | DO4018 | NA | – | – | – | – | – | – | – | – | – | – | – |
| 28 | DO5375 | Ex | – | – | – | – | – | – | – | – | – | – | – |
| 29 | DO4635 | Ab | – | – | – | – | – | – | – | – | – | – | – |
| 30 | DO1954 | Ab | – | – | – | – | – | – | – | – | – | – | – |
| 31 | DO1537 | Ab | – | – | – | – | – | – | – | – | – | – | – |
| 32 | DO4963 | Ab | MM | – | – | – | – | – | – | – | – | – | – |
| 33 | DO5696 | F | MM | – | – | – | – | – | – | – | – | – | – |
| 34 | DO4080 | Ex | – | – | – | – | – | – | – | – | – | – | – |
| 35 | DO3662 | Ab | – | – | – | – | – | – | – | – | – | – | – |
| 36 | DO4138 | NA | – | – | – | – | – | – | – | – | – | – | – |
| 37 | DO4557 | Ab | – | – | – | – | – | – | – | – | – | – | – |
| 38 | DO1249 | F | – | – | – | – | – | – | – | + | – | – | – |
| 39 | DO1287 | NA | – | – | – | – | – | – | + | – | – | – | – |
| 40 | DO1328 | F | – | – | – | – | + | – | + | – | – | – | – |
| 41 | DO2551 | Ex | – | – | – | – | + | – | – | – | – | – | – |
| 42 | DO44273 | Ex | – | – | – | – | + | – | – | – | + | – | – |
| 43 | DO1392 | Ex | – | – | – | – | – | – | – | – | – | – | – |
| 44 | DO2647 | Ab | – | – | – | – | – | – | – | – | – | – | – |
| 45 | DO2252 | NA | – | – | – | – | – | – | – | – | – | – | – |
| 46 | DO3110 | Ab | MM | – | – | – | – | – | – | – | – | – | – |
| 47 | DO2593 | Ab | – | – | – | – | – | – | – | – | – | – | – |
| 48 | DO2055 | Ex | MM | – | – | – | – | – | – | – | – | – | – |
| 49 | DO3958 | NA | MM | – | – | – | – | – | – | – | – | – | – |
| 50 | DO3182 | F | – | – | – | – | – | – | – | – | – | – | – |
| 51 | DO2455 | F | – | – | – | – | – | – | – | + | – | – | – |
| 52 | DO1663 | Ab | – | – | – | – | – | – | – | – | – | – | – |
| 53 | DO5249 | F | – | – | – | – | – | – | – | – | – | – | – |
| 54 | DO5012 | Ab | MM | – | – | – | – | – | – | – | – | – | – |
| 55 | DO4449 | F | Silent | – | – | – | – | – | – | – | – | – | – |
| 56 | DO2712 | Ab | – | – | – | – | – | – | – | – | – | – | – |
| 57 | DO2078 | Ab | – | – | – | + | – | – | – | – | – | – | – |
| 58 | DO2706 | F | – | – | – | + | – | + | – | – | – | + | – |
| 59 | DO3412 | NA | – | – | – | + | – | – | – | – | – | – | – |
| 60 | DO3188 | Ab | – | – | – | – | – | – | – | – | – | – | – |
| 61 | DO3140 | Ab | – | – | – | – | – | – | – | – | – | – | – |
| 62 | DO5626 | NA | – | – | – | – | – | – | – | – | – | – | – |
| 63 | DO5200 | Ab | – | – | – | – | – | – | – | – | – | – | – |
| 64 | DO4185 | Ab | MM | – | – | – | – | – | – | – | – | – | – |
| 65 | DO1281 | NA | MM | – | – | – | – | – | – | – | – | – | – |
| 66 | DO3804 | Ab | MM | – | – | – | – | – | – | – | – | – | – |
| 67 | DO3352 | Ab | MM | – | – | – | – | – | – | – | – | – | – |
| 68 | DO5808 | NA | MM | – | – | – | – | – | – | – | – | – | – |
| 69 | DO4796 | Ab | MM | – | – | – | – | – | – | – | – | – | – |
| 70 | DO2114 | Ab | – | – | – | – | – | – | – | – | – | – | – |
| 71 | DO1291 | NA | – | – | – | – | – | – | – | – | – | – | – |
| 72 | DO5347 | Ab | – | – | – | – | – | – | – | – | – | – | – |
| 73 | DO2096 | Ab | MM | – | – | – | – | – | – | – | – | – | – |
| 74 | DO3614 | Ab | MM | – | – | – | – | + | – | – | – | – | – |
| 75 | DO4036 | Ab | – | – | + | – | – | – | – | – | – | – | – |
| 76 | DO6231 | Ab | MM | – | + | – | – | – | – | – | – | – | – |
| 77 | DO4766 | F | – | – | + | – | – | – | – | – | – | – | – |
| 78 | DO3158 | NA | – | – | + | – | – | – | – | – | – | – | – |
| 79 | DO3037 | Ab | – | – | – | + | – | – | – | – | – | – | – |
| 80 | DO5046 | Ab | MM | – | – | – | – | – | – | – | – | – | – |
| 81 | DO2503 | Ab | MM | – | – | – | – | – | – | – | – | – | – |
| 82 | DO3152 | Ab | MM | – | – | – | – | – | – | – | – | – | – |
| 83 | DO6144 | NA | MM | – | – | – | – | – | – | – | – | – | – |
| 84 | DO1290 | Ab | MM | – | – | – | – | – | – | – | – | – | – |
| 85 | DO4719 | Ab | – | – | – | – | – | – | – | – | – | – | – |
| 86 | DO2084 | Ab | – | – | – | – | – | – | – | – | – | – | – |
| 87 | DO2629 | F | MM | – | – | – | – | – | – | – | – | – | – |
| 88 | DO2539 | Ab | MM | – | – | – | – | – | – | – | – | – | – |
| 89 | DO3500 | Ab | MM | – | – | – | – | – | – | – | – | – | – |
| 90 | DO3458 | NA | MM | – | – | – | – | – | – | – | – | – | – |
| 91 | DO4473 | Ab | – | – | – | – | – | – | – | – | – | – | – |
| 92 | DO1257 | Ab | – | – | – | – | – | – | – | – | – | – | – |

MM = Missense_Mutation; Ab = Absent; Ext = Extensive; F = Focal

TABLE 4

Clinicopathological features of 134 triple-negative breast cancer cases from the Baylor College of Medicine cohort (N=45) and University of Pittsburgh cohort (N=89). Among the cases, 2 cases from the University of Pittsburgh cohort (Pitt-TN46 and Pitt-TN75) belong to the same patient with tissues excised three years apart

| No. | StudyID | BCL2L14-ETF6 Fusion positive | Age at diagnosis | Sex | Race | Menopause status | Histology | ER status | PR status | HER-2 status | Tumor grade | TNMstating (clinical) | Clinical Stage |
|---|---|---|---|---|---|---|---|---|---|---|---|---|---|
| 1 | BCM-TN1 | No | 72 | F | White | Post | Other | Neg | Neg | Neg | NA | T2N0M0 | 2A |
| 2 | BCM-TN2 | No | 59 | F | White | Post | IDC | Neg | Neg | Neg | 2 | T2N1M0 | 2B |
| 3 | BCM-TN3 | No | 51 | F | White | Post | Carcinoma in-situ | Neg | Neg | Neg | 2 | T1CN0M0 | 1 |
| 4 | BCM-TN4 | No | 53 | F | White | Pre | IDC | Neg | Neg | Neg | 2 | T1CNXM0 | 1 |
| 5 | BCM-TN5 | No | 56 | F | White | Pre | IDC | Neg | Neg | Neg | NA | T1BN0M0 | 1 |
| 6 | BCM-TN6 | No | 66 | F | White | Post | IDC | Neg | Neg | Neg | 3 | T1N0M0 | 1 |
| 7 | BCM-TN7 | No | 39 | F | White | Pre | IDC | Neg | Neg | Neg | 3 | T1N1MX | 2A |
| 8 | BCM-TN8 | No | 53 | F | White | Pre | IDC | Neg | Neg | Neg | 2 | T2N0MX | 2A |
| 9 | BCM-TN9 | No | 49 | F | White | Post | IDC | Neg | Neg | Neg | NA | T1NXM0 | 1 |
| 10 | BCM-TN10 | No | NA | F | White | NA | IDC | Neg | Neg | Neg | 3 | T1N0M0 | 1 |
| 11 | BCM-TN11 | No | 78 | F | White | Post | IDC | Neg | Neg | Neg | NA | T2NXM0 | 2A |
| 12 | BCM-TN12 | No | 69 | F | White | Post | IDC | Neg | Neg | Neg | 3 | T2N1M0 | 2B |
| 13 | BCM-TN13 | Yes | 44 | F | White | NA | IDC | Neg | Neg | Neg | 3 | T2N0M0 | 2A |
| 14 | BCM-TN14 | No | 65 | F | White | Post | IDC | Neg | Neg | Neg | 3 | TisN0M0 | is |
| 15 | BCM-TN15 | No | 63 | F | Asian | NA | IDC | Neg | Neg | Neg | 3 | T2N0M0 | 2A |
| 16 | BCM-TN16 | No | 52 | F | White | Post | IDC | Neg | Neg | Neg | 3 | T1CN2M0 | 3A |
| 17 | BCM-TN17 | No | 69 | F | White | Post | IDC | Neg | Neg | Neg | 3 | T2NXM0 | 2A |
| 18 | BCM-TN18 | No | 28 | F | White | Pre | IDC | Neg | Neg | Neg | 2 | T2N0MX | 2A |
| 19 | BCM-TN19 | No | 60 | F | White | Post | IDC | Neg | Neg | Neg | 2 | T3N0MX | 2B |
| 20 | BCM-TN20 | No | 55 | F | White | Post | IDC | Neg | Neg | Neg | 3 | T2N1M0 | 2B |
| 21 | BCM-TN21 | No | 65 | F | White | NA | IDC | Neg | Neg | Neg | 2-3 | T1N2M0 | 3A |
| 22 | BCM-TN22 | No | 42 | F | White | Pre | IDC | Neg | Neg | Neg | 3 | T2NXMX | 2A |
| 23 | BCM-TN23 | No | 58 | F | White | Post | IDC | Neg | Neg | Neg | 3 | T1CN0M0 | 1 |
| 24 | BCM-TN24 | No | 72 | F | White | Post | IDC | Neg | Neg | Neg | 2 | T3N3M0 | 3C |
| 25 | BCM-TN25 | No | 58 | F | Asian | Post | IDC | Neg | Neg | Neg | 3 | T2N0M0 | 2A |
| 26 | BCM-TN26 | No | 59 | F | White | Post | IDC | Neg | Neg | Neg | NA | T1CN0M0 | 1 |
| 27 | BCM-TN27 | No | 54 | F | White | Pre | IDC | Neg | Neg | Neg | 3 | T3N2M0 | 3A |
| 28 | BCM-TN28 | No | 55 | F | White | Post | IDC | Neg | Neg | Neg | 3 | T2N1M0 | 2B |
| 29 | BCM-TN29 | No | 48 | F | White | Post | Carcinoma in-situ | Neg | Neg | Neg | 2 | T3N2M0 | 3A |
| 30 | BCM-TN30 | No | 59 | F | White | Post | IDC | Neg | Neg | Neg | 2 | T4N0M0 | 3B |
| 31 | BCM-TN31 | No | 58 | F | White | Post | IDC | Neg | Neg | Neg | 2-3 | T2N2M0 | 3A |
| 32 | BCM-TN32 | No | 52 | F | White | Post | IDC | Neg | Neg | Neg | 2 | T4N2MX | 3B |
| 33 | BCM-TN33 | No | 68 | F | White | Post | IDC | Neg | Neg | Neg | 3 | T2NXM0 | 2A |
| 34 | BCM-TN34 | No | 55 | F | White | Post | IDC | Neg | Neg | Neg | NA | T2N2MX | 3A |
| 35 | BCM-TN35 | Yes | 52 | F | White | NA | IDC | Neg | Neg | Neg | 3 | T2N0M0 | 2A |
| 36 | BCM-TN36 | No | 58 | F | White | NA | IDC | Neg | Neg | Neg | 2 | T2N1M0 | 2B |
| 37 | BCM-TN37 | No | 52 | F | NA | Post | IDC | Neg | Neg | Neg | 3 | T3N2M0 | 3B |
| 38 | BCM-TN38 | No | 73 | F | White | Pre | IDC | Neg | Neg | Neg | 2 | T2N1M0 | 2B |
| 39 | BCM-TN39 | No | 57 | F | White | Post | ILC | Neg | Neg | Neg | NA | T1CN0M0 | 1 |
| 40 | BCM-TN40 | No | 54 | F | White | Post | IDC | Neg | Neg | Neg | 2-3 | T2NXM0 | 2A |
| 41 | BCM-TN41 | No | 53 | F | White | NA | IDC | Neg | Neg | Neg | 2-3 | T2N2M0 | 3A |
| 42 | BCM-TN42 | No | 34 | F | White | Pre | IDC | Neg | Neg | Neg | 3 | T2N1M0 | 2B |
| 43 | BCM-TN43 | No | 77 | F | Asian/Pacific Islander | NA | IDC | Neg | Neg | Neg | 2-3 | T3N0M0 | 2B |
| 44 | BCM-TN44 | No | 22 | F | White | Pre | IDC | Neg | Neg | Neg | 3 | T3N0M0 | 2B |
| 45 | BCM-TN45 | No | 57 | F | White | Post | IDC | Neg | Neg | Neg | 3 | NA | NA |
| 46 | PITT-TN46 | No | 58 | F | White | Post | Other | Neg | Neg | Neg | 3 | T2N0M0 | 2A |
| 47 | PITT-TN47 | No | 62 | F | White | Post | IDC | Neg | Neg | Neg | 3 | T2N0M0 | 2A |
| 48 | PITT-TN49 | Yes | 73 | F | White | Post | IDC | Neg | Neg | Equivocal | 3 | T2N0M0 | 2A |
| 49 | PITT-TN50 | No | 44 | F | Black | Pre | IDC | Neg | Neg | Neg | 3 | T3N0M0 | 2B |
| 50 | PITT-TN51 | No | 46 | F | White | Pre | IDC | Neg | Neg | Neg | 3 | T2N0M0 | 2A |
| 51 | PITT-TN52 | No | 53 | F | Black | Post | IDC | Neg | Neg | Neg | 3 | T4DN1M0 | 4 |
| 52 | PITT-TN54 | No | 45 | F | White | Pre | IDC | Neg | Neg | Neg | 3 | T1AN0M0 | 1A |
| 53 | PITT-TN55 | No | 47 | F | Black | Pre | IDC | Neg | Neg | Neg | 3 | T2N0M0 | 2A |
| 54 | PITT-TN56 | No | 71 | F | White | Post | IDC | Neg | Neg | Neg | 3 | T2N0M0 | 2A |
| 55 | PITT-TN58 | No | 53 | F | White | Post | IDC | Neg | Neg | Neg | 3 | T1N1M0 | 2A |
| 56 | PITT-TN60 | No | 81 | F | White | Post | IDC | Neg | Neg | Neg | 3 | T4N1M0 | 3B |
| 57 | PITT-TN61 | No | 63 | F | White | Post | IDC | Neg | Neg | Neg | 3 | T1BN0M0 | 1A |
| 58 | PITT-TN62 | No | 91 | F | White | Post | IDC | Neg | Neg | Neg | 3 | T3N1M0 | 3A |
| 59 | PITT-TN63 | No | 50 | F | White | Post | IDC | Neg | Neg | Neg | 3 | T1BN0M0 | 1A |
| 60 | PITT-TN64 | No | 87 | F | White | Post | IDC | Neg | Neg | Equivocal | 3 | T2N1M0 | 2B |
| 61 | PITT-TN65 | No | 50 | F | Black | NA | IDC | Neg | Neg | Neg | 3 | T1CN0M0 | 1A |
| 62 | PITT-TN66 | No | 62 | F | Black | Post | IDC | Neg | Neg | Neg | 3 | T4BN1M1 | 4 |
| 63 | PITT-TN67 | No | 95 | F | White | Post | IDC | Neg | Neg | Neg | 2 | TplSN0M0 | 0 |
| 64 | PITT-TN68 | No | 88 | F | White | Post | IDC | Neg | Neg | Neg | 3 | T3N0M0 | 2B |
| 65 | PITT-TN70 | No | 63 | F | White | Post | IDC | Neg | Neg | Neg | 3 | T1CN0M0 | 1A |
| 66 | PITT-TN71 | No | 49 | F | White | Pre | IDC | Neg | Neg | Neg | 3 | T2N0M0 | 2A |
| 67 | PITT-TN72 | No | 46 | F | White | Pre | IDC | Neg | Neg | Neg | 3 | T1CN0M0 | 1A |
| 68 | PITT-TN74 | No | 42 | F | White | Pre | IDC | Neg | Neg | Neg | 3 | T2N1M0 | 2B |
| 69 | PITT-TN75 | No | 55 | F | White | Post | IDC | Neg | Neg | Neg | 2 | T2N0M0 | 2A |
| 70 | PITT-TN76 | No | 57 | F | White | Post | IDC | Neg | Neg | Neg | 3 | T1CN0M0 | 1A |
| 71 | PITT-TN77 | No | 44 | F | White | Pre | Other | Neg | Neg | Neg | 3 | T2N0M0 | 2A |
| 72 | PITT-TN78 | No | 48 | F | Black | Post | IDC | Neg | Neg | Neg | 3 | T1CN0M0 | 1A |
| 73 | PITT-TN79 | No | 36 | F | White | NA | IDC | Neg | Neg | Neg | 3 | T2N3AM0 | 3C |
| 74 | PITT-TN80 | No | 47 | F | White | Post | IDC and other | Neg | Neg | Neg | 3 | T2N0M0 | 2A |
| 75 | PITT-TN81 | No | 42 | F | White | Pre | IDC | Neg | Neg | Neg | 3 | T3N1M0 | 3A |
| 76 | PITT-TN83 | No | 52 | F | White | Post | IDC | Neg | Neg | Equivocal | 3 | T1CN0M0 | 1A |
| 77 | PITT-TN84 | No | 38 | F | White | Pre | IDC | Neg | Neg | Neg | 3 | T3N3M0 | 3C |
| 78 | PITT-TN85 | No | 64 | F | White | Post | IDC | Neg | Neg | Neg | 3 | T1AN0M0 | 1 |
| 79 | PITT-TN86 | No | 42 | F | White | Pre | IDC | Neg | Neg | Neg | 3 | T2N0M0 | 2A |
| 80 | PITT-TN87 | No | 39 | F | White | Post | IDC | Neg | Neg | Neg | 3 | T2N1M0 | 2B |
| 81 | PITT-TN88 | No | 80 | F | White | NA | IDC | Neg | Neg | Neg | 3 | T2N0M0 | 2A |
| 82 | PITT-TN89 | No | 54 | F | White | Post | IDC | Neg | Neg | Neg | 3 | T2N0M0 | 2A |
| 83 | PITT-TN90 | No | 49 | F | White | Pre | IDC | Neg | Neg | Neg | 3 | T2N1M0 | 2B |
| 84 | PITT-TN91 | No | 44 | F | White | Post | IDC | Neg | Neg | Neg | 3 | T2N1M0 | 2B |
| 85 | PITT-TN92 | No | 60 | F | White | Post | IDC | Neg | Neg | Neg | 3 | T2N1M0 | 2B |
| 86 | PITT-TN93 | No | 71 | F | Black | Post | IDC | Neg | Neg | Neg | 2 | TplSN0M0 | 0 |
| 87 | PITT-TN95 | No | 84 | F | White | Post | IDC | Neg | Neg | Neg | 3 | TplSN0M0 | 0 |
| 88 | PITT-TN96 | No | 52 | F | White | Post | IDC | Neg | Neg | Equivocal | 3 | T1CN0M0 | 1 |
| 89 | PITT-TN97 | No | 71 | F | White | Post | IDC | Neg | Neg | Neg | 3 | T1CN0M0 | 1 |
| 90 | PITT-TN98 | No | 55 | F | White | Post | IDC | Neg | Neg | Neg | 3 | T2N2M0 | 3A |
| 91 | PITT-TN99 | No | 82 | F | White | Post | IDC | Neg | Neg | Equivocal | 2 | T1CN0M0 | 1A |
| 92 | PITT-TN100 | No | 80 | F | White | Post | IDC | Neg | Neg | Neg | 3 | T2N1M0 | 2B |
| 93 | PITT-TN101 | No | 49 | F | White | Post | IDC | Neg | Neg | Neg | 3 | T1CN0M0 | 1 |
| 94 | PITT-TN102 | No | 38 | F | Black | Pre | IDC | Neg | Neg | Neg | 3 | T2N1M0 | 2B |
| 95 | PITT-TN103 | No | 64 | F | White | Post | IDC | Neg | Neg | Neg | 3 | T1N0M0 | 1 |
| 96 | PITT-TN104 | No | 58 | F | White | Post | IDC | Neg | Neg | Neg | 3 | T1N0M0 | 1 |
| 97 | PITT-TN105 | No | 51 | F | White | Pre | IDC | Neg | Neg | Neg | 3 | T3N1M0 | 3A |
| 98 | PITT-TN106 | No | 44 | F | White | Pre | IDC | Neg | Neg | Neg | 3 | T2N0M0 | 1 |
| 99 | PITT-TN107 | No | 47 | F | Black | Pre | IDC | Neg | Neg | Neg | 3 | T2N0M0 | 2A |
| 100 | PITT-TN108 | No | 78 | F | White | Post | IDC | Neg | Neg | Neg | 2 | T1CN0M0 | 1 |
| 101 | PITT-TN110 | No | 44 | F | White | NA | IDC | Neg | Neg | Neg | 2 | T2N0M0 | 2A |
| 102 | PITT-TN111 | No | 58 | F | White | Post | IDC | Neg | Neg | Neg | 3 | T2N0M0 | 2A |
| 103 | PITT-TN112 | No | 73 | F | White | Post | IDC | Neg | Neg | Neg | 3 | T1CN0M0 | 1 |
| 104 | PITT-TN113 | No | 42 | F | White | Pre | IDC | Neg | Neg | Neg | 3 | T2N0M0 | 2A |
| 105 | PITT-TN115 | No | 66 | F | White | Post | IDC | Neg | Neg | Neg | 3 | T1N0M0 | 1 |
| 106 | PITT-TN116 | No | 44 | F | White | Pre | IDC | Neg | Neg | Neg | 2 | T2N0M0 | 2A |
| 107 | PITT-TN117 | No | 58 | F | White | NA | IDC | Neg | Neg | Neg | 3 | T2N0M0 | 2A |
| 108 | PITT-TN118 | No | 61 | F | White | Post | IDC | Neg | Neg | Neg | 3 | T1BN0M0 | 1A |
| 109 | PITT-TN119 | No | 48 | F | Black | Post | IDC | Neg | Neg | Neg | 3 | T1CN0M0 | 1A |
| 110 | PITT-TN120 | No | 45 | F | White | Pre | IDC | Neg | Neg | Neg | 3 | T1CN0M0 | 1A |
| 111 | PITT-TN121 | No | 40 | F | White | NA | IDC | Neg | Neg | Neg | 3 | T3N1M0 | 3A |
| 112 | PITT-TN122 | No | 55 | F | White | Post | IDC | Neg | Neg | Neg | 3 | T1CN0M0 | 1A |
| 113 | PITT-TN123 | No | 46 | F | White | Pre | IDC | Neg | Neg | Neg | 3 | T1CN0M0 | 1A |
| 114 | PITT-TN125 | No | 38 | F | Black | Pre | IDC | Neg | Neg | Neg | 3 | T1CN0M0 | 1A |
| 115 | PITT-TN126 | No | 54 | F | White | Post | IDC | Neg | Neg | Neg | 3 | T3N0M0 | 2B |
| 116 | PITT-TN127 | No | 43 | F | White | Post | IDC | Neg | Neg | Neg | 3 | T1CN0M0 | 1A |
| 117 | PITT-TN128 | No | 56 | F | White | Post | IDC | Neg | Neg | Neg | 2 | T1N0M0 | 1A |
| 118 | PITT-TN129 | No | 65 | F | White | Post | IDC | Neg | Neg | Neg | 3 | T1CN0M0 | 1A |
| 119 | PITT-TN130 | No | 64 | F | Black | NA | IDC and other | Neg | Neg | Neg | 3 | T2N1M0 | 2B |
| 120 | PITT-TN131 | No | 49 | F | White | Pre | IDC | Neg | Neg | Neg | 3 | T1CN0M0 | 1A |
| 121 | PITT-TN132 | No | 54 | F | White | Pre | IDC | Neg | Neg | Neg | 3 | T1CN0M0 | 1A |
| 122 | PITT-TN133 | No | 48 | F | White | NA | IDC and other | Neg | Neg | Neg | 2 | T1CN0M0 | 1 |
| 123 | PITT-TN134 | Yes | 50 | F | Black | Post | IDC | Neg | Neg | Neg | 3 | T2N0M0 | 2A |
| 124 | PITT-TN135 | No | 54 | F | White | Post | IDC | Neg | Neg | Neg | 3 | NA | 99 |
| 125 | PITT-TN136 | No | 84 | F | White | Post | IDC | Neg | Neg | Neg | 3 | T1N0M0 | 1 |
| 126 | PITT-TN137 | No | 56 | F | White | Post | IDC | Neg | Neg | Neg | 2 | T1CN0M0 | 1 |
| 127 | PITT-TN138 | Yes | 58 | F | White | Post | IDC | Neg | Neg | Neg | 3 | T1CN1M0 | 2A |
| 128 | PITT-TN139 | No | 68 | F | White | Post | IDC | Neg | Neg | Neg | 3 | T4N1M0 | 3B |
| 129 | PITT-TN140 | No | 54 | F | Black | Post | IDC | Neg | Neg | Neg | 3 | T1CN0M0 | 1 |
| 130 | PITT-TN141 | No | 54 | F | White | Post | IDC | Neg | Neg | Neg | 3 | T1CN0M0 | 1 |
| 131 | PITT-TN142 | No | 57 | F | White | Post | IDC | Neg | Neg | Neg | 3 | T1BNXM0 | 99 |
| 132 | PITT-TN143 | No | 51 | F | White | Post | IDC | Neg | Neg | Neg | 3 | T3N0M0 | 2B |
| 133 | PITT-TN144 | Yes | 55 | F | White | NA | IDC | Neg | Neg | Neg | 3 | T2N1M0 | 2A |
| 134 | PITT-TN145 | No | 47 | F | Black | Pre | Other | Neg | Neg | Neg | 3 | T4DN1M1 | 4 |

TABLE 5

Histopathological features for the four BCL2L14-ETV6 fusion-positive cases from Pitt cohort

| | | | | |
|---|---|---|---|---|
| Case no. | Pitt-TN49 | Pitt-TN134 | Pitt-TN138 | Ptt-TN144 |
| Tubule formation score | 3 | 3 | 3 | 3 |
| Nuclear pleomorphism score | 3 | 3 | 3 | 3 |
| Mitotic count score | 3 | 3 | 3 | 3 |
| Total score | 9 | 9 | 9 | 9 |
| Nottingham grade | 3 | 3 | 3 | 3 |
| Absolute count / 10HPF | ~50 | ~50 | ~50 | 40 |
| Tumor borders* | Infiltrative | Pushing | Infiltrative | Infiltrative |
| Sheet-like growth pattern | Yes | Yes | No | Yes (50%) |
| Lymphocytic infiltrate | 10% or less | >10% (~30%) | >10% (∼20%) | 10% or less |
| Necrosis | Yes, extensive | Yes, extensive | Yes, focal | Yes, focal |
| Apoptosis (visible at 10X) | Yes | Yes | Yes | Yes |

*Not captured in photomicrographs but seen elsewhere in the tumor sections.
HPF: High-power field

TABLE 6

Sequences of primers and amplification conditions used in RT-PCR analyses for expression of different genes or genomic PCR for identification of break points

| Cloning primer sequences | | | |
|---|---|---|---|
| Gene | Primers | | |
| ETV6 | Forward | 5′-CTTCCTGATCTCTCTCGCTGTG-3′ | SEQ ID NO: 1 |
| | Reverse | 5′-GCTGAGGTGGACTGTTGGTTCC-3′ | SEQ ID NO: 2 |
| BCL2L14-ETV6 | Forward | 5′-CGTGGGAACTTGGGCACTCATC-3′ | SEQ ID NO: 3 |
| | Reverse | 5′-GCTGAGGTGGACTGTTGGTTCC-3′ | SEQ ID NO: 4 |
| RT-PCR | | | |
| Gene | GAPDH | | |
| Primers sequence | Forward | 5′-CCCACTCCTCCACCTTTGAC-3′ | SEQ ID NO: 5 |
| | Reverse | 5′-TCCTCTTGTGCTCTTGCTGG-3′ | SEQ ID NO: 6 |
| PCR amplification conditions | 1 cycle | 94° C.: 2 minutes | |
| | 30 cycles | 94° C.: 15 seconds | |
| | | 60° C.: 30 seconds | |
| | | 72° C.: 2 minutes | |
| | 1 cycle | 72° C.: 5 minutes | |
| Gene | BCL2L14 | | |
| Primers sequence | Forward | 5′-GCCAAAATTGTTGAGCTGCTG-3′ | SEQ ID NO: 7 |
| | Reverse | 5′-ACGAACGAGACCTCTCCTGA-3′ | SEQ ID NO: 8 |
| PCR amplification conditions | 1 cycle | 94° C.: 2 minutes | |
| | 35 cycles | 94° C.: 15 seconds | |
| | | 60° C.: 30 seconds | |
| | | 68° C.: 2 minutes | |
| | 1 cycle | 68° C.: 7 minutes | |
| Gene | ETV6 | | |
| Primers sequence | Forward | 5′-CTTCCTGATCTCTCTCGCTGTG-3′ | SEQ ID NO: 9 |
| | Reverse | 5′-GAAGGCCGGTGATTTGTCGT-3′ | SEQ ID NO: 10 |
| PCR amplification conditions | 1 cycle | 94° C.: 2 minutes | |
| | 35 cycles | 94° C.: 15 seconds | |
| | | 60° C.: 30 seconds | |
| | | 68° C.: 2 minutes | |
| | 1 cycle | 68° C.: 7 minutes | |
| Gene | BCL2L14-ETV6 | | |
| Primers sequence | Forward | 5′-AGGTCTCTGCTCAGGGTCAAAG-3′ | SEQ ID NO: 11 |
| | Reverse | 5′-GTGGACTGTTGGTTCCTTCAGC-3′ | SEQ ID NO: 12 |
| PCR amplification conditions | 1 cycle | 92° C.: 2 minutes | |
| | 10 cycles | 92° C.: 10 seconds | |
| | | 60° C.: 15 seconds | |
| | | 68° C.: 3 minutes | |
| | 10 cycles | 92° C.: 10 seconds | |
| | | 60° C.: 15 seconds | |
| | | 68° C.: 5 minutes | |
| | 10 cycles | 92° C. 10 seconds | |
| | | 60° C.: 15 seconds | |
| | | 68° C.: 7 minutes | |
| | 5 cycles | 92° C.: 10 seconds | |
| | | 60° C.: 15 seconds | |
| | | 68° C.: 9 minutes | |
| | 1 cycle | 68° C.: 7 minutes | |
| Gene | TTC6–MIPOLI1 | | |
| Primers sequence | Forward | 5′-GAAACTCGTACCTGCGGCTAA-3′ | SEQ ID NO: 13 |
| | Reverse | 5′-GTGGTTGGAGTGTCCCACTT-3′ | SEQ ID NO: 14 |
| PCR amplification conditions | 1 cycle | 94° C.: 2 minutes | |
| | 35 cycles | 94° C.: 30 seconds | |
| | | 57° C.: 30 seconds | |
| | | 68° C.: 5 minutes | |
| | 1 cycle | 68° C.: 7 minutes | |
| Gene | AKAP8–BRD4 | | |
| Primers sequence | Forward | 5′-CTTCCGCTTCCAGCCGTTC-3′ | SEQ ID NO: 15 |
| | Reverse | 5′-TCCATCCCCCATTACTGGCA-3′ | SEQ ID NO: 16 |
| PCR amplification conditions | 1 cycle | 94° C.: 2 minutes | |
| | 35 cycles | 94° C.: 30 seconds | |
| | | 60° C.: 30 seconds | |
| | | 68° C.: 5 minutes | |
| | 1 cycle | 68° C.: 7 minutes | |
| Genomic PCR | | | |
| Gene | BCL2l14-ETV6 | | |
| Sample name | BCM-TN13 | | |
| Primers sequence | Forward | 5′-AGTGTTCCCTCGCCTATCAGAC-3′ | SEQ ID NO: 17 |
| | Reverse | 5′-ACCTTCCTCTCCTTCACACAGG-3′ | SEQ ID NO: 18 |
| Sample name | BCM-TN35 | | |
| Primers sequence | Forward | 5′-GCATTTCCAAAGCACCTCTTCT-3′ | SEQ ID NO: 19 |
| | Reverse | 5′-ACCTTCCTCTCCTTCACACAGG-3′ | SEQ ID NO: 18 |
| PCR amplification conditions | 1 cycle | 92° C.: 2 minutes | |
| | 10 cycles | 92° C.: 10 seconds | |
| | | 58° C.: 15 seconds | |
| | | 68° C.: 4 minutes | |
| | 10 cycles | 92° C.: 10 seconds | |
| | | 58° C.: 15 seconds | |
| | | 68° C.: 5 minutes | |
| | 10 cycles | 92° C.: 10 seconds | |
| | | 58° C.: 15 seconds | |
| | | 68° C.: 7 minutes | |
| | 10 cycles | 92° C.: 10 seconds | |
| | | 58° C.: 15 seconds | |
| | | 68° C.: 9 minutes | |
| | 1 cycle | 68° C.: 7 minutes | |

TABLE 7

Primary antibodies used in western blot

| Name | Manufacturer | Catalog no. | Species | Type | Clone | Note |
|---|---|---|---|---|---|---|
| ETV6 | Sigma | HPA000264 | Rabbit | Polyclonal | | Target 90-210 aa . |
| BCL2L14 | Sigma | HPA040665 | Rabbit | Polyclonal | | Target 116-216 aa. |
| BCL2L14 | abcam | ab184925 | Rabbit | Monoclonal | | Target 1-200 aa |
| GAPDH | Santa Cruz | sc-32233 | Mouse | Monoclonal | 6C5 | |
| ORC2 | BD Biosciences | 559266 | Rabbit | Polyclonal | | |
| E-cadherin | BD Biosciences | 610181 | Mouse | Monoclonal | 36/E-Cadherin | |
| N-cadherin | Cell Signaling Technology | 13116 | Rabbit | Monoclonal | D4R1H | |
| SNAl1 | Cell Signaling Technology | 3879 | Rabbit | Monoclonal | C15D3 | |
| SNAl2 | Cell Signaling Technology | 9585 | Rabbit | Monoclonal | C19G7 | |
| Vimentin | Cell Signaling Technology | 5741 | Rabbit | Monoclonal | D21H3 | |
| PARP | Cell Signaling Technology | 9532 | Rabbit | Monoclonal | 46D11 | |
| Cleaved Caspase 3 | Cell Signaling Technology | 9665 | Rabbit | Monoclonal | 8G10 | |
| Full length Caspase 3 | Cell Signaling Technology | 9662 | Rabbit | Polyclonal | | |

TABLE 8

Exon information of BCL2L14 (ENST00000308721.9, Human GRCh38.p13)

| ExonNumber | ENSE_ID | Start | End | NucelotideSequence |
|---|---|---|---|---|
| Exon1 | ENSE00001812597 | 12070939 | 12071137 | SEQ ID NO: 35 |
| Exon2 | ENSE00003557359 | 12079299 | 12079738 | SEQ ID NO: 36 |
| Exon3 | ENSE00000822019 | 12087213 | 12087386 | SEQ ID NO: 37 |
| Exon4 | ENSE00000822020 | 12090779 | 12090849 | SEQ ID NO: 38 |
| Exon5 | ENSE00001346602 | 12094664 | 12094930 | SEQ ID NO: 39 |
| Exon6 | ENSE00003475500 | 12098950 | 12099695 | SEQ ID NO: 40 |

TABLE 9

Exon information of ETV6 (ENST00000396373.9, Human GRCh38.p13)

| ExonNumber | ENSE_ID | Start | End | NucelotideSequence |
|---|---|---|---|---|
| Exon1 | ENSE00001324260 | 11649674 | 11650160 | SEQ ID NO: 41 |
| Exon2 | ENSE00003678183 | 11752450 | 11752579 | SEQ ID NO: 42 |
| Exon3 | ENSE00001634229 | 11839140 | 11839304 | SEQ ID NO: 43 |
| Exon4 | ENSE00001623881 | 11853427 | 11853561 | SEQ ID NO: 44 |
| Exon5 | ENSE00001788162 | 11869424 | 11869969 | SEQ ID NO: 45 |
| Exon6 | ENSE00001649298 | 11884445 | 11884587 | SEQ ID NO: 46 |
| Exon7 | ENSE00001657880 | 11885926 | 11886026 | SEQ ID NO: 47 |
| Exon8 | ENSE00002242232 | 11890941 | 11895377 | SEQ ID NO: 48 |

1. A method of diagnosing a subject with increased paclitaxel resistance comprising:
a. obtaining a biological sample from the subject; and
b. detecting a BCL2L14/ETV6 gene fusion in the sample, wherein the detection indicates the subject has increased paclitaxel resistance and the subject is diagnosed with increased paclitaxel resistance.

2. The method of claim 1, wherein the BCL2L14/ETV6 gene fusion is selected from the group consisting of a E2-E3 fusion, a E2-E6 fusion, a E4-E2 fusion, a E4-E3 fusion, and an E5-E5 fusion.

3. The method of claim 2, wherein the E2-E3 fusion comprises SEQ ID NO: 23, the E2-E6 fusion comprises SEQ ID NO: 20, the E4-E2 fusion comprises SEQ ID NO: 22, the E4-E3 fusion comprises SEQ ID NO: 24, and the E5-E5 fusion comprises SEQ ID NO: 21.

4. The method of claim 3, wherein the detection comprises contacting the biological sample with a reaction mixture comprising a probe specific for one of SEQ ID NO: 23, SEQ ID NO: 20, SEQ ID NO: 24 and SEQ ID NO: 21.

5. The method of claim 1, wherein the detection comprises contacting the biological sample with a reaction mixture comprising two primers, wherein the first primer is complementary to a BCL2L14 polynucleotide sequence and the second primer is complementary to a ETV6 polynucleotide sequence, wherein the BCL2L14/ETV6 gene fusion is detectable by the presence of an amplicon generated by the first primer and the second primer.

6. The method of claim 1, wherein the detection comprises contacting the biological sample with a reaction mixture comprising two primers, wherein the first primer is complementary to a BCL2L14 polynucleotide sequence and the second primer is complementary to a ETV6 polynucleotide sequence, wherein hybridization of the two primers on a BCL2L14/ETV6 gene fusion sequence provides a detectable signal, and the BCL2L14/ETV6 gene fusion is detectable by the presence of the signal.

7. The method of claim 5, wherein a first of the one or more primers is selected from the group consisting of SEQ ID NO: 1, SEQ ID NO: 3, SEQ ID NO: 7, SEQ ID NO: 9, SEQ ID NO: 11, SEQ ID NO: 17, and SEQ ID NO: 19 and a second of the one or more primers is selected from the group consisting of SEQ ID NO: 2, SEQ ID NO: 4, SEQ ID NO: 8, SEQ ID NO: 10, SEQ ID NO: 12, and SEQ ID NO: 18.

8. The method of claim 5, wherein the primers are SEQ ID NO:3 and SEQ ID NO: 4.

9. The method of claim 5, wherein the primers are SEQ ID NO: 11 and SEQ ID NO: 12.

10. The method of claim 5, wherein the primers are SEQ ID NO: 17 and SEQ ID NO: 18.

11. The method of claim 5, wherein the primers are SEQ ID NO: 19 and SEQ ID NO: 18.

12. The method of claim 1 wherein the subject has a cancer.

13. The method of claim 12, wherein the subject has a breast cancer.

14. The method of claim 13, wherein the subject has a triple negative breast cancer.

15. The method of claim 1, further comprising administering to the subject one or more of capecitabine, cisplatin, carboplatin, olaparib, and talazoparib.

16. The method of claim 1, further comprising administering to the subject an immune checkpoint inhibitor.

17. A method of treating a cancer in a subject comprising:
a. detecting a BCL2L14/ETV6 gene fusion in a sample obtained from the subject; and
b. administering to the subject a therapeutically effective amount of one or more of an immune checkpoint inhibitor, capecitabine, cisplatin, carboplatin, olaparib, and talazoparib.

18. The method of claim 17, wherein the BCL2L14/ETV6 gene fusion is selected from the group consisting of a E2-E3 fusion, a E2-E6 fusion, a E4-E2 fusion, a E4-E3 fusion, and an E5-E5 fusion.

19. The method of claim 17, wherein the E2-E3 fusion comprises SEQ ID NO: 23, the E2-E6 fusion comprises SEQ ID NO: 20, the E4-E2 fusion comprises SEQ ID NO:22, the E4-E3 fusion comprises SEQ ID NO:24, and the E5-E5 fusion comprises SEQ ID NO:21.

20. The method of claim 17, wherein the cancer is a breast cancer.

21. The method of claim 20, wherein the cancer is a triple negative breast cancer.

22-24. (canceled)

25. A kit comprising one or more probes, wherein each probe specifically hybridizes to a fusion point nucleotide sequence selected from SEQ ID NO: 23, SEQ ID NO: 20, SEQ ID NO: 24 and SEQ ID NO: 21.

26. The kit of claim 25, wherein a detectable moiety is covalently bonded to the probe.


SEQUENCE LISTING (48 sequences)

SEQ ID NO: 1 - DNA, 22 bp, Artificial Sequence
  Synthetic construct
cttcctgatctctctcgctgtg

SEQ ID NO: 2 - DNA, 22 bp, Artificial Sequence
  Synthetic construct
gctgaggtggactgttggttcc

SEQ ID NO: 3 - DNA, 22 bp, Artificial Sequence
  Synthetic construct
cgtgggaacttgggcactcatc

SEQ ID NO: 4 - DNA, 22 bp, Artificial Sequence
  Synthetic construct
gctgaggtggactgttggttcc

SEQ ID NO: 5 - DNA, 20 bp, Artificial Sequence
  Synthetic construct
cccactcctccacctttgac

SEQ ID NO: 6 - DNA, 20 bp, Artificial Sequence
  Synthetic construct
tcctcttgtgctcttgctgg

SEQ ID NO: 7 - DNA, 21 bp, Artificial Sequence
  Synthetic construct
gccaaaattgttgagctgctg

SEQ ID NO: 8 - DNA, 20 bp, Artificial Sequence
  Synthetic construct
acgaacgagacctctcctga

SEQ ID NO: 9 - DNA, 22 bp, Artificial Sequence
  Synthetic construct
cttcctgatctctctcgctgtg

SEQ ID NO: 10 - DNA, 20 bp, Artificial Sequence
  Synthetic construct
gaaggccggtgatttgtcgt

SEQ ID NO: 11 - DNA, 22 bp, Artificial Sequence
  Synthetic construct
aggtctctgctcagggtcaaag

SEQ ID NO: 12 - DNA, 22 bp, Artificial Sequence
  Synthetic construct
gtggactgttggttccttcagc

SEQ ID NO: 13 - DNA, 21 bp, Artificial Sequence
  Synthetic construct
gaaactcgtacctgcggctaa

SEQ ID NO: 14 - DNA, 20 bp, Artificial Sequence
  Synthetic construct
gtggttggagtgtcccactt

SEQ ID NO: 15 - DNA, 19 bp, Artificial Sequence
  Synthetic construct
cttccgcttccagccgttc

SEQ ID NO: 16 - DNA, 20 bp, Artificial Sequence
  Synthetic construct
tccatcccccattactggca

SEQ ID NO: 17 - DNA, 22 bp, Artificial Sequence
  Synthetic construct
agtgttccctcgcctatcagac

SEQ ID NO: 18 - DNA, 22 bp, Artificial Sequence
  Synthetic construct
accttcctctccttcacacagg

SEQ ID NO: 19 - DNA, 22 bp, Artificial Sequence
  Synthetic construct
gcatttccaaagcacctcttct

SEQ ID NO: 20 - DNA, 26 bp, Artificial Sequence
  Synthetic construct
ttggagcatgaagactgtagactgct

SEQ ID NO: 21 - DNA, 26 bp, Artificial Sequence
  Synthetic construct
gcacggtggatggataactgtgtcca

SEQ ID NO: 22 - DNA, 26 bp, Artificial Sequence
  Synthetic construct
gttggaaagaaagcaggaacgaattt

SEQ ID NO: 23 - DNA, 26 bp, Artificial Sequence
  Synthetic construct
ttggagcatgaaggcttgcagccaat

SEQ ID NO: 24 - DNA, 26 bp, Artificial Sequence
  Synthetic construct
gttggaaagaaaggcttgcagccaat

SEQ ID NO: 25 - DNA, 26 bp, Artificial Sequence
  Synthetic construct
aaaaagagtgtgcacctacttcactc

SEQ ID NO: 26 - DNA, 25 bp, Artificial Sequence
  Synthetic construct
tttttctgcaatttgcctccaggtg

SEQ ID NO: 27 - DNA, 20 bp, Artificial Sequence
  Synthetic construct
tggaagtgagtttacacaaa

SEQ ID NO: 28 - DNA, 20 bp, Artificial Sequence
  Synthetic construct
ctaagagcagtttacacaaa

SEQ ID NO: 29 - DNA, 20 bp, Artificial Sequence
  Synthetic construct
ctaagagcaggttggaaagg

SEQ ID NO: 30 - DNA, 20 bp, Artificial Sequence
  Synthetic construct
agacacccagagtgcctggt

SEQ ID NO: 31 - protein, 327 aa, Homo sapiens
MCSTSGCDLEEIPLDDDDLNTIEFKILAYYTRHHVFKSTPALFSPKLLRTRSLSQRGLGNCSANESWTEVSWPCRNSQSSEKAINLGKKKSSWKAFFGVVEKEDSQSTPAKVSAQGQRTLEYQDSHSQQWSRCLSNVEQCLEHEAVDPKVISIANRVAEIVYSWPPPQATQAGGFKSKEIFVTEGLSFQLQGHVPVASSSKKDEEEQILAKIVELLKYSGDQLERKLKKDKALMGHFQDGLSYSVFKTITDQVLMGVDPRGESEVKAQGFKAALVIDVTAKLTAIDNHPMNRVLGFGTKYLKENFSPWIQQHGGWEKILGISHEEVD

SEQ ID NO: 32 - DNA, 49860 bp, Homo sapiens
aaagattctaaggtcccagtgctttcgcttcatcctagaggctaaatgggctcccctggactcgcctggcagaactaaccacctagaagaacgttattgtaagggggaaatgtgagtgctacataagtttcttataaatgtgcttttgaaatagagtccactcttaagtttgatatttcctaaaatttaatgtgtgaaggaagatgcttgggagacaatccgtttatcaggctgatatatggcagatagtctactgacctccctgagcaagaaccttaggaatgaggccaggtggcagaaacactcagggccaagggtagggagcaaaactgtgatgtaaagagacctagatggacttgggaggaggtggggttcaataggaggccacgactaatgccctggcatgcaaaggctggacaggagtcttgaaagagcctcaattagctgggcgtggtggcgggcgcatgtagtcccagctactcgggaggctgaggcaggagaatggcgtgaacccgagaggcggagcttgcagtgagcctagattgcgccactgcactccaacctgggcgacagagggagacaccatctcaaaaaaaaaaaaaaaaagaaaagaaaagaaaagaagaaacaaagagcctcaaaaatatggaaaaccagatcctaagagcaaaatgccaagggggacacttattagtgaggagtctgcaaaaacagaaaggttttagccagaagcttataggagtagacctggaacccaaattgaaaaggaacagtgcgacccgtaatttgagaaggaaatagaataggcagaagaactacagaaactatagcttcgagtctaagagcctatttacaggggtgtccaatcttttggcttccctgggccacactggaagaagaattgtcttgggccacacataaaacacactaacactaatagctgatgagctaaaaaaaaaaaaaaaaaaaagaaaagaaaagaaaagaaaaaagaatggccgtgcataaatctcataatgttttaagaaagtttacaaatttgtgttgagccacattcaaagccatcctgggcctcatgcagcccgtggaagcttgcataagagagagatttaggaccttttggcggaagtgctcgataaggattgaatagcaccataaactgaagctaaactgaaaggttttagaatttaacttaagacacctcaaatgcttttagatgaaactcaggacttttcactgtagtaaagttcagcgtgaaattgaccttgtaggggcaccagacagcagtgagcagggtgtataagagtgagggaggggttaataaatagagatggtgggaaaaggagctggcttttcgtgagttcagcttcttgcagactagactagcaagagtcaagaatgaacctaagccagactcaggtctactgggcagtgctaggcctgtctcaaccctgtttgcacatcaggtaccccatgcgtctgctgtagtctgtagctacatgggaacctaaaacatcaccgaggtttctagtgatgttgaaactcagctaataaacagggaatcccgagggttcccctctactccactcctgacagtggccatcctgcctctgcttgaataccttaactcgcctctcatgaggcacccatctcctcattaaatgattgtcactgggaagaatccgccccacacagccagatcaaatttaccccctggtaactttcagcccctggtccccattctgctctttagcactaggtagctgaatcgaatctttccatgagagtcaattccattcctcctaaatcttctctcttctaagataaatagtccacattatacaatcttaatacccaattcaatgagcacttcatgatagtctaatatatgcaaagcaccatgcttgtgccaagtgggaccctaaagcgaatattatctccttgctctttaggcaaacagggtgaagacaagaggcataggtgatggacactaaaacaaagaccgtaagtactgtaatagctacacacaacttacaatcatataggcatggggaaagagtagcccagcttaccttggaggatctggaaaggttctaagaagaagtggaattgagttggagttggaaagacttcaacaatcagatgtgagaaggacgtgggtggtgagtgcaaagaaaataggacacggctgaggaccttgttttctttttcttttttgaggtttttttcttcttcctttgtttttcgttttatttttatttttcattaacaagtagtaataatacatgtttatggagtacaatttgatgttttgatataggtttacaatgtggaatgattaaatcaggctaattagtaaatccatcacctcacaaacttaccaattttttgcagtaaaaacatttaagatctacttttagcaattttgaaatatacaatgcattattacttattatagttaccatgctgtgcaattgattagtaaagcttattcctcttgtctaactgaaactgtgtagccttttcccaaccaccgctccacccccagtccctggtaaccaccattgtactctctacttctatgagatcaactttctcagatcctacctatgagtgagatcgtgcaatatttgtctttctgtgcctggcttatttcacttagcgtagtgtcctccagattcatccatgttgtcccaaatcacaggatttcacccctgtttaaggctgagtagtattctattgtgcatatataccacattttctttattcattcatctgttgatggacacctagattgattccatatcttggctatggtaaatcgtgttgcaatgaacatgggagttcaggtgtctcttcaacatactgatttcaatgcctttggataaatacccagaagtgggattgctgtatcaggaccttggtttcttagcttctgtctgaccttgagcatacctaaaataagagccctgaacgagataatttctaaagtttagtgatagagaccaaaaggagtatacgtctgagcattccaacttaggccaaagcctaaaagtgagaagcctgcccatatcaagggagtgaggtgtgtgtttggggcaggggtagtagaagagcaagacggttcatgaattcagtattcttgtttcactgatgcagcacccatcaccattctgatcaggtcccttggggacatggacagtgtccttagaatgacaatgatgatggtaccaccagcaccaccaccaccatttctctagcatgtgggtgagccaaacacctttggaaacaccttacatatttacatttacatcctactgaatcctcataacagccctgtgagatagatagaatttttattattctcattttacaaattagaaagctgaggcacagaaaggttaaataacttgcccaagtccatagggctaatctaatagcagagctgggatttgaaggcaagtagtctcactccctgcttttcttcctcgaaaaacacaagggcagggctgaactcagctcaggggacacaatctataccaggacaaggttacaatggccctataacttcttcttcttcttttttttttttttgagacgaagtttcactcttgttgcccaggctggagtgcaatggcgcaatctcagctcactgcaacctccacctcccgggttcaagcaattctcctgcctcagcctcccgagtagctggggttacaggcatgtgccaccacacccggctaattttgtatttttagtagagatggggtttctccatgttggttaggctggtctcgaacttccaacctcaggtacccgccttggcctcccaaagtgctgggattacaggcgtgagccaccatgcccagccggcaatataacttcttttgcattctcattaactcagcctcagacgacactggcttccttggaagccactgcatactgatgattgaagtggagctgacaatcacctaaaaacccctaaatgcctttgacatgagccactcttaagccaggtctctcccattctgttcacgtgtggctgatcagttcaaactaaatgttgggttttacgttgaaccctatacaattctaggttttagcttatagtttcaccctactataattttgaaacatgatatgttacctaatgttacctctcccggtttagtaataacataaacaaaaacagttgacagttatgggggccctgactttgtgccagacaccctgctacgtatttgtttgtttaatgtaataccttatataatcatttagtctttacacaaagttaaatattattattattctcacttaataggataggaaaccaagtctagtgaagttaagaagcttgctcaaaatccaattaggggctgggcgcagtagctcatgcctgtaatcccagctctttggaaggccaaggcaggtggatcacctgaagtcaggagttcgagaccagccaggccaacatggagaaaccccatatctactaaaaatacaaaaaattagccaggcgtggtggcaggtgcctgtaatcccagttacttgggaggctgaggcaggagaattgcttgaacccaggaggtggaggttgcagtgagccgagatcatgccactgcactccaacctgggcaacagagcaagactccatctcgaaaaaaaaaaaaaataaaatccaattaggaagggactgacccaagagtcatacctaggattatctgactccagggcctgaacttttatctatgttctctgcagcgtcctcacatcttctacacacttgttattatctaagtcaatcatgaaaaatgatgaaacaggccaggcgtgttggctcacacctgtaatcccagcactttgggaggccgaggcgggcagatcatgaggtcaggagatcgagaccagcctggccaacagggtgaaaccccatctctactaaaaatacaaaatgagccaggcgtggtggcatgtgcctgtagtcccagctactcgggaggctgaggcaggagaatcgcttgaacccaggaggcaaaggttgcagtgacctgagatcacaccactggactccagcctgggcgacagagcgagactccatctcaaaaaaaaaaaaaagaataaagaaaaatgatgaaacagaatcagggagagaattcagaggaaaatgacatccctccaggtgtgtatgaaccaattactcatagagtcataaacagttagagaatgaggcatttttcacaaaatattgagggctggagaggttgaacaatttgctcccactcacacatctaggaaagagaggagatggaaaagtaaacaatccagaaaggtcaaggccctcattccatcaatgcacaattcaagtacacatacgtgggaaaccatctgaaaagttttgtaattacaatttcatctagccagcaattctccaactcattcacatgcatctcatgacatcgttgtcagaagccttgctagagtgaacgtgcacttcatcaaacacacccctgatccgtgggagatgggattcgtttgtgctgatctactctccaggaacacaagaaactcaccattctaacggttcataaatcatccatttaataccccagtttcagattttgcaggaattaacatcaatgcacggtttttgagacccgccaacttgctgaaacagtgcaagccacctgcagtcctcggataccactcagtgatggccctaggtctccctactctgggcctgagattcacttacagctgcaaaacacacctgtgcactctcctcataggcagaacgctttcacttctcagtcatttccaactgagactgttttctctggtagagaatacagaagtcaaatggactcagagtggttctgccttgtctctgccttctgttgttgagaataaaccagaaaaatcccttcttctgtcattaacattttcaagaagctccgttcagtccagtgctcttatttcaggcctatcacaccctttcatatttgtgttttcatagccactgacaaagactgtgtccttgaccaaactttagtcaggttcctctgagcccaatcaggccccatccttgggcatgcccccacccccaatgtctgatcaccctcaaaatctgatcaaattcctcatccctcatcatccccgaggcgatgtctgatcaccctgcctggtcttcagcaaaaatcacactaggtcaacttagccagaatcccctgactcccaatgtttccccttagtaattttccacccactgaccccaccttattccttggttacaaatccccatttgcctctgctgcattcagagttgagcccagtttctctccctgaccacaagaccctattgcagtggtccgtccctacaccaactgcgatagtcccggataaaatctgccttacccttttaacaagggtcaagagtaaatttttccttaacatctcctgtgggcactatttgtatacatttaaagtgagagcttatccaagagcttcctttttagcctggcaatcaagtatcctttttgcttctttctcccgatcactccctaaacccctcatctatagtaagaagcagaatcagaatttgactccaagactgtgcctgaggagcctgagccctaaatactgtgctctattgttacctgtgttatctgtaaataactgagccattgattaaaacacagttaataggctgggcacggtggctcacgctcactcgtaatcccagcactttgggaggccaaggtgggcggatcaactgaggtcaggagttcgagaccagcctgaccaacatggtgaaaccccatctctactaaaatacaaaagttagctgggcgtggtggtaggcgcctgtaatcccagctactcaggaggctgaggcaggaaaatcgcttaaacctggaaggcctcggaggttgcagtgagccgagattgcgccactgcactccagcctggacgacaagagcgaaactccatctcaaaacaaaaacaaaacacaatcaataacagaacctagagatctctagctagatgcatctcaatcaattaagcatgcaatcaaagagactgaaggggacctgaggtccagataatttaagtgatttaaccaacgttaaagattatttcttaaggggaaaatcatgactctcacatgatataaaagaacatgtggcaaatattttatttaactaatttaactggagccagcaaaatattaaaaccagctcaaaggagaaaactccaactaccacacatttatagtcagataagaaataccgaaataaatttacaaacaggcaaaactggaaaaactggttaatatctacaggacaataaacaaaggcattccacgggagactatttgcattataatatgcaaggaattatttgcattacgatcagttttctatatgttaacttctttctaaggagttgtaaaatagcataatcaaaagcaaataaataagcatatctctataaaatcagataatccccaaaggctctaacattccattgaaaggatatccagtaatctctttgcccattgcaaatctttccatttttccctacgccaaagtcaaaaagcagtaatagcaactccagaaaagaggtgaagagagccgggctcggtggctcacgcctgtaatcccagcactttgggaggccgaggcgggtggatcacctgaggtcgggagttcgagaccagcctgaccaacatggagaaaccccttctctactaaaaatacaaaattagctgggtgtggtgacacatgcctgtaatcccagctactagggaggctgaggcagaagaattgcttgaacctgggaggcggatgttgcagtgagccgagatcgcaccactgcactccagcctgggcaataagagcaaaactccattaaaaaaaaaaaaaaagaagtgaagaacatcaacaaactgggctgcatctgaaccatgtgaagaagatggtcaggtttagtggttagcgtcatgggccttaaagacagtgcctgggctccctcaaattccagctttgcctcatgtgctcgtgtgaccctgggaaattttacttaagtcttttgtgtgtttgtctttttttttttttttttttgagatggagtctctgttgcccaggctggaggagtacagtggctccatcttggctcactgcaacctccccctcctgtgttcaagcaattcttttgcctcggcctccccgagtagctgggactacaggtgtgtgccaccacgcccagctaaatttttttgtgtttttagtagcgacgaggtttcaccatgttggccaggctgttctccaactcttgacctcaggtgatctgcccgcctcggcctcccaaagtgctgggattacaggcgtgagtcattgtgcccagactcttttttttttcttttttttgagacggagtctctgttgcccaggctagagtgcagtggcatgatgtcggctcactgcaacctccgcttcccgggttcaagcgattctcctgcctcagcctcccgagtagctgggattacagtcatgtgccaccacacttggctaattttgtatatttagtagagacggggtttcagacggggttggccaggctgttctgtaacattctaccacaaaagaagtgaaaatggcctgttcctgccttaactgatgacattgtcttgtgaaattccttctcctggctcatcctggctcaaaagctcccccactgagtaccttgtgacccccactcctgcccgccagagaacccccctttttcctttacctacccaaatcctataaaacagccccaccccatctcccttcactgactctcttttcggactcagcccgcctgcacccaggtgaaataaacagccatgttgctcacacaaagcctgtttggtggtctcttcacaccgacgcgcatgaaatttggtgccatgactcagatgggggacctcccttgggagatcaatcccctgtcctcccactctttgctccgtgagaaagatccacctatgacctcaggttctcagaccaagcagcccaagaaacatctcaccaatttcaaatccagtaagcggcctctttttactctcttctccaacctccctaaccctcagcctctttctcctttcaatcttggcgccacacttcaatctctcccttcttttaatttcagttcctttcattttctggtagagacaaaggagacacgttttatccgtagacccaaaactccggtgccggtcacggactgggaatgcagccttcccttagtgtttaatcattgcaaggacacctctctgattattcccccacgtttcagaggtgtcagaccacacagggacgcctgccttggtccttcacccttagcggcaagtcccgcttttctgggggaggggcaagtaccccaaccccttctctccgtgtctctaccccttctccacctttctggggtgcaagaaacccccaaccccttatccttcacccttagcggcaagtccggcttttctggaggagaggcaagtaccccaacctcatatctctgcaccccaatcccttatttctgcaccctgacctcatatctctgtgccccaatcccttatttctgcaccccaacctcatatctctgctccccgatcccttatttccatgccccaacctcttatatctctgcaccccatcccttatttccatgccccaacctcctatctctgtgcctgaccccttctctgcttttctggagggcaagaaccccctaccccttctccgtgtctctactcttttctctgggcttgcctccttcactataggcaagcttccaccttccattcctccttcttcttccttagcctgtgttcttaagaacttaaaacctcttcaactctcacctgacctaaaatctaagtgtcttattttcttctgcaatgccgcttgaccccaatacaaactccacagtagttccaaatagccagaaaacagcactttcaatttttccatcctacaagatctaaataatgcttgtcgtaaaataggcaaatggtctgagatgcctgatgtccaggcattcttttacacatccgtccctctctagtctctgttcccaatgcaactcatcacaaatcttccttctttccctcccacctgtcccctcagtcccaaccccaagcgtcgccgagtctttctaatcttccttttctacagacccatctgacctctctcctcctcgccaggccgagctaggtcgcaattcttcctcagcctctgctcctccaccctataatccttttatcacctcccctcctcacacccggtccggcttacagtttcgttccatgactagccctccccctcctgcccagcaatttactcttgaaaaggtggctggagctaaattaaagttaatgctcctttttctttatcccaaatcagatagcgtttaggctctttttcatcaaatataaaaaacccagcccagttcatggctcgttcggcagcaaccctgagacactttacagccctagaccctaaaaggtcaaaaggccgtcttattctcaaaatacattttattacccaatctgctcccgacattaaataaaactccaaaaattaaattccggccctcaaaccccacaacaggatttaattaacctcgccttcaaggtgtacaataatagaaaaaagttgcaattccttgccttcactgtgagacaaaccccagccacatctccagcacacaagaacttccaaaggcctaaacctcagcgtccaggcgttcctccagaacctcctccccgaggagcttgctacaagtgccagaaatctgaccaccaggccaaggaatgcctgcagcccaggattcctcctaagccgtgtcccatctgtgcgggaccccactggaaatcagactgttcaactcacctggcagccactcccagagcccctggaactctggccgaaggctctctgactgactccttctcagcttagcggctgaagactgacgctgcccgatagcttcggaagtcccgtagaccatcacagatgccgagctttaggtaactctcacagtggagggtaagcccgtccccttcttaatcaatacggaggctacccactccacattaccttcttttcaagggcctgtttcccttgcctccataactgttgtgggtattgatggccaggcttctaaacctcttaaaactccccaactctagtgccaacttagacaatactcttttaagcactcctttttagttatccccacctgcccagttcccttattagactgagacactttaactaaattatctgcttccctgactattcctgggctacagccacacctaattaccaccttttccctcagttcaaagcctccttcacatcctccccttgtatctccccaccttaacccacaagtataagatacctctactccctccttggcgaccaatcacacaccccttaccatctcattaaaacctaatcacccttactccgctcaatgccaagatcccatcccacagcacgctttaaaaggattaaagcctgttgtcactcgcctgctacagcatggccttttaaagcctataaactctccttacaattcccccatcttacctgtcctagaacaagacaagccttacaagttagttcaggatctatgccttatcaaccaaattgttttgcctatccaccccgtggtgccaaacccatatactctcctatcctcaatacgtccctccacaacccattattctgttctggatctcaaacatgctttctttactattcctttgcacccttcatcctagcctctcttcgctgtcacctggactgaccctgacacccatcaggctcagcaaattacctgggctgtactgccggaaggcttcacagacagcccccattacttcagtcaagcccaaatttcttcctcatctgttacctatctcagcataattctcataaaaacacacgtgctctccctgctgatcgtgtccgattaatctcccaaacctcaatcccttataaaacaacaactcctttccttcctaggcatggttagtgcagtccgaattcttacacaagagccaggaccgcaccctgtaacctttctatgcaaacaacttgaccttactgttttagcctagccctcatgtctccgtgcagtggctgctgccgccctaatacttttagaggccctcaaaatcacaaactatgctcaacttactctctacatttctcataacttccaaaatctattttcttcctcatacctgatgcatatactttctgctccccggctccttcagctgtactcactctttgttaagtcccacaattaccattgttcctggcccggacttcaatctggcctcccacattattcctgataccacacctgacccccatgactgtatctctctgatccacctgacactcaccccatttccccatatttccttctttcctgttcctcaccctgatcacgcttgatttattgatggcggttccaccaggcctaattgccacacaccagcaaaggcaggctatactatagtacaagccactagcccgcctcttagaacctctcatttcctttccatcgtggaaatctatcctcaaggaaataacttctcagtgttccatctgctattctactactcctcagggattattcaggtcccctcccttccctacacatcaagcttgaggatttacccccacccaggactggcaaattaactttactcaacatgccctgagtcagataactaaaatacctcttagtctaggtagacactttcactagataggaagaggcctttcctacagggtctgagaaggccaccgcagtcatttcttcccttcagtcagacataattcctcagtttagccttcccacctctatacagtctgataacagacgagcctttattagtcaaatcagccaagcagtttttcaggctcttagtattcagtgaaacctttatatccattacagtcctcagtcttcaggaaaagtagaacagactaaaggtcttttaaaaacacacctcaccaagctcagccaccaacttaaaaaggactgaacaatacttttaccactttcgcttctcagaattcaggcctgtcctcagaatgctacaaggtacagcccatttgagctcccatatagatgctcctttttattaggccccagtctcattccagacagcagaccaacttagactgtgccccaaaaaacttgtcatccctactatcttctgtctagtcatactcctattcaccattctcaactaatcatacatgccctgctcttgtttacactgccagtttacactgtttctccaagccatcacagctgatatctcctggtgctatccccaaactgccactcttaactcttgaagtaaataaataatctttgctggcaggactatgctgaatctccttaggcactctttaatcagatgtcctaggtcctcccaattcttagaccttttatacctgtttttctccttttcttattccgtttagtttttcaattcatacaaaactatatccaggccatcaccaataattctaaatgacaaatgtttcttctaacagccccacaatatcaccccttaccacaaaatcttccttcagcttaatctctcccactctaggttcccacgccaccccaatcccactcgaagcagccctgagaaacatcgcccattatctctccataccacccccaaaaattttcgctgtcccaacactttaccactatttcgttttatttttcttattaatataagaagacaggaatgtcaggcctctgagcccaagctaagccatcatatccccgtgacctgcacgtacacatccagatggccagttcctgccttaactgatgacattccaccacaaaagacgtgaaaatggcctgttcctgccttaactgatgacattgtcttgtgaaattccttctcctggctcatcctggctcaaaagctcccccactgagtaccttgtgacccccactcgtgcccgccagagaacaaccccccttcttcctttacctacccaaatcctgtaaaaccccatctcccttcgctgactctcttttcagactcagcccgcctgcacccaggtgaaataaacacccatgttgctcacacaaagcctgtttagtagtctcttcacatggacatgcatgaaactgacctcaagtaatccacctgcctcagcctcccaaagtgctgggattacaggcatgagccaccacgtgtggcctgtttgttttttgagacagttctgtcacccaggctggagtgcagtggtttgatcactgcagccttgaactcctgggctcaagtgatcctcccacctcagcctccagagtatgggactacaggcatgcagtaccatacctggctaatttttttgattttttttttctttttttgtagagatggggcctcactatgttacccaggctagtctcaaacacctggcctcatgcaatctgcttcccaaaagcacacccaaaaggtgtgagctaccatatcccacctacttaactcttttgtatctcagtttcttcatatgtaaaatgggcataactgtcgtgcctactacacagctgagttaataattggcatggactcagtaaaataagaactattattgttaggcctctttggagccatggccacaacagcatgcctatctttactctaaaaattttgcccaactaggctcagaattctctctcaaccatctccaattttacagtaaaatgaacattctgtctccaaggttcccattgcttctatgagaccaaccagtgcctctttgtcagccaaatgcaaggatagaacaatgtttatcaccgtggcttcctttgacttaagagatggtattagcagcaagtgtgaaatgacctatgtttatcaggagtcttccatggaagtcatggtagtcagatctcctatcacgattacctcctgccttggtgcagggcgtgatctctgtactaagaacgtctttctccatctcatctctggccaggccagttatagcaacacagcactatcacttcaatccttctattattttgtcttcactcaaaccacagataagaggatcttcctacaggtataggggagaagaaaatttatttttcctctaccctcctaggttttttggctggggccctgcaaattagactgacgaaagatagattaacaaaagaaaaacagacagaagtttattattaacacatgcatacacacaggatcgctcaatgatgagtgactcaaaggagtggtcagaaattgggcttatagagcaccttttaaaaaaatataaatttgtaaaaaagtgacaagacaggccgggtgcagtgagtggctcacgcatataatctcagcactttgggaggccgaggcaggcggatcacttgaagtcaggagtttgagaccagcctggccagcatggagaaaccctgtctctactaaaaacacaaaaattagctgggcatgatggcgtgcacctgtaatctaagtgactctggagactgaggcaggagaatcgcttgaaccggggaggcggaggttgcagtgagcagagattgcaccactgcactccaggctgggcgacagagtgagactccatctcaaaaaaaaaaaaaaagaagaagaagtgacaagacaaagggaaagagctttaggcttctggaggtggcaaactgtgggaaggtaagtagatgggaaaagtaatggaagataaagattgccttagtgaggttggttatgtaggttcctccagcgccatctctgggctgcttaagagtccagagttgtctccagcgactgagaatcttcctgtcctccctggtagggagacatggggagcagagagtttctctttgtttctatttttttttttttttgaggcgaagtctcgctctgttgcccaggctggagtgcagtggcacaatcttggctcactgcaacctctgccacccaggttcaagcgttctcctgcctcagcctcctgagtagctgagattacaggcacccaccaccatgcccagctaatttttgtatttttagtagagatggggtttcgccatgatgaccaggctggtcttgaactcctgacctcagatgatccacccacctcggcctcccaaagtgttggaattacaggcgtgagccaccgcacctggccgagagtttttcttgtgtctgcttcatcttcattgccttcagctcaaaatgatcttaaggttaaagtgttgtactttggggagtggcatgttgaccccttcacagatgttcttcataatgaatagtgcttctcctctgctccgtttcccaaatctttcatttcaaccagtcacagccttttattgctccattccaactctgatcctatttcattgagtttcatcattgatatcaaaacaaggataaatgtcaaaccccagagtaatctggtgtccactctaaaatctgctcatctggcctttcaagtcttctttggttactcagtgatgggcaaaaaccccaacgttttcctgaggatgggctttttatctatattattcaaactcttaggcatttttatctaagatatatttccagtgatgatataaatcaattccagaatcatagattaaagtgaattcaaagcatatataacaatttcaaaaatattcatacccaataattccactctcaggcatcatttctcaggaaacaatagaaaatgtgagcaaagatgaaaagctctaccctgcagcattatttgtaacagtaagcacttgaagacaaacaaaacattattattattattttttttttttttttttgagacagagtctcgctctgtcgcccaggctggagtggcgcaatctcggctcactgcaagctccgcctcccgggttcacgccattctcctgcctcagcctcctgagtagctgggactacaggcgcccaccaccatgtctagctaattttttgtatttttagtagagacggggtttcgctgtgttagccaggatggtctcgatctcctgacctcgtgatctacctgccttggcctcccaaagtgctgggattacaagcatgagccaccgcacctggtgacaaacaaaacatttaaaaatagttggctggctgattatttgatttgatttgatttttattttgatttttttggagactgagtctcgctctgtcacccaggctggagtgcagtggtgtgatcccagctcactgcaacctctgcctcctgggttcaagtgattctcctctggatgaatatttttagtgctatcattcactggaatgatgtcgccactaaaaaattttatttacaaaataagtttattagaagttatgatggctggacgtggtggctcacacctgtaatcccagcacttttgggaggctgaggcagacaaatatcttgagctcaggagttggagaccagtctggccaacatagtgaaaccccgtctttattaaaaatacaaaaatgagccaggcgtggtggcacatgcctgtaattgcaactactctggagactgaggtgggagaatagcttgaacccgggaggcagaggttgcagtgagccgagatcgtgccactgcactccagcctggggaacagagtgagactctgtctgaagaaaaaaaaagaatcatcctgactttccttatagagagggggagggcagagagtttgtcctgtgtctcctttttctcaaagaaaagaagggacgtccaccaaatttttagcagcatttcccactgaatgactgcattatggcaatccttattatgaccctgttttacgagttttctacaacagcatgtctatgtttacaatcgggagaaaagtggtttttgcttttgctgtttaatatctaacatgtgttgtgacctgtccctggccaacttgttaggcgtggagataacatagttgaatgagaggtggacagttgctctcctcagcttccgtgtggggtttgccccttccctgtgttaatagttgctttaccaaaacctttcaggactttttttttttgagacagggtctggctctgtcacccaggctgagtgcagtggtgccatcatggctcattgcagcctcaagcccccggccccctgccaggctcaagcaatcctcccgcctcagtctcctgagtagctgagactacaggtgtgctccaccacgcccagctaactttttatttttttgtagtgatggggttatcaccatgttgcccaggctggtttcaaactccaggacacagtctgtcctcctgcctcagcctcccaaagcgctgggattgcaggtatgagccactgtgcatgaccaagactcttcaggatcctaaatggtttcagcaacaaacattgtttagttatgtaactcagaactccagtatggacatactatgtgatgtgattttattattgcttatatgcttttgtggttttattgtttgtttttctgtttgtttgttttgagacagggtttttgtttttgttttgagacagggtcttactctgtcacccaggctaaactacaatagcatgatcctagcttgctgcccccttgaactcctaggctcaagcaatctttctgtctcagactaccaagtagctaggactacaggcatgtgccaccatgctatgttgtctggctggtgttgaatacctggtctccagtgatcctcccacctcagccccctgagtagctagcactacatgcgtgagtcatgcctggctaattaaaaaaaaaaaaattaagagatggggtcttactatcacagcgtgaatcccaaaattggggttcagtctgggaggccacatgggttcttggcttcccacaagaaggaattcaagagtgagccaacaaagtaaaatgaaagcaaggttattaagcaagttaaggaataaaaggggggctactccataggcagagcagtggcatgggctgtttgattgagtatacgggtattccttgatcatatgctaaacaaaaagtgaattattcgtgagttttctgggaaaaaggtggggagttcccagaactgagggttttcccatttcagacaatatagggtaacttccagacattgccataacatttgtaaactgacatggggctggtaggagcgagtgtcttttagcacaacagtgcattatcattggcatataatgagctgtgaggacgaccggaggttgctttcatcgcatcttggttttgatagatttgggctgctgctttactacatgtcattttatcagtggtgtctttgtgacctgtaccttgcaaaccagtcctgaagaactcttgtctcatcactgtgttgtccaggctggtcttcaagtgatcctcctggcttcagagtgatcctctcacctcagcatcccaaaatgctgggcttgcaggcttgagacactgagccagaccctgtttattcttgaattgtttccagtgaatatatctcatcgtctccccccaaaaaaaggttgcaagtttttggccaggcgcggtggctcacgcctgtaatcccagcactttgggaggtggaggcgggcagatcgggaggtcaggagatcgaggccatcctggctaacacggtgaaaccccgtctctactaaaaatacaaaaaaaaaaaaaaattatccaggcttggtggcgggcgcctgtagtcccagctacttgggaggctgaggcaggagaatggcgtgaacccaggaggtggagcttgcagtgagccaagatcgcaccactgcactccagcctgggcgacagagcaagactccgtctcaaaaaaaaaaaaaaaaaggtggcaagtttttataaaaagagaaaaatcattcttagtatttctttaataccctctacattgacctcactggtgcaaaaaatattagtttgctggtgaaaattcacctcgagttactaaatatctaactttcccataactaccatctgtttgtctcttgtggatctgtttgcctaatatttaaatgacccattcttccccacttttacgaaaatagcattaattcccctttcactattcacacttccttcatcgtcacagtttgaccagcggtccagggtttatgatctcaagcatggccatatatatctatttcacttaagactctggaaagagcataaaatcagatgtaactgtttcctatttcatataatagggaattccatctctaaagttatgacatttggcctttcagtgggtttttgtggtttttactatggctgtgcttagattcctttttgaacacttttccagtggttccacttgaacacaatctttgagtcacataggagaggtgacatgatagagtgtaaagccagaaagagcacgatttggatctcagctcaactgatagggatccgtagcaagatacttcacctctctgagcctcagtttcctcatctgtgcaatgggaatcgtaacgcctgcctctcaatgttgttaagattaaatatgttggccgggcgcagtggcttacgcctgtaatcccagcactttgggaggccgaggagggcagaccacctgaggccaggagttcgagaccaacatggtgaaacccctgtctctactaaaaatacaaaaattagccaggcgtgctggtgcaagcctgtaatcccagaaactcaggaggctgagatatgagaatcgcttgaacccaggaggcggaggttgcagtgcgccaagatcgcaccactgcactccagcctgggcaacagagtgaaactctgtctcaaaaaaaaaaaaaagattaaatatgtgtacagtacctgtaccatgttaggcggtcagtaaacatttgtttaactaacaactgataaggatagtaaagaagtatcagaacacccacctagttatcccaaagggcaggaagagtgctaggtaatggatatagaattccttttagagtcaaattacacgggcaaagatcagcccctccttcaattaaaaagccaggggcagggcgtaataaggaagtggttctccttctttaagcctcttttcaggctgagtcctaaacctgaagaaagtttagagcctggggctctaaactacctgagtctttccaaacgacaagccaagaagacctgttgaaagtttcctcttaagtttcgtggagagagactcaggtatagaaatatccttactgccacctgacctgaagcagaagaaatcacagacagcttccagaccaggtaacaccaggaggcaccagtcttcatcctcagcctttgcaggaggacacagcctggtctttagctgtttgccaaatgaggcagtctaattcagggagctctttactgggtttattacaaagttcttatcatgtaagtctcaggacacaaaatgacattgagaatctagtttagcggggtttccaggaaagacgaagaaaggttatttaggtttctctttcatttctcttttgctacaaagggtttgggacattcagttaagaacacattactggttgaggtatgtgacgttactcagtcttctctgcttatgtatctggggaggtatgcaatgacatgacagccattccgtggccagggacaccactgcccaagctggagaccacgaggattcagggactgaagccagcatgggaattcctggtttgagatcagagtcctgagtacctcgtgggaacttgggcactcatccgcaggaggtctagacccccagagaattccttgagtctaaggcacaggtaaatggaagacccttctctctcctctctcccctctagaaagctccctcctccctgagctccctgtccccttctttccacaatcagagcctcggaccttctgtttggtgaatatgacctgaggctctgtctagcattcagcttctattgtgggaagttaaggcactttctctcattcctcatttcacctttagtattaaagaacagtccaggcagaaggcacgaagagcctgagaggtctgaagtgagggggagaggagaggggcagaaggggaggctccggacaacagaaccaagctgagcttaggatgtgctgtgggaggctccgtggggatgagtgtcagtgcccacgagatccactcaggcctcaactgtgtgtcctgagcacatgaagtcagctcttctgctgggacagccaccaagggattctccacacccctcaccgctgggacaggcagaggcacagcgaaccaggtgtccagcgtaactggggcactgggtggtggccgcctctgtgtgtggaaacacgaggtgcttgctgcagtttcctctgtgtgcttatccgttagctgccacccaccgggagggaagtgaaatctcacctcaacaagtgagtccgcagagcgagaaccgtgcgacagtgcgtctgccaatacctggaatgtaggtgttcgttgcagtgtggccttcccagccggcctctcagcacacccggccaggcagccagtgttcccagaggacacgctggggccagacgtggaggaccccggccacctcggggagggaaagagaccaagaaaagaacctctgtagccttgcttctgagagtgcaggtttgttacaggtaaggatcaggaaggtaaacatagggcagtgaagcagggttgtgtcctctgcggccctgaattagaataagcctcacaggtctcacccaggaacggaaatctcccctccaggttctgaaccagcattcagaggccccacccagggcttctgtcaacataagcataaaacttatggtttacctttccataaaccccaaagttgggcttcttgtatagagtacaatttctgtaggaagagctgaaagcagaagggatctaacctgtgataatactcgatcgctgctgcttgaaagtaagacttagctatacactgaacaatcaagtaatttataacaaggattagaggaaacaaaaactctgtgtttgtgtgggtgggtttttttttttctttatttttgtttcttaagagagtgtctccctctgccacccaggctggagtgcactggcgcgatcatagctcactgcagcctccaactcccaggctcaagcgattctcccccctcagcctcccaagtggctgggactgcaggtgcacaccaccacgcccagctaatttttttcattttttgtagagacggagccaaggctggtcttgaactcctgggcttaagcgatcctgctccctgggcctcccaaagtgctgggattacaggcgtgagccactgggtccggcatctacatttgttaaatacattaaaaccctcttaatcctttgtctccgtttgttctcttttgctccctgctctccaggctcttgtggcggaggcccccgctccctccgctgtagggtagcctccttctggggctggctcctgagcactgttctttctcctctctctcatcctctctcattcttagctttgcctgtcgactgggccaagtcacagcctttctggcctgtaagacaacaaaaatctcacattttactaaatggaaggactaagtactctgtcttgacttcagtggtttccccgatttctgatgttgctgctgaaatagcattagcaagcttgtttcttaaagaagccctctctccagtgacaatactccccccgccacacacacacacatgcacacgcacacacaaacatgcatgcacgcgcacacacacacacaattttaaagccctcgtgttatattaaaaaggtctggatgtttttaacatacatggaaatttcttcagcagcacagtgttactggtctgacgagggcgcacagcctcctacccaaggacacagttcctgaaggaccagggtgcacatctggcctgcagtgccctgagccactctgtccaaaaggaagccttgaatcaaaagcctcatgaatcctctcctgagaaattccatgtctggtctcttgctagactcagccttttccccaccaagtctgccgcacctaattctgacctcaccctgcttggtggcccgctttgcctgcttgcacgcacctcaggtctcagtgtgtctcagcaacagcttcccctcctagggtggccagagttagcaagcaagaatgcaagacagccagttatgtttgaatttcagataaataacaaatataagtgtcttttagcatataatattttatttgggacatatactaatatttattatatattagtctcatgatatttgggacatatactaaaaaattatttgccctttatctgaacttcaaatgtgaccaaatgtgtcctgtattctgaatgtgtctggcagtcctataatcattcattcagtcagtcatcagatatttgagcacctaccatgtatcaagcactattctagagagcccggatagctcagtcagtagagcatcaagcgctactctaggtgctggggaagccagccaaagtcccgccttcatatcttacagtatgtctactttgtttttatatacatgtacactatatatacttgaagatatgtacctttttctttttttctttttattatttttttttgagacagagtctgactctattactcagtgcagtggcaccatctcagctcactgcaacttctacctcctgggttcaagtgattctccagcctcagcctcccaagtagctaggattacaggcatgtgccaccatacctggctaatttttgtatttttagtagagacaaagtttcaccatgttggccaggcttgtcttgaactcctgacctcaagtgatctgcccacctcggcctcccaaagtgctaggattacaggcgtgagccaccgcgcccagcccagatatgtactttttaatggcaatgttaccacatttaatgagattatctttttaaaaaatgttttaatttttaaattttaaaaaatagggatgggatcttactttgttgcctagactggtctccaaatccaggtctcaagtgatcctcccaccttggcctcctaaggtggtgggattacaggtgtgagccacggtgccagccaagatgatcaatacttccttaatatcatctaaaacatacactgtactgtgtttagttctctatcatctcaaagatatctacatctttatacagtatgtttgaattacgatttaataaagatccactcatgacatctaattgatgtctctttttttttaatatgtttcttttttttctttttatttatttatttatttttgagacagagtctccgtgtgtcacccaggctggagtgcaatggcgcgatccccgctcactgcaacctttgcctcccaggttcaagtgattctcctgttgcagtctcccaactcactggaattacaggcacctgccatcatgcccagctaatttttgtattttttttagagacagggtttcaccacgttggccaggctgctcttgaactcctgcctcccaaagtgctgggattacaggcgtgaacgattgtgccctgcctgatatgtttctattctttctttctttctttctttcttttttttttgagacggagtttcgctgttgttgcccaggctggatgcaatggcgtgatctcggctcactgcaacctccgcctcctgggttcaagtgattctcctgcctcagcctcccgagtagctgggattgcaggcatgtgccaccatgcctggctaattttgtatttttagtagagacggggtttctccatgttggtcaggctggtctcaaactcccgacctctggtgaactacccgccttggcctcccaaagtgctaggattacaggcgtgagccatcgtcccctgccggatatgtttcttatgtctcttttaagctgtagcagttctctgtcttcacctttttttggcgccatttatttgttgaagaaaccggccatttgtcttgcagaacttaccagatactgaatttggatagctgccttcttgtggtcattagtttatttctctgtcccctgtatttcctgtaaactgatcattagatccagaggcttgatcagattcggatccaatgtttttggatcaggagacacataatggttgtctcacctttagtgagaccaagattggtcattgggaacagggaagtggagctggggtgagtatagggtcaccagatgtccagtacaagcagaatggacagggctgtgctgcaggatggatgaggggagggtgggatgcccggaaaatgcatgggggagggtgggatgcccggaaaatgcatgggggagggtgggatgcccggaaaatgcatgggggagggtgggatgcccggaaaatgcatgggggagggtgggattggatggaattggggagcgggaaggggtaagtggaaaagcagtaaggaacatgaccttcgaggagacactgacgtgactccttacagagcaaacgatttgcatgtaagtgaaatgcatgtaagtgaatgtgaaattggagcagtggcgggaagaaagattgtccaagccctggaataagacaggaatgtgaagaaacagcagaggaggaggaaggattgcagtcatgacccccacttcatgccatactcctcaagcatttgcttacatccagttatgtgagggaggtttgcggggcttggaagttgtataattgtatctgtgtagttggtctggtgggttttgcaacttgccctttgctgcactgggagggccatttttgtcccctccagcatcacctgatgttgcagcatagcccgctgcatccctcccaggctcacttccatcagaacgcacctagccagggacccttgctttcctggggcctggccaaggagcagttcctcagatcaacagcttcgctatcagccaatgtgagcctcttctaagtaaaggagaggagacagtgatggaaaaaggcagcatccagcaggccctcaggggagatatttcagctcctacacctccttccaatagcagaaccagggggaaaagttagagaatatccttattctatctaacaacttggacaccgaggaacaaaaaggtggcgtgacttgcccaaggatgttgcaggttagaagctgagccagccgtggctatccatccttcagacacccaggccaacttgtctgtaccacctcacattgcctcaaaccctgtctgcagttactgtcccatttctttatcatttggtcctggtatgacaaggtacagtggaaagaaaaagaggaacatgccagccagtccccttaatattactgcagcttctttatcatcacagcagcttacatgccgaaagaccgtcttaaattaggaccagaaagggaagggaacattattaagagccatattaagacagagcctgaggccgggcatggtagctcatggctgtaatctcagcactttgggaggctgaggcaggtggatcacttgagcccaggagtttcgcaccagactgggcaacatagcgagaccttgtctctaaaagacaaaacaaaaaaattagccaggtgtggtggcatgtgcctgtgttcccagctacttgaggggctgaggtgagagaatcccttgagccggggaggttgaggctgtggtgagctatgatctgcactccagcctgggcaacagagcaaaactgagtctcaaaaaaaaaaaaaaaaagagagcctggggcctaggacaacagctgaactagaaaaaaaacatagctaccacttttgaaccaggttctgtgcaggaacttctcttgtgtatcttacacccttacaatatccctgcaagatgactattattatttacaatttgcctgcaggaagttcatgtttggagagatggagtaacctgccacaggtcacaaaactagtcactggcatggccacaatctgaatttgggactgttagactgagaagcccaagctaattcttgatacgctcactgcctgggacatttcacacggtagatctgagctttctcgcatctgagactaaaaggaaaatgtagtccttaacagacagcagaaggaaacagattattagcaaaaagaactgctttcctagcaggaaaccagagaaggagttatttcatgtaagctcaagcaaaggaaattgaacaaaatcagttctttatctatcctgttattcgtatggaaataaagaagaaatgcttgcatcctgttgttttagtgcttgtactgatctttttcaaacatgcctgtgatcacatcactctctactcaaaactttgaacagctctccattgcctccatcagtgtgtttcaaattcgtaaaaagtaaaataaaataacaggatgcttttttcagatgaaatcttacatggcataccaaatatagcaagaaaaaagaaagagagagagagaggagagagaagccgggacaagctaaagcagagggtagagggagaaggaaccgaggaagggaagggtcaggtcccaggcatttctcttccctgaattggcctccctcttttgccacctccccagcaagacctttaactccacagtccacagctcattttgaaatgtactgatcaacgtgagaaaatgcgaagcttcagcctgatagtcaaagaccaccataatctggacccaagtgctagttcacttattttccctccactattttcaagccctggctatttgcaaactgcatacgtatgctgtcccagttctctcaccactcttcaaacacattctgaattcacctataatctcccctctgcccgccctgttgtagccccaatatccacccacctgtctaactccttctagctttccaaaagctttcacttgcctctttctttgaagctcacctatccctgccttctaacctggctatctgcatctctgttagacctcttacttagttcattagttcattctctctctgttgttttttgttttttgagacggagtctcactctgtcgccaggctggagtgcagtggcgcaatctcagctcactacaacccccgactcctgggttcaagcgattcttctgccgcagcctcccaagtagccgggtctacaggcgtgcgccaccatgcctggctaatttttgtatctttagtagagacgggctttcactgtataggccaggctggtcttgaactcctgacctcatgatctgcccgccttggcctcccaaagttttgggattacaggcgtgagccaccgcacccggcagttcattttctttatgtggagttatctatggccatgtctctcttcccaccctagtaagactgtggcttttccctctctaccctccagcacaaacacaatagatgttcaataaatgtttcctgagttttcacccctgtctctcctaacctgcaaagggtaagtggccctgcatagaagggcacagtgtggactttgttgttacaggcccaacatgtgtagcaccagtgggtgtgacctggaagaaatccccctagatgatgatgacctaaacaccatagaattcaaaatcctcgcctactacaccagacatcatgtcttcaagagcacccctgctctcttctcaccaaagctgctgagaacaagaagtttgtcccagaggggcctggggaattgttcagcaaatgagtcatggacagaggtgtcatggccttgcagaaattcccaatccagtgagaaggccataaaccttggcaagaaaaagtcttcttggaaagcattctttggagtagtggagaaggaagattcgcagagcacgcctgccaaggtctctgctcagggtcaaaggacgttggaataccaagattcgcacagccagcagtggtccaggtgtctttctaacgtggagcagtgcttggagcatgaaggtaggcatctgggatttctttctctcccgcttcctggtttttcccttctttgtgtccagagtggtacatctcttctcatgttgtaaagtggctttagagaaggcatgcgttgtgcctcagctttccaacttattagtgttcaatcataattgccctgcttttctcaagactaaaaggagttgctgcctgggcgcggtggctcacgcctgtaatcccagcactctgggaggccaaggcgggtggatcatgaggtcaggagattgagaccatcctggctaacacggtaaaaccccgtctccactaaaaaatacaaaaaattagccgggcgtgttggcgggtgcctgtagtcccagctactcgggaggctgaggcaggagaatggcgtgaacccagcaggcggagcttgcagtgagccaagatggcatcactgcactccagcttgggtgacagagtgagactccgtcaaaaaaaaaaaaatgactaaaaggaaatgatggaagttaagggtgctgggactggttttctttctccactgtctataaataagcttgtttcccaaattcctagtcctctgtaccagacaccaatataaaagaaggaggctaggcgcagtggctcatgcctgtaattccagcactttggaaggttgaggtgggcagatcataaggtcaggagttcaagactagcctggccaacatagtgaaaacccgtctctactaaagatacaaaaaattagccgggcatggtggcgcatgcttgtattcccagctactcgggaggctggggcaagagaatcgcttgaacctgggaggcggaggttgcagtgagccgagattgcatcactgcactccagcctgagctacagggtaagactttgtctcaaaaaaaaaaaaaaaaaagaacgagccctaaccaggggcttgcttgggctgcctgggccgctgctcttcctaagtcaactcattcgctcagggcctctgtgtttccatctgtacattgggtaccccactctctccacctcgtatggttgatgggaagatcagagagcaagagtaaaaggactcttgacaaagtacaaaccatcctacaaaaacaactgagaagcatttagacaaaatcttccctcagttggacaaaattaattgtacttaatgaaacattttcaccaggcactgaggctcatacctgtaatcccagcactttgggaggccaaggtgggtggatcggttgagctcaggagttcaagaccagccttagcaacatggcaaaaccctgtctctaccaaaaaatacaaaaaaattagtcagacatggtggtgcacgcctgtggtcctggctactcgggaggctgaggtgggaggatggcttgagcctgggaagtagaggttgcagtgagccaagatgatgccactgcactccagcctgggtgacagagtgagagcccatcacgtcgatactctcagcactgtgggaaaccaaggcaggaggatcacttgacaccaagagtttgagatcagcctggcatcatagcaagaccctgtctccattaaaaaattaaaataaaaaaattagcgaggcatggtggcacatgcctgttgtcccagctactatggaggctgaggtggaaggatcacttgagctcaggaggtcaaggctgcagtgagcgaagatcacatcactgcaccccagctctgggtgacataatgagactctgtctccaaaaaaaaaaaaaaaaagacagttgcccttcatgcgcacaccttggcataagcatcttcttccccttcaacaaagcccagtgcccaaagcccagattttttttttttttttaaggtgttttagatggagtcttgctctgtcgcccaggctggagtgctgtggtgtaatctcggctcactgcaagctccgcctcccaggttcaagcaattctcctgcctcagcctcctgagtagctgggatcacaggcgcgcaccaccaggcctggctaatttttgtatttttagtagagacagcgtttcaccatgttggtcaggctggcctcgaactcctgacctcgtgatctgcccaccttgggctcccgaagtgctgggattacaagtgtgagccactgcacccagccgcaaagccagattttaaaagaaaggccccctcctgctgagtttcattggcctgggtaacttgtcaataattagtcaagaggtggcagtaatgtctcagtttttaaatgtgacagtcctggcttgcttggagaatcagcccactcatagacagctttagatcactcactatgatcaaggaaactcccttttcccattagagtttcagatgtttagaacagtgggagttatggtcaggatcatctgagggaacctcatggcacagacaggatgaggaaatgggagtcccacaaaaagggcttaccaaggaaacatccaggaagtagcagagcctggactcaccagaagtccaggcctctccctagacttgtttcatttttacagcaaggcatctgctctctgaactctgcccagctagggcaggtctagggcaagagggagcggccccctgtggtccttccctccacatctgcctccccacccctcagctgccctcacccagccatggcttcttctgccgggggtggcatctgggctctggggcaaagaacagtttggaggcacctaaataggcttttctacttggcctgtactgtggactttgccagcagacagagaaagcttttacagaccttagtcttctggtttttcctgagataaattgcattctgtatttcagtatttactggagtgtcatttgacattttttttattttctaaaatttcaagagaagcttttatttcttttttcagtagggaaagaaaacttttatgtgagattgagaattaaagattacattcaaaaacacttaggtgatgtgttaatgtgctgaatagctgaatagtacctatggctgaataatattctccataaaaaatgaaaaatgattacctcctaagaaggctttaaacacatatttacagcttatcttcccttatcacctttctcctcacgcttgcatttagtcagttaattgtgtgttttttacttgtgttacttgtgtcggttttacattgagcgtgatccaagagagcctcacattctatttctattttgccacatacaaatcaggccttgcaaatttggtttctcctgggttcttttttgtttgtttgtttgttgtttttgagacagagtcttgctctgtcacccaggctggagtgcagtggcctgatctcggctcactgcaacctctgcctcctgggttcacaccattctcctgcctcagcctcccgagtagctgggactacgggcgcccaccaccacgcccggctaattttttgtatttttagtagagacagggtttcatcgtgttagccaggatggtctcgatctcctggcctcgtgatctgtccgcctcagcctcccaaagtgctgggattacaggcgtgagccaccgtgcctggcctcacctgggttattttaactgtaagtcactatgtagccttgtacagccactgccaggcctcagtaccgggcttacatgggaactgtgagattatgtcccaagaccaaactctgttaggaagaggcctcaggatgcagagaaatggacaataacaccagttttaaagactaccaacccaggctctttcacttgtgaatcttttgtttctccatagctacgatttgctcacgtggaatgttatagttaaggtgaggttcagcgatatacagtggaacactcaaatacccatggcttaaataatatacaggtttgcttttctctcctttaaaagaggacaaaagtttggcagctcaggcatgatatgacagctccacaaagttattaagaatttctgggaatggccaggcagggtggctcatgcctataatcccagccctttgggagtctgaggtgggcagatcacttgagccaaagagtttgagaccagcctgagcaacatgacaaaaccccgtctctattaaaaaaaatacaaaaattagccaggcgccatggtgcgcgcctgcagtctcagctactcgagaggctgaaggaggagaatcacttgagcctgggaggcgcaggttgcactgagctgagatcacaccattgcactccagcttggatgacggggtgaaaaaaagaattcctgggaattctgccatccctcagctcgtggcttccacctcaaagtcaccttatattctaaaggtggctgttggggcttcacccatcatgtctacatctcaggcaagaataaaaaggaagcaaaggaagggcaaagggtgccttctttctaggtaattcagctcccttttattttattattattttttaatcttttgtaaagatggggtctgtgttacccaggctggtctcgaactcctggcctaaaatgatggcctcgccttggcctcccaaaatgctgggattacgggcgtgagccaccgcacccagccttagctcccttttcgagagctttcttgaaactgcccccagcttctgacatctcattggcccttccctactgcaagggagcccagggaatatagtttgttcagctgggttcattgctgcccctcacattttagaggtctgttactaaggaagaagaagaaagtggatactgagtgggaaagagcagtccataaggaagtcacaccccaaccccctgccctttctctttaaaacaaatgcctcagaccacaggcgaaatgggaccttccgaccctgttggcctgctcttcctggcgccacgatgacccccaaaacagactccaaggaacagacagcctttctgtcttctccccaccccatccagtgttccctcgcctatcagacaggaccaagtctgacaaggatataatcttttctttttcatgttttaaaattaaaatatccaagatgggaattttaactagagctagtgcatctgtgggaacatgtttgcttatagtttctcctggacatgtctcactccatgtccacaaacccacagaaatcaataccctgctgtaaccccagcactttgggaggctgaggcaggaggatcacctgaggtcaagagttcaagaccagcctggccaacatggcaaaaccctgtctctactgaaaatatacaaaaattagctggacgtagtggcgggagccactacgtcctgtaatcccagctacacaggaggctgaggcagagagaatggcttgaacccagaaggtggaggttgcagcgagccgagatcgtgccattgcactccagcctgggcaacagagcgagactccgtctcaaaaaaaaaaaaaaaaaaaaagtcaataccctgtacacagttagccagattgagtaattttgtacctgttggtgatcttgtggccaccctgccttatgagaggaggaggggacagagcctttgggacccttcatggagcttcttaatgtgcaagatgggcacagggtgctttctggtggtagaaccgcatgctgattaccctagatgagctgcacaaggttgaaaaggcagcagaggcctgctttcctttctcatctcctgaggacatggccagggaagtttcccttggctttggaagatagcctcttgccatttctttctttgccaggggccatgggtggccagagttccagcggaggccggctgggctgcaataaggggattacaaagtggaaagtctgtttcgcttggttcttaaaaaacgaggaggaaagacattgatccttttcttcttgatctttcttctctccgtcattcgcttcttattggtttttgaaccctgtggactaaccatggaaaagatggctttatgagtcagatgtccaccagaggatggcatcatggacagagcagtatcttctgtggcctttccgtgaatggctggagggctgaagggaggacactgaagccttcagcttggaggaatccagggagagggatcaggagccaccttggcagctgcatatgatgtcaaaacctgtttctcttgtggctcagtccctcccaggccccctacttgcctctcttacctccagtgctgtacaaagcccagaagtgccctaggagtttgagcacaccaaccttctgtccaaaacgccactccattactgtcttcaagtcctgtgaattacctaatgttagtcttcaaatgttacatggtaaaacccagctgctccacacggcatccaagacagcttgtacaagcttttaaaagtttaggatttaggttgggtgtggtggtggctcatccttgtaatcctagcactttgggaggctgatgtgggaggattgcttgcgcttaggagttcaagaacaacctgggcaacatagggagaccttatctctagaaaaagaattttaataaattagccaggcgtggtgatgcacacctgtcatcccagctactcaggaggctgaggtgggacaatcgcttgagcctgagagatccaagctgcagtgagctgtgatcacaccactgcactccagcctgggtgacagagtgagactctgtctggaaaaaagtaaataaacaaataaaaggttagaacttagtgacaaatcaacagtgaaaattttaccacccagcaactcagccatgccatctgcccaccccacctcccctcttccccactcaggggcaaaggcgactggtctcttataggctacaacactcactgggccaatgccatttcaccgtcctatctcaaagtgctttattctacaattctgtatgcaaatacatgacgtaactcttttctcagcttgtaaattcttgaaggccgtaagttgtgctgactgcatccttgtttacctataagcattcattgaatgttttccagtagacatggagaggagagaagaggcagaactgtaccagcaacgggagttaggtgttcacttgataatggacttggatgatgaatctcattgccctgagagcagggatatttctttcttcagtgaccaaccccgaaatgcttgtcaaattatcgaatgtcctcctatgcatgtaggggctggctcttagaggtataaagcatgtcgagaaaaaagaataaaaataaaagcataatgagcattccttcaagtaaagagctaagcactcctttttgtgtctcttcccttgcaaaggggaaaggagaccctaattttgcaagacttcaaaaagagtgtgcagaaggccaattgtttcaaggtgacaatcattcctgcatgcacccctcccacatcccggcctctgccaggcaactttcagtcctttctgaagaatgaccctcccccagcacacatactcccaggcagtttccttctattctggcctggtcttttcattccaggcaactttgcataccaatgagccagatgaagttctgggaagggcctctcagcaccattttgtcttgtttcagctgtggaccccaaagtcatttccattgccaaccgagtagctgaaattgtttactcctggccaccaccacaagcgacccaggcaggaggcttcaagtccaaagagatttttgtaactgagggtctctccttccagctccaaggccacgtgcctgtagcttcaagttctaagaaaggtaagctttccttccctgggtggagtgtttgccaggcaggggcgcaggagcatttgtgtatttatccatcccagggctcggcaacttgacagtctccgctgcctggactgagggcttgacagccacaggctgtgggcaatgagctccagcttgagacaccagcccaaggcaagggggctgccccagcaggttgaaaaaacattcccagctctctgctaagtaagaaaggaaagtctcctacagagggagaggaggaaacacaaagcccgcattcagccttgtctgtggcggaaaatgcaatgttagatctgatcatgttaccagtgaccctgggccaatttgcatcaggtacagttggagcgtcaagccagggatttatattataacattgctttagggctttgtgaaaaagcaggggttgtcaggcaccagctatggtgcaagacatggagcagaactgagaagtagtgacaaggctgggtcagttgtcacccaaaccatagcaatagctgagactgtagatcccatcaggctggtggtaactcaaggcactgaggggagagtcaaggggagcaaagccaagtgccactggaagagaagctctgcccggcattcccccaccacagttcttgctcctggctccctccacaaactacagtggaggttgtgatgacgcagcctcattgtctgggataaatacgtgaggttcgttgtctcatgccaagggaatcgaggacatggacacacacaagaagtgagtttagcggcagaggtttaataggcaaaacaaagagaaaggagaacagctctctctcctgcgacagagaggggaacccaaatgtgacttctggcctgcgacagagtgcaccggattttatagacaggtttgaggaggtggtgtctgatttacataggatccaaagatcagttgaaccaggtgtgacgtttacatagcatgggggaagctggccaccccaccctcatcttattacgcaagtgggctttccacttgcccagcgccatgttgtctgctccctagggcacacgtggttggaaaggaaaagggaaaatgcggccgccatgttggacatgcctagtcccaggtggccttttcctattggcacagctgccaacattcactgtgcaagcttttagcttgcttgtctatgtctgcagctcaattttacaggctactctttgttagaaaagaaaatgatttggggctgcttttcattaaaaggaaaaccttcccgagaacttccttaccctcactatctgcctaaatcatttctttttaactcctctaacagtgaggccaaggtggatcttgggtttgcacctccctaaagaacaaggtgaaggttctccctccaaaggtatccgtgtctcccaaccccgatgcagagcggtagccacactaaaggggccacagctgccacagaggctcaggggaccgtgtgaagagctggaggagaggaggaggcctctgagtctgctaaaaaaaaacagtgtgacattttattactatcccacatgggcttgggagatggaatgccacccctaaaccaagctaaagctaagatgacctcatcctctgtgcacaggagaaatacaggcagctgtgccagcaactttccctaagagagtgttaacccaatatatcgagacagatcttactcagtgtagaacttttattttggccgggagcggtggctcgtgcctgtaatcccagcactttgggaggccaaggcaggtggatcgcctgaggtcaggcgttcaagaccagcctggccaacatggtgaaaccccttctctactaaaaatacaaaaaaattagccaggcatagtggcaggtgcctatgatcccaactactggggaggctgaggcaggagaatcgcttcaactggggaggctgaggttgcagtgagccaagattacgccattgcactccagcctgggtgacaagagtgaaactccatctcaataaaaaaaaaaaaaagtagaagaagaaggtttattttgccaagcctgggcacggtggcttacgtctgtaatcccagcacttcgagaggccgaggcggacggatctcttgaggccagaagttcaagaccagcctggccaacatagcaaaaccctatctctactaaaaatacaaaaaaattagccgggtgtggtggcacacacctgtagtcccagctactcgggaggctgaggcaggagaatcacttgaacctgggaggtggaggttgcagtgagccaagatcgtgccactgcactccagcctgggtgacagagcaagactcacatactccactgcaccctgtccccccaacacacaccatacaccaaccgtggccacgttcctatccatctgcccttttagccttggccataggggaatttattaattttacttatttatatcccaccttattgtcaagacccagcatacaaagaataaattttagtcagataaaatccatcaatctgaaagatatctttgtaaaaagaaaacaaaaacaggaaaataagacggatttgggaattaggttaatttccaaaatgtaggccagcaagactatccactctcttagatatgggccacaaatttggctctatactttctagcaactaacccaaagagtgaaatataatcaaatatgagatttatggtgtccataagataaaagcaccatctgctgaggaaaaatccaaaaaccagaagaatattccccctaaatatcttcatagacagaatcctgtgctgtgaaaccgatgtcttcaacaacaagcttaaggtaactacagcgatgaatttcacagtgagagctgggggactgagcaaaggaagaaggggaaagaaggaaaggaaggggctgtaggagaaatccctagtatggtttgtgtctgggcagaagaggcgaggtaagatttacttgataagaatgaacatgcttcttgccaggcacagtggctcatgcctgtaattccagcactttgggaggccgaagtgggtggaccacctgaggtcaggagtttgagaccagcctggctaacatggtgaaacctgggtttctagtaaaaatacaaaaaaattagccaggcgtggtggcacacgcctgtaatcccagctacttgggagactgaggcatgagaatctcttgaacctgggaggcagaggttgcagtgaggcaagatcgcaccattgcactacagcttgggcaacaagagcaaaactctgtctaaaaaataaaaaataaaaaaaaaaaaaagaattagcatgcctcttcctccagcccttaccaagccacaagcattcattgtccctggaaaaatgtaagattattttttgcttctcttaaaagaaataactggaattttccccgacagatgaagaagaacaaatactagccaaaattgttgagctgctgaaatattcaggagatcagttggaaagaaaggtatggaacaccttgaactgatgcgattgatttctgtgcccatgcactagtacacgagaatggaattgtattccaggttgcaaccttattccctttctaagtcctggtttctacttagtcctaaattttatttcccttggagtcaaaattcttcatgctggtagaatctcaaaaatgagtcactttggaaaacataagtaaaccttctggagcctccttggaatttttggaaactaaaaaagaaacttgttattgcaagaaaagacaccagtaaattaaaatttgctggatggaattattaatctcattccccatgtaactgatcctcattgaagtttagactcgcatttgctttgcagcctggatatctgtgtcccacagttgctccaaagctggaatactttctggaatctttttcccctgtagttgctggactgttgcctgtttaagggctctcttagcagttcagccagggaggtgcgctagtgttactgggagaatgtatggatttcctgccatttggaagttgagacccatctttccaatcaggaacgggtgagagttgccttcaggtaaacagttggtgagcaaaccttcaacagagtcaaaagggcttctctatgcctcctgggtccataacccacagacgactgagtagtctctagaatctgtcaatcaggggctggcttgcagacatagacccttgtttttccagaatctgtggggagggagtcagaccctgggggtggggatgggaggttctgcaggaagacaaacagctcctgtctatagctttgtcaaaagtgtgcccagaggctggtgacccagagggcgaagcacctgggagatgggacagaatgagaaacagcgagaaaaccagggatattaggttaaagaaaagagaacggggtgtgtgagctttgggaccacaagtagagggtggagaggggtgcagaggaagcaagagatggaacaggatcatgatttaagggcacccctgcatttctccagagctagtgtcatggatacacaaattatgcctagtatggggtgattgatttctccttatggtaagaatgaacctccattcctggagaataggcattccaggctgcccatgagagtcaacgggtgccttgtgagagaatgatcttcctgtcacgggaagaattgcaagtaggtgccaataccagttagcatttcagcacagaactccgaggggtcctttagacaacctcaagggctctttacaactcagagtttctgtggctctcacggcaacctccttcccagcttccctaagcagggacttgtggtcattggtcagggaggtgggaaaggcggagtgggggtgctggaggtgggacgaaggccaccgatctcaggttatgaagagcgatggtttcattttcacccccacaaattaccactttcaaaaacctcactttgcctggtgctctctcttaaggagtttggacattttacattgcttatggcaaagccttggttgttatttcctttacatgcttccccagtagggagctctgctagagacacatggtaggtgtgaccttgtgctgggcgggtgccaagctcccactgcagagtgccgtgtgtgtggctgcaggggatctgcagagccgctggtcccaagagcacacacagcccgaaggcccatcctacacctccaggagaggccaaagagccaggtgcacccgagagggacaggcaggggaggggtgaggaggtgccagtggaagccctgtctttgtggcgtttgattcttttactgttctggcacagtccccacttggctgcctgctagggttttgagcctggcctcctccattgggaaactgatgaagaggagtctgttccctgttatggaggaggctggccatgcctcctgcagagtcacaacccagacggggcctggacggtgggggtgggcgtggttcagggaaaagacaggtctgccaaaatctcccaatcgcatctgactcatcccccagatatcccttttagaaacaatatttgttgaaaacccgcttggcttatcattcctctaggacagaactagaggattttcaaataaatattttgtccctgctcactcagaaatttccgttttcacatgacaaccagcatattaacagacattgcaagaatgctttaaatgaagatgatatatagctttttgatatgagccctgatttttcaaaagcagaagaggcagcattgattttgttttaaaggcgtggtagttcaaaggaggtaaagcagagagataccaggttcaagggaaggccgggaggtaaaatcaatagggtagaataatgggaaagaaatcaatgaattggagtttttttaagtgaacattgcatgcaagggagtcttccctgacctccatgctcccgtagcatcctgaaaaatgactcgcatttccaaagcacctcttctatgtccagcactataaagtgttcaactaccctatagtatagggccaagcacagtggctcacacttgtaatcccagctcctggggaggccgaggtgggaggatcgcttgaggccaggagtttgagaccagcctgggcaatatagtaagccttgtccctaaaaaacaataataataaaagtttcttaagaaaatgaaggctgggtacgggcacagtggcccacacctgtaatcccagcactttgggaggccgaggcaggtggatcacctaaggttaggagttcaagaccagccttaccaacatgatgaaaccctgtctctactaaaaatacaaaaattagccgggtgtggtggtgtgtgccagtaatcccagctacttgggaggctgagacaggagagttgcttgaacctgggaggcggaggttgcagtgagctgagatcgcgcctctgcactccagtctgggcaagagagtgagactccatctcagaaaaaaaaaaaagaaagaaagaaaagaaaagaaaatgaagactgggtgcagggactcatgcctgtaatcccaacactttggggaggctgaggtgggtggatcgcttaagcccaggagtgggcaacatggcgaaactctatctctacaaaaaaatagaaaaaaattagccaggtgtggtggcatgcacctgtagtcccatctacttgggaggctgaggcaagagaatcacttgagcccaggaggcagaggttgcagtgagctgagaccacaccactgcactccagcctgggcaatgggagtgaaaccctgtcttaaaaaaaaaaaaaagaaagaaaacaattaccctatagtatcaatactatcaccatcctagtttagagatgacaaactgaatctgagaggttaatttccccaagattgcatagctaagtaagaggtggacccaggggtttgagccaagacagtgattcctaagcttcagatcctgtcccttacactacttctagcaaaggacatcacatttttctgcaattcccactagactgtgagccctttgagtgaggacccgcatctcagtcatccatactacctgcccattataagtgctcaggaaatatttgttaaatgtcaaatgaactgactgttccattttatacatgtatttgtgctgatgaaatgaattcaccgagcagtacatgcccattctggttctgcaggacactgccttcatccccattcccttggttgacaccagcatccagggttttccacaggatggtttgatggcctgcatttgagctaaagaatgaacttctgtctgcctcgtggagccaagctactgtactgagtgcttattcttttgtacacagctgaagaaagataaggctttgatgggccacttccaggatgggctgtcctactctgttttcaagaccatcacagaccaggtcctaatgggtgtggaccccaggggagaatcagaggtcaaagctcagggctttaaggctgcccttgtaatagacgtcacggccaagctcacagctattgacaaccacccgatgaacagggtcctgggctttggcaccaagtacctgaaagagaacttctcgccatggatccagcagcacggtggatgggtaagcgtatcctatttaaaaacaaattttctcagaactcagaagagatgggatggattttttttttttttttaaatgaagggaacacatctatgaagcagttctcatgagtttaggacacttgagtggccagagatagatcccattgatgggaacatatttttagtgattatcttcatcatcaataaatatttactgagctctccaagggtatgcagggtatgtgtggatggaaagggactggtcagatggaacaggaaatgaggagatgctgagataagaaatgtttagatagacgtaatttctgttttcacaaattttgcaggcaacggaagtctggcagggagacaagggcaggcagacagttcctcacacttgcagatacttagaggtcacggagactaagcctcatgcagagactcagggttcatggcattttgtcattgctgctttgacccagccattttgagggtgacattttgataccaaataaaaagccaacagttaaactttcatctattttcaattacctggtatcagtcaaaacagacctctttcccttttgcctcaaaccctctcccatccctcatccccagttcctcaggtctccttaagactgccatgttcaaacagctacataaggtccaccgtgtcaaaatgtactaccccacaactttaactctcaacaggcctaggcattcctaggcggacagaccttcatcccacattatccttagttctctttccactagaatggtccaggggccagcaggaggattgagtcggctctaggcatggctggagtgaccctgggacctccacacagccttggtctctttgctgatggaaggaaagtgcagctgatcttgcccaagggtctgaagtgcccgccatggtcttcagtggctgccacggtcttcagtttctgtacattgagttcctttctattttccacttttgcaccaagtttgataaccctaagtggctgatctgaacaaccaaggccattcttaagtattctaaacttaagctgccttagcttattctaaaaggaggacaggttcctcaacattattttagttctacctattcttctttccaggattccttcaccccaacaacccccttgagccctcatctgccccaaagcctctcctgacccttccagctcaaaggctgcacagtctctaatctccagcacttcatgtttgcaccataataatatacaaaagatgatcaaattcccaattgactatgtttcaagcattcattttaaaaatcattgttagtcctcttaatatattttctcagagaaattataattatcaattgggttcacaggctagctctcaaaaatctgtttaacccactaatgtagctaaaataccataaatatgctattaaaaagcagtggttctacaccaaaagcacaagcaacaaaagaaagaatagatacattggattttgttttgtgcttccaagaacaccaccaccaagaatgtgaaaaaaaaaaaaaaacaacccacagaatggggaaaaatacttgcaaataaaatctctaataaaagacatatctaaatacataatgaactattataattgaacaataaaatgtcaaataacccaattttcaaatgagcaatggatttgaatagacatttctccaaagaatatatataaatggctaaaagcacatgaaaagacgcttgtttgacatcttcatttcaacagtaggaaaatgcaaatcaaagcaacattgagctctatccactacagtggctaaaatgaaaaagaaaagcattgacgtcggtgaggatgtggagaaattggaaccttttatgttgctggttggattgtaaaatggtgcagtccctttggaaaaccacttgggagttcccaaaaatgttaactatgaagttaccaattccaggggccgggcgcagtggctcacgcctgtaatcccagcactttgggaggccgaggcgggtggatcacgaggtcaggagatcgagaccatcctggctaacacggtgaaaccccgtctctactaaaaatacaaaaaaattagccgggcatggtggcaggtgcctgtagtcccagctactcaggggggctgaggtaggagaatggtgtaaacctgggaggcggagcttgcagtgagccaagatcgcgccactgcactccagcctgggagagagagcgagactctgtctcaaaaaataaaaaaaattaaaaaaaataaagttaccagttccactcctggatatatacccaagaaagttgaagacatattttcatacaaaaatttgtacatgaatgttcatagcttccattagccaaaaagtggaagcaacccaaatgtccatcaactgatgaatgatgaatggataaggaaaatgtagtatagccacacaatggaatgttattccactataaaaataaagcaagtattgatacatggatgaaccttaaaaacattatgctaagtgagagaaagtcaggcaaaaaggccatatactgtatgattccatgtatatgaagtgtccagaataggcaaatgtatagatacagaaagtagactcgttgctaggagctgagggtgagagaatgggaataattgctaagggatgtgaagtttatttttggagtgatgataatgtcctaaaatcagaggcgatgtttgcataactctgtgaatatactaaaagtgaatatacgaattcactgaattagtgaattttatagcataattatatttcaatcaatctttataaaactagcagttctaattctagtaatcaaaaagaggaaagagaaatgaatgcaattatttttttaatttggctgctggtactggagaaaaaaaacagttccaattagaaaaaaaaaaagatgaattattcagtaggaatactaaagtggatttccaaaggctttagaaattgatggcactagttctttaacatctgtagtttcaattcaaaatgtatttcttatccaagttatcataaaccttaatcttaactcttggccacagttaagtttaatttggagcatgtcagtggaaattaggagaggggagacaaagaatgtgagagggatgactaatttttccaaagggtagaaaaaaataaaggaggatgataaagaatatttatgagctgatatgagctatggaagtgtgaaggacatgtggattggggcgtgtgctcagagtcccttaacgggaaaaaaagaagacaatgggaagtaagcctcctgggacttccattggcaccaagtgtctcgatggctctctctgctgtccttgtagtagtgtgggaggagagaaatctagtcccatgcacaagctttccagaagctcagggttcctatctcagtattccacatttcatatgagtacatctcatcttcccaactaaactgcaaatctgtaagggtgagatcatgtttttatgatggctcctttaatctcccatagctcccattcctctacataacaccatagatgttcagccagtacttgtagaacggtgaatgaatcgatcaaatgaatgaatagcaactctatctacaaggcatgccaaacaagctaccacgagattctgatttactccttggctctttgtgctctctgtggagcaccattcacatagttaccctgcagtcctgcaacctcacccattctgtagcactttgaaaggagagagttgtaaccttcatagtcattgacctttcagtacccacttttattacattttaaatacacccttgaagccctcccatcttcaaggaaagatccagcctgttagctctgggtgtgtaacgtaagaccttagctgatgtcttccttgcaggcataccaagaatgccagtcacctctccaaaccctcccccaaactaacatggtctcctgagcctcgggtcacgcctgggatagagcagagttagcagccatatgttttcacttcaacagtaaatctaacttggaattttaagaacctatttttcccctctaggaaaaaatacttgggatatcacatgaagaagtagactgaaatatcagatttgtcatcaggaatactctttgtctactgtggtcctgtgcacgttggcctcagatggactacaggagattacaacgtacaaggcagatggagcattgacgttttcaaaaccattattcctgtgactggagaggcatcaggagaggtctcgttcgtctccagctcataaaatgtagcagcatcatccttgacagtgatgtttttcaggccctccattgagaacctgaggaaatctgtaaagataagtggtgatgttgtttcaaacgttcagaacagataccatcatcctgcctttgttagctgctgtagggaaagtgcgttacagatgtctgctgacctcacaagagtgaaaagataaactgtgcatgtgtttccacttccgtttctagtactatttatttttaaactacacttggggtggcctaatacctaggaagatgttgctattcacgttagtaaacagcctaaagaaactcttaggtttactgctacatccatttgtttggagaggtaactgttgtctgtgcctttttgaaaaacttccatttggtacaaaatttttactccaacaccccctcaacccttttctcagggaccacacctcttcttcccaaggtccctgggacttcctcattctttgtggtagtacaatgattggtagcaggtaaaataaatacatagaaagactactgtcaaaagagtgtct

SEQ ID NO: 33 - protein, 452 aa, Homo sapiens
MSETPAQCSIKQERISYTPPESPVPSYASSTPLHVPVPRALRMEEDSIRLPAHLRLQPIYWSRDDVAQWLKWAENEFSLRPIDSNTFEMNGKALLLLTKEDFRYRSPHSGDVLYELLQHILKQRKPRILFSPFFHPGNSIHTQPEVILHQNHEEDNCVQRTPRPSVDNVHHNPPTIELLHRSRSPITTNHRPSPDPEQRPLRSPLDNMIRRLSPAERAQGPRPHQENNHQESYPLSVSPMENNHCPASSESHPKPSSPRQESTRVIQLMPSPIMHPLILNPRHSVDFKQSRLSEDGLHREGKPINLSHREDLAYMNHIMVSVSPPEEHAMPIGRIADCRLLWDYVYQLLSDSRYENFIRWEDKESKIFRIVDPNGLARLWGNHKNRTNMTYEKMSRALRHYYKLNIIRKEPGQRLLFRFMKTPDEIMSGRTDRLEHLESQELDEQIYQEDEC

SEQ ID NO: 34 - DNA, 245549 bp, Homo sapiens
gcgtcccgggtccccgcgccgcgccgcgacctgcagaccccgccgccgcgctcgggcccgtctcccacgcccccgccgccccgcgcgcccaactccgccggccgccccgccccgccccgcgcgctccagacccccggggcggctgccgggagagatgctggaagaaacttcttaaatgaccgcgtctggctggccgtggagcctttctgggttggggagaggaaaggaaagtggaaaaaacctgagaacttcctgatctctctcgctgtgagacatgtctgagactcctgctcagtgtagcattaaggtaaaaatcttctcccctccttctacgtggtggaaaccctgagctgcaccggccagggcagtcgtgctgggctcctcagagcaggctgttgcagttgctctgttcgcaggaaattatttggggcgagagggaaagagatgcagctcgcggtggctgctgtaccctttagcgtaaccttgctactcccccccaccgccgagcccctgccggctcctcggcccccacccccttgctttttgacaatgaaatgccggtcacaattggtcagagcaacccggattgtctccagaaccgtaaaacacccccgtaattagtgcgcttaaaaaaaaaaaaaacaaaaaacaaaaaaaaaaaacctgctccctattcctacacccaacatgtgactgaaatataacttttgttcccgaggggtggtcgtcttagaaaaaaagttgttcagtttccactgcgatagaatctttttatttttttaaaggactctctgggtctgatcagacaggatttttttccccctcgtctgattgtaattgcaagcgctgtcggagagacagtcctggtgtcagcttttcctaggcattgcacaatgaatttttttttaaaaccaaggtgaataaagttgctgaatagttattttactccgatccatatgaaagttgagcaatcccaaagtagaaaactcctgtgatggagggagaggaagaactaagcaaccaggcaaaatgaccttctgtgtttgacagtgcagagaggctatctaagcagcactctgtaaatgcagggcggtagatgtttgtaaacacactgctgggctacccagtagttgtgtgtatgcatgtctatatatctgtaaagactgtggtgctgcggtttgttgcttctaaaaagttgttgtaaagtttgccaccattgggaggcaggatgatttatccatctacccgtgttatttttcccatcgctgcatttattgtacactttattcatttcgctgttccaccagtacccaggtagaaagccttccgctcttgtgagaggctgaagctgctgaagtttgtttttggtttggcctatttaatgtgtgttcactgaatggacactacagcaacaatgaacaaataacctgttttcttcgtctgtggggagttagaaaaacccttcctggtttaacactgcctttcaattagagtctgtgtaggggaaacgcagtagttcatcatacgtgcttactaggttttttataattagaatcacattttggtgtttcttatcagaaaagatattcatgggtttgaggataggctaatgtgaaatcaaaaggtggtttgggcttctttgtttttgcctcacaaaaatgcctaatggaacttttaggcttttgtgtttgtacataactggaggtagcagtatgtatgtctgtgggcatggggcgtgccacactaaagaacttagggtactttgatattaatacttcaaaaccagtttcttttgcggtataaattagattctacacagagttcaggaaaatcctgcggtatagataatctaatttatttgacgcttcataacatagaggtaactaaatagtaggatattcaataaataaattgtatttggctcagtggcaataaacccgacatcaggaaaatctgccaactacagatcagtaactttagggcaaaaacaacaaaaaacaatgctttctgtgacgtttcttgatcctcttctatactgcatccccaccgttttgatgggcttatggtggtgtgctggctgcagactgctactgagtggtttgtttcattgtgctccagtcggaaaaagagtacaacagtcccttgccttactcacatacagaaccaatcccaagcaaaacgcgttcttaaatgcaacatgaaaattgtctgtaatttgcatatcatttttttccttattgcttacaactcagttctttcagggtgcctcttttcattactgtgtacttctatgtaattaaaatagatttaatgttctttaccagaaaggaaagaatcacttttattttggatcgttctttatggaacgagtatgtatttgcttagactgcatgtgtctaggtgaacgtatgtgaagagtatctttccatggagtttgtttaattcagtgttccaataataagcactagagatcaaggactgggattattccaggtcatctgaaatgttctggtgatgtgtgctgcgtttcatttagtccttgggatgtggtacggtgactgcataataaaaaatgtttgctggtaacccacaagcaagttgcttgcttctagagtccagtaaacaatatgcttttctgtgtagtgaagaacgaatgtcagaggttttcaaggatgagctgatgaacctgatataccgaactcctgtgacacaaaaggctctcactgtggagaggtttttcccagtcctcctctccactcctgaaaccgtcctcacttgctttcattccttacaagtagcgggtcagcaggcagacagttcctcagcggctggtgagatgcagccacagagggaagtactttgtttatttctgagcagtgcctgctctgctagaaaagagtagtgtccgatttgggtggagtttccaaggtgaaagtttctgaattggtcaatcagtgacgcctttgtaaagatggctcatgtggtggtcgctcgcaatgaatgcctgataagggcttttctgtttcttttgcactgtgtaagtttgctcccatcgcctggggaagttaatatcagacacacactttttacggtagaagagaggttgactactccaagggcactgaaactctcactgagccttattgtttctctacacgcgacttgcagaaagcaggagtgctcgagcactcctgggggggtctgtcctacaacccttgaaggttgtggcactgttgtcatggaagttcaaatgcttcaattcctgaccatttggggtcgtttaaatggaaggaaaggatcaactctctataaaaagtcaaattgttttaaagaggattgcctggctgactgtgctggtcacaggaagaaggaagaggctgtcttttccatcatcagccttcaccttcaagtcacctcttcagttctaagtttgaccaagttgcagcggaaggtggttgtggcctagcagacacggtcccttgtcaggcttggcacagcttgttagtgggccaggactaaccagaatccaaaaacgagtcacacagtgtctctagtcccctccttccctgctgtccgtatcagtcagctttcatagcagaaggaaatctggtttttaaaattttgctactaactgggagactctttcgagctccccttctggactctgggctccttgggtttagtgatgattatcttggtgtcctagtagttagcttggcagctggcatagaaatatttctcagtaatgtttgctgatgtgaatgaagtgagaggtgggaggaggccagccccctttcctctttctgtctgcgtgtaacctcagggtgacgtaacactttcatccactggcttgcttgcagaaagcactcaatagggcccactacagccttagagagggctagagcactcctggggggtctgtcctcaaatgcttattaatagaattttgatacaataatgttttataatttttttttggagacagagtctcgctctgtcgccagactggagttcagtggtgcgatcttggctcactgcaacctctacctcctaggttcaagcaattctcctgcctcagcctcccaagtagttgggattacaggtgcccgccaccatgcccggctaatttttgtgcttttggtagagatgggggtttcaccatgttggcaaggctggtcttgaactcctgacctcaagtgatctgcccgcctcagcctcccagagtgctgagattacaggtgtgagccactgcgtccagcgtaatgttttattattttgataaaattctcaaaaactgcttttgtgtcaaaaataacaaagaaggaaattgaggaaaaggtacaacaaaggggcttaaggaattgctttaacaggttcagattagcaggcttctaagtgccccccagtttttctgacaatagaatcagggaactttcaaacattcagagttgagagaccttagagatcccctgggccaacctcctctattttttacagggaggggatccagagagctaaggggtttgcttcaacccatgggtgagtgagccaggtttggaggacacaggcctacaggttcttaggccactgcattctctgggttcatggtgtgtccctataaaaggccaaggaaactcgatataataggcttcgctctctctggcacttcctagcttggtccattttaatcacaagccagctggtctgagggcacgggggtatgtgccaaggaagatggtatgtgggggtagaaatgggccactggggtctggtcaggtgtgagaagaggaagtagtaggagggaggagatgagagaagaagggcctgacggacttgtattggcaccactatttgcatcaatatatgtatatatatatatcttttacagtgcacaaaggcctgcaagttctgttttgggctgaaggccccctatctgctgttaggactcctttgaggaatccttgggtgggccgtaggctcttttatcaagggtcagacaatcctatttatactttaatttctgtgtagtaagctccctgtttcaaacagtcaaagctaaaaatgtggcagctggccctagagaagtagctatcctgctcaggataagtaagtcaagggcaaaaaataccccagatgtgagcaccagaggcaggtagagctaagtcacattctgctcctctgagtgcagctgtatttgtgcacaccaggaaagagctgggggcaaggtgggggcacagggttgctgagagtaactcacacagcagtttcatttttttcaccaatttttaaaacttgccctttggtgtggatctcccagccaagtgattcagtgtcagatgaagccccgtaaggtgtgccatatagaaatgacatgtttgttctatgtgaatactactttagagacaatcctggagcttgttttcaggtaagggtccaccacttccatatttgttgttagatgtgtgttgggaaccaatctccgtgtgtatttgttgaatagaatgagtgtgcatttactcagggttcccacgccaaaagggacccagcttaaagactgttcactgccaaacctagtatttggggacagaaagactagactaaattagtaaaaagttagaattatagcctattaaaaaacaaacacatttttattgattttccaaacatacaggaaccaaactaataaagtgttgcttactggggcatctcagggaacgttccaatgaataggcaagaatgtgttccacattatccattgggtagaaactagtgctacgatgtgcttggaactgttcctttagtgtcaaaatattaccacacattttatttgctctgttaatttgtttgataaccttttcttcttagtaaaaatacagagttcagcctggtcgttgtctaaatgatctggaattcccagagtgtggacgaggggcttgattagaaaaggtattgccccgtgttaagtgtggactctcagatgagcataactgctcactcagccctatggatttcgcttacaggaaacggtagaatgcttgcatttctgaaactttgtaaattctctgtaattaaaggtgttcgtctgctgttttagtggccgttgttctacaacaaccagttgtagcttacagctatatagtgatttattacaaaacactttcttcttctccttttccttcttcctcttcctcctcctcttcttcttcctcctccatcatcatcaccggcatcatttattgaatgctactgcatgctggtccttgtgctgttttatatacattatatacattatctcatttactcttcacagctctctatggatgcagtttttctctccattctacaaatgaggaaactgaagcccagagaggttaagtgatgtattggcctaagacctcgcaggctgtaagtggcaaagctaaggccagaatctctactgtgggactctaattccaaagtattttctatgagggcattccaaagtgcagccatttttgcgaagaaaattaggaaacttgaagtgacgggtggctggtggattgttacataagccgagatgagaggcaggaatagagctggcagaagtgcttgcagaggttctgaatttcctctgcttcctggtctcttctgaagatgggtcttcctagccattacacaagtagcggggacaaccagatggtcacacaaagccaccacccactgtggaatgaatgtctaagttttccatatttaccccagtctctaatgtcctctggtgaaattttgtctttgatgggtttcattttgggcccttgttgtttttctcagatgcctttcttttccttgtgtttttcttgcttcctccggtgcttaatggcttcttcatgaagcggttggaaatctgagagattgtgtcatcctgcccggcctcgggctagacaccttctttgcctgctttttacttcctggtcagttgcttctccttatacaacttttttttttttccttttaaagcttttcacacaatccataaacacaaatttttgtttttcttaaaccatctctcatgtacatctttaatattatgtgatttccaggaatctatttcagatattccctcattttcccctataacaaaaggaatcatctttttttgactcctgtctcgatggcttgacccaactcgcctgttctaatttcctggaatgaagagagcatttcggccatctgactttatacatttctgattttgaacgactctagtgaagagagtcttcagagcaaatgtagggaaagaaacagagaaagagagagatgtgttgtggagataatatgaggggattcagagagcattatccaaatttgtcatgaagcaaatgaggaaactgagtcttgtggcgtttaaagagcttgcctgagatcacattcctaactgatcaagccagagtcagaatcagtgttccatcctaggtccatgttcctctaacaccatagcagaggaaaaatgagaaaaaacaggcaagaattcagaaagcatagcttccttttgaagattcacatgacatattagtgttcatcattgtgacttccaagtgaaagacagagttcatgttatgagatttaagaagtgaagaaagaatgcaatgtattttggaaagatatctaacaaaaatggttggcataagaataatgttaagttttaaaaggctaagcttcagttacgtggatgaatgatttatgaaaagtactcccccatgaagacacaaaagaggtagccaatgcatctgcaagttcttgaggcctgatggcctccctgggggtattttccctttgtatgtgggagtcagatatacggaaaacgacttcatttcctgaatgacccaaaaaggacgcattgccctgctgagtgccagttggtgagtgtgctcaatggtgttagtgttgccacagtgtagttccagtcaccacgtctttcgattttgacttacattttcttctgtcttatgttgttctttggttcttacctgtaaagctgacttcccttcaggagattgtaaggtgctcagaaaagggctttacctcactgccatgctgtgaattctcctaggggaagagtacagacaatggagtagacagacccatgaatcctggttcctgaacttgaacttgttcaagctacttaatcatgaacctgttaactcatctgaagtggtaataaaaatgtgtctcttagggttgttgggatatgtacattgcccaatgtagtgctttatactgttaaaaagttagttctcctcctcctttatacatcctccataacacatggatctggaagaggctagagattttctggttctgtgcccacattatacagttgagaatacagaggccaggggatggtttgtttttgtttttgtttttgagacagagtctcgctctgtcacccaggctggagtgcaatgccgagatctcagctcactgcaacctctgcctcccaggttcaagcaattctgctgcctcagcctcctgagtagctgggattacagctgtgcaccactcacctggccaatttttgtatttttggtagagacagggtttcaccatggtggccagggtggactcaaactcccgacttcaattgatcctcctgcctcggcctctgaaagtgctgggattacaggcgtgagccactgtccccggcccccggccagggatgttaatggccttgctcaagctcgtgtggctagttggtggtgccatctgaactaaaatctaggcctcccgagtcagctgactccctgcacagtgctcttgtcactgtcccattgagttgttcacagggctggggtcactcagtcagtgctcaaaaaagctcttattttaactttaattgaatacaagcctcaacaataggagagatgagccaagtgcaattatatgataatagcaaatgccttttgcatcttacagcagaattttgaaggaatagctaatgtggttctgaataagtttccagagatgaaagaaaaggaaaaaacctaagtctggaagtactgtacagtgagtctaactagcattattactcagcgctcaagtatttgaacctattcagggatctgctccttcatacagttgcttattatttgtgactggggtgagacttgaaagagagcatcaattctccagtctgcttttgtttaacaggcagttccatgtctgtctgaccacagaacagacactgtttgctgtgggctggtgaatgttcactttcaagagcagggagtagatggcccttcgtggaagtttaattatacagaagagtttgaaaaggccaagtttgttactttggggcatggggaaaccttcctctctagcagatgtgaggggaccgaagtgtctcttttcagcagttgtattcttcttgctgtggacctaaattcgatcctaagtcattctttgtatgtacatggcagatgagaagatgaactatagactggaaaataacccttttttgaggaattgtggagaaaatgttacccacctgtccctcctaccaccatgcaataagtgtgggctacttaatactcttgaagcaagagtttaggtttggagagaaactgctcctctgatcttctcctggaaaccacaagtggaagtttgtgattttcttctcagctgccctgtcccctctcccttgggttatgacaggccgtgtaactttatctttcttaggacttatctctttgatgaggtcagtaatcctgcttcacttacgtcccctatacgtaagattgccataaccaaagatgacatgcagactctaaagcatttcctggaaaaaatgagctacagatagaagtaaggctcgtaatttaaaaaatcgaaccccagagaacagcctgtctctgaagttaaccattcccccagtactttctcttgtttccctggtgaaagtgtgaatctgggttgtggcaggggaagttctggcactgctaagaggtgaggaaggaagagggagggcccagagctgcccacctcacttgcaggtgtggcctggaagagtgccggggtctctaatgaagaatcaagttcaagggatgaagaggagttggcgaaggggcacaaaaatacagctagatagaaggaatgtgttatagtattcattagtataatagggaaattagagttaacaatttattgtgtatttcaaaataactagaagaattgtactgttcctaacacaaaagaaaaatgtttgaggcaatagatatgccagctaccctgatctgatcattacacatctgcacagatatcaaaatgtcacatgtacccccaaaatatgtacaactattatatatcaacaaaaaaaatcaaagagacaatttagaataagtatggtctcaagcctagtgactaatatgtggacaagtagatctgcactagggtatacaggaggaagtattatggagacttcataaaaagggagatggggcggggcgtggtggctcacgcctgtaatcccagcactttgggaggccgaggcgggtgaatcacctgaggtcaggagttcaagaccagcctggccaacatggtgaaaccctgtctctactacaaaaattagccgggtgtggtggtaggtgcctgtaatcccagctactcgggaggctgaggcaggagaatctcttgaacctggaaggtggaggttgcagtcagctgaggttgtgccactacactccagcctgggcgacagagtgagactctgtctcaaaaaaaaaaaaaaaaaagggagatgggggacatggtgggaatttgctaagctcttttgttaactgctttaaagaactcagactagggaaggaaacttctctcaagtttattgctggttgttatttccagaccatttgtctttcccaaactgcccttggccatcaaatcagactcctgctagagtaagcctcatggtagtgtctcttggctcacctctgtggttttgctaattattttcatgtattgacctgggatcacagaccaggcacccaaagggacagaggtgagttctttttctttgggcctctttggatactcataacccaagtccctgtgacaggttttggaaaaatggcacatctgaaggattggatccgtggtgcattttgtaattgctgtatcctcttttctttaaggtcttgctgtgtcacccatgctgaactacagtggcgtgatcatagctcaccgtaacctcaaagtcctgggctcaagtgatcctcctgcctcagcctctggagtagctaggactacaggtgcaccaccttgcctggctacttttttaaaaaactttttgtggagacgaggtcttgctttgttgcccaggttgctctcagaactcctgggctcaagtgatcctcccacctcggcctcccaaactgctaggattataggcatgaaccactgtgcccagccattttgtcctcattttgacttgagatctgggagattttaaagatccgtgtcatgagttcacttgactggtgctttgcacttttgagacccgtcactctcctgactaataggtcttgacggccactaatggccagcaatggctgggaacagcaggacactctcactttgtaccttttgatctggtctttattcaaagtggggtgaacccagtaggagagttgttgttttggggataaagtctgggtgacagattcttcaggctgggtaagtctcctgttctccccgactttgagacctgaatgacctgatttggctgactacacctccagtcccctggacaacagccagctcctttgattttgtgccatttagtccttcccttcatctgctgacgctgatactttcgggtgaggtggaaggagagagcatacctgttggtgaggtaagagtgttttccttcagagaaccaaagaaacaaaggctccctttctgaattttgtgttctgcctctttgcatttctgaggctccttgtgtctggcagggctgtgagtgaggagatgagggtgtgctcctctgcaggcagaagtcagctgtaggtcagatgtggctttggaacacaatggaatccagggggaatgttggcgagcacagcaaggctggggtgatgatcctctaatcttttgcagcgtgctgtaagagtatctttaactttcgccgagacaggcagggcaatgagcaaaaggacctcggtgttaggtaacatctcaagtagggcacctggtagtacacctgtcttgcatgttcccacccttcccaacactttatctttttgaagccatctacccttagaaaatgaacaaggagccactgacatagacagcaagtatatttacttatcttttgggtgcaaggcagtttgccctgctgggagagatgtgggataaggccccagagtggaaaaaaacaaaaaaccttaaaaatgcaagaaggaagaaaggtaggtgagccagcaggtcccactgaaaggccttgttattgcagcaaagagggaagaggtgggcccagtggcacaaatcactaacattcttctgacagtcactagccagttcttctgagaatttctctgtcttgaaaaacttggtaaaatggccaaatggagcttctccgaactagggggaatgcctgtgtgtgcgttcgttgttagccatcatgttgaacagtctgatttaagggaaagttggtctgtgtacattttcatttcagctgtggtctttgaaggttcaccatggaggaggtgtgggtggatgagaatagagtcttctgcctcttggttggcttatatgtaggttataggtgaatgtgagttcacaacccccagcctatttcttatttgggtgcacttttggatgacttgcagggctgagtttgggaaaacccagttactcttcctttatgcacatatttctgccagactaaatgccaaatagataattggaaagaattttcttcgcagccctggtccccaaattggtgtctgtgattcttcctagggtctcatattatgagagtgcactccctttaatcttttgggacctcaagtggggtctgtgtggacaagattgtgcttctttgagaaacttcttgctgcacaccagccagggtaaggcaagagttagtcctgttggctgggtggttcttgggcagttgagcatgcttagcacttgagaggtttctgttataccatttgccaaaagggacatctctccgctggtcctagtgccccagggcacaaatggcaggctttaggagaaaatgcacaatttcaggttcagtctctatctgcatttgtgtgcatgaatggactaataattgcagtgcgttgaacgatttctgcatattccaacacctagaaagtagcttcctggattagggaacggttcctaggtacagctaccaaaggctcaagcttaagcaccaacactggaggttagaattagcacgctctagggaggacagcgagagggcagctgacacaatgcctttatgggctatttttagctttagtcaggcctatctggagtggcttgtgctttcttgggcaccgtcctgcatttattccagggtttgtgcttgtgactctgtttgcatatatgccacatgtatcgcatgctcatgggatgataaattgcagaaattatggcataatctgttttctagcatatagaggaaaagattacacaaaatccagccttgttgtggcgaagaaacctaacatatagatgtaaaatatatctccttggaggcctggctttgtgatgggctatgaacggattgttaaacagcaatgtgaggggctgttagcaagctgtttctaagagcatctccctggtctggtctgaaaaggtgatgtgtacatattgtacatttgagatagtttgcctgtggtgtttatatgttggtaggttgtgtgtgtgtgtgtatgtgtgtgtgtgtgtgtcttgtctgtcttcagtggtgaactgaaaaaataaagtgttgtttacgtgcttctatgttagtgtctacattccaaactgggcaatagttttctttaagctggcttaagtgaagaggtgtttggtcagccacagggtatgaatcaaccaaacttcaaaaagcttcagtgtgtttttgataacactcttaagtatttaatagctgctttaaggatttgtgccttagtgtacttggatgaataggtaaatcacctccccatcagcaaactagcgtttgggttcccacgttggcaaagcagggttcctgttatgtgtctggtcatatttgaacttcttttcacattcaggtgacatagttgaacatcagtccgtgccagtttctgctactattgggtttttaatggaagagaactctttcttgttactacgatgccatgcttctgctgtgttagcagctgctgtattgaaaaacaggtgtctcttataatcctctgactggatcagtttgctagcctggcagagtcttattgttattgtttttatttatttatttttgtcccacataagtcagaaactgccaagagatgttgatgagtgggattagtatgagaggagatggcagcttttcttggtggaggtttctgttacaagaatctaagagtcttatgaagatagggctaaataaaaccaggatacgaaccacaggggagaaccgccaaaagaagggtagtaaatgaatggttctgtcaagtcacctctctgtcattcttcaaaggccaacttaaagccagcactgccacaaaagccatccaggactaaggttagcgaacggtgacccttgtttggtccacgatgcactaacagactcttccccttgttgcttcttactgctactggaatggaagctttgggagggcaggtgtgccatcttttccagcctgccaggcagtctggtgcatactcgtctttctcctccataccggctgtcactgccttccttctggccccagacctttcagttgtctcctctctgatcttccttcctccattttgtccccacccccaacacaacttggaatgattctttgcagataaaaatgtgatcttgttactctctgtctcacaccacagtaccacagtgtagcatgaaccacagcttcttgtcacctgcaaggccctttactgcctacctttctagtatcatctccacattatgttccaaccacataatgaacttttttctcaggaggacatggtctcgctctgtggccaaggctagagtaatctcagctcactgcaaccactgccttctgggctcaggtgattctcccatctcagcctcccgagtagctgacaccacaggcacgtaccaccacactaggctaatttttgtatttttagtagagacagggtttcaccaggctggtctcaaactcctgggctcaagtgatccgcccaactcggcctcccaaagtgctgggattataggcatgagccactatgcccatcccatgtaacaaacatttatgcctggaatcttccctcgatcttctttctgcctggcagccaatttgtcattggaagtccatttctcaagcctccctgttgctgctgggcacagtgttcccccagcttattgaacttgcttctgttattggcaacaatccagttatccttgaattatcatccacactagactgaaagcctgctgaggacaggagtctgatgggttcatttttatatttccagtgtccagctcaaggtctggcacagaggacagccaatggctattaattgaatagaatttcttaatcagtaggtatttattaagcttctgttacggacttaattggtgccaggctctttgatatgtaagataaagacctcacacagttgacatcctgagcaaatactctgtgagggttgaggaaagaagaaaggtagagacctgtgttttgaaacatatcaggactccatgtcagggcgaacatactgccacctcccatacctaaggggctcctgtgaggcataagtactgacatgtacacacagggcactcacaaatgctctccttctcagttgcctactttctagaggggaatatagatagctggagaagaagcttgagaaccttcaggctggatgtgccagaaacagcagagacagcccaagataaatgaatgtattaactgatcatccttttttttgatgtggctcaggatgcctcttgggtcagggagagagaaccagtggctgtttgttgtagctgtgtgcggtcttggccatagctggctgtggtatctgggacccagtggcaggtcaccaccgtatggggctgccagttgctcccccaccccattcctctaagagctagtagggagagagaacccggaagagcagcacggcttgctgaaacttctctgaggtctgtgattggcctttccatgtcaagggaagaatccctctcttacttgagtcagctctttccagtcttctcttgtacagcccatgttctctcctaccctatctgagatgaagatctctctggagctctcattttactcctaatgggttgcctggagggactaaaccagtgagaatgactgggcatactatgggactagaaaaacaactcaaagttatgtcttacggatggtaggacagcctcaatattgctgtttacagtggatagcccagtgacatttatttaaagagggcagtcttaagagagtgtgttagggttatgaatctgattaagtgtgatctgataagtgggaaatggaccacttgggagaaatcaggttactttcagatataagcagaccaaactgggccttccacacaagcctgaatagctcgttttctccctatttctgttgccattgaggtacagctgggattcagggcattaggatatgtgaaaacttttagcttcctgaacccaaccagaccccagcaagagtccttttacagtttccccgaaaccgtcacaagttttcctgaccgcctgctgggggctttctgttgcagacctacgcggcagctgcctgccagccctgccagatttgacttcatcttggctgccggtgggtcagtctccacgaggcttctcttggcctgtctgtccactcagggcatttaagggtcctcttgctttgctcacatgggctcctcgagggcttgtgcagtaggttgagagttgttacgggtgctgatctggaccacttagggagcagaaagcataggtctgtatatgaatgtgaaaatacacatttaagaagctgctcaggtaggggcagccacggaagttgtgctccctcttaaacactcagcgtacacacctcatcctcagacaatgtgctgcttacatttccagtgtccagcacggtgcctggcacagaggaaggccgatggatagtagttgaacagaatttttttaatcatcaggtctttattaagcttctattatggacttcaagtttcctccatgaatgctaccagaacccttgctcaatgtcttcactgttgaggctgttgagtgagaagccatttccttccccttctacttcccctccctttacagccttaccaggcccagtgttcagaggactcgatgctcatgaacctagggttcctaagaattagcatgtcacaatctaatctggagactatccgccatttagttccatggcggattatggtcttggttctagcaacctgctgaagaacaaacctggtggtttattttcttttttttttttttaaatggggacttgccttgtctcccaggcttgagtacggtggcatgatcatagctcactgcctcgacctcctgggctccagcgatcctcccacctcagcttcccaagtagctgaggctacaggtgtgtgccacgatacctggctaattttttttttttttttttttttttgagatggagtctcactcggccgcccaggctggagtgtagtggcatggtctcggcttactgcagcctccgcctcccaggctcaagcagttccttgcctcggcctcccgtgtagctgggattacaggtgcacgctgccacgcccagctaaattttgtatttttagcagagacagagtttcaccatcttggccaggctggtcttgaactcctgacctcgcgatccacccgcttcggcctcccaaagtgctgggattacagccgtgagccaccacgcccagccaatttttaaattttttgtggagacaaggtctcactatgttgcctggctggtcttgaactcctggactcaagtgatcctccagcctcagcctcccaaattgctgggatcacaggtgtgaaccgccacacccagctgagaacagacttttgaactaccacaactataggtctgttggacattgttttcagttgaaattgcttaaaaattgacctgccttggagaaaatttcatgagccttttcttaaagagtgattgaaatgccaataagaatgcaataacaagtgccctgtgtagatgtgtgggagggacaccctccccttcctccacccagcaaaagctaacgtactttccaaattctaaattgtgatgtgtgacttggtaaaggacttcgcactgttttgttttttttttttaaattgatgtggcaagccttacaaaaggatatgccgttatattctaacaaattatttctatcgaaaagagtatcatgtgagattagggctgtagaggaacagcctttcaactagtcttctatgtggtaaaggtggcacccagtttccaaagacagcacagacgccaagggtcttttctgaacttcttcgatttttatgatccctggctgggtcggagaagggctagttagtatgtttttctttcttctggatgaaagttgcaaggacacttaagccttatgcttcagggcggaatctgatcacaagttgagatcaggaagaaattttccccctgtggcttcctattgcacaattgcccggccaggtgtgctacctctgttaaacaaagacacacacaagtgcacacctccttactggctatcttcagagcaggatgttggacgttgtgggccagcagactggttcctttggcacatccctgtgctcaaagatgggaggcagctgcccgtctgctcctgtacttggctgtcctctgggtgtgctgtactgagctcagggccacgtcttcagatccctacaactctttgcaactcttactttgtgggacaattaaaattaggtctaattctgtacacacccgcatgcacatgcacacatatctttttaaaagttttccttaaacttgcttcccctttcctgggctttgcttcccccttcccgggctttacaccacatcaaacctatatgcattcccaaaacatcaaaatgactgttcacaaatcatagtagaactctttaaccataaggtgcgcagttgccaatctcaagcttggattgtaagcgacttcgaagtctgatccaaaagactgtgggaggagggggaaggaacatggatggacttctttccttcctccctccctccctcctttcctcccgtcctcctgtcctcccttcctcccttcctccctccctccctccctcctttcctcctttcctcccttcctccttcctttcttcctttcttccctttcttttctctcttttctttctttcctttcttctttttgaggcctcggctgtgctgcctcttgtgcaataggataattaccagcgattcccaggctcgcctgcttttctgcctttacatctctatctcctgtggcgcagtacttccttctgcctctattattagcttacttagctttaccattcagttctccacctcctccccgggtccctccccatcttcttccagcctttctttcccttcacctctgtaaaggtattttggggagctaccattcatgttctgttcatacaggaaccctctttttctttattaatataaaataatcttggtcctgcctcattttatcatatattcattcgtattcccaaaatagaaaccgtcgccaccagcacgtaatgtctggcctgtaaacctttaaagtaggatgtgccgaaatgagatcttcacaagcaggtgtgcctcatcccctgacctgcaggcccttcttccccggcggccattcactcatcttcacaccatcttcttgcccttctctctgcttttcgatgattctaccccaatcacattccttcccaaaatgactgtgttagtacttaattgttcacctgggcttgttcatgttgtagttcatcgtttcctttttatctttaaaattttcttgttcttcggggatacactgtattcttgtaattagagattatatcagttttcatggccaaaatttgccacgtggacataatagtatttggtatcaagcacgcctttcaggaagaagggaaaaacagtagattttgggcagcttttgattacaataatttcaaatttctaataactaagaagtacttcatgtgattcttgtagagtaaaagcttaaaaattacccttttctttgtaagtgatatgagtcttacttcgtttgggtaagaaactcagtttgaggctaccaatttttcaaatagtggtcttagaaactaattattgctgtaatttcaataactaaataggcattcctattcaaacctttactcaagtagttatattctttaactatagttactcatttgtaacatttttcactgtaagggtagtgacatcctgtttgtaacacaccctgtcctgggactaaatggaagggattgaggaaattctgctggagtctaaccccaccatagggcagcagcattttaggaacaaaagcagagaagctcacactcttctgagtataactggggagaagatttagggaggtggcttgtaggatcagttttaagaccaggtcagacatacgtacaaattctttctttgtaaatgagaagtggcctttaatagctttcatatggatagatgggtagatgaaatattattctacctaaagcatgacatttcagtaagtactacactttgaaacttgccttgcagggttttaaaaatattcttttctgggttctaatcatttataacgaagagtcactagcattttattaaaacccatattttttggagagggatattaagtgagcagggattgtgggttgttcttgcttttccttaactgtcctgccccaaaaaagtgatgggaataacccaaaccttaaaggttcctggggctttctttttccctagacagggacctcctcctctcccctgctctcatcccacttgttcctgaaaaagggtaaggttggcagggcggggaagagagaaggaaattagggttctagcttaaaactgctgctagagtggctagttctgggagctttttgagagaggcccaaggtggcagccttgtctcttttgcctctttcattaaacctggggtttgctgtacaatgctttgggtctcctgtcccaaaaataaagggctgtggcaagtatcatcctgacaactatccaatcagttctgactgagaggcacttgtaattcagaagatagtcattagatttggatagaaagaaatgagatgtgatgttcagtagatcagtagggtgactatagttcatgttaatcgattgtacattttaaaattgcttaaggagaataatttgaatgtttctagtataaaggaaagagaaatatttaaggcgatggatataccagttaacttgatttgattataaatgtatcaaatcattacatgtaccctgaaaatatgtacatctaatatgtatcaataaaaataaataaattcattagaataacagaatttagacatttagagctatagttttaaggaatttcagaccaaggcccaaagctcggttaagtgacctactgaaggtcccaaggctattattgccatattagaactagaagtcagatctcctgactcccaccagtgtatattgacttttgacccagcagttagttttattttaggctgtattttcgaaataacagattggttcaagtcaaaataatcttattcgtgtgaaaaactcaaccaggtgaactgagtgaatgaacctactttttaattgggtgtttttaggtcatgtgaaaaaaaaaaacacaaatcagaaggtcctttgtgcttcatgtttggatctgatccttttttttttttttaagctgaaaatgaatgaaggatggtagagtaggaactaggatgtacgcataaaacacttgcctgtctgccttgtcacctgcactccagtggggtgtttccagggctcattgtaaggtgagaggtgacttctggacccttgactaccctgttccttctgtcgcaaaatataatcaggaggtttctatttgacaatgtactctgagtattgtaaggaaccaactcccaacccaaatcgaatcgcctctgagatcctattaaacagaaaatgccctgaacaccagtatatgaaaatgctttgctaggatgagctcaaagcatttcatcctccaaataaaatctatctgctcacacttagattgttcaaaggcagatggtaggcaattgaaacagagagacaagctggcaaaaggaaaaataaaaccacaaatatctgcacgtgaaggtgatttacgctcttgccagcccccctcctaacttcgttaacacaacgacgtagcaattaagtagcatgggaactcgggctcagatggctgatacagttgtcctccgttaatggggtgagacaaagcttgcgtataatatacagtgctcttcaccaaaatgcagagctaaaagagattttaagagataatcagcaacatagcctgtaaatcatgctgctcataacagacggccaccttactgaaatctcactgacccccaccacacgtgttaacagcagggccactgctctgaaatattagctattaccgagtggccaagtcttaagaaagggaccacttggcagtattgaacttcaaagtggaagtaagatccccttggggtgagggtaggaaggcggtgaacagggttgatcagaaccgtggaagagtccggagcagatgctctgtgggaaaccctgaattagccaagagcaaaagttatcgccatcctactgctctgggaaggctgcctgttggagaaaacctctgtgttcccttagccttttcactgactggaggactttccagacgaaatctggcacttctcagtaaccttgaacttgaacattcagtgacagaagggaaagggagagggtttcagctgctggagctgggtaactgtgcagggctctcgcagcgggccatttgtggctctgagggtgccaagctgcagtgttctgagggtgtgtgaatgagggtgttgatcctggctgcagaaggggcagctttctgagcaggtgggagtggtgaatgaggaggggatttggcggggcaggatgtgagatgagtttactctgctgggaaagaatttggagatgtccatgaagaagaaaatctggatgactattgagggagggctgatgttggagagaagggctcatactacaaatcagaataaaataaaggatatcttagtatcaagaggaaagcactgttaagatactaaaccttctgctgcccaggtatctgaggagagaccatggaagagttctatatggaacaaatgaaaaagcagtaaagtcaggtctagacctggagtttttagtaataccaagtttacataggaagtgtctgggaagtagtgattagagggaaagtatgttggatgagtgaatcattccttcagaaatcaatattttttcctactaggccctccgaatgtcatctttgtagttgcccaaatacatcagtttctctcttatgtaagagagcttgtctcatttttattgctgtattccattttcaagtgtcttcttgggtaatgatcctgtatttgatcttggccagatattatctgtgactgttgttagagtgatccttttcctttcccccgagttagtcaaactgtgtgcgtggtctctagactgagatttggcagtgattccatggctcttgtgagtggagttgactaacaaaactcaggaatggaatggccatattttggaatttggttgaagcaagtaaagtttttattcttctaccaatgcaaatctatctcagggaatttctctttattcagaaagaatgacctccatcccacctttctctaaaaggtacaaatcgtaatgcatatttgacacattagttggcttttaccttggggaccagcagaccaccccgcagatacaatgcacggaatctagcaaacaataaagagcagtgtgctcgcgtggccagatcgttcgcttccttcctttttcgccttcatttttgggaagtccttgcctcgttagttccataggccatgagacatagcaatataaaatgccagaggatgttcaaatgggcgtgtggaggaagagggaaagcaggaattctgggccaaagagcagatggccagccagtgacctaggaagggaagctcaggcttttaaacaatggcaatgtttcttttatgtgcagttttcattaaggagtaccgagactccaagttggaggattctgaaagtggtcatagagtccctgccctcgagatttaatatccaaagaggagacagtggttttaatataatgaatatatgcagaaaacagccggagacaatagaaacactttggcaccttctgtggcgctgctaaaatgtgtgctgtagctcaaggcccataggggttaatgtaggggttatttgtgtgtgtgtgtgtgtgtgtgtgtgtgtgtgtgtgtgtgtgtgtgtttggtgttttccccatgaagccctgattgtgcgaccctgggtgagccagctgcttccgctgttttccaggaaagcatgagtgtccaccacctagagagatgatgatgtgagaaggggcagggagagacctctctggctgccactctctcactgccccaggagcggaggcccctggtgctcaggagttctttggctgtgtccggatccaccaattccccgcttccctaaggatgacgggtaaatctggtgagacccgctgccctcctaatgttggaggaaagctggagcccctcagtcaacatttatggaagggatttaatgcatcatgggctgtgaaggtacagccaggccctccgtcagctccctgagacaggccttgtctttttgcacgcatatgtctgttaatagttgtagacctagctgtagccatgggggtaagcctagtcctccagcccctgagagtttattaccacaaggagtgagaacaccaggatgtgtttttttagatgttcttctgtaacccccaagcacctgtagctcccaggtgtttttacattgatctcagaattacccagaaaacattctttgaagaccttgtagagtctgccatttgtacttctgctacaaagagcaaaaataaagcaaataaacaaaaagcccctggaaatcctatagttatcctaatacatattttaaagtattacctatcactgagcaagtgcatctatacatttcaatccttttggaaaatatactttctcaaacttcatgttgtatccctctaaagttagttagcacatattgtacattcagaagaaatgttcatttacagcaaggcataggttttttttcctctaaaagaaatttgaccaggtatggtggctcatgcttatattctcaacattttaggaggccaaggcaggaggatcacttgaagccaggagttctagactatcctgagcaacaaagtgagaccctgtctctataaaaaaataaaataaattagctggatgtggtaatgtgtgcctgtagtcccagttacccaggaggctggggcagtaggatcccctgagtctaggagtttgaggctgcagtgagctatgatcatgccactgtacttcacgctgggcaacagagtgagacctctgtctctaaacaaaaaggaaggaaggaaagagggaagaaaggaagggaagagagagagaaggaaaggaagggagagggagaaggagggaaaagaaaaagggagagaaattgatgcaggaagaagaaagaaagaaaataaaagaaagggaaagaaactgatgaaggaggaactgggtacctagaagtggttgaatgaaataatcctgtaaagtcatctaaggttcccatctcattccttgccttttatttcttgccatcacctatggccacttcttgcttgttcatgtcactttctttgtcagtttggaatgggccctgcctggctgatggctttggagaggacagcagacagtcattagagactagaagctgccaagcgaggcaacgtgacagagcacacagctttatgcctttttcttagtctactctcagcttggaaatgtaggagttatagtgacacagctcagtcagcctcttcctcccccgatcctggatcatccactctgcctctccctcccccagtcccagatcatcctgttttcctagaccattccttgccctgacaaatttctattactctcaggttaaagcccaactaaccctgaccaggctgcaaggccctgcgtgatctggccctgtcccttgctctttgtcttggactgcccttgcccacaccaactccttagcattctacagaggtgccatcctcttctctcctggcctttgcccatgccactccctgaaacactccttctatctctcttatcccagctgatggctctttcctaggtcttatctccctggggtcttttcttgaactcctcaaatctggatgagatgtccctcatatgtgccctcatgacatccgttttgaaacttctatcacatcaccttgtttgtatgcatcatttgtcaataagcccacgagatcagagcccatcttggttttgagcttatgaacatagtgttgggagtaggtttggtgctccataaatatctgttgattgaaggagtgcgtggaaaacagaatttggtctttgatttttttctgtgtttggctcagtgtctgatgctctcccgagcattcttatcagtccacaggattttgcaggactattagcagagggtaatgcatatctgcaatattgtcagacatcgcctgttgttagacgtttgcttatgcagggaaaacaatatggagtttttaatttttagttggaaattcagaatattaagttacacataacctgcagtggtttggtttttaaattgagtttctgcttgttgttaaacccaatccgaatcaactaaagggaggtctattctctctacaaggacactgtggctctctggatattcctgtaagacaggaaaaacgatcacagatcaattctagttgctgcctctgagaaagaagaacaagatagatgggtctggctttgtgaggaacctttctctctctcctccctcctcctgcctctctctctgccaataattacaaagctgaactgaactgctactgagaatccaagttctttcctggctctgactaagagaacatagatagcatatgcatctagattctttggctgtatacggacatatatacatgcacacacatacactctgtctttggcctgaaatgacggcactgcactaagacctgttgtttgtttcttaagcagatggatggctagatttttctttatatgaatctttcatgattacttttactaggacagagctaaaagaacatgggatgtgaagtccagtgagttggattgattggatgtatgcgtattgagcacctgttacatgctggacattatcagaattatccgggtgaagaagatgaggcctctgattgaagtggcttgctgtgtaatgtggttacactagtgccacttactctataactttggcggacaaattactggacctctttaaacttcccattgcttaaccgtaaaatgaggatagtagcacctactttatagtgtttccaagagcatctaccatgataatgtttgtaaaattcacttgacatacacctgctcatccttgacatctcccagtgcagtcaccggagcgcctcccagcgtcctctggcacaggtaggtgcttcccctcgtgcagttctctgcttggacacttcactccgtgctgtcagcaccctttgcttctgttcctgcacttgtgccaaaccctttggatgaagagcctcgttgtgcccatctctggattcctagtgccaagcatggggtccagcacttgacacatggctgttgctgaataaattaaagaacaaggattatggatggccaggtaccatgcatataacatccttattataagaactttgggccagaaaacagaacaaaaagggagagtgctttgaggccctggagatcagagcaaatctcggcaatatcacgctccaatttgaaggctgtaagtgtgtgctgctttttagcatggatgctgatcaactgcccgaggtaaaggaacctcttgaggtcaggaaggaggtctgtatgtttagtctggaggtcagtctacattttggttagctaacaaaactaacagacctaggtggttggcaattagaggcgcattttgattgttttatgccatatactctacaactcacatggcagcacttttttcctcctgatttcctttccctttgcaggaagaaatgtgtttctccttttcttggaatctccttattttctgctccgattatatcccttttttctgctgttcatacaatttcaagaattaatatgagttatgagggttctccagagaaacagaaccacaaggagataatgtatttatttattatgagaaattggctcacgtgattatggagactgagaagtcccatgatctgccatctgcaagttggagacccaggaaagccactggtgtcattcaatcagagtccaaaggcctgagaaccaggggaattgatggtataaatatatcccagtttggggattggagaagatgagatgagatgtcccaatgtaagctgtgaggcaggggaaaaaaagggagtgactggtgaattcttccttcctctgcgtcttgttcttgttctgttcaggcccataatggattagatagtgcccacccacattggggagggagactgctttactgttcaggagctaatctctccggaaacaccctcctagacacacccagaaataatgtgtaatctgggcaccccataatccgagtcaaattgacatataaaattaaccatcacagatattaatgtcattgaggttgaaaggagagtgttcttgtccttagtgtgtgtttgtgtaaatacaatttctaacacaatctgagtgtgcaggaaataaggaataagcactttaggggaaaagatcatgtctcaaaggatcatttttttttctttacccacccctttgactctcagcattgaaatcaaagtaaggccctgagcagctatatagtagaagaaagaaagaaaaaagtaagaccagatggttgtttggaaagaaacagccactcttagcagttatgcacagcataggtagagtaagttcttagtgctcgataaatgaatcaatggttgcactttttagaaagaccatttcttcttcggtaaagcttgctagttcttctcacagattctcttttcatccgttccataggttctgcacactggtgaagggtaggaacagagtgatcttttctttccttcttgctgggtttgccacaaagcatgttgaatggtcttgatgtttctccggtctataaataaatacttttttaaatggagccatcatgggatctgactctatttattcttgtcactctcttctctcctcccacagggaaatggcacatttaggaagagtttccattatttgtaacatttgatgatttgtgtctttcatagaccatgtatcagaggtggagaaacagcttcttaactttcagacttgcttgttcttgactccatgagccatctaggagagttaccccatttctgtaggcatccatcagtattcatcctctgtgttcacacgctttctcattatattactcttctcaagtttacaaatctcttctcattgatttcattttagtttggcatcatggtctagtgagactaacacttggctgggaattgggactaactgtgtccttggataagactcttaccattctgggcttgtagtttttctctgtaaaagttgagacatccagcttgatagttctctggctataagggcattttgacctccgaatactgaaaggatgcaggtaggggttgcaatctttcatgctctgtactttgcaagagtaatttggtcagctatctgaggcattcccatgagaaccaaatggaactactctggccccttcagatagaaactgttagaaataaaaagcttacacttgttggggggcaggcagtatattttacatctgctgataatatgctagaaccaaattgcaaagtgtacccatgtatgttatttagtattttcattgttcacatttttctcatttctaaactatgttgaatagtggaatattttgcttcctttggttttgtttgagaacctcttctccccttagtgatggtgctgtagctgtcgtcagcccaaaaggtccatactacatgttcagctaggacagggaccacatcagggcaggcatctcatataatttgactcagccctaatacactgattttcttacagccaaggattaactctctccattaaagactcatgtgaggggaagaacagatagcaaagtgtttgctaaaatgatgaactgtccctatttcaccttcaggcagcaaaatcatctgcttagaaactaattagagcattttccccaggcgttggtgatctacttctacatgcctatgatctcttagacagtagttgtaccatgttcaggaactggccataaagcagccctctccctagcctgtctccctctaatagaagaaggctgagagtggctcattccttcagatgaatctcagaccttttatttcagattcatttaagggcatttacttctctgtgatgctccttactttctgttctttaaaatcttctttgcttttggatcccagggtagacaaggaaaatgttgcaccatcatatataggtaggtaggagactttaaggtgctcaaagcctggtcgacattttctggtggcacagatgaagccattttctatggaagcctcctaggcaagactctgtcctgcatcccactgagtatagacctaatttctttctaatatggatggaggaataagcatggtcatttatcatgctgaggcagctgtaagccgtctgcatccgggttccattgcaattggaacagtagcaaatccattgggtttctttgtttttcagggtaattttctttcccttacagtgtactacagatttggggtctgtggtcttaagtgaacatttacataaagttagctatttatgattttaaacaaatttcatgcccctttctgctgtgttgttggtatttttggtttctatgtatcataaaccacagaagttcagtgacttccatggaatgatgtggaaaatcaggaagtaagactgaaattaagaatgaaaatttctgactatatgtgtcatgctcattttcaccgtttaaggtctttcagaccacaatttagtgaataaatctatatagcaaattttttaaattctcacgttttatttattattattattatcatttattattatactttaagttctgggatacatgtgcagaatgtgcaggtttgttacataggtatacacgtgccatggtagtttgctgcacccatcagcccgtcatctacattaggtatttctccttatgctatttctctcctagcccccacccactgacaggccccagtgtgtgatgttcccctccgtgtccatgtgttctcatgttcaactcccatttatgagtgagaacatgcagtgttaggctttctgttcctgtgttagtttgctgagaatgatggtttccagcttcaaccatgttcctgcaaaggacatgagctcatccttttttatggctgcatagtattccgtggtatatatgtgccatattttctttatccagtctatcattgatgaacatttgggttggttccaagtctttgctattgtgaacagtgtcacaataaacatacgtgtgcatgtgtctttatagtagaatgatttattaccctttgggtatatacctagtaatgggattgctgggtcaaatggtatttctggttctagatccttgaggaatcgccgcactgtccacaatggttgaactaatttacactcccaccaacagtgtaaaagcattcctatttctccacatcctctccagcatcttttgtttcctgactttttaatgatcgccattctaactggtgtgagatggtatctcattgtggttttgatttgcatttctgtaatgaccagtgatgatgagctttttttcgtatgtttgttgaccgcataaatgtcttcttttgagaagtgtctgttcatatcctttgcctacttttagatggggttgttttttccttgtaaatttgttttaagttccttctagattctggatattagccctttgtcagatggatagattgcaaaaattttctcctgctctgtaggttgtctgctcactctaatgatagtttcttttgctgagcagaagctctttagtttaattagatcccatttgtcaattttggcttttgttgccatgcttttggagttttagtcataaagtctttgcccatgcctatgacctgaatggtattgcctaggttttcttcaagggtttttatggttttataggtttttatggacctaaaaccataaaaagtctttaatccatcttgagttaatttttgtataaggtgtaaggaaggggtttcagttttctgcatatggctagccagttttcccaacacaacttattaaatagggaatcctttccccattgcttgtttttgtcaggtttgtcaaagatcagatggttctagtcgtgtggtgttatttctgaggcctctgttctgttcattgttctatatatcagttttggtaccagtaccatgcttatacagcatatttttgtttgcactttcattgtgaatgctaattgtttttacttaaagattcctccttgagtagattcaataattcaaactggacatgaacatgtgactttttgagatggaatcgctctctcgcccaggcttgaatgcagtggattgatctctgctcactacacccaggttcaagcaattctcttgcctcagcctcccaaatagtggggattacaggcgcgctccaccatgcctggctgatttttgtatttttagtagagacggggttttgccatgttggccaggctggtcttgatcccctgacctcaagtgatccgccagccttggcctttcaaagtgctgggattacaggcatgagctactgagccaggccatgtttgaatttttacaagactaatttaatttcacatttcataggtaacacatagacttagccacatttttcatattggtgcctccatggtagataagacattgaaactataatgcaactaagatcaaattttttaaatgtcagaataactttaataaataaatttgctcatccttttttgatggaccctaacaatgatcagtttttagccatgatgttatctgccactagactgtattattttctcatttaacatagcttcatggtactaggaatggaaggaggaaagggggcatgaacttctcaagactgctttgtaagtctatttctttctttgattcccatacctcaaggaactgagagcaaactttgagcctgagtgtaaagtttgttttcctggaaatgtcaccaggctgtaaactcagaaacagattgcccccaaaaaattccctaaagggctctttacttttttcttgtgttacctttttttcctgtggcattctgtctcataatatgaacctcatatagttgcctcccaaacttttctgtttagcttttttttttctctccaagaggaatttctttaagtacactatggaaatgtgcataaagatgaaagcctgacttttctaaacactgtgttataaaattaattgacaattaattcaacagatgtgtattgccacatggatagagggtaaaggtagtcatttgtcactgacttcgacaactgagaaaaaagtgattcaagggagttggccctgcatgtaaggaatttcaggaacaatgtggtcaattaattttacttaagatgaatgtaaacatcatgtgtacatgcagttctttcaataaatacaaaataattgtaaatgataaagtggcagataaatcttagtggcagataaaccagtgcctctaacaatccgcttctgccctgtggcattttttgaaatgttactaatttgacagactaaatgagcatccagtattatttagcttttaaaacatattatttgtgtttttttcctttaatattttgcttagaaagtctttctctactctaaggtcaaataaatgttcagttaaatagatggaatattacctgtttcatttttacattcaggccttaagattttattagagaaacttccacacatattcagaagtgatgagaacagtataatgaattatatgtactcatcactcggcttcaacagtgatcactacgtgactaatcttgttttatctgtatgcttacatccttctcacccttccccttccctattattttgaagtactagtcactcagaatttcagctataaatacttcaggttgtgtctttaacagacttaaaaaccaagcaagcaaaatgtcattattacacctaataaagctaacggtcatgttttcatattatctaattaatacctagagttcaaataaaatgcctagtatttttggggctgaagatacaacccataaaagtaaagccttggtgttataatacccaagagccaatattctaacgtgaggaaacagacaaaatgtaagtaaaatacgaacaagataatttctgctaacaatcagtgctcggaaagaaacacagtagatgacagagagagtgacgcagggccagggggatcctattccaggtcggtggtcagggaaggcctaggaggtggcatgagtgtgaggaccaggtgaagggcaggagctggcccaggggacagtgtcctaagtggagggatttgccactgcgtgaagagcttgatgggcctgcggagtgtcatctagctaggcacctttttccctgttaatgctggctttagttatagtcatttgtaatgtgtagattttcagtgtcttaacttttcaacagggtaaataggaccatccggagtacaccaatattgagctttgtgataccttttggtcgatttgttgatcagttatgcttcattgtccatgaacatttcagaaaaaaaaaaaaaaaggaaaaaaagaaatggaaaattcaggctacttaaatcacgcctttgaaacctagaaacccttaggggcacagacccttcttgtgtaataacagagctctattttctgctgggaagttccctattgagatttgggacacttcctaattttttctgggtaaaataaaagtgacaacttttttttctaatgactttttaatacttcttcttatctgagtaaaaaccaggaagtgggaagcaaatgtacaaaaaccagcaatacacacacacgtgtgtgtgtattttcatgtacatatgaatgtgtatgtgggtgataggaagaagagaggatgcatagagaagatgatctcgatcaagtttgatgtaaatgtaatagcaaattagagtacattcttgtcttaatcttttctgtaataaagataaaatggggcattatgttcatatgattgaacaaaagtggatgatttcaacctttttcttctcagccattaaaagaagaatttgaaagaaacagtctttgcatacatatacagagagtttgtcagcctttcttcagacacagccagccacagtcagactaaacattcgaagacgttacatttagtccacagtggaagtatttgttgattcacatattttctttttacaagaaaatcttcagggttagtattcacccctgaattggacacaaagtaaaattccattgtgttataagacagcttaaaatatctttgatttaatccagaaaattctatcactcttactcctgaaaaaggtgctggtattttaggctttaccagtcagctacattaaaagctgtggatagttttgcaatttatgaaataattactcttcctcatttgtcttcttgatttgcccgacatcaatgctcttgtgttcaccagggactaatgattgtatttttcttcccagtgggtgggataggataccagtaggtgggatagagagtttatggtagattcagtttgtctttatttactcaagcactcttccagggcctggagctgtcaaaggtgaagtaggaaattactgtttacatctgtcgggaattcactgttctgtctttcatgagctttcatgccctttagagaccattttgcacaccttgattgtcagaaaagggtctgggatgtttttggagtggtacagagatgcatacagcaacttacactgtcatgactttttttttgagatgcagtctcgctctgttgcctaggctggagtgcagtggcgtgatcttggctcactgcaacctctgcctcccaggttcaagtcattctcctgcctcagcctcccgaatatatgggactacaggcatgtgccgccacacctggctgatttttgtatttttagtagagatggggtttcaccatttggccaggctggtctcgaacccctgacctcaagtgattggcctcccacagtgctggtattaacaggcgtgagccaccgcacctggccctgctatgactttcaatgctctgttgtagttttttcttaaaatttaagtttgcttggcaatacctttttttttctttcttttctattttagacagagtgtaactctcacccaggctggaatgcagtggcaaaatctcagcttactgcaatctccgccttccaggttcaagtgattctcctgcctctgcctcctgagtatctgggactacaggcgcacaccactgcacccagctgatttttgtatttttagtagagacggggtttcaccatgttggtgaggctggtctcaaaactcctgacctcaaatgatctgctagccttggctttccaaagtggtgggatttaaaggcatgagccactgccccccaccccaccgccccaccttttttctgttcttttttttttttttttcagctcactgcaacctccggctccccgctccccacgccccccaccaggttcaaacgattctcctgcttcagcctcccaagtagctgggattataggcacatgtcaccatgcccggctaatttttgtatttttagtaaaaacgaggtttcaccatgttggccaggctggtctcaaaactcctgaattcagatgatccacccgcgtcggcctcccaaagtgccgacattacaggcgtgacccgcacccagcgccacaccccctttttttcttaataagaaaatgagtagacacatccctgggtgccaatcactcatgtatcaagattgctgagtatgagaaatcagtaaattaatatgaagaacttaaaacacagttctagccaaagtaatgcttaataagtacagtattgtgcaattattgctaaatagcatacatttattgtgttcctccatttaagatactttcaagcattgtagggaaagaaaagcacataagctgtagtctttagctttaacggacttgtagcctagttgagagaagccatatgtgtaagatgacgagtaatagcacaaagcagtgtgtgatcagtgccaaatgcaaatatgtactaaaggagtttaaagcacagagaaagtttgctgtgaactggaataatgataacaatggtagcaacagcagccagcatttactgagtgcttcccgtgtgctaagtactgatcaagtctcttacaggtattcgctcatttattctttatgtcagccatataagaaagatgtttctattagttttcctgctttacagctcagaaaactgagtgaggttagctaacaggcccagggtcccacaccagggaaatgtggagtctgggctgcctgttagagtcttggctgcctgactccagagccccagtcttagccacgtcagggtggtctaggagaactggatcgaagtagataccttacgggcaaaggaagccgaccacctcatggaagagtctggtgtatacaggaaaagagtgaaaggtacaatgttggggcatgattaaagggcctttagcccccgttaaaggcactttgacctgatccagaaagtagtgggagaaccagcgaatgaacttagcaagaggaatgttgggtgcattgcagggcagccactatggctcaagttggggctttctgatgctttctgcttcagtctcgtggctgtctagtggtttgattttccattagccctttctcccttggccttaagggatggatatctacaaaatcttagctatcaggcaaaacctcactgtcccactggtgataagctgggcttcccagcagttcatacgaggcctgcatcctgctgtgtaacctgcctcaagactgcagaaggcagagttaggtgtccttccccctcagggccctctttatgcttctctggatttggtgccaaagaatccacagtttagggcaaccgtagaaaccccaggaggagtgtggtagcgtgtggagaccctactaagataaccatagccttagctgctccttgaggtctggctctgggtcccaggggctgcttagaaggatccttagtgatcccctggtcaggacatcatatgtttagagagcaaggttagaggagttaccacagttttcttctacatgctgattaatctaggatttaaatacaggctccaaaataaagtgtaagtggtggtagctcaaagaagcaaagcaagcttcacattaaatgacatgggcttctgaaaatgtgggaaggactaggaacacaggctacttactgcatctataactctctttagacaaagaaaatatgtgattaggaaatgttttggcaagatctcaccaagattatttactgcaaaggaggcccaggactcagcctggcagggaaaatggggcgggggggcgggagtgtttactgggagtatggaagagcattttaaagtatggaagtagattgaacccctgtgctggggctggggacagggagtaccataatacaattttatgctgttaggggatctctgcaaaacctagagtaagtagaggaaggagtctgtttgaaattaggaatagtagggtcagggtccagatggaggtggggaggagtggggctcagacctgaccactgcttctctaggcgtcgtttgaatatttaaagacaaagcacagtacatttgctattttattttattttatttcattttatttcatttcattttaaaccatggctaactcttctccagtcttctccaggagaaaaaacttgattgtcatcctggcacaaaaatgtccacagttaggctaacaggcaagcttccgtgagcagatcaaatttgctattctggttccaacatttaactccctagctttttttaaaagaaaaaataagctggcatcaactgaaatttcaggttaggggcttggtttgaggatgtttggggaagcgggagctttctgccatgtcttttgtggacattggatgtatttccgcattctggttggccacatttccccctggggcagattcttcagaatcatttgtctcccactgggagtctttttccctcccccccccccccacccccctttgtctttttctctgacatctgccgcacactctctccagggcatacggtccagaccctcctgggagagcttcatctgcttagtataacttctagcagaagatagaactgatggattttcgtgcgcaaatacagattcacaagggtgttcggtgtcacaagcatggatttatgatgcagtttgaaaagggtcagaatattcagagatgagattaatgccagcccatgagtgctttgtggtgctgaggtaactgaagtgcatctggtggtgtccagagctcaagtccccactgcagacgccaggaagaaagaggtggctcgcagactgagaaggggcgtagggatgcacccttcttgtctccttctgcttgataatttagatagaggatggcaggtgtaaatcagtttgtggtagatgcagtgtgagctgtcctttctcccccttcctcacgtccttacaagacacctgtctgctttgtatcgattccggcactgtgttatgatattcttgtagtttcatttttttgttgtttctcttttgatattggactttatatgcaaagaatgggattagtcataatctttacttcaaaacacattatcttcatatttttatgctggttgtgatggcacacacctgtagtctgagctactctggagactgaggtgggagaatcacttgagctcaggagttcgagtctgcagtgagctatgattgtaccaccgcaccctagccaggacaacggagcaagactctgtctctaaaaaagatagaatgggctgggcacggtggctcatgcgtgtaatcctagctctttgggaggctgaggcaggcagaccacttgaggtgcagttcaagaccagcctggccaacatggtgaaaccccatctctactaaaaatatgaaaattagccgggtgtggtggcgggcgccggtagtcccagctactcgggaggctgagccaggagaatcgcttgaacctgggaggcagaagttgcaggaagccaagattgtgccactgcactccagcctgggcgacagagtgagactctgtctctaaataaataaataaataaataaaattatataaaataaatagtgtaataaccaactatgtagtcacttcaggctgctataacaaaacacataaactgagtggcttataaacaacagaatttgattctttccgtctggaggctggtagtctgaggtcagggtgcccaaatggtcaaattctggtaagggccctctcctgggtttcaggctgctgacttctcactgtgtcctcacatggcagaagggcggggagctctctaaggcctcttttatacgaacctaaatcccatccatgagggctctaccctcatgacttaaaacaccttctagaggccccatctctaaataccatcacattgagagttaggatttcaacatacgaattctggggtaacacaaaccttgtctgtagcagtgaccataaacccatccccccagacaaatctaggacctggagcataacctgcgtctgagcagattgccatccttcccacacctcctctcccttcttttctctacccaaggtaactattatctgaaccctgtatacatctatgtttccttttctgcatactttatttcgcttacatatcgttctaaaatggaacacataaaaacataagacatacacacataaaaagtatgtagctatatagatatgtatttaattgttatcacctttcaactttataaaaaggatatcagaccatatataatcttttgggacttacttttttacttaacgtctcgactgccagcattcatgtgtattgctatgcatcacggtagttaatttgccttgtctgctgtatgatgttccatcatgagccgattatacacagtttattcattcactctctcattgatgggcgttgaagttggtgctaggatcttggcaattgcaaacaggtttgccataaatatattcatgtatgtataatcagttgtccacgtgcaaaagagagtcctttgtgttcgtgactccattattttgaaaccagcagtagtactgcacactgggagaatgtttgtgggaagcacaataaaggtaagtcctcagtactttgtatttcagtttggtgtgaagaagtagcatgtagacttcataggagacaagacacttctatggtttgagtatttgtctcctccaaaattcatgttgaaacttaatgccttaatccccggtatggcagtattgacacatggggcctttaagaggtggttgggtcatgagggttctgccctcatgaatagattaccccattcatgggttaatggattaatggattaatgggttattatgggaatgggactggtggctttataagaagaggaagagagacctgagctagcatgctctgccccttaccttgtgttaccctgcactgtcctggaactctgctgagtccctcctagcgtgaaagccctctccagatggggccccttgaccttggactccctagcctccataactgtaagaaataaatttcatttctttgtaaattacctagttttagggatttggttataagcaacagaagacggactacatatcttattttaattttgttcctcccactaacttcccagccccacagtgcttggagcaactgtttctgcagtcatgacttcagtacagttaatgcctgttgacttcatcttatgttaaattctttgcaggtaaagccatgttccttataagatcagtctttttttgttcctctatgtgtctaatcaggtcaattctttgcattattttatttagcatcatctaccaggaactaggcaggcattgtcttaataaagcctggttcttgcagttggagaacttgctatctagctgggagaggcagtatgcaaactaagatttagaatgcgttatgaggccaggcgtggtggttcacgcctgtaatcgcagcactgggaggctgaggcggatggattgcctgagcccaggagttcaagaccagcctgggcaacatgacgaagccatctctacaaaaaattagccaggcatggtggcacatgcctgtggtcctagctacccgggaggctaaggtgggagaatcacctgagcccaggaggttgaggctgcagtgagctgtgattgcaccactgcactctagcctgggcgacaggatgagaccctgtttcaagaatagtaataataataaatttaaaaataaaatagaatgcattattatatgtaccttagaacttaggtcttagagagtgcctacctctccttgggagagaagacttacggtggtgggagacagtgttgcatctgtcagataaattagggaaggcacatggggcggacgatttcagagaagttaaaggcatgggagaacatttgccacgcaggccgtggcaagtggttggctttgactgtaacatgcagcgtatgcagttcctggaggcagatgagggaagtcatgcagtacttgtgtttctgtgagtctcttcagttttggggtttgtagacaggtcatgttcacttactcctaacctgtctcccacaatagtcatctgggtctgtgctacttagtgtcatcctctcactctttcccttggtggcatatccagttggctaagaggacaggcctgaccttgtgcccagagtagggagaagatcccgatcacctctggtccttgtcaggctgtttgcttggatggtagagttcacccaagttcggactcaggaacggcctgtggtcatggagtagtttttttctgtccagggtctggaccaagataactccatcacaggggtagcaacagtatgtgctggggcaattctgctacccacttctggtgtccttcagtgctttccctgcactatctctgtccccagctgcttccatccatttgctttctagtgatgaagcactgggggaagctgcagggatggctgctgcacccacatccatccctgcccatctgttctcagagactctatggctagcagagggagtgatgatttcagggaagagtaccagagagggccagtgggtagcaggagtctcacttcttggagaaaacagctggtcctctgtgaccaattctccctcaaactagggagattatatggccactgcatacacaaacacacatgcttgcgaagataggatggagttgtaccaaataaaaagtttattgaaagggatggtggtagagaaacactgtatcatgccgtggcataacagggtattatatgttggatggctttttggaaatcattttaggccaaccttattagagatctaatttatttgtattcttgtagctacctagcacagtggtaagaacacagaccaacatttggtaaaccttggtttgaactctacccctgctgcttctgacctctgtgagtctgcatattatttaacccctgaggctcagtgtcctcggttgaaaaatgggccactaatttagtataagggctatgtaagatgtaatgcacatgacatgttttataatagcttcttatggaattatctgaattggggacccatctgttgcttttataaaaagttcaccccttaagacaagtcagtccaatggccatgtttaaaccaaatgtactgtcttctgtaatgctcaatatatttttcatgtcattgagtgcagagactaggtgggaagatgatcatatacagacagaatgggcaccactctgcctaaacatggctctcgctttcaaaacagtactttaaaatatctcctccctattaaacttttccagtgaagactctttttccacctggttattgtctctgtgtttgttacagccttctctctgctttcagatattcaactctgcaaagtcgtaatcatttatccacatgtacttttgtgtgtgtttgtcccatctctctctttagaagacatgttcctgctggtgcttggaataagacgttcttttccctccccgtggagatgctatttttcctctctttaaggagctaggagagaatttggttcaacatgtcttatgtatcagtttagcactgtgcttttcttcccttagattgacgtgtgatttaaaaaaacaaaatttggaaccggtcatgtaaagatggtgtatgtcacttagtataggtcatctctgtaccctttgtatactgaattttgctgtgacctgtcaaaggataccgaagacaggcccatgcgaaacatttaatttctggcattttcgtgcatgtgagactcatctgatgtgaactgaaggtatgttatccttccttttaatgataccaaatctggcccaaattcatatgaccccttagaaaattactgtgataggaacagattctaatgaattgaaatagagatttctccacctcacgtaattaggatttgagagttaaataagaaagtcagggatagggacagggatagaatggcatggagaaagttggagccaagaacactgaggaaaagaagggagtgacagcagcaaggaccattgctgttaggaagggaattctttcatcagacactggaattctcctgccaggcactggagtttgccctagcttctgtctgcttttactggatcctgggtcctccagctgaagtgagttgtattacaatcccttccagttttgcacttcagggaagtgaaatggagcaaatcctaatggcacaggctggagggagaaggactagtttctttaatgtgactcaacacctggcaaggctgtctttttctcaaccgggtttcatctgttagttctgcaacatgcagagtcagattttgaggagtttccagctctcgccttcatggctgtccttgtgtttgtgtttgtgttctccaggcgcctccttctccactctttctccagtgctggagacctaggggtgtgcatcctggaggattgtgaggaacctcagctgctcccctcgtcctctgaccaggcggtgggctcagggagctttggagtctcatgcggtcagcagcgcttagcatgttcagttggctttttccatatcaccagtgacatctttcttcttcctaatcatcgactcatggaatattagaattaccaagcctcttgcagttcctgtggtctaattccttcattttagagatgaggacccctaaggcctggggaggggagatgataggtgtagggaaaaaaaaaagaaactttttcctctaaataagattagcaaaacgataaattaacaggagaaaaatagagagtttattaacacgtgcagcacacatcacacaggagaaacctcagtgatgagtaactcaaagtttcaagagtttttatttcttttttctgtatagcacttgtcagtctctgatattgatttttacttgtttcttaatgtttgttctccttccactagtacgtacactccatgggaacagtgattttcatctgtttgtcactgctttattcccagcacatagagctgtgcctagagtggtaggcagatcattgttaaaacaatgaatacatgaacttcgggcaagttacttaaccattctgtacttcagttttcttgtgagtaaaatagggattaaaatagtgcctacttcatggagttgcaataaaaactaagggagttaatccatctaaagtaccaataacccacctggcccatagtaaatgctcaataaataggttcccagccaggcgcagtggctcacacctgtaatcccagcatttgagaagccgaggcaagaggatcacttgagtcaggagttcgataccagcctgggcaagtggtgaaaccccatctcaactaaagatacaaaaattagctgggtatggtagcaggggcctgtaaacccagctattcaggaggctgaggcagaagaattgcctgaacccgggagatggaggttgcagtgagctgagatcatgccactgcactttagcctgggcaacagagtataagactctgtctcaaaaaaataataataaaaataaataagttccttattataattactaaatattgtatttcctcactagaatgcaaactccttgagggcagagactatttcttacatcatctttttatttctagtagcaatttggctgacacatgattgatattcagtgtttgttgaacagatagataagttatagaaaaattgtaggtaatacttctgtaaataattgttaaatttaaatgaatagatttccttcctgtctttttccctaagctgcaatcagtgaatctacatggattacatagattgtgactacactgtgccttaactcatcacttaggcagtggagtcgatcatttattttgttgccatgacttggctgaaatttcttggggactgacagcaatggcagaaacttgatgggtcctgaatgtcaccatagcctgtaagctacacaggagccacccgagaactcctccattcgtggttgtaacttcacagaaatgcttcccagtcagaggaggtacttagcagaggctgcaggcatctgaacagacaggtgcaggaaaagttgaaagggaaggttaagggagaaacatactcctcatctccgttctcaagaaacggcttaaaggttgtaaaaggattctgcttttaagcttgagtcactcatttgacttctgtgggccttgcaatttctctggtaaaattagagaatatgaccaagattatctccaaggacctgtcttactttttaagtttcctaattctgtgaaaaatgcttaagaaggcctcagactgattaagaatttaggggtgtaagtttaagtggggatactgataaaagggcaggggatgttactgtaagagcaatggaaaagtgtctgagtagtgtagcatatccatttgtgagaggagtaattaattaaaaaatgaaggcagaatccacatcttcacaaaagtttcctatctcctggtttggatgagtgtatttaatgtgaactaagttgcatgtgggctgtctaatctgggcatctgacttgtgaatccttcccataactaagccccgagagatttccaatcatgcccataaggtaatgcaatttggcaagaggatttggacttgacgaaatctcttgttctatttggcatccagaaaaggctgacttgcctaatttgaaaataaattgaaaacaaaaaaaatttaaaaaaacctctggttgttccttgtttgatgaaataccatagaaaactattactctttttccataccccattactatggagaatatgtttttaccactgactcttctaagtatctgtccttagtttcctgtccttccctgccacaggttccaggaaggctgaatcactacagcatggtattaaatcatagaaatacgtgtgtattttgtaccatgtgggtctgcttgcattcctaacatctgcattagtattagttttctattgctgcataacaaactgccacaaatttagtggtttaaaacaacattcatttcttatctcagtttctttgggtcaagaatctggacacagtttacctgggtcatctgcaaagggtctcagaaggctgtaattaatgtgttagccagggctgtgttctgatctgaggcttgactgaggaagaacccactttgttagcagaatttagtaccttgtggttgtaggacagaggattttagtttcttactagctgttggttggaggcttccctctgttccatactatgtgggcctcccaacctagacacccacaacatggcagcctgctccttcaacaactagcaagggagtttaatgttttcctgtagctcatatttctttataggtgtgacctcaatttttcccctaatacctttgtaagatagaagtagggagaaccaagatagatccaaaaaggctgaatgaaatgcctgagattgagctggtggttgcattgaacaatataaattgatgaataacaatttattaattgtgccaggcactgtgctaagagcttcatgtcagtacattcttgtttagttcttagcacaaccctctgagttaggtattactactactaaactatttaacaggtagggaagttggggattagagaggttatgtaacctgcctaaagttcacagacagaaagtgcacagcccagatttgaacacaggcagattccagaacccactatcatcgcactctgaagatctctccattagagcacgatgctgccatgttcaggattcttcctatcttttgttaaatacttaagttcctgcaatattgcaaagggataattgtaataggttgcccctttcaaagtgctttaataaaatgaaaggacaacattctttcttttctgcttgagccaggctttctcatttttctgaagacatctaggataaatggaaggaaaccagaatttagtggggtttgcatgtattgataacagttttgctgaatacacatccaagagctaaaacatgcaaactcttcaaaattggtagaatacagcttgctcattttaattcatttaaaaatgttcattttaattaagcattacccattttattattccataaaacacgaatcatctggaaaaggcagcataggtctatttttccagcctataaaactatatgtagatggatcttgatgagggatgtacaagatttaattagaaactgaaattttagagtaaaaaaatgcaggggtgtgtgtgtttcttctatatttgtggaaacccaaagagctatcctgcaggatacttgaaaatatatataaatacagaggagaaataaagtgcggagaaggtttgttgattattgagctttttcatgggtgatagaatggagcagctgtaggatggaggtaaagaggataggaaaatgctcttctgggtcagacccagccccacatcccaaaatgttgtggccaccctctgtgttatgtaaaatgtaggaaggtcctcccagctggaggaccttcgagtatactgcgttccccctcctcattcactagggaggatgcaaaggtgcaaatgggccaaggtcatgcattggacttccttcattaaactgttatgaatttgccttccctgagctcctgttggaaaaatgtcttgaacttactgttgttgtttctcctactgcgtttgtatttctttttataaaccttagtccaagtcagcccagtgctgtagttatttgtgtatctgtgtcattctccctaggatgccctgggccatttttaatcaactttgatcccctttcagtgccttgcatgaggcctggaacattggaggtacacactgtgtattgactgaatcgagtgaaaggtatttgaatatacagttgactcttgagctacatgggggttaggttgctgacccctcacgcaggaaaaatatctgtgtataactttttaaaaaataaatttgggggtacaagtgcagttttgttacatggatatattgtgtagtggtgaagtctgggcttttaatatagccttcaccctagtagtgaacattgtacccaataggtaggttttcaaccttaacctgcctcctatctttggagtctccaatatatgttatttccctctgtatgttcctgtatacccattctttagctcctgcttataagtgagaacatgcaatatcttactttctgtttctgagttatgtatataacttctgagtccccaaaaacataactactaatagcctactgctgactagaagctttaccgataacgtaaatggttaattaacacatattttgtttgttatatgtattatatactatattcttatagtacagtaagctaaagaaaagaaaatgctattaagaaaatcataggagaaaatatatttaccgttcattaagtggaagtggagcatcataaaggtcttcatcttcatcatcttcatgttgagtaggctgaagagggcttggtcttgctgtctcagggtggcagaggtggaagatgcggaggaggcgggaggaaaggcaggccactcagtgtaaatttactgaaaaaaattcacatataagtggacacacgcagctcaaacatgctgttcgagggtccactgtatttcatctgtttgtttcctactgtggaagacagggcttccaggctttagttctaaaacttccattgtaaggccttgaggaggatcctctcattccagtaatctgagagttggtaaacaaattcaaagccactgtatcactgtcctttatgtccctatgttttttttttttacttttttttgccatttcttttttcttttgcactttaaaaatcgtggtaatatatacataatataaaatttgcccttttaaccattttaagtatacaattcagtagcattaggtacattcacattgttgtgcaaccatcagcgctctccagaacttttacatcgtccccagctgaaactctgtccccagcaaacgctggcatgcccttctcccctcccccagcccctggtaatcaggggtcttcttcctgtttgtgaattatgactactccaggtctctccgtaagtggaatctcatttgaccttttgtgactggcttatttcacttagcatgatgttaaaaggtttatccatgttgtagcatgtgtcagaatttcattcctttttggcagccattgtcttaagactcaagagtttaagtgtttttagtctctgtagccagaggtagatttttcttccagctctgtgcttgggcatttcccaaagcgaaacttccaaaggcacatcctgtcttcctgagtggtattctttgaggcagctgagtagcaaaactgaggctattaatgttcgcttagcaactgagcagctactgtgtatcaggggctctgcctgtgaccagggagtacacggtcttgtagaagagaaaaccgggtactcagctaactgtggtaccatgtgctaggtgccaaaggaggaacagagggccctgcgtgatctgctcaggaccgtgagggagaggtaagaagagttcaaaggagaggtcatatttcaaacagaccttgaagctgttcaaggcagagaagaaaaggatggtgttccacgaatggcctgagctaaggcaagtggaaacacgaggtttgttggaagaatcgcaaggacagcctgatgtttatagaaagtggagagagtgtggggaatgacattagggcgtatatgaatgccatgctaaggattggggtacaggtaacttggaggcctcatgcagttagactgaccccgttcgatctgtgggttgacaggggggtgtgaatggattggaatgggagagaccactgtgtggggcaaatgggaagggagcgtggttgcagcattttaggccagagatggtgagggcctgaagcaaggcagtgagagagaagaatttgctcatggtcaggtgcatcgggcctgttgacagcagagtttgttggactgctcccacctattgctttttgtcttctgaagtttttataatttcaattccagtttagaggtctgtttttagaacatttctccgatcttattatacccatagtggcaagaatgaattgatttgtacaagatagctggacaggttcctaccttcaggctttctacccaactgggcacttggaaggatagtttttcatagtcttcatgggctaagtgattgcatgtaatatacaatagctggagtctaactgtaattaaaacaggttagccaagctacaggagaagtatcccccccatccccacccccatggcccttccccactgcctgtaatctcttggtaacaaaagaagtcaaacaaaaatttcatgcaagggtataaacatactttgagtcatcagaacctacctagtggtccatagaaccttggctcttcatgaagggcttaccaggaagacatagtcgttgttcatatgcagtttctattctgaactgtaaaatgtgagcgattactttggaaaattagaagaaaaaatgaatttactcaggatccttaaaaggttacatttgtcatgcctgtaatcccaacaattttggaggctgagccgggtagatcgcttgagcccaggagtttaagaccagcctgggcaacatggcaaaaccctgttgatacaaaaaatacaaaaattagctgggcgtggtggcacatgcctgtagtcccggctacctgaaaggctgaggtgggaggatgacctgagtccggagaggttgaggctgcagtgagtcaagatcgtgccactgcaccccagcctgggtgacagagtgagacctcctctggaagaaaacaaacaagtgacatttataggagggtcccaaacatacaaatatggaaagtgaaaatactacttgctacaggtactgaaagaaaatactcaggagtcattattcaactgaaagcagattgaggcaaaggtgctgaaatatactaaaaataatgagtttcaaagaaaatcatcttttgttaacctttgcattagtcagtccttattttttcgttttagtcactcctttacaacaatgtgtataaatatagaagatgaagagaagtgtgataaaaataatttaagatttggaacatgggttttcaaaggaaaggttagagaaatttggggattaaaatggacagactttatcatcttcaatattaaaaaaaaaaacagaaggatatttacgagcaagccaatgaaatgataccaagaagtttataacacatactctgcccttatggggctttcacctagcggccatgtttccattgtgcagttaactactcagttattttttatttcccaagagcactaagtgaaagcaaatgcatgttaagtcgtgggggctattctgaatgatcaatagagcccaccagagtcccttataactaactacccattagcagatgtggagtttccttcttggggatggaggggactgagtcggggagcttcctggatgggagtttgaaccaaataatcttaattatgtaattcctttcaattttaagatttgagggcaactttagaaagagataagtaattgactgaccaactttataaagagatcagtaattgaccacccttggagtgtaagagtcaacacctaaaataaatgtatatttctaatattttgaaaatagaagaaaaaggaagaaaggaggaaagacaagttatctagtggagacgatgaattttcaggtcatccttctagtttttattttagacttgtgtctttagttgaaattattacaacttgcatgtattttgtctgtttactcattatttcgtctgtctcaccgttccaaggtaagcaccgtgaaaacggactgtgtctttttagttctttactgtgtttctgccatcaagcccagtgttgacacatggtaggtactcaagacatgctggctgaatgaatcagtgagtagtgaaggtggatcatggggagaagagtgctgcaaataccagagagaaggagaagaaggaagtggaggggatagcggaagaaggggaagaggagtgagtttacaaggcctttttttctttttttgacagagtctcactctgttgcccaggctggaatacagtggcgtgatctcagctcactgcaacctctgcctcccaggtttaagcgattctcctgtcccagcctcccgagcagctgggattacaggtgcacgccaccacaaccggctaatttttgtatttttagtagagatgggatttcaccatgttggccaggctagtctcaaactcctaacctcaagtgttccgcccaccttggcctctcccttgctgggattacaggtgtgagccaccatgcccagcctccaagccctttcttttaaaaaataaattttattgtttatatttgagatttacaacatgatattatgggatacatatagctaatagaatgggttgctatagtgaggtaggcaaccattctcacaggggtttgtgtgtgtgtgtgtgtgtgtgggacaagaacaactaaaatctacatatttaacaaaaattcctaatagaatatagttttattaagccttaaaaaaggagatcttgccatttgccacaacatggatagacctagaggacattatgataagtgacataagccagatacagaaagaaacatattgtatgatctcacttttatctgaaatatttattttaaaagctcaaatacagagacagagaatgaaacagtggttaatgccggtggaaatggggagatgtgggtcaaaggacacaaaatggcaagtgcacaggatgagcgtgtctagagacttaatgtacaacgcaaagactaaagttaacaaggccttttggttctgttgctgttgttgcattacgtaatagtgtgctaagaacaggagacacataacgtcatctaaaaaacaggagatgaagtgggccggtgctcaaacatgctagctccgggaaacaattcaggatgatttgccactgagtcaggtttgttaattttttctacaatagccaaaggctctcaaaaaatcaaggtttcctggtttataaataatctctggaggggtgtgtgtgtttcaggcatctgactcctttatttatattctctgccctggccttcagatgattttcgagcggctttcgattagattatctgcattgggataatggagcaattttgcagcaaactgctttggactgttcgactcttctcggcctgagtagccaaaaaataataataacttgatttagtctggtctagcctgctgtagtctgacttaaggatataatatcccagaccttagacacttgacttaatacattaacatattaacaaaagggtaaaacaaaatactagaagaaaacataataaaaacagcacattatttttcgggcatttgacttgaaccacagggtgccctcataatgaaaagaatattccaagggaaagatgagaaggcaattgagacaaccctaagcccctgtctgcccactcaaaccctctggccaccactgtactctctccaccatgagaaagcaggccaggagtcagttcagctgcctgtgtttaagtgcttttaatatccatggaggcacagagaaatgaaagcaggagccttgaatctagatttttccagaatccttttgagagacttattgtagcggcaggagctccattttgggggggtcaccccctgaactttgatggttttcaaatctttggagggccttggtcttagtggctcagaggtcccctgccaggacttctttaggatggggatgataggaagcgaacgttccttctgttgaggtgcatcagccttgcatctgagttcttgatgtcctcaggcctggcttcccagttggttaaaggaatgaaaataacaacgcctttcatttgcagtgcagctttaaaaatatttgcttttgctatctcattttatcctgataataaccgtgtgagaaaagcaggatggcagtcttactgtccttgtacacatggagaaatagatgcacagaggttaagttgcccagtttagtaaagggcagagcagggactagactccatttctcttcgctggaagagcccagcactatcacattgcaagattgtttcttgtaaaacagtcctgcataaaaataaaaaagagtagagagcatgatgtgaatttcagcttttagaggctgctgtctgtttttcataggttaaatagtgaatcagtgtgctccgagcttccatgaggggcagccctccttacctggcaggttactgagttttctaatgacttcagttttaggtgctaggagatagtagggccacctgcactgttttgtgagcaggacagataagaggcaagtggagagggtgggagagcactggcttgcatcatacaccccagctgcccttgcagggcccgactgttacaagccaggtatttctaggacgattgcattcctagccatatctgttttctttcagatggggcgtacaatttaccttttgagttctgtaaatggaatcgaggtgaaatctttactctggtgcacatatgacctgcaggttgatgctgcttgtttggaaagccctctacttcacagcgaacagtttgtactgtcaggtagacctctctgccaggggaccaactcctgtgtatttgaaaaggaagacaaacctgtattttgtagaaagggtctattgttcccagggggtggttgtggccaacttcttggactcctttggctgactctgaagaaggaattgaagtcagaatgctaatcactgacttgcacaggcctcaacaatcaaacctcagggaactcagaagaaagtaaacaagagcgctcaattgcagagtcttgaagaagagcagctgtgtaaaggaggctagacgtgaaaaaaacgtgttggagaggaaaaaaaatcacattccctgcttctcaaatgggaagtcttaaaaataataaagaatcctgagcagcgggctcatttcccatgagcaggcttgctgtggcaggcaggctcacctgctagaggtggtctcctggcttagaatgggagcacacctgtgggaatcgcagcgcttctctacctgagctggatgtagattgtttgaaaatcctagttgtgcactgcgtttccatcgatccctttcatgctcccacttgttcctttagttccaaatagcaggctggtgttgagaattctgtcatcatggggcgtttttttctaaatgtgagaaatgtcggaagcatgggtgttctcagattgctttagccactgatgattttccttttagtgtatggaaagaatgattttcttctcaatttctcccctctaacctcctgcatgtagatttatggattctaatcgggacctttctttgattcagtgccaggtacacaggccccgagtcgtatgtggcaagtggccaaggggatcggtcctggagaacgtcatggcacacaggcattggccatggagtggtgatccctggctctggttcttctccgtccagtgacctggcatgtcacctcacagggccatatttacctgttaactgagaaactgaactagaacatggttatgataattttaatctcaactttgtctcctcctatcttatggcagtaaagtgaccttttagcaggatcaatttataagccgtttgaagtctatcttttcttcccttagcagttgtatttgtgattttctcagcctgattttaagttattctgttataattttgaatgttctgttatgcttccctctgtggtgagcatacgtcgaaagctgtacatttccttcagggtaacctttgtttccaacaggagagagttctcactaccacatccagccttgatctctttaacctaaattggaataagccagacagagctggggttcatgggacagatgggagcatctcaagtttagggttggggagtctcagtgaaaaaaaaaaaaaaaagaacaagaatgtagacttggaatttgagaacagataaagcagtcctcacttgtgctctgcaagggagtgctgttgtcttctaaggtattgaacagactgggtgggaagctgctgaaagactggaaatttggggacttagcaagaagtataatggtgctgtaactgcgttttcttaaggaaacttggagctgaggtgccacctccctgggagggacttttccactggtttcttgcagtctggacatcacagacggcccagactcccccctgtcttgtgtacattcccttctacaagccttgctgtgttatcacaggtaccacttgtaggccagtatcatgggggatggacattgtttcattctaggcacattcaggaatcgtatatgtaccctgcctcgcttctgacctctcaagtcaacttttggagctgttagtgacttccagaaagctgacacccagtgccactcaaccaaccctgcttttctgctggtttatttttgttattcctaccccgactcatcttgcttttcagccatcaatgagaatttattgagttactcagacactcactcagaccctgtgggagagtagaaagatgtgtaagaaactgtcccagaattttgagggacttaaaatccattcgaaaaggcaaggtctaagcacatgaaaagttacctagcaatatcagctcatgaatggagagacagttgatcagagaaagagaaatgtctgttcaggagtggtcaggggaggctgcattgaggtgatgcacacgtgtggcctgaaagcttgattcgttccccaccccccattgtattctatatgtaatatttgatatataaccaagaagctatgtgagttacaaagcatgatgatacagtgaacacctatgaactcaccacccaacataagaaccaaaatgactgatattgataccctacctatatacctctaccttcttcctcattctacccctgccctcttatcccagttagccactgtcttaaattttacagattttattccgttcctttttttcttttttgaaagcgtttgatcttttcttctctcctcctctcctctcctctcctctcttcttcttttctttcttttcgagatgaggtctcgctatgttgtgcaggctggtctggaactcctgagctcaaatgatcctcccaccttaacctcccaatagctgagaatataggcctgtaccactgcacctggctagatctcatgttttctttggaaatctagtaattgtgatataggatctctgaatgcagaacacctagggcacaagtgatgcttaattttatgtgtcaacttagatgggccacagtgctcatatgtatgatcagatattcttctggatgtttctgtgaaggtatttatggatgagactaatatttaaattggtggactttgagtaaagcacatggccctccgtaatgcgggtaggcctcacccaatcagttgaaggcctgaatagaaccgaagagggaatgccacagcagatggccttcagacttacactgcaccaccggctcttctctgggtctccagcctgctggcacatcttgcagattttgggcttgtctgcctctataagcacataagccagttccttaaaataaatatctgtgtgtgtgtatgcatttatatatacacacaccaccctattggttcactttctctggagaaccctgactaatacagcatatcacacctctctgtatgccacgtaggtagcactgcagaattaatttgcagagtattctcacatcgggaggtcagcagtatattaggcgcatatcagaaaggcatttggatgattctggcctacaccatggaagagaaaccttgattttctgtaaaagcaaggccagctttttttggagaactcttttctcccttttagtattctgtcccctcccaaagctccgtggctttctctggtcattcagagaccgttattccctccccagcatacatgcacactcatgtctatggtttcccgtaccttctctgtggagccctggttctgcctgttacaaggaaaggcatttagtttctctgacctcctgtctcctcagatgtcagtctgtatttattatgtgtcaccatgtactagatactgatttaagtgctttgcctgtttattccatggtattttcacaacaatcccaggtggtaactactatcatcttgttcattttgcagtgagaaaatggcaagaaagtcatagaaatgggatttgagtcctggtagtccgactttagagcgcttgcttctgacgcttctgaaacacattatgaactggaaattgctctacaaatatacactactgttagtatcacaatgctagtattatagtgcgcctattgccttggggccctgcgctctgctagttgatagctagggcatttttggagctgatgcaggctaatgagggtcgttgataggaaagtattgcttttcctagaaagctctggttctttgacagaagccttctggtggccacagttcctgagccctaagaagaaatcctgtagggtacctctaggacatgatggttagtgtagggtcatgagtctgctctctggtcatgtcaagaaagctgccccctactttgtgtttaagcagatcctggaggctgcaggatagcccattgagactaagtggagaccaggcatcaggtgaggaagccttagcttggagggtcagaggcctcagctagacttggtaaatatttggcacttcctctccctcacaggaccacagcaaatgttaaataatcacagcactctttagtttgaccaccactaatcgattgagttgggcaaatagcatggaaggtctttgcctattctttgggagctctgggtgtccccagagaattgagtctggcattaagttttgcctctcctttctcgttttcttttcccagccatatttctccaggttttcagcacaaactcaacccgttatttttgtttcttcagtgtctttaactctttccctgcctgttgagatggagtctcactcagttgtttagtaatggcagtacatcgagaagaccatcacacagcaagagttctaggagtttctttgcctgtgaggttagttttctatatttcatttgtggagggaacagaagtgatcccatttaagtgtggaaactttctttttggaacattttccttcctttttcttcatacatcgtggttccttctctcctgcactctcacatagataccagtatatagagatactgtttctaatcctgaaattccttttttgaagaaaagagaacattgtgtgccattgttttctgcgggagatctatgctgcgcttctgttttccaaacatttatccttaaagcattttcagatgtcagttaaacaagggagcaatggcccctgggtcctgttagggacatactcttaatgactgtaaaatgaataaggtatagaggaatattcgtctgaagtcatgatttgtacgtcatttccttgctttcttcaacatcattatttctgaaccagaaagtattttgatcaacattttggaggaagaattgtatggtatttcccaaccttccccccgtgggtggaattgcatcactgaatcacttttctctatttttttccactttctgtctcctgttttgtcaaggctccagcaaactccagccttttttctgtgttttttagtcctaaaattctagcctggcatctgcgattgtgttagtttcccatcattattttctagcctaacaaggaagcctcgtgaaaacaaagcgttctgtttaatcctcaggtggacgtttgaaccctcgtaaacatgctcccttcacccccccatcttactttttagcaatctcagagatcaaaaagacagtcatctcatccagtgtagacaaagtgaagtacattcctttcatttgcctttgctgtagtccctatttcatccatctaatccgttttaagggataaaattgcaattacaaacatttcatgtgtggtcagatttttagtattttaatctggtctgagacttaagatgaaatgacagttctaacagaggtcctgtgtggaagaaaggacctaggtgccagtctcagtccagtcccttcttagacgtgtgactttggaaaacctctcagagacccatcatctcgtcgtagatggggatggtattacaggtcgtgctcatctgtgttaatcagggttctccagagaaacagaaccaataggagatccacatcgctgttgatgtcaatagatagataggtgtgtatgtagggatggcaggctggagaccctgggaagagttgatgttgcagtcaggaggctgaacttcctcttccttgggagacccaagtttttttctcttaaggccttcaactgattggttgaggcccacccatattatggagggcaatctgcttcactcagagtctactgacttaaatgtccatctcatctaaacggtatcttcacggtgttcaggcaaatatcggtgccacgtcctagtcaagttgacacctgaaatcaaccatccccctcgcaggagtttgtgagcatcaggtgtgaatggtcaggagctgcaggcgtgcacggaggaggccactgttcttcatgcttgagccttttctgtttctgcctcgtttactttccttctctctcctcactgttacccttttcctcctacctctgacacctgcccttcacctcccacctgctacctgagatgggagccacacgtgaagtgagaagaagggagaatgaccaggtgggtgatgaatttaaactggaaagagaacagatcctgatgccagtcaggatagaggtggtctagctcttatgtgatcatgggcaagtgaacatgcctcagtgagcctctgatcagtttctgtatgaggataattatatttatcctctaaaatgaggataatgatatttatctgatacagttcttctgccctaacacatacgtgtgcattgcctttgctttcaaatgtttggtaatcacctttttagatttaaatcattactcaaattgaactctccttcagcctttgtgtgtctagactttccgttaagattagattacttgttttactatgattgtaaaccattgtttccatgaagtctcatagcaaatgttcttttgctttcctcttttgttactagtctcttgttatttctcaaacatatcaagcgcacttctccctcagggcctttgcgtgtgctgtttcctctacatggagctcagatgactgcatgtctttttcctaaacttctttgaggtgtctgttcaaatgtcttccactcaggggggctttccctgagcaccttatagaaaataaataaaatgatataatatctgctctcacgttctgttgcacttgccttgctttgtttttcttcatagtctttatctggaatatatatttatttctgtctacatcctcccactaggacagtggttcttaaccttggatgttcattagaatcaccctgggagtttaaaaaaatcctctcctgtgttctagaacaattaaattcagtaatgttataaggaagagactcagcattagtattttgtaaaactcccaggagattcctatgtgcaaccaagcttgagaaccagtgttctagtctagaaccttacttcccagaggatgtggactggtggcatcagcatcacctttatttgaaagctgagtccaattcttctctttggcaggacccacagatgatgcatatgacattaaagcttgagatgcactgcactagaatgcaggggcgtcattttgcttgctgctgtatccttagtgtcagaacagtacttagcacatcataagcactcactaaatatttatcaaatgaattaacgttggagatgagaggaaggaggaggtaataatggaaagaggacaaagaaagagaggcaagggaagacagaaagaaaacagatttggcctccccacattttctgggcacagatataacggctctctcttgctctttgctcccaatcaaaactcagccagcctcatgtccaacatctcatgggcttgcctccagttctctgcactttccaaagtgatagctgactaatcaaagatattgtgataatgaattgcctaactacgcagcctgccttcttctcttctagcatgtatttgattaggggcctgctgaaaccaattaagtaaataattcagtatgactttatgaactacctactacatgcatggcttaacacaggcagcctcaagaggctgagaaccaaagacggaaaaagcacaacttatagtccagctatagagacagacttttaggtaagtaatcataccttgaggtgaaaaaaaaaaaaaaaaaaaaaaagatctgtgatggaagacacttacagacatgtagacagggtggtgtggaaggtctacagtaagtgactgtttcagactgaggggagggagtagactcctggtgaggagggcaggggcatgtgagttgcgtatgtgagtggctggaacacaaggtggggaggcatgcttgtgtgctttggcaatcacaaagacggttagaaggcagttaaagggttggagtatggtcagttctgtgttttgaagagatgactgacagtcgtggaaggtggattggagaaggaagaccatgggcccatcacacccagtttggagatgattaaagatgatacccacgttacatgacagagccgtggcaggggcaataggatgtaaaatgtggagatcgttttgagatttttcaaatggggaagaccatgtcttttttgtttaaccgaaatgagcaggaattcttggggtgacatcaaattcgatagttctatgagcatatacactgaaaaccacgatattacggaaatataggctatttctcttttcactggcctttagatagactggattagtggagagttgagcattcgtcttttcttagagccttgagtggagatggaccctagtgattatttattttgactgctttattttgaagatgaagagaaggaggccaaggtatgaaatgattatcagaaccaggaatgcaacttgaggtcaccattactagatacatagtgagtgccaggtggtttaaatactcattatcaccttcaatcctcacaaccattgtatgaaacagggatgctatgtttattttagggatgagaaaacccaaaactcaaggaggtaactcacccaaggttttgcagctagtaagtggtacagctgggatccaaacccaggtgtgtcccagctacgctgtgccatctactcctctgccctgctgctttcctaatcagtactccctgatgctgctgaggaagagtcgaacttatagaggtcacacattgttttccttgtttttggatgcagttttgttttctttgataatcataaattgaggatttatgacagccgatttctctctgttattttggtcacaatttcacaatgtgacctttggaagtaagggctttgagttccttaaaatagctaaattgagaggataaaggaaaactgactcaaatcagagaatttgcatcaggtttctgtaggtttcttataataatcaacaagtatttagcaattcttttttcacataaatagaagagccaagagaagaacagcgtctttgacctgatggtgttaagcctgcaatctgggtaaaaaggaatttgcggaacactctggatctgatactgaaaagttgaagaaagcaacatcttgcttattacccttgtgtttaagaccatttggggccgggcgcggtggctgacgcctgtaatcccaacactttgggaggccgaggcgggtggatcacgaggtcgggagatcaagaccatcctggctaacatgaaaattagccgggcatggtggtgggcgcctgtagtcccagctactcgggaggctgaggcaggagaatagcgtgaatctgggaggctgagcttgcagtgagcggagatcatgccactgcactccagcctgggcgacagagcaagactccttctcaaaaaaaaaaaaaaaaaaaaaaaaaaaaaagatcattcggaaccgaatatgtgaagatcagggctgtgatctggtgaattgggagtgatttatgccacccaagcgagttgacctgaaccgctggggtccatcatcctgcttctgtttctgcactggcatttgtgttagtatttggtggaaggtggttcacttttggctgaagttggtggatgtgggcagagcaggattagttcctttcttctgccctctcttctagcaatgctccaaggatgctttggaagactcattattcagagctgcagctggactgccttagacatctgcatttctctatctctgaaataagggactcaaacaagagcatcactcttttcctttccagctctaaaattatgtgatttttctttcacatggtaagtgaaatagtcatctgagctgctgtagtaggttgagcttattgtatcccagatctgagtggttcaactccatgttcatgctgcccagttccaagagcacctctgtggccttcaaacagttgtacacagtctgtaaactgctcagccacttctcccagtcttctaagcaggcaaagactccttatctcttagatctaaaatcattaggtagattttgattccaagggtcaaagcacagcatggtaggcagcaagctctcatccaagcccgtatttcagcaggcaggcgatgctgaggtgctttaggccttcagtgcacacagatagatgcttcccctgagcctcagggaaagctttattttgcccacgttgtattccttcctgcatcgtatatgttaatgttgctctgccactccaaatacagcagtgtctgctcagttccattttcttccttttgcaaagagcaggcaaccagctatgccttcctgagagcagacaaacctggagccaagggggccacattttcagcttcaatagcaattgctgcagctcccttatttcatcacctattaggatgcgcaggcggcgctaagtgacacatagcccataaaacatatctgcttcctgctgatttcatggttctggaggacaggctgtgcacagggattcagacagagtgtagtaacaagcaggggtgctatggaatattcatttatcctaaataaatatctttttggcaactgcttttccctatagtgtaatacagtaacgctatggggcatgtatacatgtgtgtgcataaggaacatccgtccatctctagctgtcatgtctgagaacattaataaaaggctcgacctcatgcgcctgcaagtgatcacaagacagttactgaagcattcaaagaagcaatgtctttgtcatctgactcattttgaaaacgcatgagaatgctttcccaaggcaatttcttacaaaatacaccgtgggacatttttcctatagtttattgttgttgatgatcatcgagaactttgtacccagagacactctacactgtgagtgtcccctgatattccctcccactcagcctgatcagtggtatctgacctccaactactcgctgcaacaacccgttctctagctgagtgggcctggactttgaacagaattaacagcctggaagtggagttctcaatctcaacagccccttacctcatgcaaatttcttttgagctgaaaagaacaaacagaatgccaccttttaaaacaaccttcttcgaaacatggggggaaagtcaggttttatataaatcagagagatagaaataaggcccataataaaaaacttgaaatagatttgggaagtttccgaaacttccaaaaactagtggtccaaagtgggcaatattttttgtgtgcattgagaggcccttcctgtttccttggtccatgctggtgtcttgccccgttccaggaaccctgggctcccctgtgctgagcagcccccacacctcagcccaaagcctcaccccttgcctgaccacaccccatttcttttggcacagcttcccagacttacaagatgtcttgaagtgactgaatggagttagtgttcggcaggactggctaggtaactactgggacttggtgcagaatgtagatgtagggctccttgttcaaaaattaggaagaatttcaagatagccacagcagagcattaaaacaaacccaggttggctaggtgtggtggcacactcctgaaatcctagcactttgggaggacgaggcaggcggatcgcttgagctcaggagtttgagaccagcctgggcaacatgacgaaaccccatcgctactaaaaaaaaaaaaaaaaaaaaattagttgggcatggtggtgtgtgcctgtagtcccagctacttgggaggctgaggtgggaggcttgcttttaagcctgggaggcagcctgggcaacagagtgagactgtgtctcaaaaaaattctcagacaagcacaggacctctgtatgactgcacaggtcacaggtcgcacactcacgaagccagcccagtgctcaatactgtattttagaataagcaggaatcagtttagggctgccagacttgcagaacaatcactgtgagggctgatggcagatccctgatctgtgattcagaaagagtcattcagacaggcattgcccagagtctgggttctctctctcctgaagttgttgaggctgtgcagaatgtatgtgtttagtgtttcatccagtctgttagggtaatcagtgtgatttctctggcatctccaggactggaagcagaccagatttaatcaaagctgttgaagctgcccagggactggctaagctgccaccctgttagtgaggccgctggggagagggaaactgaagcctgcaccacggggagccgtatactggagtctggaatccatttgctccacgcaggaggcctgcatctcaagtcaggaggcactgcctggaaatctagcatcatgttgatactctttggtgaaggaaatgtggtccttgccccaagttatctcacagtccctttgggcaatctcatgcatattaaaaataccgacaaacatagacccacgaaagatcctgatcttttaggtctgtcatgtgatagagacagtaagtgctctgtatagacatcaggagattttcttccctgaaggacaagctcttgtagcattttcctgcactgagatgcagtcatgctagcctgtctttcactcaccagggatggcaaggaaggaccagctgacaagtaataaagcaaaccagtttgtaattttacagggctctgaataaagatgcatttatctctggccaacaccaagaggcctcgatgtctctcacagggaggaacttgatgggctgtggcgtcttttgggggtgctagggcagagtcaggcttgaatctgagaaaggcaagtctgtgcattaaaatccagcacgggggtgggtctggggcaagggacaagagagaagcgggaatggaaggcagggatgaggactggtaacagaccctctgcctccttccttcctcttccctcacactggctctttcagccacttccctcactgagataggaagcggtcctgtggtgacgccaagggcacaggcttctgctgtctgggctccggcacagtcattggctctgagtcatccgggaaggaaaggcatcttttaccgccccctctcccaccgtggcctcaggctcccaccatgcaggaaatgcctggtctcccacgaatcagaagctataaacaggactggtggcagttcatacagatttggcaaaggacgagaagctgtgggttattggtatcagtgattatagaaggggaaagagttgatatctgaagataggaaaaggggcttctgaggatggtgttagcatgttcaggcacagcattaatctttaagagtcacgaagagccatcagcacagtcatctccctaataagccttgagagtgttgtcaaagacacttctggatataagtaagaaaccattgagactgagttgtatggttaaacagcacagtttttagggagaggcaaaggcaactctgtgttcagctcttagttctgacacttatgaactcagatttaaattccctgagcctcagtttccctgtctgtaaaatcaggataaaaatttcttgctcaaggccaacaaaaacaagcaatggggtaaaggactccttattcataaatggtgctgggatagctggctagccatatgcagaagaatgaaactgaacccttacctttcaccatatacaaaaattaactcaagatggattaaatcgaaatctaagacctcaaactgtaaaaatcctagaagaaaacacaggaaaaacccctcttgacactagccttggcaaaggatttttggctaagtccccaaaagcaattgcaacaaaaacaaaaaattgacaagtgggaactaattaaactaaagagcttctgcacggcaaaagaatcaacagagtaaacagcaacctaccgaatgggagaaaatatttgcaaactgtgcatccaacaaaggtctaatatccagaatctataaggaacttaaatcaagaggcaaaaaaacaataactccattaaaaaatgggcaaagggcatgaacagacacctcttaaaagaagacatacaagcagccaacaaacaagaaaaaatgcttattatcactaatcatcagagaaatgcaaatcaaagccacgacgagatactgtctcacaccagtcagaatgctattatcaaaagtcaaaaaataacagatgctggtgaggctatggagaaaagggaatgcttatacaccggtgggggaaatgtaacttagttcagtcattgtagaaagcagtttggagatttctcaacttaaaacagagctaccattcagcccataaatctcattactgggtgtatatccaaaggaaactagatcattataccaaaaagacatatacacttgtatgtttactgccacgctattcacagtatcaaagacatggaatcaacctaggtgcacatcaacagtggatttgattttgttttaatgtggtacatatacaccatggaatactttgcagccataaaaaggaatgaaatcatgtcctttatagcaacatgggtaaccatgttggaagagctggaaaccataattctaagtgaatgaatgcaggaacagaaaaccaaatgctgcatgttctcacttataagtaggagctatacatcgagcactcatgaacataaacatgggaacaataaacactgtggactactagagggaggagcgggagggggttaatgagtcagaaactacctcttgggtactgtgctcactacctggatgcaagatacccatgtaacaaacctgcacacgtaaaccctatgtctaaagtaaaagttggaaaagaaaaaatatttcctaaattgttctagaattaagcaaggtaatatttataagacatccagcagagggtctggcaccctggcatataatagatgcctaatgaacattagtttccttatttttcttacgagagtaaaatattaagatgtacatattctgttaggtcatagaaagtttgaatggtgggaaaagtcagctgttaaaatgctgtgggatctctttttgagccttttcacctgtgatcaaagttgtccatcaccctggcatttttgagagctgtaatttaaggaagaaggtatcataggttacctctgtggaaattctagaatttgaatattatgcccctgctcacttgtagactgggctgtcactataaagttaggctgacttgactgtatctgataattaggggctgggagagcatggaaaattgaagccatagataatcccagtgtcttgttttagccaataataattcttctcccaccaagtctttatcagaactgctaacaaggagaaaagtcgactggtttgagtttcctcaccttttattctcccctgggtatcttcagtagaggtgctaatgaacagtgaaatggccaaaaggtgagcagctccagggaggagaagaaggaggctcagataatccacaaactgtaatcgctttgaataattgagttaactccagtaaaatgtagagatacattcacctgctaggcattgttggaagctaaattaacacactggaagcaaagctttcttttaagtttcatttttaatctaatggtggacattttggggcactgcagcctattaaatctgctaactgatgttcagacgttatgaaggagttaaactattctaagaatattcaaggcctgttgttctctcctaggctttctacgtcaaagagtaaagtcagaaaagccaaactgatttctcctaaaatgaagagaattgaagttactctcccaaaccccagttgtaggccaggccctaaactgagagaaatggacaacccaaatattttgacctttgcccattgccaggtgaggccagatccggggaggaagtgatgttttcttatctgccttaacttagccactacgtggagaagcactgtaatgtaattaatgcagaggtgctcaaattctggtgttcctcagaatcacctcaggggcttttagaaacagttgctgggccgttctctcagagtttctgatttggtaggactggggtgggacaaagaatttgcaaatctgagaagttcccggttccagggaccgcactttaataaccacaggtgaaaccaatgagaagctgagctccgaagtcagactacctgggcttgaatctcagatctctcacttggtagttgtgtgactgggtagttgatagtttacagaatctctttgtaccctcgttttctcatttgtaaaatgggaatcctaatatctcataggattgtgagtattaaatgaattaatatgtgataagcccttagaacattccctgctctatggcaagtgtgcattgtaggttagtatgacacggttataggctctctgtaacaatgaatatagtgactgatattttttgagcacttactataggccagagattgactcagtactttgcaaggattatcacatttaatacccacaacgcactctccctcctttcccatgtcctgcttgccttctttcttccactttcagttattcctttttcccttccattctttcttgctcttcaaaagccatattgtgacaaaattaatccattctaaatgaaaaagaaaatgtaccttagttataaagtatgacctttgtgagttttttcaggactgttcacctgttctttgccttttattccagcgtggtacccggcacatagtaggagtttggtgaacattaattacttgaatctcgttatccttaaaaatcaaacagggtttatctatcagggttcccttctcttcctttgtcctatcctggcctctccttgaccaacacattggtaaatttggcctctgctctttcttttccccaactgcaggggcccacatgtcaagacaaggtggcatcaccaaccattgagaaagcttttatttcactagcttctacctggaccacccatcaaaaatctttaaaacgagtattccttgggacaaggccaggtgtcattgagagtccccaggaagagcataggagtcctggtctgggagggccggacctacactctcgggggtgcccagaggaagaacagggcctttaaagtcaccctatgttctctgacccgtgcagtggtggcatactccctcccccacccctgacctgagggtgctgtgtgtcagctctcctaactcttggtctcattagggaagctggttgtcaagtactgcccagttacagttggtttaaactctcaactaagggatcgtaagcaggtcagctgcctgagtctccagttactcttgcagctcaggtttccccttgacaggtcctgataggggctacttttgcaccattgatgtggccagggcttacagcaaggtttaaggtttccatgcgcagccttcttggcaggcagctcccagagaaaggagtatggtttgggagaaagctgcaaggcctgttcgtggatccttagtattttgagccgatcacgtggagtgggcctgtgcccatcagtaccaggagactttatacttaggggagaatctgtatgcggtgtgattttcaagggtctatttcctcattgtttctctttgcttgccctatggctgagtattaaatgaacattgaaagtctactatatgccggttactctgctagatactagcagagatggagggtaggatacaaagacaagtaaggcctagtcttcaggaagcctttatttgtgctctgtgtatcagaagagggttgagttggtgacaagaataaagagttgaggggagtgaggggctgaaaaataataagccttagagaactaggaataaagtgttaacttgggaaaattgttgaaacagttccttaagatatttgtagtagatatgtctctagataagaggcttgtctggctgtgaggtccttgcacgagacccttgcaggtttcccaaatctatttggactgggtgtcctgtattaacatgaaatcgtaggcagacactgaataaatcgcaacagagtttcggttgtggctgctccagtgtcttggagtggttgctccagacacataggtcttatcctcctgccctgaggttctccaaatttgcatttgggaagtgtatagttttatgactgtttcttttatcgtgaacttacggccaccccacttccctgcccacttccccatcagcttcaccttgggtcgcggcacatctgttctccagaacatgttccctgccaggaatctgcagggagctcttaagacttatctttctttttagctgttttttttttttttccactcaacttctaggctagtgctgagtacttatatgtcatgctgtgaatatgtgtgtgaatggattggtcagaattccaaattttagttgtaagaaaattcttttgtgcagcgtgcatggctcaaaatgagagaaaagacactgacgaggacttggcagccccgagcttttccttgggatgttatttgctttaataaattttgacctattttgtaacagaccaggttggagttccctttttaacctgctgtttcaacaaagttaagtgcattgaaggtctatctcaaaggcaatatatattttaaaaatttcagaatcctattctgaggctgaattatggctttttcttttcccataggatttacatgtacactcatctgtattacttcagatgttgctgctgtagtttggatgtttctctccccaaacctcatgttgagacctgatctccagtgttggaggtggggcctaatgcaaggtgtgtgggtcatggaagtggatccctcttgagtagattgctgccctccctgtggtggggtgagtgagttctcgctctgttagttctcacagaagcagattgctaaaaagagcctggtacctccccctgcctctctcttgcttcctctcttgccacgtgatctctgcacacgccggctcgcctcactttccgccacgagtggaagcagcctgaggtcctcaccagaagcagatgctagggccatgcttcctgtagcgcctgtagaactgtaagccaaatgaacctcttttctttatgaattccccagcctcagatattccttcctagcagtacaacatgactaagacagttaacttcccatctgactccctccagttgcttcaagggtaggcaagaattgtatattagctatttctacatccagctgtgtatccacagtgcctcttatatttggcctatagtacatactaaagaaatgtttatgtactctagaaatgtactcaagaaatgaccaaatgaatggataacttggctcatgagaattcttaagggcatttgaaatggcaaggggtatgtttggaagaaggagtgtgaggagctgaaggaaaagaggggagaccagctgtgttcagaacccacagtcatttcctcaggaaacagtaccagatatccttggatgtggggaaatgatccactcttgcagtagcctaggccacaggtaattttgtgtacccactatgcacttagctcatacagtaggtcgtaaaaatgaaaatggcatgattccttcataatctaggtggggagacactataaatacacaaataatttaaaagtacagggctgagcaccgtagctcacacctgtaatcccagcacttcgggagggcaaggcaggaggatcgcttgcccccaggagttcaagaccagcctggacaacatagtgaaacttcatccctagaaaaaacaattagccagctgtggggcatgcctagagtcccagctacttgggaggctgaggtgggaggattgcttgagcccaggagttcaaggctgcagtgagccgtgatcacaccactgcactccagcctgggtgacagagccagaccctgtcttaaaagaaaagtacagattatttcaggattaagcatatgagtacttaggctagagagaggcagagaaatgactcctgttttccagccactgtcacactgcagtgtaagccatcgacacacacatacacttcacttcatctgcacagcctttcaagataggtgtttttatctccagtttacaggtgaggaaaccgagctttgggaccaagttacttgtgcaaatcacacagctgggaaatatcattgccaggatttgaatctgagtctgcagcaattcagagaaagggcacgcacagtggcttttgtgtcagggaagagacattgtctgtattgtgggggtaggtgaactacggctggaacaccagcatgcagaggacttgccttttagctctgaggaggtcgcctgcccttgcatatctataaacaaccaccctggattgagagctgttgaatggggagtttgtctgactcccagaagagagcacagtacaggagaaaccttatataacgcagacatgcccggcatcccgcaggcgggcgcaagccaaccgcacacctcacagacccagcttttgttgtgtaggcgctgtggtgtaaaaaccatcctttagggccctctccctcgtggtggcagcgtgcctgggagctttctgttctatttgtggtatctgttccctttgatcattgtgcattgctttgttcctcatctccctctttggtgtcacctgtgaagtcactggagagccataatcatcctaaagaaagactcagagaagaagaagtaagacctaagaaaacagaccctttagcctccttggagctggttttctagttaagaggcttgattaaaagcgtggggggaatggatctgtgacgtacatggtcatgaaaagtgcagccagcgttacagcacacgtctgtataggttgcatcacaggcagggaagaggcctaagggcctggtggcccgcaggtgtgagcccctctggtggctccatgacaggagaccccgcggagaaagtagctcctcttccttttttaaatttttttttatttttattttttactttgagacggagtctctctttgttgcccaggctggagtgcagtggtgcgatctcagctcactccaccctctacctcccgggctcaagcagttctcctgcctcagcctcccgagtagctgggattacagacacgcgccaccacgcctggctaatttttaaaattttagtagagacggggtttcaccatgttggccaggcggttctcgaactcctgacctcaagtgatctgcccgcctcggtcttccaaagtgctgggattataggcatgagccaccgctccccggcctccccttcttataacacttgaggcctgtgcttcagttcatgggccttatgtaaaggcttctgtcacctgtctactccttcttcaagctggctgtttattcggcacagaggagtaaattacaggtggattatgcagtgacaagcacttttatacctcctgtaagaacagtttataccaggggtccccaacccccagaccgcagactagtactgtccgtggccttttaggaactgggccgcacagcaggaggtgagtggcgggcaagtgagcattaccacctgagctccgcctcctgtcagatcagtggcagcattagattctcgtaagagcacgaaccctgttgtgaatgacacatgtgagggatctaggttgtgtgcttcttatgagaatctaatgataaatgtcatgtgcttgaatcatcctgaaagcatccctctgcccccagtccgtggaaaaatgttcttccaccaaaccagtccctggtgccaaaaaggttgaggactgcttgtctatgccttaaaaaaaaaaggtccatttactttgcagtcatagaggacgcagggctggacttgaggaaatctacctagaaatccccttcaggttaagatttatgacattagcttcactattgaattggatcatagacatctataattaaaggaaaccatttacactttattgttcattaaatgtttaaaccacctgaaaagatttttttctaattgagcctttttgcatagttaccagttacccgtatctgacttcaagtctttagccgtatacctacatggttccatagttatcaatgtttatttacaatttaatattcttctttagcattatttaatatcaaaatgataatatgtactacggtaggttgggctttacagcttacaaaacactttgacatacattctctattatttttgatggctctgtgacatactgaactgaagcaacagtttaaggctatttctaaattgggctctgctcaagtgtgagaaagcgtgacagtcaacatgaactagctttagccttatcaccgtttcctgctcaggcaggtaagatctgctgaaggaaggaaccgttgccaactggccaggtgtgccagttcgtggcctagaaggggcagctgtcagttgaactttgcccatgttttcaggattttatctcaactcctgagtagtctcatctgggtgggagcactagaattgaaggcctgttcaaaggttttgtgcctttaactggagaattttttggaagccccttagaactgccaggcagccatgtgggatcaggtgggggggactgggctgctgtgtggtagatttgagttcatgtctcataaatggactctccccgaatctgagagggttgctccctaagtcaactacttaataattaatggaaaggagatgtgacaggaatagccaatttccctgtcctgtttggtatgactgttggcaatttgatgattccttccttaaccttgaagcaatctcagtttgactgactcatactatggggaagatagtggcctttgaactcttgactcaatacagtcttgaccttggttagatactaccttgacctctggataaaactgtactgggcattactattttattgattgttctctcaacaaaacaaccgtatctttaaaaaatttttttatcttacatgtctgaagtagaaggtcacctcttttgtagtgaaaaaataactattttccccatggtgtgctgaatgcatgggttaaagcaggtagaccagctgtcctgggttttatccctatgtgttttttgctgattcacaaaggcatttaatttctctctaccacgctttctaggtctgcaaaatagaaaggggagttcatgaccatctatgcgttactgggggatttttctgtgcaagcgaaaattattgatgcaaccagaggacttgctgccagcgtcgaggggtgcattttactccctctgtaaagaggccagcagtaatgatagaaacacagtgcatttgactctgaccttatatttgacaactcttcctctacaatccttaaaaaacatgcctcaccatttaagagaccagcagcaggccttcagcaaagcccacggtctaggcagcctttgtaggcctctgggattgtggtgagaaaaccttatccctctcttatttcctgataactttacattttagaggtgtctggaaagaaaggtggcagaaaagcagcataacctgcttgacaagtcctgtacaaacagaagtccaatgaggctgctgctctggggttgccaagctcaaggaagccaattgtagtgtgcacaaaggtgcaagtcaccaaggcctcagagagctccaaggaagcctgtgcaaaggcaggatttagagttctaaggacgggttgtccagcgcagcccagcagagaacctaggtccataactgtggactctcctgtgagggagccatgttgcggggtggacatctgacagttgtccctgctctgtgaacctgcgacttcatcctacaggtgcagagaatggttttggcaactctctccaccgcagaatgtgggcggacttctgagctgggtttgattttcctcttttgctttggagtgtacaacaatcaaccctttagagcagaagtagaggaatggaagactttcccatggcccaagacccctcagtgggttgctgtttgctcttccctagaatattataatgttcaatttatgcctccaggagaccagagacacacgtttcccaaatgtgggtacaggtggctttaagctgtttctgtgagtagaggcccagtgacatgttccccagtgtaaccccctccttggtttcacatcccctctacccgattctaggccaagcccctccagtgtagttggtgacccagaacaggtgaaagtgaggctgccttttgtagcactgctctttgtgaatgggaagtgctgctggggagaaatgattttaagtgtgagctttgctgttaggggcagccctctttctccactgcaataagaagagtcatgactaagtctagaaaggcaggttttgatctttaggatactgtgatgtatgaaataaaggtatgtccttcttattcggtgttgcattgtcagcacctaacacagtgtttaattaaagcaaaaatttttgtattgaaaatataactaggtggaggagtaagtcagtgctacttaagtgtgagtatttgtcttccctgatcagatggttttctacgtactcaaaatttcatgtagtaagaatgcaggatgcttatggagaaaagtgagtgttgtttgtttctccttaatatttatatataaaagcaagagcttattaaacttttagacgtctttggaggtttgaaatttcccactgttattcttaagaagagaagagaaagaataattttttttcttctgtcttattggcctttaaggaaaagcgaatttcagaaaaacatgggacggaacaggaagtgtttacaggtttagtcatacttgttaggtatgaaaaagccccactgccttctcatattgtttcttttatgaacttaacacttcctactttgagaataccttcctccccaagggctcaaagcccttcatagtcattaatttgcatcttgctaattttcttaaaaaattaaaatgttaactaccatttagctggtattctaccagctaaaacttccagctaaatgcattttgtctgtctgtatgtattagctcagttgaaaatgtgatcctactgaaggcagtagggtaaccggcttgtcccagtttccctgggatttcagttttagtactgaaaaccccacatccccggaaccccttcagtcccagacaaaccagcatggttgggtcacccaagaaggcgaccagttcaggtagctctgcataaagagaagatgttccaccctgcagtccttaccttaaatctaggcagctaccttaagaattgacctagacagcagtgtgtgcatggctcagtgcaaatctttagcatcacctggtagaagacaacactgagcacacagtcgcagaactagtgcagttcgaggtacgtggaaatgtctctgcaaagtaatatttctggtttcttgtttttttgtctacccttttcccctttactgcactatatggtgaaaaacgaaaacaagaactgctaactacctgatttccgaaacaagaagtgtgatctcaaatatttctgttgatctcaggtttttcatctgaaaaatgaggatgctggtctcaatttctaaggccccttcctgcccaaacttaccaggaggaaagaagctggaagtagaaagcaggagaggttcaaaaagaaaatgggatgcatctcctttagtaagctctactctggagaaggacgttgtccttcaagtacaaaccttttcaccgacttggggagtttccccccattgtatcccaccccatctttttctgccatcgtgcactgtgtcgtttgaatgtgctgctttgtcagggctgggggcttccttggtgctccagcttggccacatccgacacacagaccaccccgtcctccttctgcctctagaggaccaagaacaacccttgttctgaaagcaccctgtccctgagcctgaacacatgaccctggatgtcagaagatgctcttctccatttcaattgcccaattccagctctgtaatcattatgttaaatgatacaagtttcaaaaaatatatatttctaatagactttctattatcttccttaaatccacagtgtcactttcattcttactcctcattttggggtgaaagtgtagaaagattgtggctttagaatctgacgtactttgcttcaaattctagcaccactgtgtgacattgggaagcccgttctttaaccccactcagactctgtttctccattgatgaaatagaaacatacagtatcttatgtgtctggcacatggaaggtactcagcatattaatattaaccactttgggctgggcgcagtggctcacgcctgtaattgcagcactttgggaggccaaagcaggcggatcatgaggtcaggagattgagactatcctggttaacacagtgaaaccctgtctctactaaaaatacaaaaatttagccaggcatggtggcaggtgcctgtagtcccagctgctcgggaggctgaggcaggagaatggcgtgaatccgggaggcagagcttgcagtgagccgagatcgcaccactgcagtgcagcctgggagacagagcgagactccatctcaaaaaaaaaaaaaaaaaaaaaaaaattaaccacttcattcttcttagcccttcaagcccccgcaccgtgttttctgacatatcatcttgttttcatcctttctcttcggtttaattccagagggtacagagaaggtgtaagttacaggtaaaggagagtaagtgcactgtgagtgggtgctgatgaaatatcacaacaggttctcattctgacaactcacactaggccatgcttccccggaatatttatggaactaagtggaaaaaaggatctttagcaaggatccagcaccctcaatgcagggaatgaaatgtgtttattattagagcaatccagtttccatacagctaccaatatacacacacccacataccctgttcagccatactgaagagtttacttgtaaatccaggcgaagctgatgctgagaggtggcccgctgtactgattttgtaaaaccagcaggaaaacctctgctcggccgaccacttgatgttttagttgcagccactaacatgttaatcaaaagaagcaaatacatgtttttcccttgagaaaacatggatagtggtgtaacggaaattaccttgaattattcctcctcaagtatctatccaatctagatagatggattatctgattgcaccaggagatgcagacctggcagtggctacttctattgcagcatctttaaaggaatcagatctttggatttccattttcccatttttattcttattttctccctgctaaaaaacagtttgcagaggcacttgaaagtgatataaactccatagtagataatagttgtacatgaacatcgtatcttttatcatatagtcccctatgtaattaggtgatagcttgagcctagcacaggtggcgctgctgagccatctcggaaatgaccacatctgaactgggcgcagtggttcatgcctgtaatccccacacttcgggaggccaagatgggtggattgcttgagctcaggagttcgagaccagcctgggcaacatgacgaaacctcgtctctacaaaaaatataaaaattagctgtgtgtggtggtgctcacctgtggtcccagctacctggggggctgagatgagcaaaaaaaaaaaagaaaaaaaaaaaaagaagaagaagaaattaccatatctgtatgtctatacataggtatgtggatttatcacagtgctggtacaagtacttttgtcattgatgtttattaagctttttatgctccaggcactatgcaaaattgtagggcattatctcctctaatcctcagaatcttatgcagtagggccggtgttgctattactccattttatagatgaggaaacttaggcttggggtgtttaagtaacttctatgaagtcacacagtaagtgctagagccaagaggcaaagtaagcctggatgcttccaagccgtagctcttaaccactatgctatactgctcaaaaatatttttagaactgaatgaagacatctaaggaaaaggaaaacgtggtccctaatggtcttttgtgataacaatatgaaacctttcgtctctaaagtgtcatggaagtgaaacttgaaaggctctgttcttcatcttctacttattaatgggcaaaatgtcttcgtaatttatggcatgtgtcccaaaggtgcaaaggtggcaaggtggccttttttttttttttttttttttttttactaatactaggtcctgcttccaagcctcctatttcttcacttccctgtgtaccagggtttacctgctgcttaacccacccaccccactatacgtgtctgtagtagacccaccaacattcacatggccacttctgctgaaagtacctcccaggatagaatagggtcacagacccccaattgtataagcctttggcttcttgatgtctctgcgagttacggttatgggagttggtctttggtgagattagtggtgagatcagtgggtggcacctagggtgatttattgaatagtatggggtgaggctgaagagcaaacatccacgtaagtgttgtctgttttggagtttgaaaagttgtcaccctaataaacaaactacttaattgaacagcccttgcttggcgtgatcaaagaaaagacctagactttgctatcagtaggctctgggttccaggcctggcttgtcacttacgatgatgatgatgatgatgattttttatttttttgagatggagtttcactcttgttgcccaggctggagtgcaatggcacaatctcggctcgccacaacctccgcctcccgggttcaagcgattctcgtctcagcctcttgagtagctgggattacaggcgcccgccagtacgcctggctaatttttggtatttttagtagaaacggggtttcaccatgttgcccagactggtctcgaactcctgacctcaggtgatccacccgccttggcctcccaaagtgcggggattacaggcgtgaaccatcgcgcccggccagctcgtcgcttattatgcaagttgtataacattgctgaatcccaagtttttagtctttaaaatgagaatttaaatgtcgcccttattttgattgctgggaagtttaaatgaaacagtgtatgtgcctgtcacatagtagttgacaataaatgatagtttcttttccttttcccttaagtaagttttttttccctctagcctataagctccttaaagaagatagagattttcttttctcactcatttccatattctcagcccttagcagtgccaggcacatatttgctgaatgaaagaaaaatcttttacaaaggtcttcagatcattcattcaccagacaattgttgagtatctacagtgtatcaggcactgttcttggagcagggcagtcttaaaaaacacacacaaacatagtctgtgtccccttttttagtctaactgggagagaaacattacatagataattagagcaatgaacaactttgatccttgagatgggatttaaattttaagagataggtgacatttggtgtcaagtctgtccaggtatacttgatattgtttttgatcaggtacaagaaacggcaatatactgttatctataactggttctgaaagatctgaaagaggtgtttctaaagtatttttgaggaatggtgctgcttttttggaataagtataaatacattgtaaagtatctatttcgagagggaacagacttgatgggatgggatcatagtaggtaaggtgttccaggaacttggaacatactggaataaggcttaaacacaagtcacagaactgggtgatagggcggtgagcgcatctactggagaggagatggggcagggctggcagcttgtagcaagatttttatgtagacagttagacttcaaacttcatcagtaagtcagtagatggccctaggtggaaacacaggacaaaagatgaatttgtgttggcactagaaatactttgagctcatctaatgaggctcattcattcatttggtaaatacttcacgagtgtccagtgcaagctctgttctagttgtaagggatatggcagtgaacaaggcagacaaggtccctgtgcctgcagagttttgatgataaccttcccccctgggtgtgtgggagggcctggggctcctgtgtggtttgggggtctgagaggtggatgaaggtacagtaggcccacagtcgtggaaatcaaagtccaagctccagtgaaccatcaattaagaaacaagggattgtgaagaacaagacctagatccagactgaagggaaggatttgctgaggagttcaggccagcatgggctggggtgacttggcctggaactctacaacagtcaggacagggagtcctgaataggggcacctgcagctgagtccatggatggggagggacctgtgtaaggcactgaatttagactctgcacgcagttctaagaggtgttctcaacacgtttatgcaactaaaaagtgtttattaacagtgttttgaaacaagtgggatttttgttgaggttgtaggataattcagggtcatatggagatttttattctatcagtatttgccctgtcatgtagatgagacacgttagaggcaagcctctgaaaattagattgatttatttttctagtctgaaccgaggaaaataactacttgtcttctatctcctggtgctctaatatatgtaaaatgttgtcttatggtgagacaatgcatattaattttttaggcacttgatggaagaagtggatgagggaagcagcatcggtctttgaacctaggtcagaaagaaatggaagccatggtgtgagaataatgcatcatggtttgtatggcctttttcatgtattttaactcaactgattttctcagggcctcatcagtcagtcaacgcagttttctccatctcatagccaaggagactgagactcagatctgcccctgcccactttttttgtagttttagggggaagcataaatgtggaggaagtaaatgagtgtgtttgttcaggcttagacttttgttattttttagccttgcagggaaggggaggaaagccaataacgatggctagaattggagatagaatgaatgcaagttgcattaaactgtaaggttagtctcggtaagaggttgtttatatggtggtgtcttgggtgtatcttaccatgtagcattcttcgttttaagaaggattccttccggtgcaacatcagtcacagcatagaaagcctatgctgctctgctagtttgcttgcttgcttgcttgcttgcttgctttctccttccttccttccttccttccctccctccctccttccctccttccctccttccttcctctctctttctctttctctctctctctgttttttgtttgtttttgctttttttttttttttttttttaaatttgagacagggtcttgctcagtcagccaggctggagtgcagtggcgcagtcatggctcaccagccttgcacttcttgggctcaagtgatcctcccacctcagcctcccaagtagctggaatgtaccaccatgcccgactaatttttaattttttttaatggagacggttttgccctgttgcccagactcatctcaaactcctgggctcaagtgatcctcctgcctctgcctcccaaagtgctgagattacaagtgtgacccactgcgcctctgccctgtgccacttttccacagctgctttacctacagcctgggagtcaggccagcaaatgacgacaccccaggcacagacatgctctctaggaacttcgagtcaggtatttcactttgtaaagaacttccattttaattctattttctcctagttagagactcatctttcttctgaggggtgcaaatgattctttggagaagtttaaacctgttttacggttcaaactcaagatggtagaaacaagtcatatccaaaagagtgccccgagctctaagagttgtggctgcgcaacgttttttaggtgtctccgaagtgttatggccaagttcaggattgacccagatgaggagaggttgcactgcatagtgatgatttgtgttggggaggagagctgaaactttgcaaagtggcgtcctgaaccaacttgtttttcagcttctagggaaatttgttaggtaatttttaaaaaaagagagagagagacagagaagacttaatttgatggagcctaaactccactgtcttctttctgctgagtgctaaggggctcatggtctctgaggacagagggcaggaaaacaccttcatgccctgtgtcctctgtaaggggcctctagtatgagggactctgatgactattgctgttggggaaaagaaggaaaaggaacaggggtcaacttctgtcatcatctctccagagtttatgatgtttctgaaaggatggggaatagtgcgtggcccccactcatccctgtgaggtgctgcccaggtatccaacctgcagtgcacatgctcgccactggcactggggatggtgagtgcagcagagagatgggattcttaatttattcaattttaattaaaaaacacagttcagttgttaaaaacctatttaagtgtgtttggaatagcttggatatgtgaatttaccttttcaactgtgaattttatgaactctaaatacagatcaagtatttccagcaaaaatttagtgtcggatttgaaatgtgctctaatggaaactagacaccagatttcgaagacttagtatgaaaaaaacataaaatacctcagtaattatctgtattgactatacattgaaagaatattttggccacattgagttaggtgtcctatgttatttagatgcattttacccatctcttgtttttcatgtagctaccagaaaatgttaaattacctatggggctccccttatatttctgtgggacagtgcgagtctagacagtgccttacgggatccatcagaagcattattggactttctagggaaagttgctggacttcaaaagttagagtcataatttttcccgttgtgttaacaagttgctttatgtggatgggtttatgacaattgagggttggaaaataggttttaataagagtaagttaattgagaggacagctttacctctgttaatcagaatgattttaaaatgcattttcacccactcagaaaccactgattctcatgggaacaaaaaactacattcgtgcgtagtacgaatttgtgcctgctgggtccttgtacctttacgccaggcagaaatctccgtaatcgtcagtgtagatttcatttccttgtggcaaggaatataaagtaatacaaccaaaggggaattccactgcatgaaaggaaatattagtgctcgtctagctcttgtttcaaccctataagctccaagtgattttcagagtccctccttcaacttttttgttaaactttccagattttcctatttatagatttcagttgttggcagctacatgttggaaccgttgttgaagcccctagtaagacttgatgctgtagcacgctttcacagcttggtttgcaagttgctttctaaagatgcaaagagaattactacccactctagtagtttgaacaacccagtgaatttcagatattaaagccatattcatgccactgttctcctaattctatcactgtttagaattgcattttgtctaaaggaaggatagatagaatactactaataatatcaagatgctcagaaactgttgagaattttttttacaggaagaacttaaatttccagaggcttttaaaaaaaaatttattaaggaaggagtaaataaatacgccgggttcttgagtaaactacttttacccttcagaaccactttgtacgtagactttatcatgatggccaagagtttttaaaatactataggcagagggtatactctagagaaagaagacaacattgggtttaggactgctagccccatccagagcagttatgtggttcattcagggatcttcttagcccttcaacctcaatttctacatctgtgaggtaggttttgtgctagttctagtgacatgactcgtgggtgtattaggaggatatagcagcatgtaattgaagggcttttatcttggaaaagccttagtaggctcaaggtttctagttgtttagggttctaagattttactaactagtcagctgcttggagttcaaggcatttcttcagattataatgagttgatagtgcagtgtgcaaaaataaagaccgaaccatggtaagtagcaaggttactgtaaggaaaaaaatgtgtcccactggcccctttaaaatggcacaggtgcatctctgtagcaaccttgacagccatcatgtgggatcattcccctcacccagggaacagtggttccccaatctccaatctctccattctccagcatgagaaagtggtctggagcttgatcacatgaagatggacagacacagccttttggatgcgagagagggaagggaatgcaggccaaaagtggggtgggttttcctttcctcttccttgttttttattaaccttgtcttgctcctgccacacacaagtgcagccagtgctcaggacactgctccgtgtggagctccggaaagtgaagctgagaagccttccttggggtggacatggggggagctgcagaggcaatagggagaaaataacaaccatggctcaggcactgcttggagcagttgttcatatgtaggtcaggtagtcatcattcaggaacttcctgtattacacaaataatctctattttatggttaaaggtaggacagtgataatttactcaggagggaaggacaattagcaaattgattaataaggtttctaaaacctattgtgaaagagaatcctttaacatatgtcttgatttattctgcttatttatgtcttgatttaagacagtgattcgcaactttagctctgcatattgcaatcacctgggagctttaacagcgttcatgcctggctcccatccctggagatttacttggctattgggattgtgttttaaagtttctccaggtgattctaatacacagataatagccagaatgcctgatttaaaatgtttcctgaattgttagctgatcttgagtgcatattttctatgtactgttgcacactaatagttttaatgaaggcacagaatcttatgtaaaataaactgttaattggtcttaactacagggctacaagagtttccttcttatttcaagctgcttaatattccgttaatcactgttgtcatgcacacacacttctgtattccattctcattcatttactttttagaatggtattaatgctggtggcatttgcagggtagtagtcaccttgtcagtagaacttgctttagtaattagttgtcagaaacaaaactccccagactttctacccatgaccacaatgaagggaatgatgccttttctgagtccctattcctaagaatcaggaaatttctatgatatttgacaagtataggtagaaattacttgccaaggtgattatgacttctggaaaaaatttttttaaaggaaacacaggtgtgtaacactcctaatcagtatataagattgtttgtatgttactttttcgagttataaaaggaagaggataaattctgcctccttccccagttactgtcaatcttcagtcttcttttgaattaagttagtttcccattttacaacataaagacaagtttaattcttaaggcttacataaaaaagtaatgtttaatttgggatttctcaaattcttcatcataataacaccttgcattagtagagaacctttgttgcaagaagatcaaaatattttataagcttcatctcatttttcttttctttctttcttttttttttttttttttggagacagcatcttgctctgtcgccctggctggagtgcagtagcacaatcacagctcactgtagcctcaacctcctgggctcaagtgatccttccacctcagcctcccaagtagctggaaccacaggcacatgccaccatgcctggctaatttttcatattttttgtagagacggggtttcgccatgttgcccaggctggtctcgaactactgagctcgagggatcctcttgccctggcctcccaaagtattgggattacaggtgtgagccactgcgccagacctcttttatctttgagataataccgtgtgacgacaaaggctgggtattgttatccgaatctctcacacagcaacactgaggagacattgactcatttcttttacgttgctttttaagtacagtattgtttggttttgttgaaaattattttggtagtaaacattttccagagatataaatcttttccagactcatttgcgcttgttactgtaggtagaattcatgaggcttttcctatttctgtttatttcggagcctgaagtgacccaggaattatgtgatatatgtagagcgagggaatttcttcagtttgactgtggtcctggttgtaacccagccttcttccttacagatgggggtgagggttggggtatcccttagagcctggtgtgtcttcatacaaacagctttgctggggctgcctagagcctgggccagcagtggggtagtatcaagtacagggcattattcagcggttctccacactctcctcatttgaatgagaatataaatcttctcccctggctctcatgtcacatctttaaaacctcaagtttcttattaggcatgtgtggtccatttaagctctagacatttcgaaaagataaatcacagggcaaaacatctttctagttttgaaggaggtctgagttgcagctcgctggggttgtgagtctgagccgtcgcttttctttctcttccttttacaagacttccaggatatagccgttaaagaaggacacaaaaaagagcaaagccctcagtgatgctgtggcacaaagaagaaagctacttgaggaggaggcgaagggtttacgaagaggcaaagaatgcaggtgaagggagggaggagttaatagttaaggctcaggttccaagccctttgggctgccagtgtctgcaggagccttgctttcatcctttccttctgcccaggttttaagtctcatgagttaaaacctggggaagctcccaccccaggtccactgtgagggaacgtacaagaacagtcacgtggcaatagtacatgcctggaaggtatctctcatttggggggagattgagaagcgctgtctaatggtgcccttgatacaactaaatagcaactgtctcagttcattacttgttagccccaggcagggacaacgcagattggctaattataatagccagaccattaaagggaatagcattaatggaggagaaagcagctaataaagagctgaacttggccactttgttgtgcccaggatacaagctgctgttaatttcagtacctgtttcttctgctgttagaactaggaagaaaggatttagtgaccctctttcaaaaggtggatcacaagcttagaggcattgctggataaattcaacagcaagactttatttcttccccagaggtgacgtctgatggttctgtataaataaagtcacaccacctttcttatgctgaattgatgctgctgacctgctgttagtaggacatgcacttttatggcgtttccaggagtggtgatgtgtgttgctgagctcatctaatctctgctttgagcaaaggcaatcctctatatcatggcagggattagaggtatagcctggggctgcagaatggtgtgatttaaattttcccatgcaggttcaaagcttgtaactttgcctgcgtgtacatccacactgggttccagccacatgaactgggccaccttcatagacctccctggtaaccacagcctgacgtgacaaggaatcacagttttactttccgcttactatccatgttagaaatatctaaggaaattcaggaaaaaaaaaaaagagagaatttcatggcatgtcaagaatggaaagtatcttaattaaaatgttttaaaatgggaacaataaagaagtcgatttaagttttttgctttggtgcttgtctttaaagaaattgctcctaattctagaaactagtgctttttctaattatattcagtagaaccctaggatcctgtgaggaggcataggggagtaattgataaagaaggggactgtggtgtccgtgttttatttggagctgttctacccacactgtaatttataagctctaacttaccaggggagataagattgtatagaaccagcatcttgcagttatacggaacctttaagttctcctactccagctccttcccagtgcaagcacgtgttttagagtaactgttcagcctcagcattaagtcagctttaggagaacagtaaatcgcccatttatctggagacttcagctgtttgaaaggccttccttctagtgcatcggtatctgttgctggctctgtcctctgctactacaagcaagccttgagtctctcttagacttgtccttcctatatttgaagacatctccgagaagattttaaatcttttttcccccacaggccacatatatccttaatgccttcatccattcccctcttgggaatgtgcatctttgggccattggagaggtcttgcttggactgcggccactgtgatttctgggggcagactcagctccttcttggtgtagagatctgcccactataggcaccaaatagatgttggcaggcttctttcctccttactgcttctatttatctcacctgtacttttagatttgctgctcttgggtaagcagataagaacaagggaaatgtcttccgttgttgttaaagacaatgttaggatagaaagtaatatgtttgtactcaaaaatgcccagtgagatttctagccacggccaagttatcatttgcataagcaacctggaaatgccctcttggtcctactgattgtgtcccatggttaatgtagatccctggataaattcgaatggctactcctgctcaggggtcatgccatgagaacttttctcatacatcagatagagtccccttccgtcagctgaatgcatttacccctcagtctgtgctacccataggataagcaacatgcataagaaggtaggtgccttggcttaggcagctctgttgagtcaccctcaagaatcctgctggaattctgtgaggagtgaggatgcagagcaacgaaatattcgaaggctgctatgatttttacaagcttgaacctgtctcttggaattccccattgtgaagtcattgttgactggttggcattataaagcagttcagagaagtaaagttagatcagtctgcacgcaaataccagccacaccatctcccaagctagtgactggaactgcggttttcaagtcagtttcctcattgcggaatggagataacgatgccttccttgcagggtcattgtgaggctgaaataaaataaaggcttcatgggaacacataccacatgctcacttaatgctagctctctttcctgattgtggcagtgactgtattttccagctacaattgtgacacgtgttctgagaggtgttaagtatttatatacaactacccatatgtatttatgggtaaaaggacacggaactttaaatattgagaccatcctggaaattctaggatgtatgattgcataatttgtaatgcctgctataggcctagcctcatagttgacccttgatggatgctgaaatatttcttaatcatagggagaaaaaaaactcaatgaccttttacagagatgtttgattgcagggcttattgctaatgctctctctctctctttttttttttttttttttttttaatctcactatgcaaagataatacaggtctgggtaagaggaagacttaagatgctttgacttaattgtatctgcttgacagaagcaatatactgccttctgattttaggatgtaaccatccagaaaggcagaagaaagggacaatttcagttctaaacaggaatccgaatatctcacgtcctgaaccatctacataaactagagaaagggcactccttaagactggtgaagcgggctccttggaagatccccacagcaacattgagtttggtttgtctggcgtaccctctccccaagggctgagtgggccatctgggtaacctagagaggagacttcagatgctaggagtatagttcctacccatcctcattttcacagggtacactcatttttttccccttggaagtactgatatgtattctgcaagatgaatgttgagacccaaacaccatttggcttgtaaaaattaatggcttagcatccagagggatgttgctttcagggcaggggggacactggacaccaagaagaaagccagcttgggcccctataccatcttggatgtctaaaccatcttctggtaactctctggatgaaaagaaatcatgaacttcacttctgtgatttggtggatgtggattttgctacatcaaaatttgcatttgagtctctgtggtgttctttctagccgagatgctagggtatgctctttctttctggggagattccaaaaatgttactctctgtggctgtgtgtacaatgtctatttagaaaagaagactggcatttgcatcttctctcaaattctttacgtttgtctcactgaattttggagtggacaaaatattcaatagcttttttcaagtgaaaaaaatgtcgactttcaagagtgtattactttaggttcttgtgtatatctgtgctttcctggtggcagaaacctagaaaactctagagctcaaatttcagcgtattcaattttttgttaatgtgtctatagcttgttaataaaagcttacttaatgaaagccaacatcaccaccacttgaaatttttgcagccagaagagtgtacttaaatacttgtgtattcagacttcactttaataaatttcaataaatagcttcccataggagaggtttcttctgttgaatctgtagaagttgacgacggtacaagcatcagctttctcaggcagcttttctaaacacctgcagggtggaaaagagatccttctctcaaaatactgaatattgctggcatgaccactcttgtcactggtgcctgctgttttccatcttatctgggttgcaggtttcttcacagtgatgggacaccgaagtgcaatgggatgaaagcatgcctgtggcatttttaccctagctgtggggaaatgaccactatcctgtgtccatgacactcttcctcctcaggactttcaaactaggcttcagaagccatcactgggggcagtcctggcatttataatggggatagggacaatttccaggaaaagtttctatatcaaggacatctcagttctgcagcagagaaatctttgaaattacctgcaaaccatctttttgcaagcacaaccaactttatttctgttttaattaattccaaaaatgctttatttccagaatgttaatatttagttagcagtaggctctaagtttcccaacccctcagccttttcagagggtataataataaatgtgtaaatgtgctaggggatccccgtgatttatggaagctttatattgaataattttgaagaggaaagaccttttggtaataatattcattttttgactcttctgtaatatgcaacctgagggtgggtgtgggggaacgtggagaaatgccattctcctactgagcacagactgtatacacctattttacagggaagtcacgggctcctaagcattcagtttaaaaatgagcagaattagatgggtacccttcattggcccttcatggctaggggagaaatgggagagggaacgcagctcattttggcttcagtccttacgctttcaagagacaaccctactccatacttcattattttttttaaagctttctataatggttggtgaaatttacatttcaactcttgcctacaacagttttctgccttattttctcttcccagcgctttgtgccgcctccttgacaacctgctcataggtattacaattggcctcagaaattgcagtgcttcccttttgttcagctggttggcttgataggttgtatctgcaaatttgagagcagcgtgttttattttttacagcatctaaacaataatgtaaccacatcacggcattctgagtcacatcattgacctccaccagcagtttatccccctacaacacttgaatacattaatatcactgtctgtagcattttaatttaattagagcagcggttataatcctcttgggaaaatcgttatcccccccatacacacacacacacacacacacacacacacacacacacacacacacacacacacacacacacccctactccccataatgttaaaagaaagaaagcagctctggttttcatctgtctgacaatttgctttagaggtgggactctcaccaccaccccctcctttgttggggtcgcaccagagaagcagttgtgagagtaaagaggttccctttatttgggaaattagacgttagtgtccttaaacttctctgtctagatgtctccatctgagatagtggtatttggttaatttttccaattgctgatagtctgctgatgtgccagggtagatgttaagtgcgaggttattgcaatagagtgaaaaatgagcactgtgagagtcaacctagagattcacaaaaagagagacaaaaggagtctctgtagtgtgggattcccaagagtgtgttttgttctccttggaacagagcccactcactgtacagcatccttttggggtccccaagtgctctgcagccaccaagcccagagttaccttaggtgtgccatggctactagaagagtgctcggggccctagaagcacattgtgcatttgtccgtcttggtcaaggtaacctctcttgttgctgacaagtctcttcattgagcctttgaaaaagagcttatggtgtggaaaactgtcctccagctgagtagaaaccatcatcaggaatgtgaggagatcagtgggccacagtctcttcatctctggccttccttttataagttttcatcatgaaatagcagttgtctccatctaaaattgtttggacggggtttgcgggggatggcgtcagatcccagagcaggtgcagaagtgtggtgtttcaccgtaaggtcagggaaccatgtagaagtgaaagcacaccaaggggttacttttaaagagagcacagttgaaaggaaggtccaaagcaatgataatttagtaaattacagtatatacactttcagggtaatagctagccagactcttttgtcagctggctggggatggagttcctaagaaatgaagtccagtcagcatggagggccgggtttctcccagcttgccttggtggagttggggcaggcggggatgaggcggcctcactctcatggactagccagagtgtcttgtgagtttctctttgcttcaagggaaagaagagtcattgaatttcgaaatgctgtcttgtttttttcttgcactcatgaagcaccatttgaagaccctttggctgcccgatgttttcttcaccaagtttcacttctgcaaaaaccctaacaaaacagaaagctatttccgagaataaagttaacggtagatatctagacatatagtgggatttggggggtgtcagacaagagttgaagagtatggagaatttgttaaattctaaaaacagactgtttgaaatgctcagaagcccacctcacctttaaaaacctctgaacagattttatgtttcctatatgtgtgggctttttttttttttttttttttttttgcttttttcatcccaaaccaaacaccaaaaagtataacccatccttcaagttaaagtttctcaaaatacatgtgatgcctgctaaagttcaagaaaagtgcagttgaaagttttgcctctgggtgactttttacaggacatcttaattgacccagaaatgaaacctgggatatatgtgcctgcccttcattaagaaattcaccaagaaacctcaggatgtatgtttaaacagaaatattccaactgccctacttgcttctggtgggtgccttagggaaggtatagcaattccattcggaaattataacaatatgtatatattcatttaaaatcaaaacttcctaccacaactgccagactttctaggctatgagcagaggagaagctttaatttggttataggccgaggactccgccgcttccctttgcacatttgatgagatgtaaaatgttaaagaactatctttccaaaacgtaaacactgccttccctttacttaaatgtacgattttcttttgctcttgagttttattttcatagtggcatcttactggtctttcctttccaccatatttttggatgttgtgtgttccattttacattgcccagttaaaccacagacactcctgaagtcatgtttacatgtggactgcaagtcaacctagttggcatgttgatctaagctacaaattgcactgctgttttgccgaacccaacagtcggtttcttgccattatttgcggtattttaacttaaaactcacggtaatccttctcaccccatctagtttgttttaattgatctaacaaacactgcttgtttgaattcaaatggaggatccatggaagctctcccaccccaccctttgatacttgataaggggtcaaacagtacttcttttaaattcagataattctttgaatgaactatgaaatacttcagagggaaaggaaatatcgattctgagatggagagtaaaagaacaaggagatattcattattgttgcagataattcctagcaatgaaggaaaaaattagacgttggatatttttggttgtggtggttgtatagtgagataggtggacaatagaaaaatcttccttgtggattgaagttctaaatctagaacttatctttgaagctgtcactttacttgatcagctcactatggaagtgaatctagaatcatactttgattagaagctggggatattgctacagaagagaaaacatggctttaatatagtgatggcattaattatgtgacttgggcaagtcattttttaaaaagttgtctttaagccattctcgcggacgtattttttttgttcccttttgtccctcctttttacttgtacaaaccacagggtgtacacctattgacatgagtccaaagtaaaggctgttttcaacgatggcagagttttgttatatagccccaaggctaggcatctgaaagacaactgtatacagtgtctctacggagagagtaagccggattgcttgggaagctggtactgcccatgccccgcctcccttttgctccccaccccgagatggtctcatacctccattccaagctttcattgtctctctccccctcccctcttcctgcccttatttttaacagcaggaacgaatttcatatacacctccagagagcccagtgccgagttacgcttcctcgacgccacttcatgttccagtgcctcgagcgctcaggatggaggaagactcgatccgcctgcctgcgcacctgcgtgagtgttcgtgacccgagagggacagaggattgatggcgtggggcgggggtggcaggcagtgggctccaggccagagaggggcaagctctgaggtggttttcacaggacttcgccacgctgctttggaatctttcacacccccctacccccagatacctttgaaaaatttgaggttcctgttccttgtttctcagtgtattcatttcttccctgactatgacatgttaaaaaaaaaagaaacctggaaacatgcagtatatggcagagccaactaaatctcctagtagacaagatgatttctgtgtgtgtgcaaagtttatctgtattctcaaattcacgcttactcaactttaatattttctttggtcctaaattttgctcagtttgtgagtcagactcacttctgctttgtttttaagctgtgcgctcgtcctttctttccagggccctccctcccctccctgtctgtgctggctacttttcttcccttcgttttctaggcctgcataggtcctctctcttgcctcctctttccttcacctccactctctcttgcttctcttgtgcttgctttgcctcagcagctgcagtctgtagtggttccacctttctctcccaaatcatctctaccccttggcctaccacttccccgtctcagtcctcagctgcaacttggcagaggtttggggcccacacacctgtgctgctgatactggcctccaaagccgttgacacctgctgtgtcctttttgtctgcctttctcatactgccacccagctcaggtgataaaacaggattggggcagctggagagactgaatcacctgattcccaccctcttcacagtgtccaagaaaatgccagcttctctgccttgaatgttatcctgagggcacaagactagcttatagaggacacttgaggtcaccttggcccttccagtgacttaaatggtggcacagtcttgctcaccatctctctccagcagggtgatgctctccagttcttctcctcctgttccaccctcctcagaaggttgctttctgaatgcagagaacaggagacagaggatgcaggttcttcctttctctctctggtaattcagggaatcagatgactgtgatatgggggctcctgggggaacactgccgccggcttatgccttctgccccagagggcagacggttgctgggatgtttgtgaggagttggaggcatgagaatcctttccctcctgcagcttgctgcgtccatcagtatttacgaagttgctgcctactatggtcaagacatttctgcttcatttgggcagggaagtaaaggacacagccctactctaccatcttttcctctacttttaaggaggttcgaactgtaaaggtgccagggtcatggctgggaacatcagtgttcttaggaaactggaatgttgtggcaccatgggagcagacacaactggaaaagaaacagcagccaagtgataggaaattttcctccagaagtcatcctgtgaccgatatattcacttcagaaatattcgcctcaggatggaacatactcatgccacacaggcagaacacttctgctacctaattcattcatttattcatctcacaaatagttattgattgcctctgctgtgcccggcactcttctgtgtcctgggggtgcagaaatgaacaaagcagaaaaaactcccctgctcttgtggaacttaaattctagcacagagaaaaaaaaagtatcaaacactcatcacaaacaggctgagatttctttcataacgttttattcctatagattgcaacatcccccaaggaggccctcgctcctggttcacttctggatgccttgacatatggaagctccttaactatcttccacgagatgttatcctgacaaaacaaataagaaagcaaacaaacacactgaactggaggtcaggagatcttaacttttagctagttctgctgttcgactgtgggtaatttaactcagttctcctagcatacaatactgtgtgttgacaagaaaatgtttaacagccttcctaactttaaagtactatgattctcatctgaggcatcattctccttgctttgaattgtttcttccatactaacacttgaattttttcttgagtagacagtttccttattgagccgtgtctgtgctgcttaccagcattttggtagtgccttctgtgtgcctagcacacaaccataatagctaatatttatcacgggattaccctgtagcaggcactgtgtgaagtcctttacaggaattcttttaattcattctcacaatacctgtcaggtaggtcttctttctatctccatgtcacacataagggcttagacaatttaagtaatttttcaaggtcgatcacagaaccagtaagagatagagtcaaggctcaaactcagttctggctgactccagagcctaggatcttatatccactataccaccaaaaagtgtgtgtttagtaaatatttattggatggatggatggatggatgaattcagggttgacacttagccagtgttcactggtgtggccgtagcattggtttatggctggaatctattctaattgctgttctgtctgttctatctgttgttgaatggcttgcatatcacccttcgaaggatggatgggtccccacgtagacagctataaaatatagaactgcagcacattcccggaaacctttccatttcatcagtagttagactatggattcagattatggtaatgattttgcctgaatgcctactctatggcagtctaactttatatacctctccttggtagtctgcatgagacctattgtgacaccattggttctgacatctgtattgaatcctctggaagtggaggttcccaggctcttgctgtttgccctcagctgggactttccaaatgaggctgagcttctgtcctcccgctcccgtttcagggttctgctgcttgtcatcctgtttgagaggagcatatagcccctacgatgactgaagctgtgccacacacttaaagatgcatcccagctgagtttccaaacctaaaagaaaataaatattctaaccagggaattgggttattgctgttttaatctgggattccagacaattcccagtggaatgctcatggaaagtacggggaagaatctcaaagccaagacaacagcgggacatttatggtattgctaagtccaaggcacttgtagctgcttattttaaagagatactcatcagattggtatgcctcaaagtgggttcttttcaccattttccattgattaccagataaatcctctcccttaattcttcctttctcttctttcattcttaaatcaacttatattcttgtggatgggggtaaagaggagcagagatcatattatttaaggttctcatacagagagtttctcaccattttattctgcaaacacagagctatacccaaactgaagacacagcatgacaaattagcaagttattgagaagtagagagtgatgccttttccttttatttgccagtgataaaagcaaagtgataaatggagtaaagtgttaagtgctgattagagaggacttgaaaaggactggcaaagaagcctgggcagagtgcaagaggagagcccagggaagaaaggccagaccgtgccatcctgctctgactcactgcccagattaccaggacagtgcccttcaacagtcctacagcagaacccctctgcttaccgggagagcaaacacaagtatgtttgttgagaggatagaaagggcatcattttttcttcttcttcctcttcttccataaagggtactgaattttttttgaagcttgagggtagaagagccgaagtacaattacagtagacaggtgggaaaaaaaaagttgcattctttaaaggaacataagcttttcattgtagcttgaaaacacttgaaagaccaaggacccccaagggtgcccaggcaaggtcactcttgactctacgcagagaagggctgttgctgggagcaaaggatacctttgcccagttctcaccaggggacttgtagtctcgcaagggagggatgttttctgatttgtctacccaaaggccgccctttttctaaactcatgggggagtggctgctgaagggggcagttacagcacctcacgaggtgctgccagcctgctcattggcagaggcttaccttatgaaacactgggcctcagacagtgatgctcaggccccgccttaggataacaatataagtatagttcagattgatggggcagaatttaaataattaaatgtcgttattttaaatttcttttaaacatgatcatttcacatttagttgacattgcgccatttgcccgcagactgtgcattttgcagtgtccatttggccttctaaaatccctgtgaattatatttaaaaatcggaacgtaggactcattcatcatatttaatagggttagggtttatgatgttgcaggttaataaatagccataatagccgctcggcgttttctggagtcagaacaccagaaatagtcccttggctctctttcctttccttgtgaatctttcaaaattcttacattactactcctgcttgtgtgtgtttaataatttaatttaatgccttcctttggaaacattacctcgctgctcacccactcggctgttttgccttctcagcagagtaactgagtttaagacagccacgtgacaggcacagctatattagaaccagcatagtgagaagggatgcagtgcctctcttttctaattgggtagagcaacaattttatatgctttcacattatgtttgttcccaaaaaaaaagaggcaaaattgggagctgaacgtgatcatcattattattttatatttttatacataacaagatgcgctcatacttattgtctcacttaattgtacagcagtccttgaagcaggtgctattgattctgtttgatgagctgaccgtgtctccgataggtcaactggattgtccaggttcacacagcatgtgaaaggccgaatgtgatttctacttcttgaaaccacctttcagcttttttaggagaaaacataatgacataaagagtttcatggacttgaaagtagaggcactgctgaccgctgaagtctgggcagccacaggtgcagggactgggaccggtgtggttctagtgatgccacctgatcatgaagttgtgctgaatcctcagcagactggcttctccctcgtagtaggatgagggagaggaatcaggataacagcctgtccctgtggatggtttattctggttttgttcaccagtcctgctgcctccccgtttgttctcttgcgagctcaggttagagttgaatttctccctacaaccagaatgccagggctggtcctgtaggccctgatgggccagctctgagagtgggctcaatccagtacccaattccccagcattttgctaggggtgcccttctaggagcagggtctcactgcttttggaagtctccaaataaaaaaccatggtaatagaaagggcttgggtctggatagacgtgaatggacttgaatttcagggttggctctattagagagatgatttaacctctctaagactcaagtttctaacgacacctactaatgagggcttcttagaagtatcagagctcatttatgtatagcacctggcatagtgcctggcctgcagtggccaggtagctcaccaaagctccatttctgtttccggttatggtgaatagcctgttgaggaggtggcttccccatgcaggagccaagggaagcagacatgtcgttgtctgtgtgtttcattaaagaaaggacctagggttctgtctgtgtattattgagactaatcttcctaagtcacaatggaaataagctttgctttcaaacgatcccctgtgatttccaggggctcagttttgctctagggcactccatcccaggaccttttcctacccacatgtctctccctcagtctggaactttcttcccgttctcgcctgaatctgtgaatctccttcagccttccccactcttctaatacctatcaagtcctcccctgtcctttgctatcacatcagtgttccaaaaatggcctctcctcacagctgcctgtgacgtgcagtgatatcaccagcagagttgagacaaggacaaggagaagagatttaattctctgtcagtgtcttacaaagcagtaatgacttctaacaaagccgaccagccactagcattttctactttgttcccccttaagccttcttttgggtgccttcactttttacatccaaatgcttgaagctttctactttccccgcggcacatgtggctgtacgagggccctggttagctgcttcaaacagcttgaagagcaaaaacagcccacagcagaacagccaggatgctttggaagggaaatttcctttttgggtttgctgtctgaaagaatttccgatgcaggcagacgttaattcctagctccatgcttcctgataaggaagcagcttcaataaatgcagctatttatagatgtctttttttttttaagaaaaaaaatatataaccagaggtttactgaccttttctttcacttcacatttcatgcagtgagctctgtgtacatttaaggcagttcctccagcgtcagctctgacaaagtggccctggtttcgctgtgggggatggggaggggaggatgaagatggcacggcccctcattagtgtcacgggctctccctttcctcagagttctgaagtagacatttggaaactgttgacaatgggttgtccctacctggtgctctgtgttatttatgtgagcccagaggtgcccagctttattctagactcaacactgaaaggttatagcagactaagagaaccctttaaaaaataaaagacgatgagaaccagtccattttatgaaacttgaacagcaacaggaaatcagaaactttatttccatttcttcttttcttctgtccgttgcttcctcttggattttcccaaccctcaatacgtctgtggtctttctctcgtcttgatgggtttccgcagattgaggagttcctgtccatgagctccacagtcacccttacatgtatctacctaatgctttctggcaggtgtgataaaggttcagtgtgttggcttgaatgtgcttctctaagttacttctcacctcctctgattatcagaaagcattggttgagctgagtatatacaaatacagcagtttccatgctcaaataactgatattttctcccattttgcttcatgtgtgagttacaccacattagtaattcaaacttttggggtttctggcaggatgaccttgacaacttcatcagaggtaaacgttagcagcgacgtttagatgttgctgcttttgctgttgccatgggtggattctgattctcagttgcctgtaccatgtgtgagtgttttgtggttcatttttctgatctcttctgaaggaggggtagagcaggacttgagagatttaaactggccctggctctggccacatgctgactgtaaattcccaagtataattagtcacactgccccaatatttactgcctccatcttgggtgtgtggacaaaaccccattctttcaacattggtctccatttctgcttccagcagatcatggataccagtagctctcaaatggggctgtaagattccagtaatagaaatgcagatgatgggtacggataatggataaagaactgtagatgtggggtggatgacgattggatacctagaagggaaagttgactgcacagggctacgcagtgaataccctattttaccaggtcagctgaggctaaattctctacttctggggtgggggtactaactgaaatggttcactgaggagggtgaaggtttgtaagggaagatgtaacaatacttttttaaaagcacctaggacaagttttagggtgttgaatagcaccatttaaaaattataggggtatccagcaatttatggagctctgattgagaagtatcattgttaggattttattacccatttgttttccagaagtagctcatctttgcaaatgagctttagttttttggtcattgtaccctacctgccagctcacaggagcctgtgtttctccttaggagccactctgaggtcaaccgtgtttaagatcataaagccactctagagaatctctgtggcattgttgtgtggaagaagaaataatgatcactactattattatatatatgtgtgtgtgtgtgtgtgtgtgtgtgtgtgtatataaaagtatatgtgtgtgtgtgtatatatgtatatatgtgtgtatatgtatatgcttcatatatattatcctgaataataacaaactctgcacagtagatattatacctgaggctcaggtaaaccacttgcccgaggtcacccagagaaagtggaggagccccaatttgaatccgtatctaattccaaaacccagacttttattttccacttcaccctattactctttaaaagtctgaggagcaaatgaaagttgtttcattctatctattggttagaaggagaatatccaagctaacaggctgatttttttttccaatttttaagcataatataaattactgacaagagggcccagatggataaaatcagagctataaaccaaattatcttttgaacttgaaggaagataaatttccatatccaatcctgtatgcacactgttatctgccagatagtctcaggtatgtgtgtaaattctggagtgttagaaacaaaactaaatgaagtaaaaagaggttccttgtctattattttccatcttttttaaagagatgatctacctcacagatggttattaaacagtaagaaagatgctgtgattgaatgaagattgtgttactgagtttacaccttaaaacactgaacttcctgataatttgtgttaagccaacacgcttcagaaaaattgcctgacgtttggtgccttagtttacccacatggcatgtgtgccagaacacgctacaacagggtaagagggcagagaacatgctcttgtaggggtcagttccttgagctctggttctacgtggctgattacttgggtcatcccagcagttcattccatctctcaggactgtggtttccacatctgcaaagtgaaatggcctgagtatatgattcctaagttctccccaactcaatcgtcgcattgttctcttattccagaagagaaacacaaggggtatcagcagtggaacattatctgtaaatgccagatttcatgatcattcacttgatttttaggactcaacatcaaattttagtgcagatggtggtactttcatgacacaggaaattgtccttcgttttacctgtgattgttttgggaggaagccctcctgcaaggaaatagggaaatccaacacccagctctgttgagcatttccctatggttcctgtgattgaaatcgggttaatgcttttgtgggatgagaactggtgccagctcctaggccttgcagggcttctaaagactgtccctgccactcttgccttatgctggttcccaggaccttacagccagatcacaggcctgctcccctgttctcatagccccctttctggcctgagaattgtctgtggcttggccctttgccccatgcaacatgcacaggtgtggcttctgctaactatctacttgaccccaactgtgtgattagcggcaatcccctctgcaactcaaggcgtgtttgcctttcctgtaacctgtaaaaaatttcagtctgcgtttttctccctctggagatggtatccactgggttttaggctttacagagccagctgatctgcgtggatgggccacactatcctcccttagccggctccgtagcacatgcacaattaatactgcattaattaattatgggagaaaaaaagcacagttggcaagggaatgaaggctttcagcttctaaagtgaaggctttgatgctgagcttgaaagtacatttccatccgcctcagccagcagagagaatctgtgacacttataccatcccctgggcatgaaaactcacacccaaggaaagtatttctaaatagaggccctcaactagttgctggaatcatctactcattgccatgggctgtgaaaagggggtgtaagattagggagaaaagactcgcccaaagcagcagtgattcgtgaccaaagctgatgaggggttttattttttctccttaagtagctcttaagtcaccaacatcaggaactctttgtagagaaacaaatatgagtaagtgatttggtgccagccctcttgaggacataaaatgactctttctccttcctcctcctcctcgcatgccttgcttcaggaaggaaccctaaggtggtgcaggttcccaaacctgttattcggatgggttggaaggttcatctctgttaggcatggtttcccactagatatgaacaaagtcggggagaaaatgctttcctttttcattctgattccttcacgccattgttcttgaactcctcagctcccacactgactcctttcacttctcctatgaggtggtagtgaagccacctcacaaagtcccctctctgaacagacgcagttctcgttcccctatttacaactctgtcaagcatttttcgtgggggctcttgctgaccttaaaatttggccgtgattcaaatattcatccaggtatctggggttttattaaaggtcttttaggaacacaaaagtaggaattccctgagagaggttttcttcacttgagactcatgggttataaatgaaactactggaatgccctgaccaagtgctgagaggagcttcagagacccacaaaaacctaaaaagatgtgaaatttgtctctgatcaaatagattttggaccttatgaaagaaaataagcttttcttttgattcttagagaaaaatttttgcttcagatgaatccaagtgtgtgtgcacgcacgtgctttcgttgggatcgtcttatagctgttattccagatacctgcttcacaaagcttctgtgattattcctactactgtgacgattcctgccaccccagtgatcggggaaaagaacttgaaatacaactgggtattgtagcattggcactgtgcagacgctgtgtagctctctcatgtctggtacttctttgtttgctaaaggagtattgagattcagaaagtggctgccttctaaacctttattgttcagatagcattactttggacccaggacagagggcttatagttgactgtgaacggggtgaatgttatacttaagcagggaatgtggtcacccagttgcaatgatttagagtgctttgaacataaatatctcccacgatgaatgaccagggtgcctgaaattccattgttgagacattccttttggatcaggcattatttccacatagctccacttttaaaagtaaatcattatggcaggagatagttatggagagagataattaatacctcaagtccctaatatcttaatgaaaattagcttatcagatctcctgaattttttgaagtggaaggaccgtggccagcacggagtgggtagagttggaaataagcactataatttgcttagggctggccttctctgtggataagacagatggaaaactagatggaaaatccaggcctcctgatttagacaaaacattgtgagcctggagctctgaggccaggatgagcagtctgctacttactgtgcgtggccattgctctcaagacattgccatcatcagtcttggccaaagaaggtcaggtctcctctagtacactggcgatttgagaatgtgcaagtcatctgcatttctttgacaggagtagtgaatttgggagtataaatctgaatccttaagcctttcaaccattctcactaaatgtctttacttcagggatgatcacgtttgatcgtaatgccaagatcttgcagatgtataaagggcatggtaatccaagtcttgagttgaagtaagtggtcaggaaaatcttgacccttggggaaggagctaccagagctgggtggcctatgtgtgtgtgacagggaggaggtgtgcagaatttgggggaaagatcctttgtagttctccatgtggaataataacttccatttattgagtgcttatggtaggcgcttttcacatgtattgtatcatttaaccctctaggcagtcctgtgaactgggttttattatcctcatcttactgatgaagagtcaaacttaaagaggaaaaggaacttgaccagagtcacagaggtggtaggtgggagacaggtccatccaatatacaccttgccttccaatagaagccaaagccctattgcctaatctcttcactttaagaacatgaagaatagtgttgagttcccctttgagatctgactgtggttaaagcttcacaaatttcagaaaaacagaataactcactaaaataaaaatttaaagctaccccacagaaaacatctaccacaacccaaaacttcagggtttatatatccagattttagtgaactatatgactctccctagtcccttgctctgtctcctggtaaatactgtccctttcagagccctggcacatacatagtgtaataagatcagaccccatccccctaccattccaatgcaatctaattgcaaagtgaaaatccagggccgtcttagatcctcatctgtctacgtctctcttggtccccactgcttgatacgcagtctcaggttcaataccattctctctttttgtgctttaggtctctgtattgtttccttttcaccacttttttttttttaatgaagtgattttaaatggaaaattaaaaacttaactgtgaatctggcagtttcctgctaatgcctctttgctaagaagcttcataaaatactgtatcttattcacttcagcacagctgctgtgggctctcaccacttaaccattcacaatatgtctgagagattaatttttgtaaggctgtggtttgagatatctcccccttccttgttttacttaaaatcctcagtgggaaagcgtgcatggggaaaaaaaaaaccaccaccaccctggataaaaggtactttggtaacatacgtgctttaggaactataatttgtcattttattttcttgtgccctcttcatttgaaatgctgcagcctttctgccagaagtctcaagtttgtgtatgagactgagggggaaacgagagtaggtaccttgtgctgagactgtcctgggacagtcccgtggtgggtgtgagcgtctgtgtgatgcaggtatgcttatttcttcatgaaagccgcaggtactggttgcctctcgcagggttaaaactcttggcggctcacaggaggtgatgaagagcaagggcacgggagaatgaagaagcaggaggaaggtgtgggccagggtcggtttcggtggggtccccacttgctgggtgaaagctgaactgtttatccacacatcccagcagcagcagaaagccaaggggtgttttcaggactcagctgccaaagtttcttgaagcagctctgctcaaaggcatatgctgggctcacttctgagggaaatttcctttttggggaagttgaagcaaaggaatttcctgtgtgggcagacagagcttcctgacttgaaggcttcttggaagggaaacagtttcataatgaaatgacaaattaaagacataccagccgggttcacttcaaaacagctattgagagtgcaggcaaagccctattatactccctcacagaacaaaaataaatcccatcctgacaggtttctgtatgccggattatatccgtccccccacacctttcttcacagccttcttcatgcttcccttcctcccacccccgccattagctgccaccagctccatcttgaacagctctcgagagcagccggcagccaggccgggagcctgtttcttgctcccttgctttgtggctttgcccatccctgacctgcgtggaggccttataggttatgtgacagccctatgctgccaagacacagaaatgacctctttccttccccttttgacataaagcaatgattgaagacgtcttcagcaatggggtgggggcgggcagtcaggggagaatgcagccatttcctcaggccggcctttaaaatcttgaaggcagtaaatgtaagtgctattccggtcttgggagctgggtctgaattctttgctttgttgaaggaaatagggctctgtgctgggtggacgatattcttgtacctgaacatgtcattttcttcagttccatcagcctgcatgtggtgctttgttttgtttctgaaattgacatgatgatgccttaggaactaaatttttttctttaatggaacaggctaggagaaagaaggatggcggtatcatttggggcgtgagaacagtcattattaattaggcttaagttaatgaaataaaaaaggcacaggctttgaaatcagttgtctaaatttaagggccagctctaacaatatacaaccagcaatgtaaccttgaacaagtcactgaacctcccagcctcagtttccttatctctaaaatgggtacagtcatacctttttcacattgttgtgaggaaattagattatgatttcagaggcactttgtgaattcctataagccttcatacttcttattaaaccactattgggatctctggagactgaaatatagtattaggctaagacagcagaattgtagaatttgtagatcaatttggcaaatatttagtagaagtcttctatgtgtgcagcatggctcgactctgtgggggaaaataaacaagtggaaagtttcaggtggccgcatggtatagcagaaagaatattggacttggggccaggaggccagggttcaatcctggctgtcactcagttgcctgtgacttggtatgaactgtttaaccttgatgagcctcagttgctcatctgtaaaactgggagaaatcaaactctatgaccttaataaactctgtccaactctaacattgtataatcagttttaagaggtttttgcctttagcagctgtttataggatttattctaaagagtagggaagaaatattcattgtataagagcttgtctgatgaagaagattgcatgaaaaagctacacttttttaataagtatatctgatgtaaacaggaaaaatgggtgcttgccagatatgtcatctatagcaacttgttttgagggggtctctgaatgctgacatgaaaatgactaggatcatgggttgaacgtgagtttgctcaagagaaggttctcttggggctttggtttacttcctcccagctgctccagggaacactcaggatatttccaaatggctgctcatttaagccattcccgttatggttctccttgtccaaattggcttccatggtacagtcccagtgttgactggttctgctaatttgattctttttttttttaatgtgctgatatgtagttttaggactgctgcattattgttcctctatctttccgtttgtattagtaaagaactattagcaattttgcctcagtcttctaagcacgctttttgattggagaaaaccaaggtaccagaaaacttagataccatgtggaaagtcacagtttactgggtgtgtactatgtgttactagtgctttaataaatttcctcattaattttattctcccagcaagcctggcaggtagctgatagtatctttgctttacacatacagaaactagggcttagagatggtaagtaattgggtcaaggccacagaactggctggtagcagagccagaatttgaactcatgtctgcctgctccaaagcctgtgttctttatcccctcagtgcacccctactgttttgactgctgttaaattgaccttatgcttagaaccgaagtcaaacccgtaccagggctgcaaaaagaaacaaaattaacctcatagtctgcatactctcgaaatggcaaaaaagcctgcaactttccgttgtggaaaataatatacctgggtgataaactgagcaaacatcacccttgcccttgcctctgaaagagagcctgttctagttcatcctggggccagcttggcctggaaccccactggccagctctgtccacaccttgcatattaggacagaaatctgcctaccacattccagctacataaagaaggtggccagcacctgactgcctctttctccttgtgaaattccttggaaataccatttgtgactgcttgggcttgttctgaagaaaattcaaatcttgttctgtttaacaaggattgattgaagaacccgtaggctcagtgctagtacaaagataagcaaggaaggaactcagaataagagagaaaaaacttggataataattatcaaaaccagtatttattgagcacctactgtgcccagtagagtagtaattacaggaggtaaatatctaaaacttacatagctcttcacagaacatacatctgttcttgcttctgtgtttgattttattagctttcctctgtacacataagcttttagtgtatgtcctattattgaagtccagagttttattatttttcacttatttgggcatagatagttccagaatttacttgtcttcctcattctcattgttaatctcatttttcttccctggagaatatggactttcaaaactatttttcaatcaaggccaaaatatttcttaaaattcacttctatttaaaacacagataacatccaggagaaaatctgagaagaaaaaaaaaaacgattttgatggtacctgattataaaagactcacagtactaagttattctttggcacgtgtagcaaaatactatgtaggtgtcatttcgagttcatagtaaggggagaaatggtgtcatttttcctgcagagaaatttaatctttcactttaatctttcattggcctatagcagccagtggggtcttttaaaaaattcttttccttcatattccccgtttgagcttaaggtacaaaaaaagaaaatactcttgaaatgctgtgaccagatctagactgtaacatgccccttcactcctctggtgaggaagtgcgggaaggtggaagacattgaggggttgattatttacttcgtttggaagtaagaagggaggtagctcttatttggaataagagctgtcttcagaacaggtgtgtgctctcctgaagcccagagctgtgactccctgggtgtgtctcttacctgtgcccttgggcaggtggcacgggagaagaatacgataagaggaggatggtacagacagaggagtcacaggctgaacaggctgtcaccggggcaagatgactgacaggtggatggaattcaccatcctttcctttcaatcaaaaaaaaggtagaaagttgctcataagtaagtttgagttatatcatcttcttcatagccgttgctcttcagacacaggtgctggtaatcaggaaaatgcaaggaaacagctctggggaaaaggaacaagcctcgttggccttgagagccccagtagaccatcaggccttgctttggatattgggaggtgacagtgccttctcacgtgcctgacagtttccctgcaccaccaagactctgtgtgactgaggccgtggcccgggtttcaggatgggcattcagaggaaggaagttccccctccttcacttacctcacgcttactagatgctgggttgcctatgcccatgttaaaggaagccagataactttctgtccccatgtgatccaggcccatcctgaagcctgggggttctgaacataagtatggcggttgcagccttccttctgtgacttctggaattcctgacagagagggtcatatctagccactgtcaaccggatgctagacttgttttgttaggcccttcctccttcccttcctctacttatcttccaataaccttaggatttggaggaaattaaaataaaccttcttctagaaaggtgtcagggacccatgcacaaatgtagcctttttgatgcttgagaaagaatgcctttttgacttccgtgaataaagaataagtcacggctaatgagtaagtaacagcacgtgcctgtggtcccagttattcaggagactgaggcaggaggattgcttgatcccatgagttcaaggctgcagtgagccatgattgtgccactgcactccagcctgaacaacagagcaagaccccatctcttaaaaaaaaaaaggaaaaatgcctatgcaatggctctgaggcaccctcatggttgatccaatggccttgcctagtgattttgacatgctttaacttgtgaccaacatctagtctatttgtctcccattagagactggttgtgacagcattgccattgacctgccatatataggaactccagtgtctgaaaacatagcaggactttgtaggtattaacattgagcagtgctttcacattttacagcttaccgtgatgagcaagatattgtgtcaaaaatagtgcattgcagatatcaataagaggtagggtaggcagtgatttcagtggtgtcccactccacactgtaattttgtttctttggtcaactaatgtaagaactgagtctgtaactctaacctagggagccattgctgtgtgtcaactttgttgctttaaaatcttgagacaccttttcagtatctcccactttgtctgagtagtcatacttttaatgatatttaaaagtcaaactctaaatactttggagctaaatctcagcagagataaacttcaaactggtgggagtgggggtagagaaacccttctgggagtcagaatgagatacttgtgtgtctggactggtggcaggtttgtatgttaccagagtgatctccatggtaacggatcaacatggcgccaatgacaactctacactaagagcttcaggttcccatgagaaagtggatcctcacagcaaataggaagaaatcaagaaatatggatccacactccccttccctgtagaattctggccgtgaagtttcagttcatataaccaacaccatgtcctatttaaccacttgggtccctctgagtaaaccaagactcttctgagaatgcagtgttctctcagatttcaactaaattcatacaaagtttaatcagacttgcatagcaaagaactatattgcagtggagcaagaatgtaacttggagtcaggaagcctggcctccagacccagctctgacactgtgatatgaggaaatcacctcgcaggttcttattttccagattctcaaattcccaccagtgctgatggtcaatcatccaattatattgaaagcccttagtatgatacatcagcagtaggaaaggtagatgactaaatataatacagtgtgccttttcctacaccatcaaaggtatgataatgttgtgatcatgtgtataaaaactttcagacacctaaaaagtagaaggtgtgattcatctctgttaaagcttaatctctgagcataacaaccaacatctggaagctaggcagccaacagaggggttaatttttctggaaccttggttgcaaaaggttattagtttgcagaaagactcagagatcactgtttatcttaaaccacaagagaatgtttttgaattcagaagaacagtgatatagttcggtaatttcatgaacctcttccctgggaaaccttatgactcaacagtcaaaggtgtccgaatagtaaagatggttttcagtgatcaggtctgtgcccatgcctggccttggatagactctgaaatgagattctttgtttgattgatggggtgatggtttctgttgtgtacatttgaaggaaaccagtttccccacccaaaatttctaaggagtttaatctttggggtataggggagttaaactacactgagtcaaggaagtaattgattgcatatttcctctaaaagtcagctatggacttgatattgactaaaacaaactagcagttctcttccaccaccaagtcagagcgtctgttcaccattctgcatgattaaaagaacaactaagaatggtaatgctccatttaaactttccttgtgattctctctggtctaccttcaatcaacaaaagcctaaaatttttattttagaatcatgatgttggaagatctttagattgctcataccataaagctgtttctaagattggtctcgggaaggtccaagatcaggattgaagtatgaaaaatcttggccaggtgcagtggctcatgcctgtaatcccagcactttgggaggccgaggcaggtgggtcacaaggtcaggagatccagaccatcctggctaacatggtgaaagcccgtctctactaaaaatacaaaaaattagctgggtgtggtggcggacacctgtagtcccagctactcgggaggctgaggcaggagaatggcgtgaacccgggaggcagagcttgccgtgagccgagattgcaccactacattccagcttagtgacagagtgagactccatctcaaaaaaaaaaaaaaaaaaaatgaaatcttgatcttattgccttgtcacctttttgtcttaagtttactttgttggtataaagaattttttttaattcagcaagagctcaaagagccatatatatatatataaatttttttttaattaaaaatctttctgctcaagtctcaactacagtgagactgaatggaaaatgcaaaagctcttgacatgatgtcggaatgaacaagatggagaggttgaatttgagaacatggtttccttattgtgaaattccagtttcagaacattggcactgtctggttcaaatataaatacacattattttattgtaatccgctagtttaaattgtttattctgcttataaggaagagatccatgatagctgtgaacccctttgaaggagactttatccatggctcagtccaaggtgtttgaagcacttttctcatggcctggctggaaagactttcagaaataagcagggttctggtaggaaaatgaaggcagttgaggaaaacagtgcacatatttgtaagaggactatacttggtatcttggaaaacataaacagaccctagactgtctaccccaagtctgggaaggcggtcctctgaagctgaggttctcctggaactcccttccacagggctgcgttgcaaatgccattgtggaggcatcacccgatgtggtggacctgaacattggggaaatgcagtgctgcctgctgctgcgctggctggcatctgccctcctccgtctcctgggtgctgccacagagaagagagagagagtgaagcgggcagagactggctgttgccatcacacaactgagggcggacctggagctcagccggctgagggtttgagagaccgttgggagcaagacccaaccacaaggcccatggaagaggaagttggcagatctcagaaaggggaggaaaaggagaggtgcttatttgttcatcttgtttttgcttttaaggaaaggaggagagttaaatgagaaaggagggcgagttgagaaggttctgagcagtttcaggtccacggttgagcaatgttcttcctggtcaacaaactgcctggatttcattttggcctgacaacagccccaccacctagggaactgcacagagatcctgtgtgttaaccacgcaagagagtctgcagttagaatactggggcttgtgcagagagaagaattcagaaagtcaaaggctggatgtaggtgaagagactatggactctgccttcatgtcatcagttaaagtgccctgcacacctgcctgtcctaggtcctgtagggatgtgatataacaagcatgagctataccttagtgtctgtgttccagaactttgttggctcccgaagcacctttccctcctcccaagaggaataaacaaatgtaaataaatggtgcccaggttttcatttggctcacaaaagattatataaaactcatagtctgtctgaagtatgttacctgtaaacctgtttcagctaaccaccttttctagcacaatcaagctggtaaaatacatgcctgcacgccagccacggtactgaggaccacatcaaaactgctccttgaaggaacttggagaaaatgaacaaccagcaacttgttagctggtctctgaatgacagagggtactgctttagatgtcccccaggaattctgagtttatgcagcagagggaccgtagccccggggtcaggcaggacaaggggatcagaccttcctcttgctatgtgtggatcattattcagctgaatgtctctaatcaggaactctgggcataaaaacacgatctctgcagacttgtgtgtggctagacgccctctgtgtgctgtatttgtaattctctgtctctgtatggtttcagccttggaagaagctttgaaccagtgatgagtttgctttccgcagtcaaaactgtggtttgagaatttcactgttaataattgggagtaaaggaaaaagctgggctattgaagggtaaagccgatgtccctaggactgtggcctcaggaagatgactggatggcggcagcagaactgatggcagaaaccagggaagctgatgttgtattgcagcctccctcacacagattctaatcctagaaagtggactgtggaaaaaaacttaactattcacaacaaacgtttaccagtcttatttagtggcaggccttgtgatgagtgctggggttaaaccataagtcaaatacagacactgctgtctttattattcattaaaatgtctttagggtaattttgtatttacatcttcagcaaatattaactgagcacctgtgatcaaccacatagctgctaggcactgtgaataccgtggtagacaagaacagacgcttttcttgttaatccgtggaatttacaatctagaggggagactccacctaccccaccagatgtgtgcagaaggcgccatcactgctttagtttcaggtctttgctcaggtcattgctcacactgagtccacatttataagttttcctgtcgggtgagctgccgaagggccagggaacgtgataaaccatacaagcagcgcatccaagaagtggtttctgagcagtcactgagacagcgagggttccctgcactgagcaccaagttattactattttccatacacgtcaatatgttattgattgcttcactgtgctgatctgtttttgtttcatgtggctctttgggtctgtctaacctggtattttcagtctcaccagcatttctaaagttctagagacccatttcatccaccttacaccatgtctaccacagtgccaggcatcaagcctgctaattgtgcttgatggtgattattaccagccccggatttgtaagagtcaccagcagtggcatttcaagatttagcctgggagctcacggttatgtggaaagaggggctcaagactaagcccagggaggttttggggtgatagaattgattctttacactgaacgtgaatcctcagtgatgttttgatgaggatgcgggacattgtgctatgctaaagcccgtcacagcagcattaaaaatgtcccagtatggagacctgacttacaccttggctcaatcattaaattaaataactcaccaaagaataaataccaacaaatactagaaatgttgatttttagaataataaaatatcagtttttgttttttgaactggcagggaatttttttttttttctttttgtagagacagtatcatgctatgtttcccaggctggtctccaagtcctggcctcaagctatcctcctgctgtggccgcccaaagtgctgggataaaaggcatgagccaccacacccagccagaattttttaaatgttaattttcaatacacatgtagctatgaggagatggacacattctttctatctgacacacctggtcactccaaacgttcagcacttcacatgggaggctacatatggaagtgaaatgaaaaatcaccagcagccgcagaacaaaaatataatccagtgagcataacttttatggaagcacttcagcaatatgggtaaaggacctgaaaacacttttgctctttggctcactaattccttttctaataacccatccctagggaacactctccgatgctgacaaaccttcttacacatggttctgcagagcagcagaggccaccaccagcaaggttcaataaattaaggcaggtcattatgtgggactattatgcactaattaaaaatcacattttttaggccgggcgcagtggctcacgcctgtaatcccagcactttgggaggccaaggcgggtggatcacaaggtcaggagactgcgaccatcctggctaacacagtgaaaccccgtctctgctaaaaatatttaaaaaattagccaggcgtggtggcaggtgcctgtagtcccagctactcgggaggctgaggcaggagaatggcgtgaacctgggaggcggagcttgcagtgagccgagatagcgccactgcactccagcctgggtgacagagtgagactccgtgtcaaaaaaaaaaaaaaaaaaaaaaatcacgttttttaaatgctcatataatataattgaaaaggcagggtacaaatctctctcttcagagtcatctcaattgtttttaatccataaaaataaaccaaaaggctatggaggaaaatagttattgtggttatctctaggtagaaggattgtgggtagaatttctttctctttactgtggagtttttttgttttttttttcttctggcattgcccaggattatttctgtccatcagggcgtagtgggtcacttctgtacccagcagcttgcacctagatttattgtgtcaaaatgcaaatctgggcgtgcattttccattgtcccctccccacctgcgcttaaaacccctccttgtcttcctgctgtaagtaggataaatgcaccagctgtactgggcctttcccatttcccatgctgatcacattctctcgcctctttcttctggctacaacacacttcttcattccaaacccttccccctgacccacctccaccccccaccctagcaagattaattccctgtaattctagttcatccttttggccccagtttaagccatttattcatgccaggctttacagaagactcaccagcacctccaatgggcttccaaggtgctctcaacttcccttaacacaggttcccagtttatcctcaccactgaagcccaagagctgggactgtgccagattgatcaccagtgcattcccccagcacacagcaggactcggctcaccctggatggttcttaagcatttatgaatgggtggatctcacctacgtgacctggaacttctctgttctcagagcaagccatgtcaaatagcagaaaatgcaccggctttggagcctactgcaggactagatgtctcagggtcgcagtttcctcatccctaaaaaggggaatcacgatgcttatctcatgcagttattgtgaggttacaagagttactgcttatgaagggccctgtccagcatctggcacgtggcaggtcctccgtgaatgacagcgccttcctgggcagcctggcgagcgcacacgcgtgtgcacacacacacactcctgtgtttaatatagccctgtctgttgtgagccacttgaggagaggtgtccctagaagaaacttttttcccagaaaatgaggcctggctggtgctcatcctgagttcctggaaggcagtgttttcttgtaaccatctgagattcctggggctgggtatagtgcctgatgctctcaatatatgcgtgttgaatcaaaccagaagaagcaggctgaggcactgagtgaaaacctatgtggatgtcctgctgccttttccttgtggagggtagatcattcccaactctgcctgaaactttgatgccttattggaggaagagaaaggtaactcccttgttgagccatagagctcttagtttttctttcttaaaaaaaaaatcactcttctttcaaacaccattaaattagtccagttatgtacactgggcttgtattgcatggtttatataaaagctaagttctttctttaagattggagccctccgtgtgagtgatttcaggataaaaggccttagaggattagacacattattcgtttgtaaaactgattgctcaagtgatacagccctagggctgcccccttattgtagcttcttgaacactgtcaaactgtcagacttgggttggtggttattttatctcgcaaacaaccaaaaccagatttacattgtaagtagatactagcgtggcttttgtccttcctatatctaggccctgaatacttcagagccagattaaatgctttgctctattttttcatagatttgggcttcgaccaaacatacaagttgagcctgttgacttctctctttgtggatgcacacgtgcgtctctgcgtatatttgtgcatgctttcccgaataagtcctcttttaaaactcagcctttcatcattttagaaatgatttgctcttttacagcttgtactttttatggttcttagaggatcactgcagcaaatatatttggcagaaattgagcacacaagcccttccccagaactcagtactgaatttgttcagttcccatcttaaaaacttgaaactcctttgacttttcctgtgatgtgcttatttctgatctgaaactgtagttcaaagctagacttggtaatcattcacgtcaagaaaggaatggattgctcaaaaaagaagtcagactctaaaaatagcctccataccgaggcccacttttctacctagggaatcaagacagaaatatttccaggattgaaatgacatagttcaagtgacgcaaccatttggctgcctgctatttttctcttttctgccagaaggaaaatatataatgtagaaaaattcccctagagttgttaaaataatcactgaaataacctttggtgcttagtatggtaccctggaaaacttggatctctagaatggttcatttcagagcttttccagatagccaagtttttattagaaaattggttggtatagacggtgctcactgtgactgttcagtgcatgatgagaatccttctttacagagataaaaagtgtgaagagaattgctgagaagtaaccattcattcccttctgcatttgcctagtgtgctgtgtaattaaatgagctgataaaatttacaagaaataaagctttatgcaattacaaatagatgacatgactttttaaaaggttttgcccatatttttcacacacgctctacctttagccttctctgctttcacacagatgctgaaaaacaaaagagttaattagaaaccattccttttctctctttctggtacagagaaggcttccactaagtatgtgctgaataaatgacccagtctgaaagttttaatttattacacagtatattcatagaactgtaactgttacaacctctagtttgatagatgtagctgtttttgtttttttaagttgagattatattaatatctgtcacttgggttgccagccttttagaagaaaatttggggtttatattagaataggttttaggttcagtagcctgtatccttttttactgccctgggacaacagcaaatattcacctaaattcattctgtggtgtaacactttagaagtataccagttcttccttgcaaataacaacaaaaagctctgaacaacttaatcacagagaaaaaaaaagcttttattggaggtatattgggcagctcattggctgataaggtagcagaagcaaaaggatacttggcagcccggagcttagctgagatggagccataaggacagttttattaggatgccaccacagctcggctgccagtggacacatgaattcgtgagactagcacctctgccataaataacatctcaccatccctggggaattggtcactcccttaagactcaaacactgtggcaagtgcatctaattggctgagtccaggttatgttccagggaacaggggagaaagctctgccaatcttgggattcccccacatgcggtatgtggatgctgagcagacaaaaatatagaagtctagtagatgattgtaagttactgtatagcagtgtgtttcatcttgttcttacgtcctgtggaagctttcttcatggcgaagttaagcagttccacatctgcattcccccagatcagcacaccaaaggtgaacagcttgggtttccccctactttcagttacaggtacaagatgttagcaaactatgcaaatctcaatgagttttgtttcctaattctgtttgcatgctaggaaacttgcctcatgatacaacagtaaacccctgctttaagaaaaacatgaggctggtttaatatgtacaatgtccaactcttaggaagtgtttatttttcaattgcctttaataatttctcataaagatcgatgagtctaagaattgtgaaggacctcagagatgatatatttcaacttgtcatccacgtcacgactgtctctttagcatcccagagatagcaggacagtcctcccctgcttgtgtgtattcagtgggaagtacttccctgcccccttttcgtcccgttcttggatggtgctagatgtgaaaaatagagttgaaactcacctcactgtcactttgctcttggttctagttttgccatgtgaaaccagatagggtgtattctttctacattaagtctagagacagaactttggaatcagatatggatttgatgctcagctttgccatttacccaaaatgaaacagttactttaacagtatttttcgtctaatatgtgcaaactaattgctgaatggataggcagttattttattaatgcaacaaaaggttatgttagcattttaatatttgtgttgtattcacattgatcgtttttacttggactatagccaaagtcagaactcttcttttgtggattttttaaacttaaaatatatgcctttacatttaaccctattacgttttatttaagttggctgaggtaatgcctatcatcccttctataaactcctctttcctactttatggcttctatattagatgagtactttctctgtacctttatgttaattatttggaagggaaaaagattgaatgggacgggacctgtgaaagatccctgtggcataccgctagagactgccttccaggtaggcatagttttaaaactataaagttaactttatagttaacaagtatattgtgattatgttttctggcaaataaatctctttgctgccatatgccataaatgacacattatcatttttgcaaatatgcacaatggtactacatatcaattgactgtcagcctaacccataagaaaaagtcaggatagactgtcaagttatttgctgaaatcagatgcactgcttggctctctgtaaaatctttaccaagtaccctctttcactttactcgtctactcatggcaaatctaaaaccttctacttcagcttaggcaagacatcttcttgggtcagtggtagatgtgaacaatcttcgtgggtcagtggtagtttaacagtttgttaaactgttgttaatcaatgtatctactctcggtgattttaagagagaaaagaagactttttgtttttgtctgagaggagtttttaacctggaaagacagctgaaacgttatctgaaatgtgaaatgttaaacattttattattaagagacagtgtgttttttattttactcacacagtatgtgtgcatttgtgttttctgcgactgcacatttgtaataataggctcatattttgtttttcattttaaaattggctttgtaggatgttcactaatgtggaaagacagagggattggggaatcactgggaaatgcttggagtcagtctgttatgtagatgtgcaaggtattgactacagagtgagacactcttgcttggttatgtcctatttctctctgaattttttaattctcaaaaagtaatttagaactataacaccttggtttgtatgagtgtaaaatgatttataagataacctcaggtgtgttgacagaagaaatgcaattaaattgtttccatgtttaagaaaaatatgggaggtatatgtattataagtatacatctattgactggctaaagaagaaaagatgactagggtatgccatgctaaggagtttagattttgtgctggaagtttaaataggggagagaagggatgtgtgttcagaaggcttttgtaggtagcagctgtagcaatgatctaggtgagaaaaaaataagtacaagccaaagcagtgtcagagagatggagtgaaagagaaacattcaagtgattaagcagattgattgggcaaggtgagtggcagatcagatgaggagcaagggatgggaagcagaaaagacaagtctccgtggagcccattatctagagtggcaagacgagaaggaagagcctataagagggctccccagagagacaggagggaaatcacagagatcaaggaaggagaatttcaggaaggaaagaatgatcctgcccaatcagatgctgcatagaattcaggtaagatgaggactgaaggtgtctttggcttttccaagtagggaagtcatcagtgacccagtaagcacaatttctgtggcggggagaggcaggtggaaacctgattgctgtgggttgaaatgtggaagaaagtgattatagatgactctcttactggattggaatcgagagaaaagataggatctggaaggcaacagcgggtgaagggaggtttttgcttttgttttgtttatttaatggacggtatttagccatgtagaattgttggtaagaatcagagagttgaaaataaaggagaaatatggggtgatttatggaacagagtccttgtagaagtgagaaaaaataagaggcaaagtaaaaatggaggtattatttcattttcaatggaaagatggacacttgttacactgaaatcagaggaaaggtgtgtgtgaacataagacagttgtttgtatgggggcaggcttcataacttgtggaaactcccacttcacagcctttttgtttgtggttggttgtttttttgtttgtttgtttgtttgttttgcctgagaagccaaagatacaggaggtggcagtgaaggaaatgggagagtgcctgattgagccaagctggggtccccccaccgcgtggcatcgcctcccagtgggaggcctggagtttgtattgatactaatttgtctgtctgtgtggtttccccctgtgctgctcaacaccgtagtgtaggaggggtgaaaggagatggtgagacccattctttcaggaaatattaactgagcacctgctatgtgccaagtgtgttctaaactatcaagatacaggccaggcacagtggctcatgcctgtaatcccagcactttgggaggccaaggtgggcggatcacttgagatcaggagttcgagaccagcctggccaacatgttgaaaccccgtcttcactaaaaatacaaaattagctgggcgtggtggcacccgcctgtaatccactactcaggaggctaaggcaggagaatcacttgaacctgggaggctgaggtgggaagattgcttgaaccctggaggcggaggttgcagtgagccgagatcacgccactgcactcgcgcctgggtgacacagcgagactccatctcaaaaaaaaaaaaaaaaagatacagaatttaacacaggaggagcaagccatgtgactatgtgagttcaaagtattccaagcacaaacaacaataaagaccttgaggagggaaagtgtttgatgtattaaaaggaacgcgaggaagcaaatgtgtctaaagtgattggcaagtgaggaagagcggagttcggaaacaggagtggggcaagaggacttgggaggaggctgtaggccactgtaaagatactgtgttttttgtcattttctgagacaggctcttgcctcggctggagtgcagtggtgtaatcatagctcactgcagccgcaaactcttgggctcatgatccttccgccgcagcctccagagtagctggaactacaagtgtgcaccccgtgccttgctaattttttttttttttttttttttttggtagaaatgaggtctcactatgttacccaggctggtctcaaactcccagcctcaagcaatcctcctaccgcagcctcccaaaatgctaggattacaggcatgaaccactgcgcctggccgggactttggcttgtattaataatttgccaatgagaattgtaagcaatggattgacatgctctttgaattttaaggaatcattctagctattgtgtagaggataggaagagggagagtgggagaaaggagggggaagtgtgaaagtagagaggcctgttagggatctcttaataagtctggcaagacatgatggcaggccgtaccaagcatgttcctacctcaggcctttgcacttgctgacctcgctcgctgggatgctcttctaccaaatatatacttgaaccactgcgtccctccttcagagatatctgctgaaatgtgaggtcttcatagtaaacctcaccccttcacttgtataccctttccatgacattttttcctctataacatttatcaccatttgacacagtgtatattttacttgtttatttacttagtgcctgatttcctgactaggatgtaagctcaagaacagggatttttctgtttcgttcattgctgtgtccctgatgcctagaacaatgcctgcagtgaggaggtactcaataagtatttgttgaatcaattagggaatgaagctcttccatctccattatcaccaataccacctctacagttataattagtgagaattatggcagtgctaatgttattttttcatttccatgtgtgcctttttggaaagacttgctgcaacaagcttcacatatcttcctctgcagagtagaagtatgtcactaagccatggagttgatttcttggctccttgtatagcaaatagccttgcatttggttcgtgtcagtctttgcatctttttttagtcacgttgcctacacccttcacaatcctctagcttaaactcttgagtaactctggacactggcgttagctggaaacttgagaaccagaggaatgatactgccaataacatgccttatgtgcttggcatctaattttggcacttgttttagcaggtttgattgcataatttagccattggctacaaaaaaaaatggccacatatcctgggaggaagggaagatttggaggtataagatggaatctgtcctagggatttgaggacgggctacggttcttgttttttttaaaatattccttcattttcaatactcttgggattattccaggccttatgcattgaccatcactcaatattacttcctttggggagtatggtatgacgccatcatttctggtcttttcatatgaagtctccatttttattatatttttcatgcctgaatctcaggttttgcaatattttctctaccacaaaatctgttatcaattaagtacaacttaatttgttttttttttcacttttccctaattaaagatatatatacgctaattcagaataagtatgaaatataataactggaaataggacatataattgacataatcccaaaataaaaacaaataatttgtagatttagtcctcatagtctcactggatcaatataggatttctgatcaaatatgatgcattttgagaaccctcagtttatgtctagaggagagtcttttatttttaatgtgcgggatttaagttgtaagtgaagaggactgcgtcaagaggcaggatagtagttctaccccaaaacagatgtgtgatgttgaacgagtcacttggagaaggatgccatggagagtattgggccaggtgatttctaaagactcttccagcattaaaatacattaattctttgcatacatccatgccttttgctgcttttgcagctttcagggttaatgagagctgtcaggagcagtatgaccttagagggaaatggctcatatcaagagagcagatgccaatcccttctagtactctccctgggcagatgacccactccctatttaaatccttggctttttttcttctctgataagtgcctgcataattgatactgttgccagtgatcaaacgctgaaatgactaaatgatagagctgaaaaagaccctaaggtgttatctagtccaattatgccccctaaaccccaaagagtctgaaagagtaatggggcccatgagccaagtttagtatttcaaatgtctcgtggtaattctatatttggagacaatattgtaaggttaagcaagctgtggaagcatgaggttgctctgcttatggctggagttataaatgtgaaaatggatcagttattcctagtaactgctcttgagtaagcagaatctttcaaaatgtcatagctttgttgtctaatacataatggccactcaccacatacggctttcaagcacttgaatgtgactgatccttactgagttacgctgtgagtgtaaaccacacatcagatttcaaaaacttagtataaaaaagtgaatatttcattaatttatattgattatctgtttaaatgatattttcaatacattgggttatataaaatattttattaaaagtgatgtcgattgtgtgtttttacttctctttactgtggggactaaaatattttaaattccattatatgggtaacatttctattgggagtactgctctagagtaagagatttctgggtgtgaaaaggctgagactcactccttgttttgcaggtgagtaaggtgggtcagagaggttaggccatttgttcaagatcacacagcaagtaggtagccaagttaggagtaggccagcatactttcctttaccaaaaatggccaaagggatcatagtgatttttttacatggagtctatgacattgctgctttgggtcaaagccaaaaatattgtgtagaatattagcagttttctaaagccctaagcccaagttgtatcacccagtatcaaatccaatcaaaaatatattaaaaagaatacaaggaagaaaaggaaagaggagaaacctccctatagaagacccagtaagcaatctgttttcattcttgaacagatttctttcacaccagtgccctggtattactggaaaaaagtacattaattacgggagtaaagcacaataagaaaacatttacaaggagtccagccctcatccatcatgcctttccctccaacaaagcaagaatctctttaacaaatacccattaaaagattaagcctaagaataaattgtttcttagtccccaggcagttcatttgacatcatttccataggattagggactccagcaatatgtgtttagcacagggctatagtcacactgcatagtattggtctaaaccattccaaaatatttttttagatctcgaactaataagacttttatgtggctggtaattatactaatatttcacccacaaattgggcctttttccccctccacagtgtgtaaccaaacgcagcaattctgcctttaaaaaaaacgaaccaccacctgttaaaaagtggtctaggaagtatagcaatgtcatggagctcttgaggatgtgtataattgaatagaaaggaaacctagcatctggcgcactcagttcttagtcccttttcctctgagccatcatttctcttctgataactgtatttggttcttataattactgatactatgatgtgtggagtttaaagtacccaacagttgtataaggcttgttacaaacaccttcaatgtccccactcccctcccttcactcttgatttctttttgatttcctgctcaccagggcaaccacattcaaatcttttaactgattcttttgtcattcacctcctccaattcttaaatcacgtgcttgtattggtttttttttttttttttcatttctaaatattatctagcattatttcactgtaggaaatgacagtttgtcctttcttccccctaccccaacccctcccatagatgcatcctcgtaatcataattttggacagaacaatttagtgtttacatattaagattatataagtgccatttgaagctgagtcacctactcttttatggttcttcttttccagtcacttttctttttccctggagttgataatgatgcttttatagtatatttattgtttttgttgttgttgttgctgttttgagacagaggctcacattgtcgcccggactggagtgcagttgtgtgatctctgctcactgcaacctctgcctctcaggttcaagcgattgtcctgcctcagcctcccgagtagctgggattacaggcgccagccaccatgcccagctaatttcttgtatttttagtagagacgggttttcaccatgttggccaggctagtctcaaactcctgacctcacgattcgcccgcctcagcctcccaaagtgctgggattacaggggtgagccacgatcgtatttcttgttttaatgtccttcttacttggttttttatctccttcactaattcatccgccctgcctcactctcctctcagtagttcagatgccataggtgtgatgagccttgaagcaagagcttctacctgcttcggtctgggcttcagcttggtgcccagctatgacctttgttcactttttctctccttcgggtgtgagtccctttcctgaattctggccttaatggaggacaatttccaatagcttcctgagaaagggtaggggggaggcaactttattgagaccttgcgtgtctgaagaatgtctttcttctttcctcatacttgatagattatttcctcatacttgatagattatttgtcttactgtatgattttagacatggggtaattttcccccagaattttgaaggtagcactccattgcattttttttcagtgccattgttgagaagaccatgccattccgatctttaatcttttgcataaacctttgtttgttgacttattgttccctggaagcatttttttattccccagattctgcagttttacagtaatgtgccttagtctgggtctgtgctaatcctgatgctctttcaatctggatattcatttcctttcattctgggaactgtcctcctgttttcttcattctttctccagaactcccattattaggatgtaggtccaataattatctttatttttcacctcttaatattttaagttcctcttggagagattcctcaaccttttttgtcagcacttcttttttttttgtttctgatatcatacttttaatattcaagtactttttttgttcgacttgttcttcattatagcatcttgtttttgtttcatagttgcaatatcttatttctctctggatgatatttttctttccctgagttgtctgttttccccacgttgcctttgtctggttttggtctttgttttgctgtctgccttctatactcaaacgtctggtgatccctgcatgtctgcccatttctgagagttgatggacccctcaagcactgaaggcaagttctccgtgcctgggtgaacgtgctctctagggtggcctgcccacacatttccttgggatgccattgggtaaatatctccaggtcttttgtctttggctgcttagattcccaagagatggttgactcttctagtagccatcctggagggtagattctagctgccaacctcctaggagcacagtgggagaagaaggggtagggggcacagtgagcacagtggtcaacatatgcatgttcttttaatctttcatataatttccgttaattccacctagtcattttggtaccagaggataaacttttttttttttttttttttgagacagagtatctgttgcccatgctggagggcagtaacataatcttggctcactgcaaccttcacctcctggattcaagcgattctcctgcctcagcctccctagtagctgggatcacaggcgcgcgccaccacacccggctaatttttgtatttttagtagagacgggctttcaccatgttggccagcatgtgggtctggaacccctgacctcaagtgatctgcccaccttggcctcccaaagtgctgggattgcagctgtgagccattgcgcccggccagagcttaagcttctaatctgccatggtaggggaggagccgtgactcgactgtccagattttcacatgtaactagtgacaccagtccccaatcctttcacggattctgccatgtgaatttggttatatcacatctttccttactatgagtttagaattccgctttcttgggtttgctaagtaaattaccaccggttcttctgctttcaagtttctggtttgtttttctttctttttctcccctccctccacccccttatctcttctggtgtctttgtccttgtagatttgtgccttcttaaaaatctcttcactgttactttagaggatttgggggaacgagaacagagctaaaattaacttcctcctctgatgaatgttgacagtcttcctgccatagggatgttgtccaaaatacgtgttcatgtgtatgtcaaactagagcttctcaggaggaaagctctttgtccattgaaattgttgtcactattgatagtctttttacccttagaccagtacaacttgagtcacacgtgtggcccataaagattcctgtaaggtcgtgcataagggacaagtagttgtagctttgtattcgtgtccagttgatgaaaacaaattgctatatctcctttgttacaagaaatctatggaagaatctggatagaacccagatagttatatttttgtttttacactgacccctttaattcaataaagacatcaatatgtgaatacataataactgacacatactactatataagtgttcattgatattgttatatgctgtttttttttaataatacttgtaaattttccaagagttttatctaatgtttctcaaacccagctgcacaacagattcacctggtggtcgtttaaaaaaaaagatttcatttcccaccttgggcctactaatcttcagggtgggtgcagtagcaaaacagagcttccattttctggagaagaagtgagacattttgagaaccccgttgggagggtcgtacacatcactgatggtcatttagaaagacagcaagatgaagcaaggagccagctctgagagctgcggctaaaagcaaagcctgtatccacagcaaatagataaatcaatatacacgtgtagtataaatgtatagaacacctggaaaccaaggcactatgctgggccctccttctgtacagaacacttaggcagtatgttgccccctttctttatgatgttttgcccctttgtttcagtcagattttacctggataggaggatgagcagaagaggctggtgtggatgtgcattttgttcctgctctcccagtgacagacattgaagtgttggccggagctagctaagaatttactttagacaaaatcctgaataggacccacacaactctgagacggattacatttaaaaattaatcctcggccaggcgaggtggctcacgcctgtaatctcagcactttgggaggccgaggtgggtggatcatgaggtcaggagttcgagaccagtctggccaacatggtgaaaccctgtctctactaaaaatacaaaaattagccaggcgtggtggcgcacgcctgtaatcccaggtactcgggaggctgaggtgggagaattgcttgaacccggaaggtggaggttgtagtgagctgagattgtgccactgcacttcagcctgggcaacagaacaagattccatctcaaaaaaaaaaaaaattaatcctctttcagtgtcaggtcccatttgctaatggggttcaattttcattcatctactgtaaaaccaaacaattcttatttgcagctgagctaaggggcagaaaaaggaggaaataaaaagcattaagttttatttccatgtatgcaacccagaaggttctaaatggttactttatttgagcaacttactttattaatagcaacttcttgtttaactagagcagtgttggaaaaataatttaacagaaattttagttagggggctacgggaggggtgggagatgaaaaactgaagaatgacgaaattcacatccactaatgtttacatttttaaagctttaatgtctgtttttaaggaatagttctttaatttaaaaaaaaaaaaaccttaacatattctctgtatactctagtgttggaacaatggtgactgatatcggagctttcagttggtacagtaataatagctaaagctcacccagcactaactgtggcagatattgtcctaagcactttacatctgctagctcattttatcctcccaagagccctttgagaagtttctgttattacctccaatttaagatgaggaaactgaggcaccaacggcaagcttgctcatgagtggtggagctcccctgttcatttaaccatgttgctgtattgcctggtgctgcttaccacaggtgcctggcccataggactttatgtcctggactgggcaaggttatagaagatgttttagttattagacaaagaaagccatgctgttccttccacacaccatctcattcaaaaccaatggtcactctgtttgggggccactaggagagatgaggttcagagaactccagtaaattaaggtcacaaggtcacacagatgaacagtggctaccactatcagctaggattcaacccaggtcggactctacagctggtgggatgcctcccttctcttagtatatgtgctctgttttgccgctcaggaggcaggaaagaaagatcaaggatgagtaggtgcctgtactcctagttaccttgcgtagcgctatgtgactcttacttgtatttcccaggcataaacctggctgcatggcaaagaaagccaggaaggcagcgttctccttttttgtgtagaagaaaatactcttgcctcagcagcagagaactgtgtccacctactgtttattttcctccccctttttctttttttcttcatcaaaaagatatgaatgctgagatcatcccagagaaatctagaataaataccatgagaaaatagtccctctggaaggcgtcttgtactcctgctgtttgtaaatgtgaaaagctcagcaaccgggcagctggatgggtataaatatagcccacagcactgacgcccgctgaatcctgctttcttagaggcacaatgggaacccacggaagtcagagaaggagcagtaaagcccagtcgatcaatcagcctctctctttctctctctccatcctggaccttagtgtgggcgtgaggatagaagcagctttaagagtgcacttaaaatagctcttggaactggacattggctaagctcaaagacaaggaagatgcttcctatgaaaccgacctctgcctgacccttacctggggctattttgaccattctggtccgtgatcttagggctgtgttctttctcagttcagctgcttgggcacaagcaggttttctagtgcacgctccaggcgtttggcgcctctctgctgcctccgtactcagagatcttattctaactagactcagctgtctgaagcctggtgattaaaaatagcctcctggtgctagctgtggaatcaagggagagatactcagtgagcagagaggtcagcttgacgcaagacacctgactgtcatacctgtgggtttccttgcacctgagttttttatctcttgaggtattttccttttatactctatcagtgcccattttttatggctatgcaagataaaccagtcatttcagatttgtggggattagtagaagtctgcattttggcaggagaaaaccattattttacttggtggttcccagaaataaagtctctagttatcattccatgcagacatcaatgagatttgccattgaggcatacatttatttgttttctctttctcatttggtggtaaatacttcaaagtaaagaaccatgtcctctacttccgttgtaactgcccacaaaacttaataattcatcttacatgtcttgttttgtgaccgttgaactgaccgtaacagcctttctctgatgtccatggcatgccattagtaagagtcacaaatactgtgatactcgcctaatccatttttaaagtctgttcagggatagtctaagagtacaaggcatatgtgcatatttgcttttagttgtttcattcaatgtaagctggatctgtgccttacttttaacttctaagattgcttccttcaaattggacctcagcatataatatttataaaaataaagcaggaactgttatttggttaaagcagaatatacttaaaatattaaacttgggataacttagagaaagccctgaagttgaagagcttggggctctgtcagtccgctggtggccggagtgccccagcaccgggccttcctcacaaaacattctgcctctgattccctatccttggaactggtgcccagttagcaaagagcaccagctgttctggccttggggacgatgcatggaggaggggcaccctccaaggaaaccaggccaatagagccaaagtgaacaatttcagtcatcattgggaaccatgatagattatgtagcactttaatgattgatttcctaaaaaccattcatgcagtctttggaagttcagcttgtcacttactggcgtgccctccagggcttctgtccacatcgatttcaccactacatctccacccatggaaggcatgggccaaacccattcatattccagagaacaaggtgcagtttccatggtatacagaaaggtaatattgtacagtgggcaataacattccagttccggctctgctacttcctacttctgagcaacttggaaggttgcctaacctctccaaagacctagataaaatggggttaagaatagtgtccacttcatagggtggttcgaagaaataagtgaaaatataaatgtaaaacacttagtacagtgtctgtcacacagcaaggactcagtaaatgttagccaagaggggaggaaggaggctgccacccttatctgttagctcagcttcaaaaagcctagcaaaataacatcatgaatttgatctgagtggtcacagcaaatccattccctcactgatgaagctcaaattaccttggccacattctttagatttttccctttgcctctgaatgaagcattcacttgacgtagataaaatacagacacctcagcctccagttcagccactgtccagtcaagcaggaaagtacattgtgagtgagtcttaagtgaaaaggactagttatgatgcatttgagacaagaaagtagatcgagctttaggcttttccttttcttgccggaaaggaaaaaaacaacattgcattaatcatcatctggtcctcggccaagattcgtatttacctgttatgtgccatattcatactcaccctgtaatgtggcagcctcgtgccatttatcattccaacagcccataacagatgaggagacacagatgacttgtccagggtcatcaagcttgtcaatggcagagtgaatgttggagctcagaccttctgactctcaacccaatgagatacaaataaaatagtggaaaccaccagccagccctcccaagtgtttaggggcattaaagacactggccatcctaaaggcgcctcttactcttatacatcatctaccactgaaaccagagcttccgctgtgcactgtccatgcgtggggccgatcaaggacaccaaggctgtgcaatgacaggtttcttctctataaactttccttgtctctttcttttaagacattgaatagcactaagggataatttcttccttttcttttttttttttttgtgtgtgtgtgtgttttgatttttgacacaggttctctctctgtcacccaggctggagtgcagtggcatgaacatggctcactgcagcctccacctcctgggctcaagcaatcctcctacctgagcttcccatgtatgtgggaccacaagcatgccgcaccatgctcagctaatttttaaaatttttgtagacacaaggtctcactatgagctaccatgcctggcctagggtaatttcttaaagtagagctttatctccttgtcgagaggcatttagatgtcctttcttcaatgaaagattctttttctcctctaaagaacccagaaataatttgttggtcctttactatgatgtccctaacagatctaatcatgcctctccattttgagaccattcattctttcaaacaccattatttttattcagacagtgtacctagcaatagtcccggcactgggaacacagaaatgaaaagttactctctttaagaaattcacagttccaaaacctcggctgtacccacagagtcagggtcatagggaagtttcagtaccagatagtgggaggagggccagggctgccagtcgttggtgaatgtggtaaggtggcaggctgttggcagttgagagctggcatccagcattaaccttagaaacaaagagctcaaggagcagaaatccagccacagagactgggattcggtcataggacttctgttattgagaaggactggaagacagagctgggtaggaaccaagataggcatcccattacagcaaaggcttgtttttaggtggcagacaagggcacagaaaagccagcaggagtgactcaatactggttccctgaaccactggtcccaagcttcctcttgataaagggtaggcctggtcccggagtctgggtggagctgaacttcaaagtaaaggagggttaagtaagcctaactggaaaggttctgggccgtaggggcagacagccaagcatctcactgcagctaaaacccaagtaaaataaataggacttatggtatgcacaaatctctcataccagagaaagtggacattccaagtttgtgtgatttgaaggaaaagattttagttccgtggttcttgtaggttgttactagattgctagaatgcttataatataagatagttttgtgtgatgggtttatttgggaatgtgatgatagaaacaaaaggaaattcctttgttttttattaccatacaaggataagattttgagattatctttccaaggaaaacttatcaactatctattatgtatcaggcacaatcctttgtactgggattccaaagacaaataagatagttcttgacctcaaggaacttgtaattcgttggggaagacagataagtaaccaacaaattagaacgtagggtgattggagtgtgtttcatgtcactgcgccacagaagagaagcagttgacttaggagaggagaaagtttaatttgggccatgaaggaaattaaaagtaaagacctccaaattggtgggcagccattgaaggattttaggaaagtgatgtgctgagggatggattataagatcgggggcagaaagaccagttacaagggaaaatcatgctggtcatgccccagtacttacatttttctcataccacctcttagagcccctgaaagtgtagactccactatggagaagcaacacaaatgtgaaatactagcttgcgagccacgtgggtagctctttcctcttcaggaaaggggaatgctagtgtctagaagtgggagagattgaaatgattctgtaatgaggaaataagttacgttagggcacatatataaagaaacagcagaaaaaatgtaaaatctcaatcactctgtagccatgacatttataaaaatgaaaattttacaagagtatttctaaatgctttgaagttggcccccaaaaagcctgcctaccttcactttgagttacaaataaaatccctagtgttgcatagctgtgtgagtggtatttacattctcacacatttcagatctcttgagtccattttgctagtatttgttttccttggagataggagttaattcagtcacactttcttcccctgacaataactgcatttttttctctaaggtagctttccctccaaccgttgcttgttgttcttacccaagacccagtatctgccaaaacaaatgtatgtgagtgataaacgctgagaggaatgtctgttggaccacaggaaatttcctgttcaggaagagatcttgggaacttcctgtggaggagtgcattgtgggacttttggctgcagtcccttgaccacagctgcaattaactaaaggcagagggggaggggagaggaagaagaggagacagctacccacagacattcttatccatgtagcccgaggagagaatgggctcatacaggttcacaactttgcaagtgaactagatgtctagtgcagaacaagatcactgtgcccaacctcctttactgccatccatctttcacagccttcccggtgctttcctagggctcctaaaaacagtcactgtcacattatccatgtgcattccccagtccctcataagcacacatgaagtgtccatgccctctgtagccccaccaactgtttggaatctcttgattagcttctagaatccaggcattggagttggtggtctttctccatcccaacttaagtatgaaagagaggagaggagaacccgtggctctttgacataccttcatgtgaccctaaaagcattagggaaacgttatggaactgttacgaaaaaagaggttaataaatccaattgataagcacaatcaaaaccaacaggagaaccatgcattcttgtgctgcaatgccattacgaaatgaagtcctcgttaatacctcatactccccttcttggtccccaaactgggcacgtctactttgcattcaagtgcctgatgtgtgtgcgtgtgtgtgttttattttttattttttatttttttgagacaacctattgcctaggttggagtacagtggcatgatcactgctcactgcagcctcgatggcctgggctcaagtgatcctcctgccttagcctctgggtccctgggacagacaggcataccaccatgccctgctaatttttaacaattacttgtagagacaaagtgttgctatgttgcccaggctcttgatgtttctttacagccttcactcagtgcttttatggagtccatctctgaagagcagggcttatatttgagtttcaaaaatctaaagtctcagctggtggcaatttacatctgataatatatatgaccttggaagtctctggcagacataggaaaggggagtggacatctaaggactgaaaaatatcccaggccattttctgggtcatggaaaagtaacctgaccttcttatcccacctcctattcatcatccatctcacctcccatccccatcacattttaaattttaattaatttttcattggcagataaaagtcatatgtttatggtatacatacataaaacatgatattttaatgtatgtatacatgtggactggctagatcaagctaattaacatagccattaactcacatacctgttttttgtggtgagaacatttaaaatctaccatcttcataattttcaaacatacacagtgttattaaccataatcaccgtgttatacaacagatctcctgagcttattcctcccatctaactgacgttttgtatcctttgaccaacacctccccacccgcatcccttcttttactctgcttgagttcaacttttagattccacatgtaagcgagatcgtgtaatatatatcttttcattactggcttatttcacttagcataatgtgcaccagattcacccatgttgctgcagatggacttcaaaatgtaaaatattcttcatgagaatgaagttacccccccagaaagggggaccagttactccccagaaagaattaaaaagtagccgggcatggtggcacacacctgtaatccagctaatagggaggcttagttggggggatcacttgagcccaggagtttgagaccagtctcacagtctcagcaacatagccagaccctgtctcatatttttaaaacaagaattaaaaagcaagaatagtttgcagcaaacattttctgagacttttctagataatactaggtttagtccgccctccaaagtagtataattacatcatggttttaaaattaaaagcaaaaatataggtgaagcaaaagtgagctttattcatcgaaaatttgtcctcactcatcccatgtgactttttaaagtgtaattctaaggagactcccagcatccggatggcaaatacgtgaacagagaaacataaattaactggaagggaacaaaatagacctctcagctgaccaattctttgtagctttgacattggcctttaaaagaacgaggcaaacttttttaaagaaagttatctaatttaatgcaagaggtaactctctggctatgacttagggtaattaggcctattttctacattttggacgtgtttcctatggctggaatgtaaaagcttatttaatattgatgtaaccccaaaatttaagtaattggaatgcactatttctatcacattctattgagctgaagtataggttgtgctctgttcaattcaggttgtcttgacaaagactgagaaatagaatggccctccaagggcatacctgactattctgcggataacacagtcacgcaggcatttgctatagtgaaaatactgttatggttaagctagtggtatcttgtatttcaatgataccttacggtttaccagttttcatgtaatatattaacctatctcatttaatcattacagtgttccttggtgtacagatgaggaatcaaactcagatatgacgaatcaaactcagataggctaagggtggaggtagaccaggagaacctgcacccgcatgcattcttctcactgcaagttcaggcaggccaggagatgaaagccagcatgagccagtgaataagcaattatctgacaaacccaaggccagcagaagtaagataacatctttgttcccctccatttcttcctatgccatccagccctcaacccctgctaccttcaagccctgttgatactcacgccctcctgcccacattccttcggtggggcactgaagttttggtggaggggagcagagcgaggaactctttgtttgtgcagtaaggacttccttacaggaaatttcctgtgatggaaactctggggaaacttcctgttggttaggccactgccgggggctgataacctgaaccacttaaaaccttcagggctctgctgctgagaggatgggatggcccttccctgcttctccaagtctgggagggcaagaaagtccaagcgtacagaagcctcaggaatctccctgggatggtattttccaggagctaaaagctatggctctcatcagctgatcttattctagaagaccagccctcatggttaaatggactgcttagatttataacgagaatggtacctgcagcgacatactagctggatgcattaatagggctttggaaaccatctcaaagttctgggtctggttcttcatacatcattttaaaacatattcctgttatattactctccagtgttaagggtttatgagcactgcaaacatttattttcagctctcacttatgaacttggaattctaattaatccttgtcattgtcagacatggttgacactgtatgctgtgtgggtcacttagcaccaagtgtaaatcatctggggtatgtcttgcaggacgaccaatcttaagccatagcacaaaattcttaaatagactttacaatcatttcacctagtttggacttctggtggattattttgttctttgtcagcactccagcaaagataattactcctaaatcatgccttggattgaactgagctttgaatgtctccaatgttgggaattccgtaggttcccatagcagctctcctggcccagctcttagtttgaaagtttggtctcctaattagcctgaattttctctagtgacctataagcccattattgtaagccctgtccttattgtgtcataaaaataattgactccttgtcttcaatctatttcatataggtagacagctgccacatcaagaacgtcttgtaagtgcttgatttaccaaagtccatttctccacaagtttcttcatagttgttttcctggattgggggttattttagccttactttctaaaatgactggatttaatggaggcctctgtcagtcccaactctccaaatttctgccccagcttggtgctctttgctttgtttatagtaaataatcatttcagcatcagaataaatcagtgaactggacatgtctttaggtggacttttgagattatattcctggtccacattgcaaattagggaagaatgaaagaaattataattaactaggaagaaatttgtccgctgagaagtctcccttgttagactcctagaaaatagagaccataatattttataattttttgtcacagtttccatacatatttgttgaatgaatcctaaaagtcaaaagggagctgcttattggatttgagttgcttctagaacatgttaaaaattatgtgaattttctcaatgtatttgtaagaataagctgtcctcgtgattgagtattgatcttcaaaatggagaattatcagtggtgagtgatgctgcatgtaatgtacttataaaggataggaaaaatattttgtaaaaatgccttgggataagctttaaatataaaaatccaaaatggtgatttagtttggggcactcagaggaagaataagaatagggcctgtttaaattttttgcaacatgattactcctggttttacatttgattttgtattatagaatgagattaactggaatatgttataaatacagaaagtctgtcattataaacactagtcagcaccttttgtcttcataagaataattgctattttcaatgcatacttgctctaaaatagaccatattgatgtcataatatgtagactgagcaaaaatattattagtatcatcatcatcatcatcatcttctgatcttcatcaaagttgtattaaaatattttggtattctaaatatcattatcttttttgaaagcctacttagggtaatagaaggatcaggaactttggactctttgacagatttctagttttttctatttttttgctattctgtaaattctttcaaaagatttttatcaggtgtttactgcatactctgaagtgctgggatatatcagtgaccaaaacagatatgacccctgccttcatgttgcttacagtttaataggggagataagtgtctaccaaggaattcacactagccagagagcacattgcccacacagcatccagagtaatctgttgtaagataaatcagactgttacttccctgtgccaaaacctccactggcttccagtttcatttggaaaccttctgagttctcagtatgaactgccaggccctgtgtgatctgaaatttgtaaatctcgtcaacctctccaccctcatcctccatcaccttgtgttctgctagttcctcttcagccaccctgatctttttgctattcttggaacacacaagtttatgttctacctcagggcatttgcacttgctgcccttctgcctatgaagctgtttctccagatactctcatgttagagtccctcaccttatccaggcctctgcttaaatatcacctccttagagaagcttccccaacagcattatccaaacgacaccttccttcactctccatcacctttatcctgcactatttttcttcttggcaattttcaccacctgacatgtaattttttaaaacactggatggtgacccttaggtatctattcaaaatggccattcttgtgcacagctggggtatgatgccagtgggtccacctgtagggtaattggatgggcctggctgtctcatgggagactccagagtcttttcttttgggaaggcaagtttgtccaaaaagaaatcctgtgggctcctccttagaaagagtcaatatatgatggcacttgagcttttaccatgaagatttccactgagttcccctatattccagagctgctcacccttgctccctcctgaacttggcgttctcagattcatcctgattccagcaagggaaatgtcctttcatctgcaaggaggtgcaggttgctgggctgggggagggtggaatacagccatgccacagatgctttattagtcttctctgttccctgcctgccttcagaggcacttggaacccccagtttctgaggttttatgaagttctggtaggaaaacgagtttgtttattggtatctccccctgcaggcagattgagttaagattttctgctcagctgagtcagataccaattgtccgtctgttccagcttccaaaattttgtgcacctccctcaactgccattgtttcctctcccattctgttgtcttgtgggtttgtaccttttttattcctctatctggtttagtgggacgtcagacaggagcagggatagacaagatatgtctcatcaatgtagttacatgtggaggtaaaagcattgatgtagcagtacaggatgctttgaaagcataaaatacagaaatctggtctggtgtgtcgcatacatttaagaggagacttgaaagctaagtaggagttaattgggcaaagaagaaggtaggattatttctggggaggggaacagagcctgtgaaggccctgagactggaagaggatttgttctacagggaatggaaagcccagcccaactggcattcattgaatttgctgtggaaaagcagagtgacacattgctttggtgaggattgcagatggtgaggtagaacaagacagtcacgttaaggattttgatctttatcctaagagcaacaagcatccgtgtctgtgttacctcagtaaccttggacctcgtaaactctgggttcttttataaaccaggcacagtaatatctacctcacagagctgtgtgcattgcatgacatcatatacacactatattgacttaagactgtcatatttgtttgctcaactcatggatatgaagggcagactgtataccttgtcaggtacttttttctttttattgtatcccatgggggaaatggatggtagattggtggaggtggctagaattgtaaagaaaaatatgaaattcacatgaaggctggagttactcatatgacaacaacagcttatggggcccaattctattgctcttgcgtctgtcacacagttgaggttttggaatgaactacgtatgccatccttgttggacagtgacaagtcactgtagccccctaggagaagaacagtataatcaagatgtcaggttgaggaataatctttgtgatcaactgagtgaaaggtacaaagtaagaaacaattgagaggcattgctctgggcccaaaagtctagtaacaggcaattagaggttaaaggataagcatttgatagtgttctagctatactgcaaaatgaatgctgcatgaaggctaactgagaagcttatcagttggtagttgggaactaaagaggaagcaggcaccaaggaaagatggaaagttctacacacctttgttagttacacatggaggtaaatgcattgatgatgaatctgccatttttaggttttcatgtttaaaaagtttcctgacaatgtgtctagcaatgtgctagacactgtagataatatacaataaaatattaaagaggcggctcgtcctcaaggaactttcaagcattcatacatttctgtttgattgtaacatatttattattttaaagaactggccagagagttgcatgttctgtgtagggataaatagcaagtttcaggcccctgcatttattggcacttacgtacatggtagtcctcacacctttgccggacagcaccaaaaatcctgcggagctcaactctgtgacctcaccctttgagcctggaaatcagcatgaactcatgatctgacaatcccgaacaagctaagcatttcttaaaagaaagcagtgacttttaataggtctcactgatggtgttccaccgattaaatcagtcaaacagaattgtcacattgaagattatcttgggtttctagaattcattctaggttttaaagatgcagacagattctgagggtcacaaacgtcagtttgccaagtatgtttaggctgtaaagttatccatgaaaagaccgtgtggtttgcatgagattgattgtgatcggatgtaactacattgctttttacagctcagtagaaaccactttcaattgcaaattgtagcagaagcaggcgtctacttaattttgatcttaaagaatttcaaagagaataaatgcacactaaatagtggtggattttggtagtatacgccaacatcactttttatttaacacatgcagaaatgtgtactggtagagaaaattggtagagtgaattaaagtacagacagatccacattgactttaaatgaatacatgcacggtcagaaaacggtaaccctgggcaggaatctttaagttttcagggctcattagacagtgagcaggtgaggctcaaggctgagtttgcaactgtgctgtgaaaactcaaaaatgaaagtttttttcatttctgagcctgttgaaaagtgggtaagaagggaaagggcagtctcataataaaataatatgtaaaggctaaccaaataacccactttagtctttacttactggcaggcagcaacatgtcctatttacagagggaggctttacctaagtggttatatgtaaatgaaccaccttttcaatgaaacaggatgcacctgtctagaggtgtctgttactagagcaatggatgtttgattggactgacaaattttatttcctttttggaaaaaagaatgtaggtggtatcaaatcaccctttctcctggtaacaataggaattacttcttgaacttgagaaaccagtagttgcttgacctatgttttttgcatttagcatagtcttctaaagtagtaggagcatacgtttttgtctttaattccctgcagaccccttccccaatgatcatcagcaatacatccacccataaatcaatgaaatcataaaaaagaaacataaaattcacttgaaaaagaatacctacaaggcattcctagaattctcaatgtaagtcattttttgccagcaaagctaattgttctcaaatatttgataacaaggatggcatacgtagttcattccaaaacctcaactgtgtgacagacgcaagggcagtagaattgagccccataagctgttgttgtcatgtgagtaactccagccttcatgtgaatttcatatttttctttacaattctagccacctccaccaatctaccatccatttcccccatgggatacaataaaaagaaaaaagtacctgacaaggtatacagtctgcccttcatatccgtgagttgcacatccgtgattccatccagccacagatagaaaatattcagaaaaacaaaaaacaaaaaaaaaaaacccacaaatgcaacaataaaaaaatacagactttcagaatagcatataacaactatttagatagcatttacattgtattcgttacaagtaatctagagatgatttaaaatatacaggaggatgcacataggttatatgcaaatatacaccattttatataagggatttgagcatccttggattttggtatctgggcggggggtcctggagccaatcccttgtgtatgcagaggaatgactgtactggaaagcaaagtaaagctacaccaaagagggtaatagcaggctataaagtactgagagaaactaagagaagaaagtgaggtacctctgacaaaggagtgcttggaagagaaaggatgaatagaaaaatgggatttcagggaattcattgatgacaggatgacctacttaaaaatgacaagggccatgtgtggtggctcacacctgtaatcccagcactttgggaggccaaggtgggcagatcacttgaggtcaggagttggagacgagcctggccaacacggcaaaaccctatctctacaaaaaaatacaaaaattagctgggtgtgatggcacgcgcctgtagtcccagcttcttgggaggcatgaggtgggagaatgacctgcgctcaggaggttaaggctgtggtgagccgtgatcgtgccactgcactccagcctgggtgacacagggagaccctgtgtcaaaaaaaaaaaagataggaagtgagcaaaagagaggtcatagataatggcatgatgaaacagaggaaaaaaaggataagaaggaggaagaaaaaatgtacaataaaacgagaaggaaggatgcaaaatgaatataggaaaaacaagagaactgaagacaagaggaaaaaaggagatggtagaagggaggaatgaaagtaagtaaaataggaatgaagttcaagatctatttagccatagctaaaggaacgtattatcagctattgatttcctcttctaataaagaggagaatgatatggatagtccagaataatggatcatttgcaaaacattaatgtgtcttgacttaaggtacatgtttataattacctcgaactgtatatttgtcttcttccatgtgtggttttaaattattttaccaaaatggggaagccaggagatacatacttgattcagtaaaaccagctttttattcgtagtttacttcctgctgcatccttttttcggctgagagggatcaatgtctgcaggtgtcccttaatccataattaaaccatatgcacatctaccctcccagtttccctgcccaccataaaaccttcatcttggctattgcttggaaaatggggagggaatagagcttctgcttttttttttttttttttttttttttggagagagtcttgctgtgtcacccaggctggagtgcagtggcgtgatctcagctcactgcaacttccgcctcccagattcaagcaattctcctgcctcagcctcctgagtagctgggactacaggcacgcaccacaacgcccagctaattttttgtatttttaggagagacagggtttcaccatgttagctaggatggtctcagtctcctgacctcgtgatccgcccacttcaacctcccaaagtgctgggattacaggcgtgagccaccgcacctggctggaatggagcattttacttgagacctctgtgagctggtcctgagtatgagtagtgaattccaacccacttttctcctaatccctgtcgtgtcctagagtgagaatatacagaggggttggaagtgggtttatgttcatgggctttctagatggactttcaagtccaagtagaagtccaacttgagagctcgtgtagaaagcccttgaatgtaaacccgtgaagaaaagcacttccttatttcagaaagagcacaaaagaccagcaatccaaaggcatttcgaagaccttgaaagagctgggagtggcttaggtatggagggtgcatgtgtgagtacaaatggcccctcagctcagaaccaagcatctgtgtgccatgatccatggttcctaaatatcatggagggcaaaaagtataaatgtgaattaatgcaagagaaaccacactgaaataaaaaatacctgtggtctcttgagattcaagttggcaaaatccaatctaaaagatgtctgactatcacaattagtgaagtccacaggtcagattttcctctctgttcctgattaggattaacctggaaaggtggattgggtccatctttaataataactacaccttctgaaagatttgaaactattattatctctttgtttgctgctgctttcatagatttatggtaggacttggggttagatggatgatccaggtcacctattccagcctcacactctgcctctattccaaatatgacatcactgagcgatggccacccaccttcccttggatacctctaacaatgggaatctcactgtttcacaggcagctcttttttttttaaacacctccaattccaatttttaaacacctccaatgtccagaaagttctttaaactcttaaatacatgacatttattaaaacaaagcctcaaaccactttcagagtggtggtacagcaaataattgattcttttcagggtttttcccccctgtgttttttgtcttctctaactcaaatcattgcccggatattgcttataacaggagctcagcctcaaccagtctttctgcttttaccacatgctctagtggaatctagattaccggactcatttcgaatctaactaaaaatgagaaatgctctgtttatataaagcttggcaatcaataggaaaaggaaaggctgtttagcaaaggccagtgataattccaagaagcagtgtcttttggaatatgctatggtgacctggccactgcagctatctgcttatctgtctggtttcagatttccagaaaggactcgctgccttcgaaacacttcccagcccttccttgggtgcctggatttctaccattagcagtttactccttgagcatggatttttgcagaaagacatgtagaccagggagatggctcattaagctcttggtggtcatttcaccagagcatttagcatttttaaatcccttcctggatgcttgttttagtgatgatggtggtggtgagatgaagcagggatgtgtagatcatgaagggatgggaaaggggatgatttggttggaacgaagcaaaataacatttctgagtgcctgctaaggtccagcacttttcacacgtaaaattttcacaacaatccctgtgagttgggatccagtttctacagatgaggaaatgaagacaggagactagatgacctgaccaggtcctaggccagtcaggaacagagctgggatttaaaccagagctgcctggtccaaaacctgtgctggtcctgctattttatgacacctagaagggaaaagggcagagaactggaaagctgagatggaatcacctacttaagaattttgcagaaggctaaccctgtctaaaagcatcgctttgcaatcagacacatgtggcagtaaaaatgtcccaatgctaactcattttaagccttggttttagtctacctaaaatttatagaacactgtaaaatatgctacatgtaataagatatttattaggagtcttcttgcacttgttcagaatgctttttattatgtttctgctcattattgtagatattctcccattttggagaaattgaccgcgtgcacactcttcactggagttttgcacaaatcttaccagaaaactggtgaagatcagtcctgaggccccttccgcctctaattccttgcagtattttggctgtactatgtgctgttacactagatcacaataagcagctgccagtttcctcacaggttcccttggggaaggatatgggaataattcttaggaaccccccacaaattatagcaaccaatgcagtaaaacccctttagctcactcctccccctgtgctcactcacatactcaaatatatttagtgctttccttcctctcactccgacacttttgggagagccctgggggtggttgagtaccttgtccttgggtggattttcttgttttctgagactggctgccatcagacttcgggagaggatcttaccagaggcgggtctcttcttcccccgccaccgccctcagtattctttcacagccaccacaaagccacgctttctctgctaagtacacattcctaggactgtaacaaaaggcttctgtgagtttgaaagctgcaggggaggctcctgcgtgaaggcctaagatttaaagggtttcatttctcctgtgctggggactgtattgagtgcagtttgaaatctgcttcaccaccacaaggggaaatagaaatcagccctaaagctgcatggagaccccggctggtctttatcctcatagcttagtgatttcctgccacccaagaggcagtgaggggctccctgacattccggaggccccaggaaccctggctgcggtgacccggtgctggggagggagtgcatggggtaggctcctggttgtgagccttcagttcgccgggatgtctatgctgtgctgtgtggctacttagccaaggctgtgcatgccagccggagccaacagcttcagcaaacctgccctccggggagagtggatttcattgttttcaaagagatacaggaaatcagaattttccatccttccatggagagacttggattggactgagaagagaaccagcattaaaaaggaagtctccttccctttcttccctgcccctcctcaccccagccccagcccacccgccaggggcacaccctccctctgccggcctttgggtggaaaccttcctgcctgcagcacaggcagtgtgaactgcggggctgtctgagcaacccagctcttgccttttcagtagcagaggtaatgtggccacgactcagaattatgggaccccagatgctgaggaagtaaactgacttcttttcagctcacagcagacgcatagacaacagagtagatttcatgttctaatgaaacagtgtagaaactctgtccttggtggctcagtactgtgtggtatgagcctctgcctccctccctccctccctctttccttctcttttcctcccccaccccttcacggcctgccctttcctgattcttcagaactgacaactataaatccagcccaaattcctttaaacaaagggtcaccaagagcacgaccttgaaaagacttcttgtgatatgcacttgcagagttgcatattgttccttggatttaggacaggccaaagaaatcttccactttatttaagaagagcctgtgtggttcgaggccagggccgtggacataggattaaccacaataataatacaatgaagtacacatgcaaaatgaaaacttaataatgaagaactactgattttttccagggagtatcaagcacttccttgtaaattcttacaaggcatgaattatttcctagaaatgatttaggctcttttggaaaagtgcattgattcttttaacatcataattccagttacggtaaaacctccattatctgattttctgtgaattgcaaagctattattaactggtacattttatattttaccagtacagtgacagctgatatttataatggtgaccccaaagttctgtaaaatgccgaattacccactcaggaatcaaagaaagattaaattgtgttaaatagagtttttcatgttaagtgtcttattttattcagatcttgttttagaaagtccaagtgtacatccaccataattttcgagggcagtaccaacttaaaatgttttatcttttcttggctttgtaaacaaaaccttgtaaaaaaaaattttttttttcaatactttgggttcaaactacagatcattcgatctcaaacctgacaggtaaacaaatttttttttcagagtaagtattgcagacatgaaagatcataaatgtgttcaccaaagtgagtgtgagacagatcaccccaaacactctccactatttctctatgatcttagcccagctgtgtgtgttgtgagtcatgcctgggtctgacaccagcattcagaggctggcatagcttagcatccacatcataaagggctcagatcgatggctctccttgtcaagctctgcagccctctctcaggctttgtgagatgtggtttgcatcattttcctttaaggaaatgttatgcagaaacataaatgcagtatacaacgaggaggctttcaccaagaaattaaaagggtacagatgtctaggaatgcaggggttaagacaaaaatgtactgcagggaaagaaatgtgagcagaatgcccctcttttcgcctgcttctgccttgcatccagcccttcccggggagtgtgatgtgaactgccatccaaggtggctgtggtacaggggcagggtctgggggctgcttctgcagcttcagcgcagaaaacccagagctcagtcccagcaaaagcctaaaggagaaggaaacagaggccctaacacaacagaaaatgatgcaccccactttcctgtggttcatcagttcacatacttcccaggagaagtcaaatcacgccccagaataggatacctacttaggacttagtgatgttttatagtcatgtcgaagaaccttcacatccatggcctcatttgatctcacagcaagatgggcatgattaaaaaaaagaagttactcccactttccagatgagaaaacaaattcaaagacagtgagtgccttagctgagtcctatccaagcaaggcagggggcaaagccaggactaaaacacaaggttctaattccttctccatgatttcctgatgcccatcataccctaatagaataggatcaccctgcaaggcaactggtccacatggagagtcctcttggaggacaccgacccgtggtgccacctctgatttgcaagttggtgcattagacaagccctggaatgacagccagaagatctctattctgatcctggccctccgtctacttgggccactttgatcaagccatttagccccgttcccttcaggtggctcagctggaagtaagaatgataatgcccatgcagctgactcacctggtgactgtgaaggccaaatgagatgatgtgtttgagagcactttgcaaactgtggagtattgccagccatttctcctaatttggctcagggcagaaagacatccaggtgaagagcttgatgtgtgtgatgacttaggcttggggatatttgtgaacaggacacaacaaataaattcgtttgacgggcagagggacagtatgctgggtatagtagaaactcttggaacctgcagttattgaagggtttaaaactcaaaaatgatacagaaggcagttctattacagtttctgcttttttgctgttcaacacctctgaagatggtgcgtttaatccttcctgggatggcaggttatagatgacttcatgctgaaagtgacccgtaaggaatatccgtatattttgaagtgtatgtggttcaccaggcagaaaacagtctgccaagtctctcatcccaggcagaaggggtggcacatgcgaaggccagagaatgcagtgcctctgagaactgtcagatgtcagtgtggctggagccaggtctgctagagggtgaggttggaagttaaccaggagtttaggaggccctgggaagcctctgaaaatatttaagcaaaaacagttagctttatgtttttgttgttgttgttgtttgagacggagtctcgctgtcacccaggctggagtgcaatggcgccgtctcggctcactgcaacctctgtctcccaggttcacaccattctcctccctcagcctcccaagtagctgggactacaggtgctcgccaccatgcctggctaaattttttgtatttttggtagagacgggggtttcaccatgttagccaggatggtctcgatctcctgacctcgtgatccacccgcctcggcctcccaaagtgctgggattacaggtgtgagccactgcgcccggccagctttatgtttttaaaagatcattgcctcgttcttggttctcagctctctgatccttttagtttttattggatagaggagagaccttaatgtatacagagactaaggaacttggaattggaaatctgcctggctccctttccctcaacgtgcctgcagtcagccatctttctccagctaagccaaaggatggtgggggcaccacacaacagagagctgagttagaaagggtgggagggaggtggctgaagggtccccactgcagttgacaatttgaaataaactcccactttccttcaatattttcctatagcaaatggaaactttgaccagataccagcttctttccatccttccattagactcacagcccagttaggatgttcacagtcccaagccagggatagcacatgccattaccatagtgggctggatagggggcaggaagcaatgctttccagcccttagaagcctctggtgatgtgctgtgaacatggtgagggaacccgaactttagagtcaaacaggctggtctgcaaaccccggttccagcactcactggactcaggatcttcccaagttagttaactcctctgaccatgtctgcattttctcatctggaaaatgggaacagtaatatccaccctccagagctgttatgaagatgaaattgaaagtgtctccaacgcaccaggtatatagtataatctcaatttatgcttttgcccttagatgccagaagttaatattctttcaatacttattctcgtagaagcaaaagagatcctgtttgtgtgatgttcctacagcctctgctgtactgtattatgaaagctcaagaaataggagttaatgatgaggcacagcaggaagaaaaatgtctctaagatgccaaacagggaaaatttaggaagaaataagaaccaaaaaataaatcagttgatcagggtaaaaaatgtcttatgctctgaacttataactctatggagtaatttccactaatgatctcagtgtctacttaactaatcatgtacttttctagccagaaagacagtacttgtagggtacgttccattttaatgcacaaatcattgttgcttttcaaaaagtcatctttttttccctggccacacacagcaccccgaggcctggccagcccccatgccagctcaagagcatgaactcttaccggaatgggttctgtggggagtacaaagtcaaacataagtaggcttgcattcagagctcactgcctagcgggaaaaaagacatatgcactcataactctaatttaacacggcgtcctagaaattccatatccatcttcaaagttctctggaagcaaaaagacaggaggaaagacatttttagaaggaggcttcctggaaggagtgacttcagtttcaggccttaaaggagatgtgagtttccacatgcaaggagggaggaaaagcatgttgcagacagacgaagcaagtgtgtgagcaaaggcctgcggggtgacagggaggggtgttgggaggtgtgtatctggatgaaggcaggtatggaaagtgctgggaaaaaaggtctgggaaagggccttgggggccaggtgataaggaagttgaatgcctctttgaagagtggggttttattttatgtgcctttggtggggagttgtgaaggtttttattcaaaagagcagtggggctgggcacagtgactcacacctgtaatcccagcactttgggaggccaaggtgagcagatcacgtgaggccaggagtttgagaccagcctggccaacatggcgaaaccccatctctactaaaaaatacataagtaacaaaaattagctgagcgtggtgatgcatgcgtgtaatcccagctatgcaggaagttgaggcacaacaatcgcttgaacctgggaggcggaggttgcaatggcctgggattgtgccactgcaccccagcctgggcgacagagcgagagagactctgtctcaaaaaaaaaaaaaaaaaagcaatgggaatggttcttaaggaagatttctctgggcacactgtatgttctcactgaatatttggtgtggtttgggttttataccagttacgatatggggcttggtatctctgagcctaaaatctgtgctttccaaggacagtagaaatgctagtgttcactttgctcagtactcttccctccacttggcactcatgctgcttctcaagccttggggtgtccctggttctggagtccctcctacccctgcagcccttttctaagctatagtctcacccctcctctgcaggctcccaggcagcctcacctcctcctgcagagggatcagctgtcaccctgctcctccctcctggccctcagcctaccctacccacatgcattcaccatcttaccagtggaacaggcgtccctctcctgtatacagaggtcccttcccctgagctcttctgcctcagcctccccagtgggcttctcccctccttcacagtcagcctctttctcatttgcacctagttccattccatcttcaaagaagcccacttggattgcacatctctcttcactaccacaaatctggttcctcacagccaagttctggaaagagttctccacatggtgtcctcctcctcacctcccattagcgcctcaaccctccgtgatctggtttctacgcctgctgcccctctgccctggctgcagtccccagatgccgaatccatctgccatctgctgcccgaaactctcctggcttgcaagataccactgtcctctgacttgtccccggtcttgtcccctgctgttccctcctttaagaatacaccttttctaaattgcctgctgcctaaatgttttcctctggaaaactcacctactccagcggcttcaacgatcatcagtgcgatggggacttccaaatattttttatctctagcctgggcctttctcttaaataacagatgcatgtgtacaactgcctactgggcctctttacctagatgttgtacagttacctcacattcaacaaagctgaatatatcccccgccaatccatcctctgtgggatgagatgggcgggcccctctattcatctagtttcaggaatcaggagcttggattttgtcattgactgcttcctccacattgaattaatctcttaagttccatcaattctaccccatggtatcttcagcccatcctttctccatcccttctgctttagttcaggctaacaatatctgtcatctccatttctgcaacagcctctgactggccgccttgtaccagtcttgccctgttctaattcattctctactctgcagctggagtgatcctttgaaaacaatatccgattatgttacgcccttgtttaaaattcttcagtgactccttgtagccttaggataaagcctgaactccttagtacagcagacaaggccttccaaggccttcctaataatagctaacacttatgaagttcgcaatccataccccacactgttctaaggtccttacacatatgtcaacatacttaatgctcataattcctgacgtaggtcctgttactatccgagttttacagtggaggaaatcgaggcacagaaaggtaaggcaccttgcacaaggtcacacagctaggaagtgggagaaccaagattcaaacccaggaaacctgactccaggcccacactccttagcaccatgctgcactgtctccatcatgtgccgtagcttctcatccattcagaaatattatattgagtactgaaaatgggctagaagctgcttgagacactggggatatagcaggaatccaaacaaaggtccgttcacatgctagtgaagaaaatgagtaagaaacagcatgattagtaaaattattaggtagattagggatgaatgctaagagaaaacaaaaataagcaagaaagaagaccgggggagtctgagaacagatatgcgatgttagaaggagtgtccagggaggacttgaaggaagtgaggatgtctaggagaagaatatcagctgaaggaacagcaagtccaaaggccctgaggtgggaatagttcctggtaggtctgaggaacagcagagaggtcagcgtggctggagcagaatgaaggagggaaaggatgatggcaggaaaggatggtgggagctgggtacagacaggtcaggtcatatagggcccagaagatcattgtaaagaccttggcatgaactgtgagtgagagataggaagccactgggggattctgggcagagtgacatgatgaaacttacaaacagaggagtgatgtgatttgaatcaatctctggcaggtctgttgaggatagatgcagggaaggaacagtggaagctggcagaccagtcagggaccagtaacaatgatccaggcaagagaggatggtggctgggactaatgtagtagcagcagaggtggcggaagtgggcagagctggatacatttggaaggcagagccaataacacttaccaatggaccagaagggggtgtaaaagaagaaacaaggggctgggcacggtggctcacgcctgtaatcccagcactttgggaggctgaggtgggcggatcacaaagtcaggagatccagaccatcctggctaacacggcaaaaccccatctctactaaaaatacaaaaaaaaaattagctgggtgtggtggtgggcacctgtagtcccagcttctcgggaggctgaggtgggaaaatggtgtgaacccgggaggtggagcttacagtgagccgagatggcgccactgcactccagcctgggcaacagagtgagactcagtctcaaaaaaataaagaaagaaagaagaaacaagggcgactggaaggtgtggggccttggccacagaaagaatggagtttgccttctctgtgatgggaaagattgggaacagcagatttagatgagtgacatctggaggttggttggcctcattaagcctaacgtgcctattaagcatcaaaatggagatgttgaccgggcacagtggctcatgcctgtaatcccagcactttgggagtctgaggcaggaggattgcttgaggctagagtttgagaccagcctgggcaacatagcaagaccccatctcttaaaaaaaaaatgtgttttagattagccgggtatggtggctcacttgtagtcctaaccacttggaaggctgaggcaaggggatcacttgaagccaggagttcgaggctgcagtgaaccatgatcccaccaccatacaacaactctgttgtctgggcaacagagggagattctgtctcttaaaaacaaacatggagatgcattgtcacctcccaccctcacccccatttgaacacacactcacatatatctcacgtcctaatcctactgaactactcaacatttccccaaaccggttgtgctcttttcatgtgtgcaggccttgcatccgctgttcctctgactggaaagctgttggcccatccagttgagaaagtcttccctggccgtgcacgaatgcactaacttgtaaatgtctctttacattcctttcttcccttccagaccgcaaaatccagtctcattcaccttgacatgccctgcgcctgatccgggacgcgaggcatagtgggcatctcctaggttagacggatgggtgagtgggtgagtgaacggagaggtggacaaaatgagtgctcagcagttagacccaactgtgttttgatggtagtgatgggaattcccctggcacatggccaggtgagatgatcattcagcctgaaatgaaagaaacagcagatgtcaggaagtttttgttaatcctgccttgtgcgttagaactctcccgcatacgtcccctttcccgcacactctctctacctttctcccccatttctagctcctggtcttattcatctgtagcatcagtaaacatataccctttactgttcacatccaggtctgtaggggaaattttggttataaaaccggaagagtgtttaacctaatgtcctgctgcgggcttgctttccacgggcctccgggtctgttaaaatgatcaggcctcattatttaggaagctgcctcagccaggcattactaatttgggcattactaattcagatgcctattcaggcaaatgagcaatgaatgtaaagtgcttttgtcagtgaattgcaagtgaagagaagagggagtgggggaagtaaagggaaagtgatagaatgtttgagctttgcttcatttccagaaaggacagccattgcttcctggaagctgggcttggcaacaggcatggaggaatgggaaagatttatttccctttgccaaagctcaataattcagcttgagtatttcaaatcaatacatttcttgcaaagtttccaagggctgcattaaaacagccatttttaaaaattatcagtacatgacagcatcctccgacgagactggcatgtcagggatgagtcgagtcttccaaatagacaagactccagtgagaaaggaggctgccacaagcaggaaaagcgaagttagagctgacagcaagaccaaccgcacctgtagggcctctggcgttcacacacctcattggttttgtgggggctctttctgagatgagcagctggatcgctcctcctaagggcttgcatgggggaatctgtgactataactccgtatttctttgaagatgcaattcatcactgaacttgggaaagactaacttggggaggtctccaccacgatctaggattgctaagatgtctgacaggctgagtcactcatacttttgtgaactattaaaaatttcatagttctgacatctggacatcttgactttcggtgataggagatatggaaagacagtaaaaggctgtgtctttactgttgttttaatccccactagagagtcctttttttctttttttttttttttgagttggagtctggcaccgtctcccaggctggagtgcggtggtacgatctctgctcactgcaacctctgcctcccaggtggaaatgattctcttgcctcagccttccaagtaactgggatgacaggctcccatcactaggccaggctaattttttgtattttcagtagagacggggtttcactatgttggccaggctggtctcagacccctgaccttgtgatccgcctgcctcggcctcccaaaatgcgggattacaggtgtgagccaccgcacccggccagtaattccttctatttcgatgtttcatcttcactagaatcgttccttagttctttcggtgggagaagccattcagccccccttgactgccagtgccctttgaggctctcagccatttgattcttcctgtgtatcccctgtggttgttgtctctgtttctagagtgcagtggttcctgcactttgggctgtaaccccctttggtggggccagagaccccttagattaattatcacatttctttcacttactgccacaaaacccagcagcgattcactaggagaacccgaagattggtgctcttctttatgagacaccaaaggggctctgtgtttcccagcatgaagtcttcagtgacagagtctgggaggacagcacaggcagcagcagtgtaactctcagaatccccacggaagagcagagcagctcaatagcaaaaccaaaacccagcgacgagacatccccaagtcccaccttaatgggacttttggtagcctttggaacagggaaaacccaaaaatagccaagaggtattcaccagaaagcctgaaagtgggagtggttttgctcgttcctatagtagtgagggcaagtggcgtcgaggaggtctgaaaggtgtagagttctctgggccctgtgaactctgccactaaccagccacgctgatggccagctttcaggatagctgtggagaagctgctgggagtagaggcacattgaaaaggttagggatggccaggcatggtggctcacgcctataatcccagtactttgggaggccgaggcaggtggatcacctgaggtcaggagttcgagaccagcctggccaacatggtgaaaccccgtctctactaaaaagacaaaaattagccgggcgtggtgatgcatgcctgtaatcccagctattcaggagactgaggcaggattgaaccctggaggcggaagttgctgtgagcctagatcgcgctaatgcactccagcctgagcgacagagcaagactccatctcaaaaagaaaaagaaaaagaaaaggatggggcagcagagacttagatgcaaggtctcccaaaggaaacagaaccaggagatttcagaaagccagctgccatttatttgaatgctacatgaaaacaatagaagagagggtttgctaaggctggaaccatgccatttcataaaaatttcggaacattgtcttcagatgaaaatgagcaacagaaaagtagtgaggtcaaatcccacacaaagtttttataagataaaggaacagaaggagcagaatagcatccctcaagaaagcgtgccaaaacaacatgcctttagaacaattcaaaaccctacttacctccaaatgagctaaaaggtaagaagaaaaagatacgaagcgtggggcaaacacaacatcaatcagaattagaaaaacttccaaatgaaatgacagactctggaaagaattaaaagtaaaagaaaaatcatttcagaaatgaagagttaactagtgagaatacaagatgaatctttacaaaaggtgattttttaaaagagaaacacaagggggaagcaggaaattttttaaagaagaaacaagaaaggttttaaaaacaactcaagaaaaagtgacaaatattgaagaacggcaaagaaatttcaccacgtggataataggagtctctggggaaaaaaccacaaagcaagctttcgcctgcatcgctcctgtctctgcccctgtcttcacatggccatctcctctcctctgcgtctctcctctgtttccatataaggacacctgtcattgaatttggggcccacctgcataatcttgagatccttaacttaatgacgtctgcaaagagccattctcaaattaggtcttattcacaggttctggggattaggacacggacatttctatttggcagccaacattcagccatctccagtcactgagctagtaagtgacaaaaacaggatttgaatttcgttgtttcattttattataacgtggttaagagagattttaaatatttccattaagttagcaattcttgccaacaacagtagaaagcatgcctttttttttttttttttttttaaagtcagagcctcattctgtcacccaggctggagtgcagtggtataatcatagctcactgcagccactcctaggctgaagcaattctcccacctcaaccttctgagtagctgggacttcaggcatgcactaccacacctggctaatttattttgtttttcaaggagatggggctcttgctatgttgcccaggctggtctcgaactcctggcctcaagcagtcctctcacattggcttcccaaagtgctgagattacaagtgtgagccacaggacctggccaacagcatgcttttacaaatagtagggtagcaggaggccagcctgcagaccgcccagagacctcaccccatcttacgctctgcccaacctcaggggagcatttttgcacttggtggattagcattacttgctccatccatgatgagccactcatgctcttcacagattttccagactcccatgtcaaagttaaaaaacagacatcggcctctttccagaactcagaatattcacatctatattgttaacactcataccttttccagcacagaatgaaaagcacctttgaagaatcgtaagccatgaatgatgacgggccgaagggagtgagggctcctacctcttgtcactcctaccatcctgcctccaggacccctgccacctacttgggagtctgcccttaaaactcaagggtcacctgactcattcctccccactcattcatctgactacccaccaaagctaagtgtgaacagcctgtattgatttatcattttccttttgcctgcttcctctggtacagagatcttcatgctttcatccaaacgaatgaataatgtaaaatcccaggtggggtgacagagccaaggagaaaagtgttctgcagcaagggagggcctcccagtatgagccttggagtctcagataagcccagggacaaagcatgagacatccagttacatatctgtggccctggaaggggagacggggttcgctttgtggtggtgtttagctttcctgactccaagtgaccatcttaagccaggggattagtaacctttacaactctaatactagggaatatattaataactggtgagaaaaaagattacagcttttttattgataggtctataaaagccatttccgtatgaatgtcctcagaaaggaggaataaggggatgagatagaatagaatagagagaagtctgtctcacacacacacacacacacacacacacacacacacacacacaaatgcaacacaattatctttaaatagaagttgcttctccaagtttgtttttttttgtttgtttgtttcatgaggcgtcagcaacgctgttaaaatgatttcatcctgcctgtgtgtcacagcagtctgccccgcgaaatgctgaatcagcagcagcccagacagcctttcggtggagggtagattcgacacccaaaggcaaccaaccgggcctttgaaagcacttggaagcactctccaaagagccagggaagaggacaaggaagaagggggtgcagtggaattctccttaaaaaacagcatgttctagaaaagccatgctttggaggtgctgtaggtcagatttgcctttgggtccctatgtgtattttctgaggtctgttcatgtaaaagttcaacacctctttcgcactcccttctcccagcccccgcgccctgcccccacacctcctgtccacctccacctcctcctcctggaattcagtgattatcaggactgtctccatcctgagaagaggctttttaaaatttcgcaggagaatagagacacccgagagggctgggaacaggcacagctgagccacacaaagccaagcctcctccatgcaagtgacggacagatcaggtcccagttcagaatgagaatgagttttactctggttacactttgcacaggaatgttctgcagactgtgccgggcaccagagatggtccgtgtgcggtttaaaagagcaggaggggaaagaattttcatccctctttattcaggcaccctcaggagagtatccatgttctgtgaatcatgcaaatcctgcaggatctttatgttctctgtaccccatccatttctgctgaggattacaaacattcttgcaccaactttcctccagcagtttcgatttctcaataaatctgtttgaggaacatttcaactcttaatatctattttcctactttttttttcttcttaaactcaggcttctttgtaagcatacatatcagtaagtttgataataaaaatgacctttctgccctgcagtataggaaatgctgccttaatgcctctttgctttagaaactcatccttgtatagtatctctccttgcatagccaagttgcctcattgtacgtaaattaaggttatttgcagtgattgaccctaactactatgaagaaaaactttcaatatgttagcaatgatgtctataataactgaagaagggacattccctatttaaggtcaggactgtgcgtttcaaataaagaaatgacagctcaaacatttcattgtacccccacttctaagtgagaagagggcacccttcgtgtctctcttttctgtttttctccctgaaatatcaggagagacatatatctctcttaaacctgatttggttttcgcattacctcacaggtacatattgtcagaactatttgcatacacgagactaaaaaaaatactaaacggattctccagggatccatggataaacttgaggtagtctataagctttgaaatcacaagcaaatatttgtgtccatgtgcctgtgcattttgaaggggggtttgtttatgaactctggctgaattctcaaagaatcctgtaccttcttcctaacgctgagaagtacttgctctaatctcttccccactccctttttgctgtcccctccccattatcttttttctttttgatatggactttaattaaaatgttgatgcttacgtcaccatacattttctgcattaattttgatttttaaaatattgcgttacatgacttaatcttgattactgaaactttttaaaggtaccccttaaattttgctcctggggcaaatgtcttacttgcctcagcctagcctggggcctgagcgggcacctccttcagccttctttcctccaacctctccctgatccaggctgttttccgcaaccccacctgagccattttcccagcctgcaagatgacaattcagtcccctcctcaaagcactgtactgggcctcatagctcctgccagagccgtcatgcaccttccaagaatgcttgctccagcttctgcagctccagggagctgctgaaagtattttggtcttctggagacacgggacaaagtacaactcatctactatccagtccttgtcctacccaaccccccaaaatgcattgagcacttgctctgtaccaggcactgtgcaacatctctatagggatcacctcatttcatgctcacatttgccctaagaggtaagtactattctggactccatcttaccgctggagaaacagattcagacgcgtaactaacccagcgctagtaagtggtgcagccggtgttttaacccaggacaatttgactgcgaaacccaaggtctgatctaaaatgctacatggctctcaccacattgggcttgctacccagaagcaaaaatgagaagaactcttctaagtcacgtttgtgaggctgagcggcaagctctgggtcagttttgctgtcaacaggttttctgctaaaaacccgtttgtagctaaaactagataaaagtcagaaaaatccacaaaatgaagtttataagaaaaaacatcattcgagggaattgactgtgactttggatctgaacatgaacatagagtgttcttgttcttggactttgggctgaaagacatgagtataaccttccgacctccacttggaaacttagtgctgagatacaggctaaccttgaggacagaaataggaaatgttttggaacttccaattccctagtgacaggatttgtttgcagaaagcagctttggtcagatagcacattcaagattacaggcagacccagaggagtgagatgtggagcttgtcagaatgcatccaaggctggcctgggggttttgagcagatttgcagagagcaggccggtggggaagggaaaggaggcagaaaagtctccggaaaagtgcctctgggaagcagcttgaagggctctgaggacacttggttttcttttgaagggcagagtacacagggaagaactctaagtaaaccctgccctcggcaagcagaggtcagaaggtgctctgtgggcaccgagcagtattcgggatctgcagctagaccaggcctgccatgagaagttgagtttatggtttattattcctcattcccttaacatttttacagtgcattttagagtgcaaggggcttttacatccattatttcctttaacccaggggaagttcactgggtaatacagaaagcctggagctggagttttggtcttaggttcattgtgaccttgggaaagtccctcgatttctgaacttcagtttcttctgaaaatacagttgtgccagctttagctccctcgcagaatagatgatggcatgacatggatgcggaagcatctggtaatgttttgaactgcacagagctgagtgtcctgaccatcaaagccagagctggagtaagtgatcagatgtgtcggggaggagaggggtgccaatcacagactgcagagctggctcagatgtggactgcctaggaatggaaatctttggctgtacagtctggcattaaacctggggactgttatgtgccaggcaccgtgtgcaaatcactgcaaaatccccttcagctacatgcatggacatgttaactcttaacacatgtacctaacaattcacaatttcatctcccagaaggtggaagatgcattgaggagcattgctaatttgcactcaaatctgacaaatgaggaattgtctggaaaagtagaaatgatagcagagggtgagaataaaaacatgattttaggattggggaagggaaatgagagacaagaacagatgtgttttcttgacttaagacaaatttttaaaatgtagggaaaagtatttatgatctcatgatctgaatgctgagacacaactcagagtcatttgggctgctcttgaaaatgaaatcctgacttatcaactgcatatgacccttattggaataagaaggaagggagacaactaaagaaactgatgaggcgatattggggatattctctgattagctcaatttttaaatagtttgtgtgtgtgtgtgtctgtgtctgtgtgtgtctgtgtgtgtgtttgtttgttttgaaatagagtctcactctgttgcccaggctggagtgcagtggtgcaatctcggctcactgcaaactccacctcttgggttcaagcaattctcttgcctcagcttcccaagtagctgggattacaggcgcctgccaccatgcccagctaatttttgtatttcagtagagacagtgtttcaccgtgttggccaggctggtctcaaactcctgacctcaagcaatccaccggccttggcctcccaaagtgctgggattacaggtgtgagccaccacacccagcccttaaatagttttttaaatataaaagtaaaacatgcttaatagagaatgagaaataagaaagaaaaaaggaaaaggagacagggaatatcacctacaattttactgagtgtcaattctttgaatatttttacgtttcattgtatttttctatgcattgagattattacatatgtacacatactatttttcaatatgaatacatactcttcatcatgttactggctgcattaattttctatcatagatattgtcagtaactattacttatttccttattgctgttcctagtttttcattattaaaaataacactgcaatagacatctttgtgcatgaaagattcttccacatactaagttatttctttaaggtagattctgagaagttaacatttacagggttcttaaaatatcactaagttgtttgccagagagatgatgccaatttatcttctcactgttttgtgagtaccagtctaatgagctccgatttttaaaaagatgtatataatggatgaaaaagcaacttaatgaaaagaaacatataaaggagagtttctgagctctagaattttaaaacgtaagtgagctgaaaaccatggaatacgcttaagaaaacaataaatgtgggcagcaggataagaataagcatgatgtcagcctgctgatggggttggatggggtaacatcagcaaatggcaaagaaaccaaaactgttcaacttctttctctctcccatgaaggagaatgattgtcagactgaaacaggtagtgcaaacattgacagaagggaattgaagcctgagatgggtgaattgattgtaaaagcaaactaagttctgaataaatgcaaggctcttggcccggacaaatgacatcccagggtcgagaactcagaggggtgaaagataagccactggcagtgatccttcagggttgtgggaaatggaagctgtactaaaagaataggaactgttggtaacctgattttaggaagagaagctggggaaaatgtgggtacgaaataaaacagctaacctgatggaacttccagataaaattcaggacagattttcaaacatacatgttacgagtggaagacattatcactttggagccagccatctgctcctcatcctctctcctcatcttctttaataaggttgctctaagtgttgtagttgctgtatttcaggatatcaggaaggcgtttcgttgaatttctgatgttgctttcatggatgagcttggaaaatgtccaaaaaccagagtatttagatggattgatcattgaacaactcttcccaaaaggagtcaataacttgatcagtatccgtccaaaaggaggtgactggtgtgtcaccagggtactcccatctcagccctatacgatctgcactttgtcagtaacctgtggaggtgacagatgcttagcaaagccacagatgacgtgaaattacagagtatagatgctagaaatcgatggaaatctggagaagactgcagcaggctgggatgatggttaaaaataataagataaaatgtaatatcaattaatatacattcttacagttgggttccaagtgctagctatatatgttcaggtagggtgagattataaaggtgattataacaataacagtacaaaagcaatagctaatgttttttaagtgattgctctatgccagatattggcctgtgtgtttcatatcatcatcatgtgtataattttactctacactagttttatgtcatctgaaaattgtataagtacattatctatgattcaatgatatttaattcacaggatgaaagttgtcctcatttttcagttgacaccagcaagacacagagaggttaagtaacttgtacaaggtcacacagctgggaaatagaggttccatgcttgtgctcagtgcctgcatgctgaactaccgtgaaactttttctttttaaaatttagtcataaatttaatttgatatcctgccaaaacaccaaataagcttagagagttaggtagtttttaacgtgggctcggagccaggtcacctggattcaaatgtcagctcttccatttcctggctgagtgactttgggcaagtcgtttaacttccctgtgttctagttttctcctccataaatggggatcattcctgtcttacaggattgctgcagggattaaatgagttacatatgcaaagcatttagaatgactcctggcacatagcaatcacttgcttacaagtaagtgcattgctgttattaggctacacaaaaagcactatggtggggtgttaagagttgacactgtagagctagaccacctggatctctgtcatggcaccaccacttccatctctgtgtgctacagttttctcatctgtaaaaaaagaataataatcattgcagtgataagattcagtgtaaatgttcagtaattgctagctgttactatcaatagaagtatattgtccagagaaagaaaaatgctagtcctgttcctactgctgcgtttaggaattgaagccacatagatctctggaggaggcaggcaggttggtaagggtctacaaactgcagtggaggagttatctttggaccaattaaggacgtttcatctggaggcaaaatgactaagcaaggacgtgataactgtcctcactgtgttgagggttacaaaattggtgtgggtcattgaatcaagttatgagacaacatgggatgttgggaaaagtcaggggatcaggggcaggttgatcctgagtcagtctcttaacttccaggcttcagcttccatctttcacattttatgattctgtctccagcacgtagaacctagagcaaattcctgtttatacagaaagtttggataaggagtggttttgctgaatggctactgtagcagaaaagcttgaactgagtgttgctcaaatcaataaaatcacctgatgactaagccctgttttctattaaactgaaaagagaatgcaacagacttcactgggcttttgtgagaattacatgaggtaatatgtatgaaagcaccttatcaactctgaagtactaccccgatgcaagcagttatgttgattattgccatagctagattataacttttccaagaattagaatataatgaaacacacttaagcattaatctgtcttctgtgattcagaagatccttcaggaagttacaaaatctcagggtcatggccatgggcatggagtaattctgtagaaaaagatcctggctgtaaggtattctttggttttttggattcaaattaaataggagcatattgactacatgaaaccctgaagggaatagagatcacatactctaagcccgtcattgtacagatgaagtcactgagccacagagaagctgagtggcctgcccagaatcaaatgttaattcatggcagatctgggaataaaatccaggctcctgtctcccaacatgtttgtcatcattagtttaagaacatctcagaaagtctgaaacagggtcaaaatgtactgatgaatgtggctgcccattaaagcatggaattgaagaaatattagaagtaagtttaagggcctgggtgtgtcactgaaggatatgacacaatatactggacctactcgggagaggggagaagtacagtagaatttttagtaactttcttaatcacagcagttcactttctctgtacaaaattcttttcaaatattctctgaaactaattggcattaaaaatgctgatcttagtaaatggccctcaagaggcacgctatgcgcacagggaatagtataatataggtgcagagtaatggtctctctccttctctcctttttttccttttcctaatgatcattaacatctatattctttgactttgttgaattaaagttgcaagagatggccgtaaataaagcaagttatgtatgtgcctccctcattctgcccatgcagcttctgttccatttgcagctgtgagccgtgtaagactctcaagaccaagcacccatgagtgcctgggagcaagtagcctgtgaagggaggactttattcctggatgcattaaaagcagagtcccacctacacctccctcctcttcccatttataaatctacttctccagggatctacagattttttggctatatttttagtctgttgataatttgagaaagtctccaaggatctaagtcttggatgggtaggagtcaattcactcagtcttttttgaatccctaatggatgttcaaaaccagtctcaattctggatctggatgggacatgcatgtcgaagtacagtgtgatggataagagcacagattctggaaccagattgcctgggttcagatctccgcttctgtcattactagggttatgtgaccttgggcaagtcccttaaatgcgttgggcctcagtttcctaatctgaatccctatcatagggttgtgtgagcatgcagttgaatgagctaatatctattcatataaagtgcttagagcagtactaggtgcatagtagtgcttagtaaatgactattaagtaaattaaatcagcctgattagaattgcctccaggtgcaaggctctatcccatttagacacgctaacacacaaggtcttaagggctttaggtttaaggaaaaggggctttttcccccctcaattctacaccacaacctttttattctattgaattacaaaggaaataacagtgggacttgactttgaacaccaaagtttatccagcctctttccagctcggtgaacgtgtccacctacttcactctttatgctaaaggaagagcagcgacgctgataaatgtattgagtggcctcgagcaggtggcttgctctgtcaagatcacttttgtatgactgctgggatcaccacagtctcccctgtaacttgctgggttcctcttgtacataagccgagggcatttgcatcggttcctggtgagggggtttccacacggtggccctggttgcttcaggggctgggctgtgggagacattaatgggaagtgaaaagagaaactgttgtgcttaaccggcagagctgtttggagtgtgactcttcttgtatttagggaaaagattactcatgagtattttcttccatagaatataaagaacctcaaattactacttgcatcccccacccccttctcacccacctccctccgttgcagcgtgtcctctgcagaaacttcagcattttactctcaattcccctcccaagcaggattcagttctgtatttgctttggggcccatgtttccaataagccaccctgtactttttcctgtttctgcctgagcatcttctgctcactggacctctcacctccaggcaccttcctaggcctgaggaaccagaccttcacatgtgtcccctaatgggaactccctgtgtgaaggagaggaaggtccataataacaaatcgcagcgccattcttgttcaaaggtgacaccattgacagtaaggagttaggtttaaggtttttaccagttcaaaaactgagattttgctgagtaaccctcactgccactcccacctccctgtgattctagatacgtgttttcaaggtcattggagagagctggtacttcccatatccatggactgtttttaataggcatcactttctggccagtcaggagtcaaaaacaaaaacttttagtggctgtattttattctaaagatcataaagcagtaatgcaccataaattattatttgaaattctaatttgtttccatgtgaaaaaaaaattatcctaaaaacaccaagaaaaaagctcttaaatgctcatctctaatgatttccagtgcccagtagttctttttttggtgcactacaaaaaaggaattttggttttgaggggacttccacaggtttagaggaagccaactaccattcctcaaaactacactttgcaaaggagccacttggaagacaatcatgcccagaggccctggacattttccagggcatttgagccgtttgttccccacttcagcaggtaggaagagcagccccatatagcaaaccagattgttctttggcatgcctggtttaggatgaaaggtaattttttccaggatcaaactgagggacacttttataacacacttgtgaagtcttcgtagcaagtttaagtaacttatagtgagagacggaagctcctcttagcaacactgatgccattgctctgtttgagctattgattcacaccatgtgattttagagaatgaagatccattgaacatccagcttcttctgacccagcagttcttaaatttggtctcatgtctccttttgattcttaaaaattaatggagaccccaaagagcttttgcttatatgggttatagttatctgtagttaccatattattacataaattaaacctgggtatttacaaaatatttatgaatttagcaagtaaaatagccaactctatattaatgcaaacatattttaaaatatattttcccaaaccaaaaataatttgtgagaagagtagcattgttacacatttttgaaagtctcttaaaatctggcttaatggaagatggctggacgctcatctctgtttctgcattcaatctgttgcaagatcacatcatatgccttctagaacattccacctaagaaagaatgagagcgaaaaaggcaaataattaatattgttatgacaagatgttttacttcataggcaccctacaaaggtctcgggaaccctgatgggtcccaagatcacattttgagaaccactgttctaacttatcaaagatttctcacaacagcaaaataaaataaaactatagacttacagtcaggctcccagagagccaacctgagccaacctctcagcccctgctctgtacccccctcctgcccacttggttcctactcagagttcaagtaccaccccaccaggaagctctaatgtcagtgagtagaataaaaaggcagaagaccttttatccttactttgccctcttattgccagaggccagcctgatggtttcccagaggctgagtgaattgctcttcctggaagaagtttcagggaacatttcattatttggcagctctggaaggtcaagctcagctctggaacattctttcttcagaaggacgctgggctgcaatcagatcagctctgaatctgaggaccctccaaccacactgacacgtgtcatagttctgagcccctgtgggtgctgtgggcaccctggaacagcccctggaactgggcaaggaagggcaggtctctaagacctaccatcccttaaacacataaaagatctttttctcattttgcctctaaataaaaagttaatctgacagcacaccccagagtagggtttggtgtacaaactgctccctggactcctcttggaggctgcttctgggcttcgagaagcccccagtggggcggctcaaacaagggctggagaggggactcagagttgaacataagggctcttgagatgtggagactcattttgttaactattcagagaccttctccctttattccagctgtctaactgacaagacctttctctctttctttctgcctcatgctctctccaacaggcttgcagccaatttactggagcagggatgacgtagcccagtggctcaagtgggctgaaaatgagttttctttaaggccaattgacagcaacacgtttgaaatgaatggcaaagctctcctgctgctgaccaaagaggactttcgctatcgatctcctcattcaggtgagagtctggactcttggcatatgcccaacttggaaagtctcttagttagtggttggtctttaacacccctcacccggcccaagaaatcccaacagtgagtcatcacaaggaagggatgacgatggaaggaagttgaaggaagattatgctagcatgagccagtttgagggaagaagaggaagtgtttacagaccaagggggagaattttaagtttaggagaggaacccagtttagtcctacgtacactggaacttaagggctctaactgtgccactaagaagaaaaagaaggctggccgtggtggctcactcctataatcccagcactttgggaggccaaggagggtgaattgcttgagcccaggagtttgagaccagcctgggcaatgtggcaaaacctcaactctacagaaattttaaaaattagctgagtgtggtggtgtacacctgtagtcccagctactcaagaggctgaggtgggaggattgcttgagccaaagaggtggaggctgcagtgagctgagattgcaccactgcactccagcccaggtgacagagcgagaacctgtctcagaaaaagaaaaaaatgccattttggccttttgactctctaaaagagatagctgagagtgaatgcaggctgtgaactgctcaaccgaaaacaaagagaggaaggagaaggacgtagaagggctagaaaagaaaatagaactgggggaactgcagataaagactgggaaatgggagatcctagtagccaaggccagtttagaaaggcttcaagtggtttcccttagaagtaggactcgtttgaggtaatgcatcttgtagcctgtagaaatcctaaaaaatacaagcagaggtctcctcgggctcatctgactcagaaagcttgtctaattaagggctcctgataatgcattagaagatttccattcagtaacaagcatctctacctgcagagtgtattgctcatgtggataaggcttcaaagatttgtctcagtttcatttcctaaaccctgtggatatttagaaggtacctgtctgggaactacatatgtcaaaggggaactgtggtctcaggtttatgcctcttgcgggattaattactgaagtactcaaggccctccttttactgaatcccactgcagacctcgactctgatgtaacttgacaaagggcgaggatcctgcagaaggtcttggcatagtccctaactacgttcaacctttatttactcctggagacctgccagaagttaataaactaaccgtcatgaggaaacgtccctcattacaggagatgagataaagccccagcacccgtatctccactgttcttcccagttgccacagaccgtttatatgaagaaatgctaaagaagttccccagaagtagagatgttattgaaacttgaaagtttatatcttcaaaattgagtaatttggaagggacacaaacatacctgaaacattttgtctcatatcatcaattctgtatgtaatctcacaataagaaaactataaaacaaccatgtaaataaatcagactatcctaaatagttcttttgaatttattacaaaatacttcccttttctattccagtggaaactcagagatattatactcaaagaggcaacgttatatatctggaggaaaaatccctacctctttgtgttccaatgaaaccattcatgtaaagcaagcaaaggaaaatacagtggtgggttttgacttgatttgacttggaccttgggcaagctttcctcccaagtctctataaatttccccttcatcctatctcaggtgcacttttttatagctggtttattttcctccacagtgacctcttagcattatttgtgaaactgggttctctctgctcagagctatgaacaaagatacgtgaaaatctgtccgcagttccttgccactttgtcgagtctttgtcaggtgggccatgtggaagatggtgctgctctcaaggaagttactgtccagttagaggcaggattgcgttgatctaaaaggccaaagaaggctggaggagggagagagcagagtagcactggtgtcgggagagaaggaatggagttggtttggaccatgaagaatgggtgggattgagtaggcagagggtaattggaacagcatcaccaagtatatttgggtgatggtgagaatgcccacctgatgagtagaagaggcagaaaaggcaggaagccctgataagcctgatgcactagaatggatgtgaggtgctatatagatcagttaggaggtggcaatagaaattcattccatgagtattcaccaagcaactctatgccagaggtacacaggtacaggctaagggagttcagagattagaggggccatttctagctggcttgtcgaacaaacctcatagacaaggtaatggagcctgactttgaaaactttgaaaggcaaacaagaaggagatgaggaagcatgttaagaaggagatgtaggccgggcgcggtggctcacgcctgtaatcccagcactttgggaggccgaggcgggcggatcacgaggtcaggagatcgagaccatcccggctaaaacggtgaaaccccgtctctcctaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaaaaaaaaaaagaagaagaaggagatgtaggctggtgtacatggaagaggaattcagagacaagactgcacgtgctagacactccctttgaaacatggtcgaacatcactcaccatgcctctccggcttgtctgattgcctcctcacatttcaaactggtggactgtctcctggaaggcttctcttgactcccatccctacttccctaattccattccactttcctttctcccatcttgttctacctccatctaggctaggtgctgtttctcctttatcttagagcattctttccctctgtcatggcccttgacacagatacacacacacacacacacacacacacacacacagtgttatacattgtttgctgattgtttgctactccggatccctcccctagagtggaaagttcaatgtgggcagtgattgtgttctattaagtcgccaagtctccagtacctaacaccagggcatggcttccaggtggcactcaacaaatgtaggttgaatgatgacccggtttgagagtacccaggttaagaagtttgggttttattttacaggtaattggaaccactggaaagttttgatcagggaaatatgattggagctctctttttgcaacagacattttccaccagtgcgtagggtagattcattggcttacttaaagcaggaaagctagttagagtgtccttgctgtgatcctaatgggaggcagtgaagcccagactggatcagtgagatcaaaataatactcaggcacaaagtggctagctaagatggggagctgacttggaagtagtggttagagttcatttttaaagcaagcaatgccaggacattcaagaaaacttgtcctgatctgcagttggacatctaaaacaagaggcaggccagggctgggaataatagatgtgagcatcatacatcgtctccattatgtagaattttatgagctggggataaagataaatttatacggaaggtaatttaattaaggcacccactagaaagccatggacagaatttcaaggcgatgcctgccagcagggcctgtaggaaaacttaccacagaacacaggagtattgcgaatgggtttataagacaactaagttggagttgagaagacccatctgatgaaggggtcactcagcaagagggggaaggccacaggcatcctccttaggcaggggcaagatctagattgaaaatcctttttattttccagatgtaacatttgccactgtttcttctgacatgggctttctcctttagcagtttggtgtagtgaaagtcctcaggcctggcttctttctacacctggcccttcacaaatcacgtcacttcacttggccttattttcctcatccacaaaatgcagttctcaccacatggatcctaagctcccttccattcttaacagttcagcccaacttcaacactgaaacagtgttaagttgctagcaaacaatgtagctttctatgctctctatatatggaaacaacattctttaacacaactcctctccaatgtagagaggagagcactggactcaatgagttataattacctggaatacgattataccttaagtcaaatacagtagactccctgaatgccatattctttgaccagcttaacaaatttatcgcatctacttccaggacaagaatcctccattttcaacccctaggataaagcagggaacatttgcagtgtgctttaaataattatatggtctctaaccagaagatttcaaggttggagacaaatggaatattttctttggaatagtctcccagttggaggatataacagatgacaagtttataacatttgtaaagttaaagaataaatggaagggatcccaattttataaaacgtcacctggacagaataattttacaaagaccttgggctactagatagttatcacattaactggttctgaagtgatttaaattcagagaggtatttgagtacttgactactttgagactctgagaaaacacattgacaggagggaacagacaggttggcatttatattaacccttacttagggttatccattgcatgacattcaggtggaatggatttaggtacccattattttctccgaacaaggaaacccacaagttattatcaatttaattcatgttacactcacttgctatcaatgctaaaaaagggaaagaaacatatgttcagagttccttcaattcagcaaaactgtattggggatttgctaaatatattgaggattttagtagactaaaatctgcagtcttttcatatactagagtgtgattttgcaaattgtttcttccctttccaacccaccagaacctttgtctttggaaatatgctttgcttatatttttcaaatatagtaaaattttctttcaaagttaaactgaaaatactggtctttacatagtatttcttgaatcaaaattggcctattaatttctattaagcaatggtaggtttgccagattgaagggctgtgtcaactggctattttagaattaagtcagagcatccaaggatataggtcacagaagggaaattgtagccatgaaatgtgggagttgtagcaggcatgacccttgtctgaagacaccagagttggctctttctttactgtgtctttgagcaagccaaaaaaaaaaaaaaagtgtatcacacatcagagaaatgcttcgataggtcacttacgtgaaagtcattttatttcttttcttttttttttttagatggattctcgctctgtcgccaggctggagtgcagtgccgtgatctcggctcactgcaacctccgcctccagggttcaagcaattctcctgcctcagcctcctgagtagctgggactacaggcgtgcgccacaacacccagctaatttttgtattttagtagagatgaggtttcaccgtgttggccaggatgatctcaatctcttgacctcatgatctacccactttggcctcccaaagtgctaggattacaggcgtgagctactgcacccggccatcattttatttcttagctctttgcctgtcccatttgtgaaatggaggtgggcacccaccctgttgtcatatacttgaaaaatgcaagttttagcaatgttaaggttctactttaaacttactaaacttaagaagttaaaagttggagagacctaggtagccttgcagatgacgctaaacattggttatacaagttgatgtgatggtaacgtagggaaaaccaactgcacagtgcttcctttaaacacatacttatttcataaagttctgttttctcgagagcttattttgtttatctcaccttatagtccatatccagctgtaattgtcttactgtatctgttttctaacagatttattcttgtcttaaatatgatacttaaattttcccacaacctgttccttttttctcagggtctctgtgtataagacataaatattaaaccaaatttcaaatatttgttgctaagtaaaagactaatttatttttgtggtcacagaaggcactccagattaaattaaaaggttaattcaattggcctgcaaaaatgcactgaatggaatgcttagaaactaggaccaggctgggtgcagtggctcacacctgtaatcccagcacttaaggaggccaaggcgggcagatcacggggtcaggagatcaagaccatcctggctaatatggtgaaacctcatctctaccaaaaatgcaaaaaatcagccgggcgtggtggcacgcacctgtattcccagctactcaggaggctgaggcaggagaatcgcttgaacccaggaggcggaggttgcagtgagccgagatcacgccattgctctccagcctgggcaacagagtgagactccgtctcaaaaaaaaaaaaaagaaaaaagaaactaggaccaagcagagttagaaaaccaagcagtcccattacaggaatgggcatgttatgcactggtgttttcactgttgaatttattatgtaacttaagggaccgaagcttagggcttcatcctgaacccatcaaaggcagaaagggcccaacagagagaacattctgacatatctaaaaagtacctctcccacaccaggcatactcttactttaaaaaaaaaaaaatggccagagggaatctgaagcctctgaataaaaatcaaagagaagagagagctgtttgcaaattaaagaatgtgttacttagataatctacgtgtaaggatatgtacatagcaaatcatcacctgaaaaatgcagattttaacaatgttagggttctactttaaacctgctaaacttgagaagttaaaagttgcaaaaccccctgggagaggctctaggtagcattgcagatgatgcgaaacattgattccagccttctgagaacaagtagcgtataataagaacgtttaaactaatcatacactttcactgataattctattcctaagcaaatagccgaaggaaaccatccaagagtgggcaaaaaaaaaaagatgcccttaacaattctatttatagtagttaaaaactggaaattccccaaccacccccactgtgggactagctaaacaaacttggggagagtaaatatcatgggatacacggtagcctcacagccattcattcactcactcactgattattttttcagtagattttgcagggctctgcaggtcatgttcaggattttacatgtttgtttgaagacaagtagaagccatttgttttttgttgtttgtttgtttttgtttgttttgttttgttttgagatggagtctcgctctgtcgcccaggctggagtgcagtggcacgatcttggctcactgcaaggtccaccttccgggttcacgccattctcctgcctcagcctcccgagtagctgggactacaggcagaggatcggcatggcaaggtgcggcggtgaaaagtggaggcggggcatctgttgggctgcagtctacgagcaatagttgtgacattaactaaggtgaggcccactgagagacgaggtggttccagaaacagataaaaggcagagtctgcaggtcttggtgaccactttcatgtgagatgagaaggaagagaggccttaacgattactcccaggtttctggtctgatcatctgagcatttgctgacctaagaaacaccatctcatctgtagagatgtttgtgttaggcacatcaaatgtgtggtgcctatatgacctccaggaggaattacttaggagatcacggaatgtggttctggagttcagaagaagtgccaggctggagtagatttagcatagacgagccgattgaagccatgggagtagatgaggttgcccaagaagaagggcaggtgagagacctggggaaccttactactgaagggctggagaaggaggaagagcagagaagtaggcggaaaaccaggataagtggaaatctctgaagccatggagtattctaagagaagggaagtagagattgctggtgagaatgttgttgagaagtcaggcagttccctgagtaccaaccattgccctttggatttgacaacagggaggtgacctgagcaaaagcagattggagaagggtgggagtgggaaccagaatggaaggtgaggaagtggtgagaatggtgttggacagctcccctgggaagtttggctataaagggaggagagaaacaggagcagttcctaacagggaggatggggacaaggggcatgtacatgtggacaggaaaaaaacttgaaactgcttaaaagctaaagggaagaaacccaatagaagaagttggcaatacgactgcaaatggggacagtaagagctaactgtaaactggaaatgtctgtatgaaatgatgtcaactggggaaaaaaattccatccatatgaatgtgactttgtaaaacttacgcatgtgctttaaatacaaaagtactgaaacaaagaacattttttgccagattttgtgagacatgtctgttcacgcttatgacaattgcaagagacaccatccattgagcgtttatgatggcagacgtggggctggtgctctgcacgtatcatctcatttaatccttataacagccctgccaagtctgtggcatagttctcattttacacacaaaaatattgaactaagccctgaactatggctcggagaagaaaaatagcttccctaagggcatacagctgacaaagagaggaacaagattcaaacttgggactcccttgctccagagcccaaattctttcctctaaaattaagtaacttaaataaatttaacccccctcaacagcatcttgcatgttcttggtcctcagacattagtaagattgatcagccaagtcacagcagtattgttcactcgctctctcctgtccatttggaacacattacaatacaataatggaatttaggattctcaaagaacgtgcctggcttttcatgtaaagatcaccaatgcaaaactgtgttttgtttctgtgtgtgcgtgtgtgtgcgcacgcgcgtgtgcatgcacccaggagtatatctagattttatgggcctgaggcttacggtttgggcaatatgaatctttctagaactcttcccagggacctgcgcaagtgaagggccccgaagctcactagcttcatggtacatcagcctctgcacatgcgtgtgtttacatgaactgtgtatgtatgcgtgaagatagatggaggaaccaacgaaaaccatggttctttgaccagtagggctcagttccattgaatagtacaatagaaatactagttagtaccagtcactgtgtttcttttctgggaagagagagaagatttattaaaaatcaaatgggttaagaattttctgaaaaatgccctttgaatttcagccattagctaagttagatatacagtgctgtggcgcatgctctttattttcagtaggcaaataaatataacatttttaaatatattctttatcccagagactgttttggcgttgtttgtttgtttgtttgtttgaaacaaggtctcactctcatgcaggctggagtgcagtgatgtgatcacagctcactgcagcctcgacctcctgggctcaagcaatcctcccagctcagcctcccgagtagccgggacaatgggcacgcatcaccacgcccaactaatttttgttttttttgtagagatggggatctcactatattacccaggctggtctcaaactcctgggctcaagtgatcctcctgcctctgcctcccaaagtgctgggattataggtgtgacgcacagcacctagccagattcttttgttagtaacaccttttagatgtgatctcctccatatatgaaaattggaagaatacatcaagagagcaggctgggtgtcgaggaatgaagggacactggaaatgaaagaagatggagggctgtcttcatttacttagcaacctgctatagacaagacacatattctgtgttacttcacaagccaaaactaagaaccaaagtacagggattgcaggggaagccagattccacctcagcagagaaactttctatcaactagagctgtccaaaaatggtttgcttgcgccgtgactggctacccatcactggaagtacccagccaaagccacttctttctgggagaaggggaggtgggagactcttagtgagcaaagtttgagtcaatctaggaaacctctagaatcctgtctagatctaatattccaagttattactgggacacactatcgctgccaatcatatgctcacagtgcacgcacagcaagtggtccccgtccgcatggtggtgatgtgtttttgctgagccagccgcattccctgacggagcccctagttgctgaggaaacatgtctgtgcatggactgactcagctaaggccacactcctgggtcttagaactaagttttttcgtgacccagtacccaggacctcgggtccatgttcccacccgtagccgcctcatggatagcatttaagataactcccaataccttattagtctcctaataggcttctggtctaggtaggaaactggccgagtccacttgacccgcccctccccctgcttcctcctgcacactcgggaagaacccctctgaccctgctcccttggccagcgtcagtatgggaggctgaagcaagcccatgcagaagcctgggaggtgtgcggcagaccacgtgtcagacccccagaggcttagcccaccaggtcaggatgagacgtccagagaacccaagactaggagagtggcctcagttaaccttctttaccctttctggaaggggaacgtaaagctggccgtgacccagatattcctctattctccatgtgagacctccttagagatctacacacctgtcccaagccactataataatatcaggaatagtaatgatgggagtgtttgtggtggtggtggtagtggtggtggtagtaaaatcaatgactagtgtttactgagcttttcctgtttgcctcacgccatgatgagcttgttacatagaccatcttattgaatcgttacaacgatctcacaaggtagatgccatcaacactgccattttatagatgaggaaactgaggccaagtgacattaaagggcatgcccgaggaggcaccagtctgactccagagcatggcctcttcaccatgctgctgtctttcctcagtttctctctcatgaaaacacagaaggaaaagtcacctgcctgctctccagcccttgtttgagccaccccaccgggggcttctccaagcccagaagcagccaccaagaggggtccagggagcagtgtgtgcaccaaaccctactctgggtgcttcttgacctttccctcctttaaccctcatagcaaatgctggaggtcggtgttattatttgcattttacagagaagttcagggacttgttcaaggtcacctggtgcacaggagttgggactcccaccgaggcagccgtgcccacctctgcagtcagcacctcccaccaccacaccagcccgcctggttaagtgaaccaagctgaacaccctcacagacactaaatttatatttcctggcctccccttccaaaatggatacactaattactggacctttttttttttagtaatagtaatcatcaggaacacatttttcaatgttttcatttttctattccgcatactaccaaaaaaaccagataaaacccagcaagtgtgaggccctgtaaattagcaaaagttcaggaagtcattacacgaaaccaaaagaaacaaagtgttgattaggggcttatttttgtatgagattaaaatatgaatcattaaaataggactgtatttcaggcttcttatccccgcctcatcagagacatcaccatgactactaggtttggattcaatgaattccgaagccgttctcagctctcaaatgcagtcatgtgtgccatggttctgccttatacatagcagtttcaaaagtctggtccaggacacctctgcatcttcctgcccatctccttttaataaaatgcccaagtgacatggtgtctgcagctctttgaggtattttgctttatttagtcatgatactaagtagtggcagaaagttatactaacagcaaccaaatttcaaaggtagaggtaggaacaaagtcaaggcaaaaacacaaggttccctcaggaatcccttagaatagggtatccgtggagttttatttagtgtcagtgatagaggagcaggagtttgtagaaaaaaagaaattaatatggaacgtttctcaggtccatgctgagcttgaaagaaaaaataatacagcaatatcttcaatccaaaatatgccttgaatttaagttggtaattaaaatgtttgtttaaaaaaagtcgctaaaattagagacaaagacccattgtttgttattactgctgactgcatatctgtgtgtggagaagggtgtgaggagacagtcacaggcctgtgatattgtaaagatttatcacccacccccaaagggttttgaggcacccacctgcattttcagcttggatctaagtcaaggggtgggacaagaccatgacaggttttggggtttgtttattttttgctaattactcgtggacacttcaaataccaaatatatcagaacagaaaggcacctgctcaacccagcccctgccatcctctggcaatggcctgacatctctagatgattttctagagattgtggaaccttggagcaatgcctgtgtgcatttttaaaggagggaagcatcccagaacatgccagttccttatagagtatcttgaatagttaccacctctaaccggacttgtgaatttttttcatgtttattcttttcccgtctaggattgagattggttaaaaaggcatatttttctctgtagggaaaatttctgtgaagaatctatgagttgtcattggagcattacaaatgttcttaatgacttcataattcttcgagtaatctgaagtgcaagctaagatagatgtaaaaagaacatcctcacaaaaaataagagcaataaattgggatagtttaaattccctgccattgttcggcagctactgaagcagttcagtaatgtgggaaaatgttcaaaacctaatgtggagggaaaagaggatactcaactattaaaagcccgcttctaactatagaactatagatagtattaacagggcttctgtatcttagtgatgatgggattctaggtcatgttttgttttctttctgattttctgcctttttaaaacatttttggggccgtgcacggtagcttacacctgtaatcccagcactttgggaggcctaggcgagtggatcacttgagctcaggagtttgagaccaacctgaacagcatggtgaaaccctgtctctactgaaaatacaaaaatcagctgggtgtgttggttcgcacctgtagtcccagctacttggagggctgaggtgggaggatcacttgagcctgggaggtggaggttgtagtgagccgagatcgtgcctctgcacttcagcactggcaacggagcgcaaccctgtctcagaaaaaaagtatacgattttcataatcaaaaaagtttgatttacaacttcacatcgctctgaccttttgagtttcaagtgctgcattctgtgccccatgagcttggtgagggtaggaggtggcaggtgcgctccaattgtatctttggcaccgtgccaggcacttagctgctgctccgtagatcgttgttggaaaaacatctttccatttctcgatttccctttcctttttctttccaggtgatgtgctctatgaactccttcagcatattctgaagcagaggaaacctcggattcttttttcaccattcttccaccctggaaactctatacacacacagccggaggtcatactgcatcagaaccatgaagaaggtactggaagaggtttctcttttcttgcctgaggtttagacaaatccaggaagtttaattgttaacttgataagctggtcttcttcgtgtaagttcacttttcattcagtagacaattattgagtgcttactatgtacaaaatacactcttggttccctgaagctcacgatcaaagcgaagcagtgagatggatgtacacagaagcacagaagcgtaaccatatagtaggcaccgtaaaggtgtggtggaagggtccatgacagccaggtgcaggaagcagggtcttctcataaaggaagcaactctggagtatgccttagagcagtgctccccaccctttttggcaccagggaccagttttgtggaagacagtttttccaccaacagtggaggtgagggatggtttgggatgattcaagtgcattacatttattctgcacttcatttctattattacattatcatacataatgaaataattatacaatccaccataatgtcgaatcagtgggagccctgagcttgttttcctgcaactagacgttcccatctgggggtgatgggagacagtgacagatcatcagccattagattcccgtaaggagtgtgcaactagatcccgcgcatgcacagttcatgatagcgtttgtgcttctatgagaatctaatgccgctgctgatctgacaggacgtgggactcaggcggtaatacgagcagtggggagcagctctagatacagatgaagcttcactcacttgcccccaccgctcacctcctgctgtgcagcccagttcctaacaggccatggactggtaccagtcagtgacctggaggttggggaccccgccttagaaatccagcaggattctgataagcaaggatgaagtgaagaacattccaggcaaagaaaatgacgtaagggtggggtaaggctttgaaatagagagcaattagatgacttggattataaagcttatcttctcccactcttttattacagaaaatttcaaacatactccaaatagaataaaataaataaacccttctgagccatcacccttgccttcacaatgatcagcattttgtcattgcagagctagtccaaggagcttagcttggaaccagagtgtggagaactttgatttctggaatatggaggaaagaaatgggaagtcattggagagttttttagcaaggggtaataaaagttggaagaaggaaagaaaaaaaaacccaaatctatgaactcagtgttgggccagggctttctgcatagggcaccaaaggaggctggttagagttcactcagtggcggtgtttatttcaagtgtcccagttgttcctcttaatcattggtagaaagcaaaagaccagaatttaatttaggccctggacctagttccagaataggtgttggggcacagaaatagagtataaaaaccttacttgaggccaggcgcggtggctcacgcctgtaatcccagcactttgagaggccgaggcgggcggatcacgaggtcaggagatcaagaccgtcctggctaacatggtgaaaccccgtctctactaaaaatacaaaaaaaaaaaaaaaaattagccgggcatggtggcaggcgcctgtagtcccagctacttgggaggctgaggcaagataatggagtgaacccgggaggtggagattgcagtgagctgagattgcgccactgcactccagcctgggcgacagagcgagactctgtctcaaaaacaccaaaccaaagcgaaaggaaaccttacttgagcaccattgcattaaacagttgaacagcttgagcactcagtgatggatcgtgcctctgagggtcatgatcctcctgctcggattggtcagaccacagacgtccaaacgtgcatcagtttcttcatgtgtacttttccttaggtgcagattactagggacctcaaaccctggtcccttggggcatttccttaaaatgattgctcatccccaagagcagggctaacaattttgttttgttgttggcaaaccaaaaaatgagaagtttcttgtttttcctaattaacacttcacaaaagagagcctgactccaggcgatgacaccacctccttgtgccaagcatttcacagcagggcccctcgcttccgtgatggagaacgaacagcatttcgtgcacgctccctgccttcctctcatcctttcctcccctcactctacccttgctaaatttagcagctgccctcctcccccaattcagttattcattcttttgctcttcctgccatgtgcatttccggcttgcataatagccaccagccactccagagaagaggaggtgggtttactgaagcttgacattaaggcaaggaatatattacctagcctgcctttcagatacaaagggtatgtgatgggttttctttctgtctcttagtctttctctcattattttttttgttaatctcatttggaatttagccccattttattcctgagtgtgattggaggcttatggtctctcctcctcacagatccttcctcttttccctttggcctgatttgactcagcagtaggtgtgtttgttgttctcatttgcaggagctcagcaatagagaaagctggcttactctgagagctgctcgcaaaccaagtaaatacagatgtgccaccgcatacgctgtaatgagctttctctcaagtattatgatgttggtggttgtgagctcagtaggtaagaggatggagctaatgaggccgaggtcaagggtgtcattgccctgcagaccagttagctctgcttcagacccagctcttaaaagtagtgcattttcagagaccattccttaaagctcatggccaggaagcaaggtctgagagcatgtccgggcgcatgtgctccagtgaacaggtgcacacaacaaggagagaagtgtaggtctttatccggtggtatccagggagaagagctttccaaggcacagaagtgatatgaagaggtggccctctccctatcctcctgcctttcagctatatggtgcccttgatgacaaagagaacaagtagagaggtgcatggatcagtacaagtttcctgcctccagctacttcctctctctctcccttcttctccactgaaaagaaaaaaatccagctcaacataaattgtttccagaatcaatactgttccatttaattatgaaggataacagatgattgatgtgggcatatgttaacatacagaaagatctggttctgaaaaaaaaatctgtcaagtgaaattatacatatcaaatattacttccccagtgatttattttacataactgggaatacagttccatataaaagtatgttaacccaagatgacacatgttgctgccaacttggcataggtaagtaggtaggtgggtaggtaagtaggtggataggtggataggaatgacagaatggaaagagaacaaggcccaactcagctgttaagcagcctggcacaatgaagcatgcacggtcaagtgcaaacagaaggcatcattcaaccgccttgtgatttcacctcgtcgtggccacaaacttaaccagttcagacattcataatttcacaatgggcatttatatcacagtacaaatgtgtcttgctcaatctagcaccagttccccacatgtatcctctatgactttcttacattgaaacttacattgaaatcatgccttcgttagtagccacgtggcagtgctagaagctaggaagttggtgttagctcagaggttcacctccatgccctgacagtagacgaggatgatcatcatgctaattcaaactctctatatgtgaacatttgttacctctataatgtactccattctgaaatcacatcatggcttgagcaaagcagtcctcagattacacttacctctagaaaaaccagcattttcactctttttccttacttattagttatgggaagggagggtgattttatataattaggttaaccttataatttttcacccataccaacacacttatgacagtgacacagggctccagcaataagtaagccaggactacatatcaaatattcaggcaaaaccagaaccagaaccatcctcgacaaacaaactggggtgagtgtgtgtgagtatgtatgtatattttaaaggatgagatctggaaaccgggaaaccaggtcctaacccttcattctgccagaacagatcataagggctaagtttctcattcctctggctctgtctccccagttataagaagggggttaggctaaatggtttctgcagtctcttacagctctcaaatccccacattacgtccaaggaacctgagtcttcctgggtgatatgaagataagacatattcccttccctcagggtcttgagcatctaaaatctagaaaatcatagcagtgtcctgcctggtgttgctcagaagcgggagaaaatctcttctttcctcatttgtctcaccttattcagaacttcagataatgttgcccctctgagagtggcagcaagagtagcgtcattcacttatttcatcataaaaagctcagtttaaggacattctttttccttgactgttgtgactcattttatctttgtaatatgaattagaatgcaggtaggtagttatgtagaaaggaggtaaaataaacaatgatattacttccttcacaaaaaccaggaggcaaagcatctttagaaatatcaaagcagtaaatgactataaaataaatttattttgtcacgtgcagttgtgatgataacttcttttcacaccttaggttcttttattttgggttttatccccttaaatatatatatatatttttttttaattgtcatttgtcctctagtgttagaactactggaatattggccccccaaaaactcaacggaaacataaaataagcctcccttctgggctaaagtctctgaatgagaccagctccacagaatcaccccatgggactggatatgcagtctgtagggagaaggagcaagatccggatttctcaaacgttctcatcccatcacccccttggagcttgataatttcagctctttagttgcatcctgtcctctgccccatcaaagagattatggggcagtgattcttaatcactttaatttgcagatattcacacatgcagggctgagattgtttctgtagcactggatgcagttttaaggtggcagttctgctctgggacagctctttatcacaaggtaaacatggaaatcgcaagtgatgaggagaactttcaagcatcgaagtgcaattgatctcaatttttcttacaactagcacatgggaatggctcaataattgagagaagtcatttctttgtgaaagatggagagggtggcatgtgttctgcgatggcccacagaagttttatttatatgattgctttattgaggggaatcgtacccctcctgccccaccaccaaacccaagtgcccaaagactgctttggcagaaggaagttgtatacaattttaatgagataattaaagaagatgaggtatatgaatctggttttttttttttttttttttttttttttttttttttttttgagaaggagtcttgctctgtcgcccaggctggagtgcagtggtgcgatctcggttcactgcaagctccacctcctgggttcactccattctcctgcctcagcctcccaagtagcagggactacaggcatccgccaccatgcccggctaattttttgtatttttagtagagacagggtttcgccgtgttagccaggatggtctcgatctcctgacctcgtgatccgcccgcctcggcctcccaaagtgctgggattacaagcatgagccaccacacccggcctgaatctgcttcttaataacaaaataatagctaacatttattgggtccttgttgtgtgccggccctgttctaagctcttatatctttgaatcatttaatcctcaccacaaccctataaagaaggtgatattattacctccactttatagatgaagaaataaagattaagagcaataaagtaacatgccccaaaatcaagtagcaacgctgagatttaaacccaggtggggttgatgccagtgcctgcattcattccactgcgcagaatgaggagaccaaaatacattttcataaatatttgtagtgtaggcgccctgaaaatagagcacatgattgaaagacgagtagcagtagtggagatgagaacgccaaagcagttttcaggtcagctgactggggctacagtgtgtggcaggtcctgcttacattactgccaaatttgtactaaacaatgtcaaagatgacttctgggactccaaagtatgtctagttcataatattaaaaactcatgctgagttggaccgaattcacccttctagatcattaactcccttgggatgggccgtcttagcatcacgaaaatctcccagcctgtgagaactgtgggttctgagccaggcaccggcttctgagagcagacacactggctgtcacctaccccctgaggagccagcccctcagtgcaggtcacgccttgaaagctggcccctctctacagcggcttagtaggtaacaggtagacgcattctttagctgctcaagtcacatcctgtcactggttgtaaacccactcgattctctagtcttgctggaatcgtgttgatgactgcagggtcaaggaggctgtgggctggggtttgacatctttgttacacatattaaccacggaatacatgacagagccagagagcatgccggcacgtatatgcgtgcactctacatcccccgactacctgggttctaatcccagctccccagtttaccggctgtgtgacttcaggcaactgccttgggtctcaggtctctacctgtaaaagaaggaaaatgacaggacctatctcaaagggttgacataagaaagaaatgagtctccagcctgggcgacagagtgagactctgtctcaaaaaaaaaaacaaaacaaaagaagaaagaaatgagttaatacggaaaatgcccttagaatagccaagcattcggtaaatgttattcattatcatgattcaatatttctggctgttatttggttgtttccccccctctgtttgtgtaaatttttaattgggataaaatacacaacatttacaattttaaccatttttaggtgtacttcagtagtggtaagtctattcacaatgttgtgcagttggtttttccttccacttttcttaaccattctcaatatcgcaactacatccactttgtgcaaacatcaggtttgtgcaccttatacgtagggaagggccattgcctccctcactgttatccaagaacacctttgaggattcatctgcactatgatttctgtgggaaaatacatttaaagtgccagtcttcattactctcagaacacggcctgtttggaagtaagcactgcttgcattttatgtccccttgcagtatctggcgagcgctgggtcctccatgacccctttggaacctttgtattaatcttctggctgtctcctttaagaactacaagattaacctttttgcagccctttctgttctgggattcttgaatctggcagcaggtaacgacaggtcccatcagaaacaggtcaagatcttgacttcctgccctccctgggagcagggcccacgagcaccacttaacacctttttcccctgacccagcctcattccagccttctgcaagtcctgctttcctctccaggacctttgttcctgctttgcttcctatatttcagggcctagggcagcatgaccttttaccttctgacccttgcccctgctgcagcatgtgttttgtgacggagtgtagggactggattgtgcagagaccctctttgcatctgtctgggccctcggcctattacaggagggacccctcacccttcctgcctgcaccagggtcctccaactggaggctgcttttagccctagggctagcgtgctctcttaagccagaagttttaggggcaagagatccctaatatgcatgaatgactctcacaagacaggcaggaagtctccaagagacttcctttacccctagggaaatgccatctgatctttgagataaggggctgggagcagtttgatttcatctccagagaaggaagccctcagttaaggggtcatcttgctcttttccccactccagggcctctctgccttatctctgtgccacctgtgaccccaggccttatacatgattgagaggattacccaaacagacctaattgacttattggaaatccaatcctttgattttttaattcacttcttttgtgctgctttatggagacaaaggagactcccctatactgcctgtgaagagcaagacagtctgcagccccgactgtgaggactgtgaggcctcctgtgtcagcagatgtgcatgtactctctgtactgatcccctggaaaggtggcctgtttgaaagcagtcctgcccaggcctgcagtaatgaagattaagcctcagagtactacaggttacccattttgttttgtttagcccctttcatcaaatgcttataaaattctaggagcttacctagggtttattgttgataacctcaaacatatgtgaatcatccactccaaactagggcagttttcttctcaggccctccaagggagttcggtgacattggcacattgtggggatgaataggctgaaatgccaggtacacagttagtggaaagactccaaaccaagcaaaggttgaacgtcaggtaactggagtagggggtgtattagttagctagggccgctgtaacaaagtaccccaaacggggtggcttaaacaacagatatttattatctcacttttctgggggctagaagtccaaggtcaacgtgtgccagagttggttccttctgagagagctgtgaggtaggatctgttccatgactctcttctcgtttctggtggtttgctggcaagctttggcattccttgccttggaggtctctgccttcatcttcacctgcattcttcccctgtgtatgtctggaagtccagagtatactttttataaagacaccagtcatattggatgtttaaaatgacctcatcctaactaattacatctgcaaggaccgtctttccaaataagatcacatcctgagggtactaggggttaggacttcaacttatgaattttgggggagagatagtcaacccataatgggcataagagaccgggagtggtatatgaaatatagtttgaattccacctcaagaccttgagcttatgacttctgtctagctctgtgtatcattcatacttcccactaatttttaacaaaatgactgggagagggagaaagtgaaacccagtggactgaggcttgagtgagcattacctcctgggcctgttgatttgcgtctccctggtgaccttcttctctgaccagcatccgtgggcttagggaaagcacggtggtggaaatgtgatcctggtgtgtgagatgcaaacttgatcttagcctttgctctccctggaaacttatcggtcaaaactttcactaaaggcagttttttgtctgtttgtttaggtttactggttatttattatttatagcatggttaaattgggtcaaatttttatgaattgtaagtagagaaacttttctaataagaggaaaggataagaaatcagacctttttgtaaagaaaattaccccacttaaagtcctggtttattttaggccgtttggcagtgagaagaagggattatttgtgtctgacagaggtctacaaggaaaaaggtttgggatcagaaaacacttccaaccagaatgacggttccctgccagtccttggtctttcttcctcccttccttctgtgcccacatggactaattatgtctggctttccctccctcttccttgtatatttcccttccatatcaagaccttcagaatgagggcaatgtggataatttcccagttttccagctgggcagctcaccaagaacaaacccagacatcaaggccagaccaaaaaaatcttcttgttttagacagaagactcctacatctgggggatttaatgcacccactgtccgctcagtgcatttcctgtgtccggagcttctctgcctgccggctctggtctcgtaagtttccatgccctgctgccaacttataataacagaggcggcaggcagcaggctgagaatgtcatagcctactccatacatgggcactgctttgcagactgaaacaaagcttgcagggtctcattgcaggcctctcagccttgacagtggttgtgcccagaccaccaggagcctggaattaaataccctcggatccagttctctactgttaatgttatcagcttcatcaagtacttcctctttgagtgatatctgtggagaaataaataaatcaagctcctccaagtcctgttcctgcctggatcctgagtccaaagtaaaatctgagttggattatccaaagcactatcaccctcctgcattgttcccaaggtctgggcaggaacggagagtgaaatctctccaggacattacatcagttcctcctttacccttggcatgaaagttcttcctctcctggtcctcccagacttcctaaggcctgccctgagtgcacacccacacactaagccttgaagcccaggacatcaaagaaagcctgcaggtaaccagattactgaccaacgcctgtgaccgttttgaaatttataagatgggaaatggattcaggaaatgggatttttcaatacatggtcttcatcatccttaagtatttttaagccttaataagcttttaagtaaagagagacattctgtaaatggcatttagaagaaaatataggaaagttcaaactggaactgtcttcaatagttgtttacagtgatcatattctgtgacaaatgaaaagtggaccagggacaaaagataaatacatttctatgggcagcagaattgacatccatattatctataaaaggaaagtttgccttgtctacaaaagattacagatcattttagttggtgcccagtaagcatttctatgcatttgggttaaagtgaggaagttagtgcttgtctttccaacctatatccttgcaaggttaattaagcactttaaaaaaaaaaaagctttttataacaggaaatatcaaacctatacaaaagtaaagagaattgtgtaatgaatcaccatgtacccatcaccagttttgatgatcaactcaaggccaatcatgtcttcctctgtggtctacctcctctctccttcaccatcccagattattttacagcaaatctcagatatataatttcccttgtaaatagtatcaacaggctgggcgcggtggctcatgcctgtaatcccagcactttgggaggctgaggtgggcggatcatgaggtcaggagatcgagaccatcctggctaacatggtgaaaccctgtctttactaaaactacaaaaaattagccaggcatggtggcgggcacctgtagtcccagctactcaggaggctgaggcaggagaatggcgtgaacccgggaggtggagcttgcagtgagcagagatcacgccactgcactccagcctgggtgaaagagcgagactccgtctcaaaaaaaaaaaaaaaaaaatagtgtcaacataggtctcaaaaaaagactataaaatcataatagaaccatcatatcacttgaaaactagtagttcctcggtatcatcaaataatctgctagtgttcactttttgccaaccatctcataaatgttttttacagtttggatcagaatccaaacaacatctatacattgcagatggttaacgtatctcttaagtttttttcaacctataggttcccatctgcaaccttcttgtgtgtgtgtttgagcagtccctctcttcaggtttttttaaagaagtcagccataattagtatattcgtatatataatatattatatatattagcgtaggcttatatatactatgtaagtatatacttatatagtattagtatatactatatatcatatatactataatcatatatagtatatatggtgtgtgtgtatatatatactatatatcatactatgcatatatagtatattagtataatctccccctgtacatagtttctttttttcctctggttgctttcaagattttctctttatttttggttttcagcagtttgactatggtgtacctgggtgtggtttttattttacatatcttacttgagattcttggaccttcgaatttactgttgttgataatgttgttaccaaatttgaggacaatctagccattattttttaatttattctactctgtttttttctatattctccttcttggacaccaattacatgtgtgttagaccacgtgatattgttccacaggtcactgaggatttgttttgtttggatttcatttttactgatctgtcttcaaattttctaaccctttcttctctcatctccgttctgctattaagtccatctagtccggtctttcatttcagatattatacttttcagttatagaatctttattttgcctttttttttaattccatttcccttttggcagcctgtatgtgtcatcatttattacaaccatgattttcttttagggcttgaacttatttataatagctgctgtaaagtttttctgtttgtttgtgttaactccaacatctgcattatctcaggttctgtttcttttgatggcctttcctccttattatgagtcacatgtttgtatttcttctcatatctagaaaattgtgactggatgctgagcacattgagacactggattgtgttctattccactgaagagtattgacttttgttctaggaggcaatcaaattactggccagttactttgaatctgtagaggcatggtcttacgtttcattaggtccaatctgtttctattttgccttttgcccttagcagatccccttgtcctagggtttttactcctacattgtggccattctggggtaacaaacagcctgaagtgtttacagagcctttgaacttgatgagactcaaactctgaactctgtctccactgcagtagccagtagctgaggtttccgcttagatcttttgattatacaactgttgcttttcactgaactccaaagggtctccctggcacttgggctattcggtaatcagacaagaatttgaggaagcttttacatgccaatttaatcactctcccctctgtggctcccactgtatgggatttgtttcttcaattttctgccattctggcagctacctactctatctcaagcaactttctgtttatcttctaactgcccctgtgctgcacggactggggaggaacatgcagcatagggcagttaggagatcaacagaatcagggggaaagctgtttaaatatggacctcactctgtgcagttcccttctttcaagagtgaaatcctctccggtttttgtctgcccttgataccttcaaatagttttttcaatgtatttaatatggtttctttcatttctacccgtaagaaggttagtttggtacacgccactctgctattgttgaactgaaattgcccctttccttgtttagataagtggtggcagatcataaatacttcatcttgcttttcgtatctaaccatagattctgatgctagtccttagcaaaatatcaagatactcctcattcctttttacttttacgtagtactccattttttagagatatatactataatttattaaatcagtcttctattgatagctattccaatgatgaggcacttgaatttatggcagtgggttatacagtagtcacaaaataagccctttgccttcaaggagatgatcttttaatattgtgaacaatatttttgttcagtgatcatatgatagatgagatctagtagaactttcaaaaaagaaaaaagtccgcattttcttcttaaccctgtgtaaaacttaaaaccactatgtctgtctctgtttactgaccctatcaaattacggaattcactttatgtgttcttcatatatgtatattatcttaatggacaaaggtgatatgtttgacttgctttacttagaactgtggacaattctttgaccagaacaaggacagcaagcatgcacatccttttggtgagactgccctgggaacagatgtggccagagagagttcattgagttgggtgtggagccgtgtagaaatccccatttttatgggcagatttctgtccttctgcaccttagcagggacttctggagccaggtgtgtgccggtccattccccgacccagccctcagagctacttctcttgctgagctgtgtacacttctgatgttatcctcgctcacatgtcaccagcttgcaactttgagtgggatgagttagaggtgtccatccatcctgcttcttccttacttctgcctgtctctgagcactcaggagcaaagtgctcccaaaagttctttcaagttcagtaagattgtcattctgtaaattggggccactgtgcatgcttagagcaggttttggtgtgactttgtgtcatagtttctatattgcagtgacagatgttgtcatgagtacacaggttctagaatctgactggagtctggttctataatctcctaactggctgtgtgaccttgggtaagttatttaacttctttacgcctcagttttatcatctgtaaaataggggtactagtggtatctcttggttaggagtgtcatgagtgtcataaggttgggggtttttttcttttttcttctttttttttttttttttgagacaaagttttgctctgtcacccaggctggagtgcaatggtgtgatcttggctcgctgcaacctccacctcctgggttcaagcgattctcatgtctcagcctcccgagtaactgggattacaggcgggagccaccgcacccagccaggagtgctataagttttaaacagtgaaatccagcatctaagatatagttattactaataattattactaatattattaacattatcactaatactaataactcctagagttagagaatatgaaaattttagttgtgaaaatgtaagtcaatgaaagcatctagagagaaacctgtaagaacccttccacctctgaaagcgacgtagaagagttaaatgatggccaaaagatggggcatggaagtttggcttccttgtgcctacaggccacaggtaggtcattaacggaatgcttgtagacatccttaggttgtctggcaatagaaaaatagtttcaatatgcacaccttaaggccaggcgcggtggctcacgcctgtaatcccagcactttgggaggctgaggcaggcagagcacgaggtcaggagacccagaccatcctggctaacacggtgaaaccccatctctactaaaaataaaaaaaattagccgagcatggtggtgggcgcctgtagtcccagctactcgggaggctgaggccggagaatggcatgaacccgggaggcagagcttgcagtgagccgagatcgtgccactgcactccagcctgggcaacagagtgagactccatctcaaacagaaaaaaaaaaaaaatatgcacccttcaaattgttaagcagtgaaaaagacactgcattctggggagaaaggtcctttacacatacctacacgctcctccatttaccgcctgtagagccgcagggagtttcctgtcctgccaactcactggggtctgtgattgtctttccctctgctccacagataactgtgtccagaggacccccaggccatccgtggataatgtgcaccataaccctcccaccattgaactgttgcaccgctccaggtcacctatcacgacaaatcaccggccttctcctgaccccgagcagcggcccctccggtcccccctggacaacatgatccgccgcctctccccggctgagagagctcagggacccaggccgcaccaggagaacaaccaccaggagtcctaccctctgtcagtgtctcccatggagaataatcactgcccagcgtcctccgagtcccacccgaagccatccagcccccggcaggagagcacacgcgtgatccagctgatgcccagccccatcatgcaccctctgatcctgaacccccggcactccgtggatttcaaacagtccaggctctccgaggacgggctgcatagggaagggaagcccatcaacctctctcatcgggaagacctggcttacatgaaccacatcatggtctctgtctccccgcctgaagagcacgccatgcccattgggagaatagcaggtgagtgagttcccctctcgccgctccagcatcatggggacctgacaaagtcccactctcccctgtgatctttgcagccagcctcgcaccattcccaattaggcgccctccaaggctctctgagggcaattggaggcttctgcttggatgaggctaaatccctaatggcttggttaatgagccgctgggatggagtagttaatgagcctcagaaatgttaagaaacaaatgtcctacgtccagcttacaaggagagtcacatcagaatcaaggctaagcgaaaacatttaaaataaaaggtttatgagcatgctaatggccctgtcctgaagtttccagcagctaattaatgaccagacacagcataaagaagctttgtctcagattcaggcctgtaattccatcctgtgagcggtaacgccatgcagccttcaccttaggagcgggtaggagaggaaaacaggattatggtattggaggcaggtgttgctgggccatttggacagaggaaaccactcccagattctctttcatttattataagaagcccaaatttgcttacttaagggaagaaaccagtggaaaccagtgggaaaaatatctacacattgtctgtttcctaggatgctcataatttacatgaactcattcttcagaagcttaatcctctaaccatgtgacaaggttagctcttttcgtagagcatttctccattagatatttgttgagcacctgctgtgtacaaataaatgcattaggagtagggatacagagctagtttaacatgcaatatctttttccccaaggaatttagaatatagtgggagagacaaaggtgaaaaccactaagatataaggcagaatgaagtcagccccatttcccattcattttatgtaccagcattcattgagcacctactgtctgccagagtaataaagcgctgtagaagcaaagcagaaggaaggtgtgttttgactggagggaccaagcaaggctttgcaggggagagagcctgatactgaagtgactgtgctttcgatagtgggaaggaggcctggaggcaggagagaacggacctgttcagggaagggtgagaaatgccccctcctccctgagcccatgacccggtgagagctctagctgaccatcaaggtgctgccatgctgatggcagtggaagaacaggctcctacaagcctactttacttctgccttgaggtactgtcccttttttttttttttttttccacactctgttgcccaggctggagtgcagtgacggaatctcggctcactgcaagctccgcctcccgggttcacgtcattctcctgcctcagcctcctgagtagctgcgactacaggcgcccgccaccatgcccggctaatttttttcatatttttagtagagacggggtttcaccatgttagccaggatggtcttgatctcctgacctcgcgatccgcctgcgtcggcctcctaaagtgctaggattacaggcgtgagccaccgcgcctggccggtactgtcccttttttatttaaatcatgtatttctagatttttctctggccccaagtgcaccaccagtgttgtctcagtttcactgttataaatcatttacatcagggatgttgaatccagaaaataaaatggcaaacaaaaatgacttatatgttttaaaaactatgaaaaaggaaatgatatatgtttgggattccattcaaaacgatcctgcagagttagggggtgagatgtgggtggggcctacataaaacaaggctagccatcaccagctagtggctatatgtctgggtgatagatgcatgggggttaatattattgatctctctgcttttacctgtacattagattgttttaatcagaaaatgaaaaaataaaactggggaaaaaaagaatagtagataaagaagcaaagagcttgggcaaaacatgtgaatttccccaaataattggagaaggaggatgaggtcagtgtattgctgtgaattctataaatgacccatctcgtgctttccaggcaatacccttcttgtctgtataccctctcgtcttgtttcttgcatggtgtgtgcagcagtacttgacattttgcagataccttttgcttgtttataattctcaggaccgctctttaaggtagctcatgactgcagtattcatcttatgtggaagggtcatttgtacaggggaaaccacagaggaaactgagccccagagaggcaaaggcctttgccaaggtcagataggggccccatgaccgaggtgtgcaggacaacccttgctgcccgtggctgctgtccatagtgctgccctcgtgggcctggcttgcttcccaagatctctcctgtctacctcccaggtgacactgccttcaccacttcttgggaatctaccataagcccatgaatcaagaaagacacatggtttagacttgaggagttatttttcccttatttagtaggtttgtaaatttcaaaatattagtctcttaaaattatattactttttttttttttttttttttgagacagagtctctgtcacccaggctggagttcagtgatgcaatctcggctcattgcaacctctgcgtcctaggttcaagtgattctcgtgcctcagcctcctgagtagcagggattacaggcacacgccaccatgcctggcgaatttttttgtatttttagtaaagacggggtttcaccgtgttggccaggctggtcttgaactcctgacctcaagtgatctgcctgccttggcctcctaaattgctgggattacaggcatgaaccactgcacccagcctatactactatttttttcatctcattccccaatatcagtgtttgatgatggatgcggcctcatcggttaggaagcttggtggaatactgcacccaccaaatgatgagcctgggtgtctatgggtgcccgctctctcaagggcacacagaaataggcaatatgtgcttttcttgatggctctaggaagtcaagaatattcatgtaaaataaccctggggcctagatcttttgggggaatgcgttaactccttcagtataacacaatgtcctaaaactaattaccagtgctataatggcatattcaaaagtttttaaatctcccaatatatattttaactaatgcaaccaaatccaaatttaactcattccactgaaaggcattgtttttctccgaagaaggtaggactgaatcatccctaaagttccttccagctctgccagtctgtagtctgacagtcttgattctgtaaccctcccaagaaaacacaagagttttaagcacctttgtttctttgaggatacccctgtgtgtactccactcttctgatgtctttctggactcctcgatccttgtaaaattgattctgtttattggaggtgaaattcattgggtgattcctgtgtacaacgtaaaagttctgtcattaactgaatagccataaaaagcctttaaatctccgtaaagcagctgttagatgagaaaacttagttccttaaagcttttcaaatgagcacagcacaaaaagaaatgaaggtgctgctgaatgtcaaactgatgagtactatagctggagggtatagatggaaatagtactcatgaatgtggatcacaaaatgaagctaaattcttactattcagatatgatttgttcaggtgccctagaatttatgcatttggacaccttctgtgtataaggcactgttctgcattcttgatggggtggggacagcctcagagaggtataagacacagtctttatcatgaaagagctgagcacagttaactataaaagcgaagcacatgacatgtatcttagaagggctcctgttaagtaccacaggaggtcagagtagggatgtcagggcaagctacatggagggctggcttttagctgggcttggatgaatgcttagaatgtggacaagtggagattagagaaaaacaggtcattccagatggagaaaacttaccaggagcaaagcaagataaggctcggtgtattttttctatttcaggtagaaatgtaaatctacaagttgagatctattgaggtcatttaaaaatctatcatttttcaggctgggtactgtggctcacgcctgtaatcccagtactttggaaggccaaggctggcagatcacctgaggtcaggatttcgagaccagcctggccaacatggtgaaaccccatctctatcaaaaaatacaaaaattcgtcgggcatggtggtgcacacctgtagtcccagctaccgaggaggctgaggtgggagaatcgcttgaacctggaaggtggaggttgtaatgagccaagattatgccactgcactccagcctgggcaacagagtgatactctgtctcaaaaaaaaaaaaaatgtgtgtgtatatatatatgtgtgtatatatatacacacacatatatgtatatatatatgcgtatgtatatatacacacacacgtgtatgtgcgtgtgtacacacatatgtgtatgtgcgtgtgtacacacatatatgtgtatgtgcgtgtgtacacacatatatgtgtatgtgcgtgtgtacacatatatgtgtgtatacacatatatgtgtatgtgcgtgtgtacacacatatgtgtatgtgtgtgtgtatatacacatatgtgtgtatatgtatatatgtgtgtatatatgtatatgtgtgtatatatgtatatatgtatatgtgtatatatatgtgtatatatatatgaaatttttcataaattcttttcattatctattatcactgaaattccatcataccattaagatacatgcagaattgttctcactcataggtgggaattgaacaatgagaacacatggacacaggaaggggaacatcacacaccgggtcctgttatggggtcgggggaggggggagggatagcattaggagatatacctaatgtaaatgacgagttaatgggtgcagcacaccaacatggcacatgtatacatatgtaacaaacctgcacattgtgcacatgtaccctaaaacttaaagtataattaaaaaaaaaagatacatgcaaatttatgtctgttaggtatttaccatatggaaaaactaaaataatcaagaaaggaaattcagtaggtggttgacagtttaaatacaaatcacaaatgacatctttttaaatataacaagtagcttatttttaaaggtgtcacttactttgcaaaggataaggagaactgttattaagaatttaccaggttggaatggctagtgtttatgggagagagtttagcacaagacggagtgagacaagattgctggggtcttattttgaatccagcagccaacgctgtatggaatagtgaaaaagttgcctctttctgtgcttgttttctacttggtatctttaagaatgggctatttaggagcaaggagcctagcagaaagcatctgtcaatcttctaaaactttacctacaaaagttttactggcttgtgtcccaccagtgtgtttcaaagccagaaagtatattggccacaagcacctgtaggtggctgaggtgtgactgtagtcatccgtgagtcatgttttcagtatgtattgaaattccttatttggacttgatttcaaaggtagaagggccttttacaggtgaaggaactgagattgagacacatcaagagtctagcctcgagcttctgttcagtgtttttcccaaaattattgtttccttctggtctgggactctagagcaaagattgaaaactgtggcccaaagtgtgagccaaatccagccaccatgcgttttttttaaatcaagttttcctggaaaacagccaaacccatttatttggtattgcctatgtctgcttccatgctgcagtggcagagttgagtagttacaacagagattgcatggccagcaacacctaaaacatttattagttggccctttacagaaaacgtttgatgctctctgccttgcaacattgtgtacgtggtggtcagtgtgcttggaatcataggtgcaatgtaggatgcagacttagaaaacagtgcccttgctcccaaggaattatttgcactttagatgacaaggcatgtttgagacatgtgaaaggaacaataacaagttatgtaagagaggagacaggttctggactgcaggctatattttcagaccaaccagaatagttacgataatatcattaacataaataaaatactaagtatttagctacgtaaaagaaacgatttatttgcactttggattatacagttcttcattcattgattcaaaaattattgccagtctgctcatgtgccaggcattgttctagatgatagggcactgcatctctaaatgccaaatttcgtatccttgctggtggaaggcaatgtcctcttgctttatgccatgacacaaactaagctcctgctttaactatagacagctaaatcttaaacgaacacttttcagtcagtttagttttctcttaaagtactttaattccgttaaaacctgagccaccagatttcctgtctgcctgtctcttccttggccttaatccaaaactctgctgaggaagaagattcgttggtacactgacaattgttttataatccattcatttcataacccctcatttgctacccaaaagtggggacctaaatctgtacagatttacgtgttcaaaggccatggcctacccaaatagagaatatctcctttcttgtgtttctctcacaacttttgctgaagtctctccatctttgtgaacatgaacttctcgatttgtgaggctatcgctggtcagttgggtcattaatcctacagcaggcaagacacagaatttcctctcagctcaagatctcttcactcccaaccctcttagaacacacttcgcagcaatccttggaatctttaaaggtagccttttgtgactttaaaaaatacttttactatctctcaaggttacatccttaacagaaacaaaaagattctaatcagcgagacatcaagtctcttgcattgcttatttgtcacccaactaaaacaatgtactttcttgatttggttttattgactggatgaattatatatatacaaagattcaattagggaagcctctgaagtaagatgaagaaaaaagtatgaaactccagagaaagcagtttgaggcttatcctcaggggcttcaaggactgggagaagtcactgtattctggcatcttgcttctgtctccacttcctcccacagtgtttaatgctgctgtgaatttgtttcaaaggaagcctatagacttttttttttttttttaattttgtacatgagcctcaggagcagccttgacagctaaaagacctcgtaaaagagaagtgtgaagggattgcatgaaagtgctgggggcaagtctcagccctcggcctttgttggaaggtgtggggtgtgcagacacatgtgcaccttcgcacacatgcatctgtgcataaatatctgtgtttgcaggaaagaaaagatagggttgtgggatgtccccagaagccacagggattccagtgttttagtaccaggtgaaagagccgcttcctcccacactatctcagggagatgcccttctgtagacctctgtagcccagtaccctcaagagctcctggagacagatggccccgaacagtcctagggaagcagtgtgggcctgtggcaaaagcttggactgcgaagtcagcatgattggaatccggttcttgactcccaagcaggtgcataatgtccctgagcccttctttccttatctgtaatattgagaatagtaatacccatttctgggcccttttgactgttaatgagacaacatgtgaatgcatggcacaagaaagttaattgctacgtgtcactgtccccccctgcctcactgctgagctgccatcctgcccatctgagagggactgtgtgggagctgtgggtacagccagcacaactgcctgcagagccacgcagggcactgacagggaaggcctgtgtggggtgcccttcaaggccacctccgttttcttatttccttggagatttacttttgtgctcctgcttcccctccctgaagctttgcctgctgtccatatttggaggggatgaataagatgagcactggggaggggaggagccatcactccagcaggaatattcccagcggctgagtcactgcatttgattttcagtgtgaacaactgagaaatcaaagagccctaaaagaggaggaagtttcaggacaatgtttttaaaagataaaaaaacatcaacagcatggtgtctgagtggaaaataatagaaggggcttgaggagactggaaattccagtttgtgtaagggcgcacagtgctgtaatacagagtcctacctccctgcagtccctggatgtgaattcccaggctgcacccacacctgagcaaagcggaggctgggccttattaggttctgactgcgggctggatctcagctgcaggctgtgagtttcggataccggtgtttccaagccttctccacaccttatgtaatcacaggtgaaaaggtccagcctcatttgcagctgtggttcttctgccccaggaagccaggaagcgtagatttttgtattatttgcctccacttctccctcccccagcgtgccaccgtgcacatatgttttctgttttcctaagccacggcaaagcacaagcaattgcccattgtaaacacacctctgcgtatcaacacacacctggctgcactggattgaccaactttctgcttctaggtaatggctgatctagaatagaggaggaggaggggaaaaaaaaaaaaaacaagcagcagatggccccctgaatgttgtaaaccgtgtttctctaagcagggtttctcaacactctcgatatttgggccaaataataatttgtggggttgcttactatattgtaagagattcagcagcatccctggcctctacccactagatgctaggagcaccctgcaccccagtgtgacaaccagaaatgcctccagatattgccacatgcccccagttcagaaccatatttgtaaagcttagtagcaactttttttttcatatgctactttgcgtgttttgttgctgaactggaaataaacgttaatttttaggtaataaatttcaaaaaatgtaacagggacactcagcctctcttctggtaattatgcccatgacactgtcatttgcaggccttctttaaaattccacttgggcagttaaaagaacataaaatgactttcttagaagctttcctgaaatttaaattaaccacaatcattaaacattccctatgtcaataatttgactttcctactcaaagttttttaaggcatcagttttgtctagcgtggtttttttctttagaaaccatgttggttatgtgtgaattgcccatctttttacaatctctgctcaataagtttatacaatgagtctctggtaagagacagacgtgggagtgaaaatatttctccttaaccacaggaagggattttttacaatgcctttagctgcggataccgagggtacctcacccagcaggaaggagagggaactatacttgggctaaacactttcataaactacttctcatggaaaagaaaatccaaatagtggaaatttctttaaaatatccatgtgccttgagatagaagaaagccctttcgtttaagaaaaatgcacttaaaattaatgaataccttgtgatccaaaaacattttatacatatacgttcaggtgttttgaagtggagtgaacttctatttatagagtgtgttgcgctttattatatagtattatgcatttacattgatgatcaaagaacattgtatctgtgcaggcatgggttccgaaaaggctgaaaaattccaggtgagccgtggcaacaacctctaaaggatctaggaacctaaagaggatctgccttttatttaatagaaatcttagaaacaaaaataactgacatggcttgaatgtcaattgtttaaaaaaaaaaaaaaaaaaaggaaaaaaaatctattgtgtggtcaggcagttatttttatcatctaggcataatttttaccaaagaaacaaaacaatcaataatgttataaaagccacttatactctgagcttgaaacaactgctttatgccacaaaattcctattgctgtctccgttagtgactaattcaatatgtcctgaatgtcaacgtatttttattactcagctaaagttggctttgctggattaaatgtcatttaaaatgatcaattggcagcttgtttggggttgttttcacatttatgaaaggaaaagtttgccggatacaaatgttcttgaagctgatagggaaacggtgttctctggtaagccctgcccttccttttgccatctgataagtagataacattctaggtcctagtttgatgcctccgaaaaaccaacatggcagcccagtgcaggttaagcccttacattggagaaatggtctggttcacgtttcactgttttatgagaagtagaatgtaccattacagtaaaagagaagcaacagaaacatgcagtgaaaacgcccatgcttgtggttagagaactgtccgctttctttttgctctacagtaggcttctgagtggagtgccttccgcctagagtcaagagggctgccatgatgtcacccaccttcatatgggcaaaatgtctttatatgaaactgtaaaagacaaccaggtcgtttcttaattgctgaaatttattaaaatgaacacactagaaaatcacattcccaatccagaggaatccaatggcgtcgtcagcctatttgcaatggaaatgattgtaaacctatagggagacataggaggggctgcacacaaactactttcgttcgtcctgatttactggcttcttttccccctagagatacagtctcactctgtcactctatcgcccaggctgagtgcagtgggttgatcatggctcactgcagccccaaacccctgggctcaagcaattctcccacctcagcctcccaactagctgggattacaggcatgcaccaccacgcctggataatttttaaattttttgtagagatgaggtctcgttatgttgcctgggctggtcttgaactcccggactttcaagcaagtctactgcccctgcctcccaaaacaaacattgggattataattataagccaccacacccaaccctaatttacaggcactgtatgtgataatatgtgattttgtcagatgttttccagttggaagagattatggaaaattaaaatgtgtctgccatttgatagtaggacaaatactgaaatattaagccacagctgagttctggccttagtccttttggggctgctctgacaaactgccgtagactgggtagttggtacgcaatagatacggattgctccacagttctgggggctggggagatcaaggccctggcagatgaggcatctggtgagggcccactttctggttcatagacagcaccttcttgctgtgtcttcacttggtggaagggtgaggggtctctctcaggcctcttttaaaagggcactaatcccactcacaggggtggggcccccaagacctaatcacctcccaaaggtaccttggtagttagttaggatttcaacagatgaatttggggaggacatgaagattcagaccacagcagatctctattttccctttatagagaaagtaattcaagacaaggagatgcttacaagggaaaaagacactttgccactttttactgctttgcttagaaactagaatcatcctccttcaaatgcctgtaaaacgagctagattattctcagtctgcagtaggacactgatttctcaaggtacttaagatgcttgggttgctcatcttctccacacccccacctcagcccatcccctggcaatacagaatgcagccatggcaagacggggtgggcaatagaagtggctggacagcttctgttgttagaatggggttcaaagtccaggactatggtgttgttgttttaagaaggaggccacttgtccttttccaaaacacaatgagtgacacaccacattaatgtttaaacacagataatacagaagaaaggattccataagggatactaaagatttagggctgctgtacatttaatggtagttccttgaaagattaagcttctggaaaatgccacagttttctgccagtgtcttctgctattcaaagagaacgctggagttcagaaaagatacactgagcaggtgtactttgttctggaaagtcctaagattttataaacatacacccattaacgagtttctctgagactagaaattgcattgggaagtgtgtcatagcatgggttttggggtcatgcacagccaagaatcctgtgtgtcattcataagctgtgtcctcctgagtaaatcatcaaaccctctcagccccagcttcctgatctataaaataggaatagcaacagcacctgcctcataaggcggcgtaagggtcagatgtaaccaggcttgtcgaagccacagcacagtacctacaaatagcacccactaaataccgttgttatttttattactttctcttccctcccattatcactaatataataatcctgtaaagtaagtgctgttattcccacttcataagtgaggcaacagaacctcaggaagattaagaagcgtgtccagggtggcccaactgaattccccttgtaaatatacccccagagaagggtgtatgtttctctgcaatgcggatgtgttggctgtaatatattccaagtctcaactggacactcccctttcaaatgtgccaggcctaggccttgcccaaagtaaaaaagaaagaacattgtagtgcaggagtccagagtgcaaatccttgtgacctcaggcaagttatttcaacctctctgaggatcagaaataatacaggtaaagtgccgagttcatacttgtaattcagtagttgatcttccatcaccagccattctgacaagtgcctataaaaagtgggcatttccagaaccatgcagtgaacacctggaaattggtgctgctcctcatctggaggccagttaacctttaactcaacctagaaggcacacatctgtccccagctgcctctccaggttctgctggagggcaaccacacaccttcacagcacacatccagaaagtcatagaacaaggggaaggtgtacatcatgtcttcttctttttttttttttttttttttttttttttttttttgagactgagtctcactttgtcaccgaggctggagggcagtggtgcgatctcgacttactgcaacttccacctcctgggttcaggcgattctcctgcctcagtgtcctgagtagctgggattacaggcacacgccaccatgcctggctaagtttttgtatttttagtagagacggggtttcgccacgttggtcaggctggtctcaaactcctggcctcaagtgatccacctgcctcagcttcccaaagtgctgggattacaggcgtgagccaccacgcccggccatgtcttctcttttaatttactagcccacagccaaaaaccaaggcccaacaccatgcctagtccatattaaatcacctaacacttaataaaatatccattgcctgactaaatgttcaaacatcagttctgcaagtggagaaagtgacttttgcactgggttcctttctgccatgatccactatatcaaaacattttttaaagacctccaaaatgtgatagaatgagaatatctgtatctctttttgaaaccatgctagccacagttcccacagtcagagtcaccaagccacatagtttttcttttcccaccatttttcactcctcactcctttttgctgtgttttagcctaccccagccttctcatctttatccatccatctcccaccaacaccaggataaccttttcactccactattttttaccacacagctctctctgtaaccatcaggaccctctttccacccttttttccttccagcctgcatttaacacacattcagagcccattgattcagttagaacttgcctcccgtctttcacatgggttctgcccaggtttaagtttgggtcacgaccgtggacttctgggtcactgtatcatcatctgtggagggaggctgtcgcctggatcatctctgaggcctccggtgggtcaaaggtcacacaacaggacctccctccatctttctgggcctccgtttcttcccggtaaaacaaggaagttagttagacaatcagtcaacccaagctaggcagaagcagttgctggattcttttttgattcttctggttagtgcctcaacaagaaacattttcaacagtgttttcttgcccttttcctctgtagactgtagactgctttgggattacgtctatcagttgctttctgacagccggtacgaaaacttcatccgatgggaggacaaagaatccaaaatattccggatagtggatcccaacggactggctcgactgtggggaaaccataaggtaaaagggcagcagatatctgctccataaactagtgccaaaatgaagtccttatccctggattggaggataatcgtctgtcttctgctttttacgtctgcccatacttatttagtttattccttgctgggcaagcaattaggcagttgcaaagggaaggaaataattaaagatgcatcttgccatttgttcctgagaccaggtgcccattaactcacatatcctggtttgctgggctgcatcatgagtgactcttgtttccactccccttgacctgatcccgccacctccactgttgttaggaactgggtaaaagggaagtacagcaagactctctgactcacaggggacctttgggaagtgccactacttttctgctcagttagaaactcccatgttccccaaagcccatttctttatagaaaatacttagattctgactgtaaaacatcccctttataagcaagcgatgcaagaagtttgccccacaatttcccaaactgggaggagggaatggaattgaataaagtcttttgcagtaaggaagactgctcaagtgcgcagttgtaaaaagcgggtaaccgtgatccaggccagtcgataggtggacctgaagcaaagcctgatgttggcatggtggcacaaccccaatccgctgagtgagtgaaaaggcagggaggcccagccaggtgtgcccagacctgcagccttctaagaaggcagtgggtaatgcagaccatgaccaaatatggcaaggccccactgacagacacttccatgtgtgagtcactgagaaaaatccctctgctactcccactgtaccattgcctcgggcccagaaccactgtattcttgcttccacaacaggaatttaaacaaaagaattgtcttccttaaaatgacagaccagaaacacaagtttgagaaaaggtcattgcaacaaaaaaaaagaaatgctgtgtgggtcagaaggtgaagggtgacccagagatagggggaacctgcaggaaattcctcaggcggagcagagagcggtcttgggaaaaggaaataagcgaggctgctatgggatctgctttattagctagggctccaggacttcggacaaagggtgattcaccttaggagtaaaccttggtgacagtgaatataaaaagctaaagggtggggacagatgtgcctggaaggcagccgattagcaggtagcttcccaaactggcagggccacaggcagcagctgaagagcttttattttaatagctcccgcagtgccttttctgaggttcatttcattgtgtctttgtgctttttttctcccttcctcctttgaacaaacagaacagaacaaacatgacctatgagaaaatgtccagagccctgcgccactactacaaactaaacattatcaggaaggagccaggacaaaggcttttgttcaggtagcacttcctttttctcctttccttcttttgggaggatgctgttttctttaaataacggggagtggggggagactgttaagatctctacctgcctatcggatacccaaagccaatcattgctacaaactggataccaggtactggtttgttaggatttaaggaaaatatttttttaacttatgtctgtcctgtaataaagccaaaactaaatgaacagaatctgactcacctttaaaataacgcagtgaccaaataatattatgtggcattaaagtccggtgtagttaaaagcacaaagtcaatggcaaaacaaggaattaaacctagacttccaggttaccatcgctgtaaggcaactttctgaaccagtatttgtcaaaatgcaggtaatgacccacagtgagtcatgaaatagattgaatgggtccatcagagctttttttttttaatgaaataaggataggataggatagagttgaaaatgccagactgcatgacatgtagcaagaatgacagttgattcatgaaaatgttgttccaggtgtgtatgtacgtgatgagtcatgatgtaaaatgtatttcttataatgcatcgtagtcagaataagtctacaagctgctgcactagaatttttctttccagaattcctcatagataatgagttccaaccaagttagcctgccaccacatagattgtaattccaagtactatgcaagagaggagaaagaactggaagaagaaggagggatctgaaggaccaatcaggagatgtagaagtagacctctggtgctggtggtagtcattagcggggccaactgcaggttggtagaagtgagatcaacaaacacaaccttgggtgatgagttttggcaggtgggccagaagagagtggcagatggggctagagatgtagattgtgaattatctatatacaggtaggaagtgataccatgtatgaacagatgagctcactgatatgattaatttattggaaagagcagagaatctggtctttagcattgggaaggaatggtaacttaggaatagtaaaagcaattaagatcttcaaaagaaacgggaaaggctcttcaggaaagggaaacagaatagaaggaataaaaaaagaaccctccattctctggctctaccgtatggcttttcttcatccttcaacctaaacgcagtccttggctccctttcttcccactctatacctttttcgtggatgaactgatcccgacacacaacttcatttatcacctcagtggttaggctcttaaatctctatatccagctctaaaatctccctgaaattccaaatttgaatgtccaagagtttcctgtaggctgtgtccccttaaatatcacatttaagtatttcagacatgacatcttcaaaacagacacttgtctcggattagaaattccacccagaggccgggcatgatggctcacacctgtaatcccagcactttgggaggctgaggtgggcagatcacgaggtcaagagatcgagaccatcctggccaacatggtgaaaccccatctctactaaaagtacaaaaattagctgagcgtagtggcatgcacctgtagtcccagctactcgggaggctgaggcaggagaatcacttgaacccgggaggcagaggttgcagtgagctgagatcgcgccactgcactccagcctggtgacagagcgagactccatctcaagaaaaaaaaaaaaaaaagaaaagaaattccacccagaattattgaattctttactcatcaggggcatcactgctgtttgaagcagccaactttgaaactacagttccctttgactcctcctgtatcccagtcattgtgctagatggaccattggctcttcagcaccattcccactctattctgtgttgcaggggctagaagcctgggaactacatttcccagaagggactgttgccagaagttcccaggttggagtctttcagtgacaagcagtttcatgagatctgggagactagaaaaaagtaaaggcttccctggcagtggaagctgatgtctggacttcatgattcacatcgggattcactcaacaacgtctttctgtctctagtccactctgctgcctcctgaaaccataatacaaactgccagctgccctacaacactaatacagttccctatgccagaaatgtatatgactaacttttgcctttttaatacctacaccctcaaggctcaacctcaagtctgttgccctgaatgaggccttcttcagctgctccaacacacatagctgtcgtcttccatcatcacctgggtttcctggagcccttaactacactacaatttgctttttcttttcttttcttttcttttttttttttttttagacagagccttgttctgtcacccaggctacagtgcagtggcacaatctcgactcactggaacctccacctcctgaatttaagcaattctcataattatcatgccgtagctaatagtagctggaattacaggtgcctgccaccacacctgactaatttttgtatttttagtggagacagggtttcgccatgttggccactcttgtctcaaactcctggcttcaagtgatctgcccaccttggcctcccaaagtgcttgaattacagatgtgagccactgtgcctggccaacttgtttgtttttctagagacagggtctcactctgttgcccaggctagagtgcagtggtgtggtcatagctcactgaagcctcaaactctgggctcaagccatcctcctgcctcagcctccggagtagctagagcgactacaggatttatattttgaatacaggttgagtatccctcatctggaatgcttgggaccagaagtgtttccagtgtttggattagggatgctcagcctgtagttttatgtgtcaagtccttctccccgacaactagattataagctcctagaaggtagaggattctattttttatcattctatattaaacctttcagttaatgcttattgacagcctggtgtggaatatcagaagcttaatatgggaaagtcgtcaaaagaagaaaggggtggtttggggggccaagcataggacatctgtggagttggcttagaggggctctttattgacttcggatgtggagggagaaattttagttaggggataaaagaggagaaaaactgcagatgctacagatcatactgctggagaagtttgccagtaatgagcaggaaaagcagtagtagccagaagattgataggcttccaataaaagattttcagtaagagaaaggaatagtgctattagtgagggagagattagtccattttgaggaaatgaatgttagaagccagacttgccacaaaatgaaggtttcttggtataagacctagtatgtaatcaagtgaggtgttgaattgtagatactctttacaagatgtttgagtgttgaagaagatggaggtttaagtgttgaaatcatggactagtgccattaagcagctctttgatgaggagacagaacacaaagaatgagtaggactcaactcgtgagaagagtcaggagggctccagataagggaaaagctaaagatacatgggagagcatgcgccatgatttatacaagagccaagaccgctagactgcattgggttcatactgatgaacagtggaaaataagagcttcctaggtaaagatggcagaaagacccgatgagcagagcaaaggatgcacctttgacttgatgagtggtcattttaggcttttgcatgtggaggaatgagagaaaatgtaagctctaagatagtggcgcagggcagagtatatggggagagaaaggtgtgcaggcacagaaggggactggaacgggtgctcctacagtgtaggtggaacatctgcaaccaggctgccttcctgagccatgtaaattatggtcaaaacatgtcactgatgtcacttccagtttaacccattgaacaggaagatgaggcttgctgtacaccccagttgcaaaggtagacagactaaagagggttcattgctggcattttgggtgaatcagtgataacattgtaaaagatttttttttttttttttttgcccttccaattttccagaagctccactaagttgtcctattgtcagcatttttcacttaacatttaactatatcctttgaaatttaagtaagttttgaataccatttcctgggctcacagtctggaatggaagtggatatacagctctcatgagtctgtaaaatctaattctgtaaaagaaagaacatatatacatggtctggaccaaggctttctcgtgtggtcatgcattaatgcaccacgtgatcccaaagggtgctcactacgtcacagacagcctagacttttttttttttttccaataattaggatttcactttgtcacccaagttagagtgcagtagcatgatcatagctgactgcagccttaacctcctgagcttaagggattctcccacctcagcctcccaagtagctaggactacaggtgcacaccaccgtacccagatttttttttttttggtagaggtggggtctcgctatgttgcccaagctggtctcaaactcctaggttcaagttatcttcccaacttggcctcccaaagcactaggattatatgcatgagctaccatgcctggccctagtctttttaaataagcattttaccactttttagacagtcagagaaaggagtaccttttcctaatttgcactaagttaacattaatctcggggttcagtagctctccagctgtataagatgatggagtctttctttatatacaggctagagttcagagtgaagacagctttaggaaaatggaatctcttacctcctccacttcttcttccaaaggtttatgaaaaccccagatgaaatcatgagtggccgaacagaccgtctggagcacctagagtcccaggagctggatgaacaaatataccaagaagatgaatgctgaaggaaccaacagtccacctcagcgggccagcagcccagggaacccctgcccaccaggattgctggaagtgtgacggagcaggcgggctgaggagagtggaaaaggaagcgacccagaaatggcagggacacttctcttgcagaccaagagggaccctggagcaccttagacaaactacccagcacaggcggggctggaattctggcggagggcatgagcctgggactccatgtcacgtttccttctgatttggaatctctccatctgtaattcctcaccctcacccttccaccgttgttagtatcatggtgtttttgtttttgtttttgttttaagaacctgcagtttgactcttcatcgttcatctaggggaagacatctgatgttgttttcctatggaaatatatatctattatatatatattttttgcaaatctcacaaagtgcggcaagcccagctggtcaggaaagagaatacttgcagaggggttcaggttcctctttttcctgccacgtggatcaggtctgttcctgttactgttgggtcttggctgaaaaaaaaaaatgcttttaaaaaagataaaatgaaaaggagagctctctttttctctctcttgctctgttcttcccttggtcccctctgtcctcccgccctgcctgcagttgagattcagatgccttctgacagagttcagcctcttggagagtcttggggattgttggcacctaaacagaatcagtgacccgggtgctttgtggccagcagcacagaatcaaacccgcatcccagcattgggccacccatctgagggaggccaaaatcatcacagatgctgctgtgctgcagacagatacatgctagtccagagagccgcccctgagatggctgtgagaaccatgtgtctaaggcgtaagataaggatggaaggctgtccaagttatttggaaggcctcggcagcttgggattagcttgggagcgcagcgctgcaaagtggaaaatatgaaaagaccacacaggcccagcagtccagaaactgggcaaaaatattctgcagtggggatttatttttccaaagcaggtaacagaggctagtgagaaagaaaagctcctctctgctccattccaaaggccatcttgtggtcagtttcatgccctcacctgatttttttttttttttttttttttcaattcctaaccttttttaaagtttcctggtctccactggacacagagctttggagacggaggatcccagagggcagtctcagttgcaatcagtgtgtgcccagcctgggcagacaggaaattcctcggatacattattttttctttctttcatagctgtgtctcagaaaggacccatttgtggctctttttcacctcaaaataagatcgatggtatcttgtaaaatgagggtagtgccacttcttagtatttttgaaagctgttttagattttttttttttttccttttctagccatctaaattgactcttccaatataggtctcagaaatccaatatttggagtacaatttcttttaatccagattacacctgccttacaaagcaccccctccttgttcccctctgtttcctctactcagttgggggagaaactcacagctcctccgggatacatatgtgccctcagcagcagctcccaggtgaagttaccagacccctgggcttctccccagctttttctgagttgagtcagacatgtagagtttgggtcacacaggcaagaggaattttccctcggccttactgacaaggacaccaacctagggtgcaaacagatggactatggttcaaggacactggaattgaggagctgatcaaggctctcttcagccttgctctgtccctgcctcttatcagagcacaggtagacacacgggcatagccagcccactcctactgtcacaggcgccccaccattcaaccttccgggaggtcagggaccttctatatgaggcgagtgggtctcagtctgcttgaatggtgatgagattctgctggatctcagcacgctgcaggtgtcttttgagagcattcagtaggacatggtgatccctatttcagcctctaagatgactggtattctatctgaaatgcagagattaagccaaatacctgatgtattgtgaaagccactgattttaagaatggagagaaagggattttttactgcatccctctgtatgaatatgaaatcagagaccagggcatgatgttgctaggattagagcctctcagtctggcctcttcacccaagtgcaagaactcagtctcttactgttcaaagaatcttaacagttgaattatggagggaaattcccttttgccccaagcatttctatatttaaagcaatatcccaggagaatatgttagacttaggatgataccttcagccacttgaagaagaaatagaaggcgctcattccaatatagtctttatttcccattcagatacaggttgagcatccctaatctgaacagttaaaacccccaaatgccccaaaatccaaaccttcctgaacgctatgacaccatgagtggaaaattccacacctaacaaacacatttgctttcttatggttcaatgtacacaaactgttttatatagaaaatgatttcaaatatcataaaattaccttcaggctatgtgtataaagtatatatgagccataaatgaattttgtgtttagactttgtgtccatccccaagatctctcattttatatatatatatatatatatatatatatatatatatatatatatatacacacacacacacatacacaaatattccaggatacaaaaaaaaacatttaaaaatccgagacccagaacacttctggtcccaagcatttcagataagggatatcaatctgtactaccaataaggatttcgtaattcccctaactgcaaatgtcctcttcatttgttctttatgagaaaacccgggtagtgccagcacctggatacagtatttacaccctgcagaccctaaagatttcagattcagttagcaaaccttgatgaagcacctgctggacactgagggacccaaagctcaatcagccataatccctgctttcagagtttatattgtacctgcctaatccacccggcgtgactcatttcaacactaagtactaggggtgttgtcaggagacaaatctgaagtcaggagaggaaaatgcaaaggagccctgccgtgtgatggatgtgcattctcacttgggtcttgaagttctcattcctacatctcaagctagccaggcagtctcctctctatcagaagaaagcactggtaattggctagactggctatgttgaaggtaacatgaactctaagatcttgacccagggcgacttggttttgcttaaggtggcatcaccaatgttccaaatcctttagggagatgagggtatccccacagaaaaagaggaataatagaccaatggattttctcctttcaccagtatgtttggaaccctctgatccaatgtcttttgatactgatctcttgtccaaatgagaatgtcgctttagctgaaattcaaatggctgtgacaatttaccgaaatgatgaagtaaccaccattcccacctttcactgcctaggctccaagtctgaatacatttttgaaataggaactcccttttgcaaaaaagaaacctgggtgtcagggaggtgaagtgacttgccctaggagcagacagcatgccaagaatggaattaggctcaggatccagcctgggctcaccctgtgtggctcattcccacccaggaaactgaagataaaagatttgggaaaacacaccaagaaaaaggggcagttttctttgcccaagcatttggtgctagttagaggctgttcactctctcctgctcctcttcggagtagaaataaaggctgtgacacaaggaagccagtggggtgggagggaggcaccataatccctccctaaaacccacagaagactaacctgatactcttttgacccaactgcatcaacactaaacagctgcagaccccctgaatctttcacacatgccaagtgaacattcttgatgatttctctttgtgaccgcaaccacctgcaaaccagaacgactctagaatttccttccccgcccccctttttgtttagtttctaatctcttgtttatgaggtgtggggtttataagggactgaatcaaatgaatgtaacaaaaaagaaaaaaaaaacaaaaaaaaatgccttttctcagggccagtgagttgcaaataatttttaaagaaaagcctataattacatcatctcaataaattttttataaaaaaaaaaaaa

SEQ ID NO: 35 - DNA, 199 bp, Homo sapiens
ctcttttcaggctgagtcctaaacctgaagaaagtttagagcctggggctctaaactacctgagtctttccaaacgacaagccaagaagacctgttgaaagtttcctcttaagtttcgtggagagagactcaggtatagaaatatccttactgccacctgacctgaagcagaagaaatcacagacagcttccagaccag

SEQ ID NO: 36 - DNA, 440 bp, Homo sapiens
gcccaacatgtgtagcaccagtgggtgtgacctggaagaaatccccctagatgatgatgacctaaacaccatagaattcaaaatcctcgcctactacaccagacatcatgtcttcaagagcacccctgctctcttctcaccaaagctgctgagaacaagaagtttgtcccagaggggcctggggaattgttcagcaaatgagtcatggacagaggtgtcatggccttgcagaaattcccaatccagtgagaaggccataaaccttggcaagaaaaagtcttcttggaaagcattctttggagtagtggagaaggaagattcgcagagcacgcctgccaaggtctctgctcagggtcaaaggacgttggaataccaagattcgcacagccagcagtggtccaggtgtctttctaacgtggagcagtgcttggagcatgaag

SEQ ID NO: 37 - DNA, 174 bp, Homo sapiens
ctgtggaccccaaagtcatttccattgccaaccgagtagctgaaattgtttactcctggccaccaccacaagcgacccaggcaggaggcttcaagtccaaagagatttttgtaactgagggtctctccttccagctccaaggccacgtgcctgtagcttcaagttctaagaaag

SEQ ID NO: 38 - DNA, 71 bp, Homo sapiens
atgaagaagaacaaatactagccaaaattgttgagctgctgaaatattcaggagatcagttggaaagaaag

SEQ ID NO: 39 - DNA, 267 bp, Homo sapiens
ctgaagaaagataaggctttgatgggccacttccaggatgggctgtcctactctgttttcaagaccatcacagaccaggtcctaatgggtgtggaccccaggggagaatcagaggtcaaagctcagggctttaaggctgcccttgtaatagacgtcacggccaagctcacagctattgacaaccacccgatgaacagggtcctgggctttggcaccaagtacctgaaagagaacttctcgccatggatccagcagcacggtggatgg

SEQ ID NO: 40 - DNA, 746 bp, Homo sapiens
gaaaaaatacttgggatatcacatgaagaagtagactgaaatatcagatttgtcatcaggaatactctttgtctactgtggtcctgtgcacgttggcctcagatggactacaggagattacaacgtacaaggcagatggagcattgacgttttcaaaaccattattcctgtgactggagaggcatcaggagaggtctcgttcgtctccagctcataaaatgtagcagcatcatccttgacagtgatgtttttcaggccctccattgagaacctgaggaaatctgtaaagataagtggtgatgttgtttcaaacgttcagaacagataccatcatcctgcctttgttagctgctgtagggaaagtgcgttacagatgtctgctgacctcacaagagtgaaaagataaactgtgcatgtgtttccacttccgtttctagtactatttatttttaaactacacttggggtggcctaatacctaggaagatgttgctattcacgttagtaaacagcctaaagaaactcttaggtttactgctacatccatttgtttggagaggtaactgttgtctgtgcctttttgaaaaacttccatttggtacaaaatttttactccaacaccccctcaacccttttctcagggaccacacctcttcttcccaaggtccctgggacttcctcattctttgtggtagtacaatgattggtagcaggtaaaataaatacatagaaagactactgtcaaaa

SEQ ID NO: 41 - DNA, 487 bp, Homo sapiens
acgtcagtttctgcactgaaactctcaagatcaatgagcaaagagctttctcagttctgcctttcagtttctctcttccaggaaggaaaacattcgagagagcgagggagagccgcgggagggcggggggcgggggcgccggctgcgggtgggaggagagaccgggaggccggccgggctgcgtcccgggtccccgcgccgcgccgcgacctgcagaccccgccgccgcgctcgggcccgtctcccacgcccccgccgccccgcgcgcccaactccgccggccgccccgccccgccccgcgcgctccagacccccggggcggctgccgggagagatgctggaagaaacttcttaaatgaccgcgtctggctggccgtggagcctttctgggttggggagaggaaaggaaagtggaaaaaacctgagaacttcctgatctctctcgctgtgagacatgtctgagactcctgctcagtgtagcattaag

SEQ ID NO: 42 - DNA, 130 bp, Homo sapiens
caggaacgaatttcatatacacctccagagagcccagtgccgagttacgcttcctcgacgccacttcatgttccagtgcctcgagcgctcaggatggaggaagactcgatccgcctgcctgcgcacctgc

SEQ ID NO: 43 - DNA, 165 bp, Homo sapiens
gcttgcagccaatttactggagcagggatgacgtagcccagtggctcaagtgggctgaaaatgagttttctttaaggccaattgacagcaacacgtttgaaatgaatggcaaagctctcctgctgctgaccaaagaggactttcgctatcgatctcctcattcag

SEQ ID NO: 44 - DNA, 135 bp, Homo sapiens
gtgatgtgctctatgaactccttcagcatattctgaagcagaggaaacctcggattcttttttcaccattcttccaccctggaaactctatacacacacagccggaggtcatactgcatcagaaccatgaagaag

SEQ ID NO: 45 - DNA, 546 bp, Homo sapiens
ataactgtgtccagaggacccccaggccatccgtggataatgtgcaccataaccctcccaccattgaactgttgcaccgctccaggtcacctatcacgacaaatcaccggccttctcctgaccccgagcagcggcccctccggtcccccctggacaacatgatccgccgcctctccccggctgagagagctcagggacccaggccgcaccaggagaacaaccaccaggagtcctaccctctgtcagtgtctcccatggagaataatcactgcccagcgtcctccgagtcccacccgaagccatccagcccccggcaggagagcacacgcgtgatccagctgatgcccagccccatcatgcaccctctgatcctgaacccccggcactccgtggatttcaaacagtccaggctctccgaggacgggctgcatagggaagggaagcccatcaacctctctcatcgggaagacctggcttacatgaaccacatcatggtctctgtctccccgcctgaagagcacgccatgcccattgggagaatagcag

SEQ ID NO: 46 - DNA, 143 bp, Homo sapiens
actgtagactgctttgggattacgtctatcagttgctttctgacagccggtacgaaaacttcatccgatgggaggacaaagaatccaaaatattccggatagtggatcccaacggactggctcgactgtggggaaaccataag

SEQ ID NO: 47 - DNA, 101 bp, Homo sapiens
aacagaacaaacatgacctatgagaaaatgtccagagccctgcgccactactacaaactaaacattatcaggaaggagccaggacaaaggcttttgttcag

SEQ ID NO: 48 - DNA, 4437 bp, Homo sapiens
gtttatgaaaaccccagatgaaatcatgagtggccgaacagaccgtctggagcacctagagtcccaggagctggatgaacaaatataccaagaagatgaatgctgaaggaaccaacagtccacctcagcgggccagcagcccagggaacccctgcccaccaggattgctggaagtgtgacggagcaggcgggctgaggagagtggaaaaggaagcgacccagaaatggcagggacacttctcttgcagaccaagagggaccctggagcaccttagacaaactacccagcacaggcggggctggaattctggcggagggcatgagcctgggactccatgtcacgtttccttctgatttggaatctctccatctgtaattcctcaccctcacccttccaccgttgttagtatcatggtgtttttgtttttgtttttgttttaagaacctgcagtttgactcttcatcgttcatctaggggaagacatctgatgttgttttcctatggaaatatatatctattatatatatattttttgcaaatctcacaaagtgcggcaagcccagctggtcaggaaagagaatacttgcagaggggttcaggttcctctttttcctgccacgtggatcaggtctgttcctgttactgttgggtcttggctgaaaaaaaaaaatgcttttaaaaaagataaaatgaaaaggagagctctctttttctctctcttgctctgttcttcccttggtcccctctgtcctcccgccctgcctgcagttgagattcagatgccttctgacagagttcagcctcttggagagtcttggggattgttggcacctaaacagaatcagtgacccgggtgctttgtggccagcagcacagaatcaaacccgcatcccagcattgggccacccatctgagggaggccaaaatcatcacagatgctgctgtgctgcagacagatacatgctagtccagagagccgcccctgagatggctgtgagaaccatgtgtctaaggcgtaagataaggatggaaggctgtccaagttatttggaaggcctcggcagcttgggattagcttgggagcgcagcgctgcaaagtggaaaatatgaaaagaccacacaggcccagcagtccagaaactgggcaaaaatattctgcagtggggatttatttttccaaagcaggtaacagaggctagtgagaaagaaaagctcctctctgctccattccaaaggccatcttgtggtcagtttcatgccctcacctgatttttttttttttttttttttttcaattcctaaccttttttaaagtttcctggtctccactggacacagagctttggagacggaggatcccagagggcagtctcagttgcaatcagtgtgtgcccagcctgggcagacaggaaattcctcggatacattattttttctttctttcatagctgtgtctcagaaaggacccatttgtggctctttttcacctcaaaataagatcgatggtatcttgtaaaatgagggtagtgccacttcttagtatttttgaaagctgttttagattttttttttttttccttttctagccatctaaattgactcttccaatataggtctcagaaatccaatatttggagtacaatttcttttaatccagattacacctgccttacaaagcaccccctccttgttcccctctgtttcctctactcagttgggggagaaactcacagctcctccgggatacatatgtgccctcagcagcagctcccaggtgaagttaccagacccctgggcttctccccagctttttctgagttgagtcagacatgtagagtttgggtcacacaggcaagaggaattttccctcggccttactgacaaggacaccaacctagggtgcaaacagatggactatggttcaaggacactggaattgaggagctgatcaaggctctcttcagccttgctctgtccctgcctcttatcagagcacaggtagacacacgggcatagccagcccactcctactgtcacaggcgccccaccattcaaccttccgggaggtcagggaccttctatatgaggcgagtgggtctcagtctgcttgaatggtgatgagattctgctggatctcagcacgctgcaggtgtcttttgagagcattcagtaggacatggtgatccctatttcagcctctaagatgactggtattctatctgaaatgcagagattaagccaaatacctgatgtattgtgaaagccactgattttaagaatggagagaaagggattttttactgcatccctctgtatgaatatgaaatcagagaccagggcatgatgttgctaggattagagcctctcagtctggcctcttcacccaagtgcaagaactcagtctcttactgttcaaagaatcttaacagttgaattatggagggaaattcccttttgccccaagcatttctatatttaaagcaatatcccaggagaatatgttagacttaggatgataccttcagccacttgaagaagaaatagaaggcgctcattccaatatagtctttatttcccattcagatacaggttgagcatccctaatctgaacagttaaaacccccaaatgccccaaaatccaaaccttcctgaacgctatgacaccatgagtggaaaattccacacctaacaaacacatttgctttcttatggttcaatgtacacaaactgttttatatagaaaatgatttcaaatatcataaaattaccttcaggctatgtgtataaagtatatatgagccataaatgaattttgtgtttagactttgtgtccatccccaagatctctcattttatatatatatatatatatatatatatatatatatatatatatatatacacacacacacacatacacaaatattccaggatacaaaaaaaaacatttaaaaatccgagacccagaacacttctggtcccaagcatttcagataagggatatcaatctgtactaccaataaggatttcgtaattcccctaactgcaaatgtcctcttcatttgttctttatgagaaaacccgggtagtgccagcacctggatacagtatttacaccctgcagaccctaaagatttcagattcagttagcaaaccttgatgaagcacctgctggacactgagggacccaaagctcaatcagccataatccctgctttcagagtttatattgtacctgcctaatccacccggcgtgactcatttcaacactaagtactaggggtgttgtcaggagacaaatctgaagtcaggagaggaaaatgcaaaggagccctgccgtgtgatggatgtgcattctcacttgggtcttgaagttctcattcctacatctcaagctagccaggcagtctcctctctatcagaagaaagcactggtaattggctagactggctatgttgaaggtaacatgaactctaagatcttgacccagggcgacttggttttgcttaaggtggcatcaccaatgttccaaatcctttagggagatgagggtatccccacagaaaaagaggaataatagaccaatggattttctcctttcaccagtatgtttggaaccctctgatccaatgtcttttgatactgatctcttgtccaaatgagaatgtcgctttagctgaaattcaaatggctgtgacaatttaccgaaatgatgaagtaaccaccattcccacctttcactgcctaggctccaagtctgaatacatttttgaaataggaactcccttttgcaaaaaagaaacctgggtgtcagggaggtgaagtgacttgccctaggagcagacagcatgccaagaatggaattaggctcaggatccagcctgggctcaccctgtgtggctcattcccacccaggaaactgaagataaaagatttgggaaaacacaccaagaaaaaggggcagttttctttgcccaagcatttggtgctagttagaggctgttcactctctcctgctcctcttcggagtagaaataaaggctgtgacacaaggaagccagtggggtgggagggaggcaccataatccctccctaaaacccacagaagactaacctgatactcttttgacccaactgcatcaacactaaacagctgcagaccccctgaatctttcacacatgccaagtgaacattcttgatgatttctctttgtgaccgcaaccacctgcaaaccagaacgactctagaatttccttccccgcccccctttttgtttagtttctaatctcttgtttatgaggtgtggggtttataagggactgaatcaaatgaatgtaacaaaaaagaaaaaaaaaacaaaaaaaaatgccttttctcagggccagtgagttgcaaataatttttaaagaaaagcctataattacatcatctcaa